SEQUENCE LISTING (184 sequences)

SEQ ID NO: 1 - protein, 107 aa, Homo sapiens
MARAALSAAPSNPRLLRVALLLLLLVAAGRRAAGASVATELRCQCLQTLQGIHPKNIQSVNVKSPGPHCAQTEVIATLKNGRKACLNPASPIVKKIIEKMLNSDKSN

SEQ ID NO: 2 - DNA, 321 bp, Homo sapiens
  source 1..321: mol_type = genomic DNA
atggcccgcgctgctctctccgccgcccccagcaatccccggctcctgcgagtggcactgctgctcctgctcctggtagccgctggccggcgcgcagcaggagcgtccgtggccactgaactgcgctgccagtgcttgcagaccctgcagggaattcaccccaagaacatccaaagtgtgaacgtgaagtcccccggaccccactgcgcccaaaccgaagtcatagccacactcaagaatgggcggaaagcttgcctcaatcctgcatcccccatagttaagaaaatcatcgaaaagatgctgaacagtgacaaatccaac

SEQ ID NO: 3 - RNA, 321 nt, Homo sapiens
  source 1..321: mol_type = transcribed RNA
atggcccgcgctgctctctccgccgcccccagcaatccccggctcctgcgagtggcactgctgctcctgctcctggtagccgctggccggcgcgcagcaggagcgtccgtggccactgaactgcgctgccagtgcttgcagaccctgcagggaattcaccccaagaacatccaaagtgtgaacgtgaagtcccccggaccccactgcgcccaaaccgaagtcatagccacactcaagaatgggcggaaagcttgcctcaatcctgcatcccccatagttaagaaaatcatcgaaaagatgctgaacagtgacaaatccaac

SEQ ID NO: 4 - protein, 99 aa, Homo sapiens
MTSKLAVALLAAFLISAALCEGAVLPRSAKELRCQCIKTYSKPFHPKFIKELRVIESGPHCANTEIIVKLSDGRELCLDPKENWVQRVVEKFLKRAENS

SEQ ID NO: 5 - DNA, 297 bp, Homo sapiens
  source 1..297: mol_type = genomic DNA
atgacttccaagctggccgtggctctcttggcagccttcctgatttctgcagctctgtgtgaaggtgcagttttgccaaggagtgctaaagaacttagatgtcagtgcataaagacatactccaaacctttccaccccaaatttatcaaagaactgagagtgattgagagtggaccacactgcgccaacacagaaattattgtaaagctttctgatggaagagagctctgtctggaccccaaggaaaactgggtgcagagggttgtggagaagtttttgaagagggctgagaattca

SEQ ID NO: 6 - RNA, 297 nt, Homo sapiens
  source 1..297: mol_type = transcribed RNA
atgacttccaagctggccgtggctctcttggcagccttcctgatttctgcagctctgtgtgaaggtgcagttttgccaaggagtgctaaagaacttagatgtcagtgcataaagacatactccaaacctttccaccccaaatttatcaaagaactgagagtgattgagagtggaccacactgcgccaacacagaaattattgtaaagctttctgatggaagagagctctgtctggaccccaaggaaaactgggtgcagagggttgtggagaagtttttgaagagggctgagaattca

SEQ ID NO: 7 - protein, 98 aa, Homo sapiens
MNQTAILICCLIFLTLSGIQGVPLSRTVRCTCISISNQPVNPRSLEKLEIIPASQFCPRVEIIATMKKKGEKRCLNPESKAIKNLLKAVSKERSKRSP

SEQ ID NO: 8 - DNA, 294 bp, Homo sapiens
  source 1..294: mol_type = genomic DNA
atgaatcaaactgccattctgatttgctgccttatctttctgactctaagtggcattcaaggagtacctctctctagaactgtacgctgtacctgcatcagcattagtaatcaacctgttaatccaaggtctttagaaaaacttgaaattattcctgcaagccaattttgtccacgtgttgagatcattgctacaatgaaaaagaagggtgagaagagatgtctgaatccagaatcgaaggccatcaagaatttactgaaagcagttagcaaggaaaggtctaaaagatctcct

SEQ ID NO: 9 - RNA, 294 nt, Homo sapiens
  source 1..294: mol_type = transcribed RNA
atgaatcaaactgccattctgatttgctgccttatctttctgactctaagtggcattcaaggagtacctctctctagaactgtacgctgtacctgcatcagcattagtaatcaacctgttaatccaaggtctttagaaaaacttgaaattattcctgcaagccaattttgtccacgtgttgagatcattgctacaatgaaaaagaagggtgagaagagatgtctgaatccagaatcgaaggccatcaagaatttactgaaagcagttagcaaggaaaggtctaaaagatctcct

SEQ ID NO: 10 - protein, 94 aa, Homo sapiens
MSVKGMAIALAVILCATVVQGFPMFKRGRCLCIGPGVKAVKVADIEKASIMYPSNNCDKIEVIITLKENKGQRCLNPKSKQARLIIKKVERKNF

SEQ ID NO: 11 - DNA, 282 bp, Homo sapiens
  source 1..282: mol_type = genomic DNA
atgagtgtgaagggcatggctatagccttggctgtgatattgtgtgctacagttgttcaaggcttccccatgttcaaaagaggacgctgtctttgcataggccctggggtaaaagcagtgaaagtggcagatattgagaaagcctccataatgtacccaagtaacaactgtgacaaaatagaagtgattattaccctgaaagaaaataaaggacaacgatgcctaaatcccaaatcgaagcaagcaaggcttataatcaaaaaagttgaaagaaagaatttt

SEQ ID NO: 12 - RNA, 282 nt, Homo sapiens
  source 1..282: mol_type = transcribed RNA
atgagtgtgaagggcatggctatagccttggctgtgatattgtgtgctacagttgttcaaggcttccccatgttcaaaagaggacgctgtctttgcataggccctggggtaaaagcagtgaaagtggcagatattgagaaagcctccataatgtacccaagtaacaactgtgacaaaatagaagtgattattaccctgaaagaaaataaaggacaacgatgcctaaatcccaaatcgaagcaagcaaggcttataatcaaaaaagttgaaagaaagaatttt

SEQ ID NO: 13 - protein, 93 aa, Homo sapiens
MNAKVVVVLVLVLTALCLSDGKPVSLSYRCPCRFFESHVARANVKHLKILNTPNCALQIVARLKNNNRQVCIDPKLKWIQEYLEKALNKRFKM

SEQ ID NO: 14 - DNA, 279 bp, Homo sapiens
  source 1..279: mol_type = genomic DNA
atgaacgccaaggtcgtggtcgtgctggtcctcgtgctgaccgcgctctgcctcagcgacgggaagcccgtcagcctgagctacagatgcccatgccgattcttcgaaagccatgttgccagagccaacgtcaagcatctcaaaattctcaacactccaaactgtgcccttcagattgtagcccggctgaagaacaacaacagacaagtgtgcattgacccgaagctaaagtggattcaggagtacctggagaaagctttaaacaagaggttcaagatg

SEQ ID NO: 15 - RNA, 279 nt, Homo sapiens
  source 1..279: mol_type = transcribed RNA
atgaacgccaaggtcgtggtcgtgctggtcctcgtgctgaccgcgctctgcctcagcgacgggaagcccgtcagcctgagctacagatgcccatgccgattcttcgaaagccatgttgccagagccaacgtcaagcatctcaaaattctcaacactccaaactgtgcccttcagattgtagcccggctgaagaacaacaacagacaagtgtgcattgacccgaagctaaagtggattcaggagtacctggagaaagctttaaacaagaggttcaagatg

SEQ ID NO: 16 - protein, 96 aa, Homo sapiens
MQIITTALVCLLLAGMWPEDVDSKSMQVPFSRCCFSFAEQEIPLRAILCYRNTSSICSNEGLIFKLKRGKEACALDTVGWVQRHRKMLRHCPSKRK

SEQ ID NO: 17 - DNA, 288 bp, Homo sapiens
  source 1..288: mol_type = genomic DNA
atgcagatcatcaccacagccctggtgtgcttgctgctagctgggatgtggccggaagatgtggacagcaagagcatgcaggtacccttctccagatgttgcttctcatttgcggagcaagagattcccctgagggcaatcctgtgttacagaaataccagctccatctgctccaatgagggcttaatattcaagctgaagagaggcaaagaggcctgcgccttggacacagttggatgggttcagaggcacagaaaaatgctgaggcactgcccgtcaaaaagaaaa

SEQ ID NO: 18 - RNA, 288 nt, Homo sapiens
  source 1..288: mol_type = transcribed RNA
atgcagatcatcaccacagccctggtgtgcttgctgctagctgggatgtggccggaagatgtggacagcaagagcatgcaggtacccttctccagatgttgcttctcatttgcggagcaagagattcccctgagggcaatcctgtgttacagaaataccagctccatctgctccaatgagggcttaatattcaagctgaagagaggcaaagaggcctgcgccttggacacagttggatgggttcagaggcacagaaaaatgctgaggcactgcccgtcaaaaagaaaa

SEQ ID NO: 19 - protein, 96 aa, Homo sapiens
MCCTKSLLLAALMSVLLLHLCGESEAASNFDCCLGYTDRILHPKFIVGFTRQLANEGCDINAIIFHTKKKLSVCANPKQTWVKYIVRLLSKKVKNM

SEQ ID NO: 20 - DNA, 288 bp, Homo sapiens
  source 1..288: mol_type = genomic DNA
atgtgctgtaccaagagtttgctcctggctgctttgatgtcagtgctgctactccacctctgcggcgaatcagaagcagcaagcaactttgactgctgtcttggatacacagaccgtattcttcatcctaaatttattgtgggcttcacacggcagctggccaatgaaggctgtgacatcaatgctatcatctttcacacaaagaaaaagttgtctgtgtgcgcaaatccaaaacagacttgggtgaaatatattgtgcgtctcctcagtaaaaaagtcaagaacatg

SEQ ID NO: 21 - RNA, 288 nt, Homo sapiens
  source 1..288: mol_type = transcribed RNA
atgtgctgtaccaagagtttgctcctggctgctttgatgtcagtgctgctactccacctctgcggcgaatcagaagcagcaagcaactttgactgctgtcttggatacacagaccgtattcttcatcctaaatttattgtgggcttcacacggcagctggccaatgaaggctgtgacatcaatgctatcatctttcacacaaagaaaaagttgtctgtgtgcgcaaatccaaaacagacttgggtgaaatatattgtgcgtctcctcagtaaaaaagtcaagaacatg

SEQ ID NO: 22 - protein, 134 aa, Homo sapiens
MAQSLALSLLILVLAFGIPRTQGSDGGAQDCCLKYSQRKIPAKVVRSYRKQEPSLGCSIPAILFLPRKRSQAELCADPKELWVQQLMQHLDKTPSPQKPAQGCRKDRGASKTGKKGKGSKGCKRTERSQTPKGP

SEQ ID NO: 23 - DNA, 402 bp, Homo sapiens
  source 1..402: mol_type = genomic DNA
atggctcagtcactggctctgagcctccttatcctggttctggcctttggcatccccaggacccaaggcagtgatggaggggctcaggactgttgcctcaagtacagccaaaggaagattcccgccaaggttgtccgcagctaccggaagcaggaaccaagcttaggctgctccatcccagctatcctgttcttgccccgcaagcgctctcaggcagagctatgtgcagacccaaaggagctctgggtgcagcagctgatgcagcatctggacaagacaccatccccacagaaaccagcccagggctgcaggaaggacaggggggcctccaagactggcaagaaaggaaagggctccaaaggctgcaagaggactgagcggtcacagacccctaaagggcca

SEQ ID NO: 24 - RNA, 402 nt, Homo sapiens
  source 1..402: mol_type = transcribed RNA
atggctcagtcactggctctgagcctccttatcctggttctggcctttggcatccccaggacccaaggcagtgatggaggggctcaggactgttgcctcaagtacagccaaaggaagattcccgccaaggttgtccgcagctaccggaagcaggaaccaagcttaggctgctccatcccagctatcctgttcttgccccgcaagcgctctcaggcagagctatgtgcagacccaaaggagctctgggtgcagcagctgatgcagcatctggacaagacaccatccccacagaaaccagcccagggctgcaggaaggacaggggggcctccaagactggcaagaaaggaaagggctccaaaggctgcaagaggactgagcggtcacagacccctaaagggcca

SEQ ID NO: 25 - protein, 397 aa, Homo sapiens
MAPISLSWLLRLATFCHLTVLLAGQHHGVTKCNITCSKMTSKIPVALLIHYQQNQASCGKRAIILETRQHRLFCADPKEQWVKDAMQHLDRQAAALTRNGGTFEKQIGEVKPRTTPAAGGMDESVVLEPEATGESSSLEPTPSSQEAQRALGTSPELPTGVTGSSGTRLPPTPKAQDGGPVGTELFRVPPVSTAATWQSSAPHQPGPSLWAEAKTSEAPSTQDPSTQASTASSPAPEENAPSEGQRVWGQGQSPRPENSLEREEMGPVPAHTDAFQDWGPGSMAHVSVVPVSSEGTPSREPVASGSWTPKAEEPIHATMDPQRLGVLITPVPDAQAATRRQAVGLLAFLGLLFCLGVAMFTYQSLQGCPRKMAGEMAEGLRYIPRSCGSNSYVLVPV

SEQ ID NO: 26 - DNA, 1191 bp, Homo sapiens
  source 1..1191: mol_type = genomic DNA
atggctccgatatctctgtcgtggctgctccgcttggccaccttctgccatctgactgtcctgctggctggacagcaccacggtgtgacgaaatgcaacatcacgtgcagcaagatgacatcaaagatacctgtagctttgctcatccactatcaacagaaccaggcatcatgcggcaaacgcgcaatcatcttggagacgagacagcacaggctgttctgtgccgacccgaaggagcaatgggtcaaggacgcgatgcagcatctggaccgccaggctgctgccctaactcgaaatggcggcaccttcgagaagcagatcggcgaggtgaagcccaggaccacccctgccgccgggggaatggacgagtctgtggtcctggagcccgaagccacaggcgaaagcagtagcctggagccgactccttcttcccaggaagcacagagggccctggggacctccccagagctgccgacgggcgtgactggttcctcagggaccaggctccccccgacgccaaaggctcaggatggagggcctgtgggcacggagcttttccgagtgcctcccgtctccactgccgccacgtggcagagttctgctccccaccaacctgggcccagcctctgggctgaggcaaagacctctgaggccccgtccacccaggacccctccacccaggcctccactgcgtcctccccagccccagaggagaatgctccgtctgaaggccagcgtgtgtggggtcagggacagagccccaggccagagaactctctggagcgggaggagatgggtcccgtgccagcgcacacggatgccttccaggactgggggcctggcagcatggcccacgtctctgtggtccctgtctcctcagaagggacccccagcagggagccagtggcttcaggcagctggacccctaaggctgaggaacccatccatgccaccatggacccccagaggctgggcgtccttatcactcctgtccctgacgcccaggctgccacccggaggcaggcggtggggctgctggccttccttggcctcctcttctgcctgggggtggccatgttcacctaccagagcctccagggctgccctcgaaagatggcaggagagatggcggagggccttcgctacatcccccggagctgtggtagtaattcatatgtcctggtgcccgtg

SEQ ID NO: 27 - RNA, 1191 nt, Homo sapiens
  source 1..1191: mol_type = transcribed RNA
atggctccgatatctctgtcgtggctgctccgcttggccaccttctgccatctgactgtcctgctggctggacagcaccacggtgtgacgaaatgcaacatcacgtgcagcaagatgacatcaaagatacctgtagctttgctcatccactatcaacagaaccaggcatcatgcggcaaacgcgcaatcatcttggagacgagacagcacaggctgttctgtgccgacccgaaggagcaatgggtcaaggacgcgatgcagcatctggaccgccaggctgctgccctaactcgaaatggcggcaccttcgagaagcagatcggcgaggtgaagcccaggaccacccctgccgccgggggaatggacgagtctgtggtcctggagcccgaagccacaggcgaaagcagtagcctggagccgactccttcttcccaggaagcacagagggccctggggacctccccagagctgccgacgggcgtgactggttcctcagggaccaggctccccccgacgccaaaggctcaggatggagggcctgtgggcacggagcttttccgagtgcctcccgtctccactgccgccacgtggcagagttctgctccccaccaacctgggcccagcctctgggctgaggcaaagacctctgaggccccgtccacccaggacccctccacccaggcctccactgcgtcctccccagccccagaggagaatgctccgtctgaaggccagcgtgtgtggggtcagggacagagccccaggccagagaactctctggagcgggaggagatgggtcccgtgccagcgcacacggatgccttccaggactgggggcctggcagcatggcccacgtctctgtggtccctgtctcctcagaagggacccccagcagggagccagtggcttcaggcagctggacccctaaggctgaggaacccatccatgccaccatggacccccagaggctgggcgtccttatcactcctgtccctgacgcccaggctgccacccggaggcaggcggtggggctgctggccttccttggcctcctcttctgcctgggggtggccatgttcacctaccagagcctccagggctgccctcgaaagatggcaggagagatggcggagggccttcgctacatcccccggagctgtggtagtaattcatatgtcctggtgcccgtg

SEQ ID NO: 28 - protein, 337 aa, Homo sapiens
MDRAKLLLLLLLLLLPQAQAQHHGVTKCNITCSKMTSKIPVALLIHYQQNQASCGKRAIILETRQHRLFCADPKEQWVKDAMQHLDRQAAALTRNGGTFEKQIGEVKPRTTPAAGGMDESVVLEPEATGESSSLEPTPSSQEAQRALGTSPELPTGVTGSSGTRLPPTPKAQDGGPVGTELFRVPPVSTAATWQSSAPHQPGPSLWAEAKTSEAPSTQDPSTQASTASSPAPEENAPSEGQRVWGQGQSPRPENSLEREEMGPVPAHTDAFQDWGPGSMAHVSVVPVSSEGTPSREPVASGSWTPKAEEPIHATMDPQRLGVLITPVPDAQAATRRQ

SEQ ID NO: 29 - DNA, 1011 bp, Homo sapiens
  source 1..1011: mol_type = genomic DNA
atggacagagccaagctgctgctgttgctgctccttctgctgctgcctcaggctcaagctcagcaccacggtgtgacgaaatgcaacatcacgtgcagcaagatgacatcaaagatacctgtagctttgctcatccactatcaacagaaccaggcatcatgcggcaaacgcgcaatcatcttggagacgagacagcacaggctgttctgtgccgacccgaaggagcaatgggtcaaggacgcgatgcagcatctggaccgccaggctgctgccctaactcgaaatggcggcaccttcgagaagcagatcggcgaggtgaagcccaggaccacccctgccgccgggggaatggacgagtctgtggtcctggagcccgaagccacaggcgaaagcagtagcctggagccgactccttcttcccaggaagcacagagggccctggggacctccccagagctgccgacgggcgtgactggttcctcagggaccaggctccccccgacgccaaaggctcaggatggagggcctgtgggcacggagcttttccgagtgcctcccgtctccactgccgccacgtggcagagttctgctccccaccaacctgggcccagcctctgggctgaggcaaagacctctgaggccccgtccacccaggacccctccacccaggcctccactgcgtcctccccagccccagaggagaatgctccgtctgaaggccagcgtgtgtggggtcagggacagagccccaggccagagaactctctggagcgggaggagatgggtcccgtgccagcgcacacggatgccttccaggactgggggcctggcagcatggcccacgtctctgtggtccctgtctcctcagaagggacccccagcagggagccagtggcttcaggcagctggacccctaaggctgaggaacccatccatgccaccatggacccccagaggctgggcgtccttatcactcctgtccctgacgcccaggctgccacccggaggcag

SEQ ID NO: 30 - RNA, 1011 nt, Homo sapiens
  source 1..1011: mol_type = transcribed RNA
atggacagagccaagctgctgctgttgctgctccttctgctgctgcctcaggctcaagctcagcaccacggtgtgacgaaatgcaacatcacgtgcagcaagatgacatcaaagatacctgtagctttgctcatccactatcaacagaaccaggcatcatgcggcaaacgcgcaatcatcttggagacgagacagcacaggctgttctgtgccgacccgaaggagcaatgggtcaaggacgcgatgcagcatctggaccgccaggctgctgccctaactcgaaatggcggcaccttcgagaagcagatcggcgaggtgaagcccaggaccacccctgccgccgggggaatggacgagtctgtggtcctggagcccgaagccacaggcgaaagcagtagcctggagccgactccttcttcccaggaagcacagagggccctggggacctccccagagctgccgacgggcgtgactggttcctcagggaccaggctccccccgacgccaaaggctcaggatggagggcctgtgggcacggagcttttccgagtgcctcccgtctccactgccgccacgtggcagagttctgctccccaccaacctgggcccagcctctgggctgaggcaaagacctctgaggccccgtccacccaggacccctccacccaggcctccactgcgtcctccccagccccagaggagaatgctccgtctgaaggccagcgtgtgtggggtcagggacagagccccaggccagagaactctctggagcgggaggagatgggtcccgtgccagcgcacacggatgccttccaggactgggggcctggcagcatggcccacgtctctgtggtccctgtctcctcagaagggacccccagcagggagccagtggcttcaggcagctggacccctaaggctgaggaacccatccatgccaccatggacccccagaggctgggcgtccttatcactcctgtccctgacgcccaggctgccacccggaggcag

SEQ ID NO: 31 - protein, 114 aa, Homo sapiens
MRLLILALLGICSLTAYIVEGVGSEVSDKRTCVSLTTQRLPVSRIKTYTITEGSLRAVIFITKRGLKVCADPQATWVRDVVRSMDRKSNTRNNMIQTKPTGTQQSTNTAVTLTG

SEQ ID NO: 32 - DNA, 342 bp, Homo sapiens
  source 1..342: mol_type = genomic DNA
atgagacttctcatcctggccctccttggcatctgctctctcactgcatacattgtggaaggtgtagggagtgaagtctcagataagaggacctgtgtgagcctcactacccagcgactgccggttagcagaatcaagacctacaccatcacggaaggctccttgagagcagtaatttttattaccaaacgtggcctaaaagtctgtgctgatccacaagccacatgggtgagagacgtggtcaggagcatggacaggaaatccaacaccagaaataacatgatccagaccaagccaacaggaacccagcaatcgaccaatacagctgtgactctgactggc

SEQ ID NO: 33 - RNA, 342 nt, Homo sapiens
  source 1..342: mol_type = transcribed RNA
atgagacttctcatcctggccctccttggcatctgctctctcactgcatacattgtggaaggtgtagggagtgaagtctcagataagaggacctgtgtgagcctcactacccagcgactgccggttagcagaatcaagacctacaccatcacggaaggctccttgagagcagtaatttttattaccaaacgtggcctaaaagtctgtgctgatccacaagccacatgggtgagagacgtggtcaggagcatggacaggaaatccaacaccagaaataacatgatccagaccaagccaacaggaacccagcaatcgaccaatacagctgtgactctgactggc

SEQ ID NO: 34 - protein, 114 aa, Homo sapiens
MRLLILALLGICSLTAYIVEGVGSEVSHRRTCVSLTTQRLPVSRIKTYTITEGSLRAVIFITKRGLKVCADPQATWVRDVVRSMDRKSNTRNNMIQTKPTGTQQSTNTAVTLTG

SEQ ID NO: 35 - DNA, 342 bp, Homo sapiens
  source 1..342: mol_type = genomic DNA
atgagacttctcatcctggccctccttggcatctgctctctcactgcatacattgtggaaggtgtagggagtgaagtctcacataggaggacctgtgtgagcctcactacccagcgactgccagttagcagaatcaagacctacaccatcacggaaggctccttgagagcagtaatttttattaccaaacgtggcctaaaagtctgtgctgatccacaagccacgtgggtgagagacgtggtcaggagcatggacaggaaatccaacaccagaaataacatgatccagaccaagccaacaggaacccagcaatcgaccaatacagctgtgaccctgactggc

SEQ ID NO: 36 - RNA, 342 nt, Homo sapiens
  source 1..342: mol_type = transcribed RNA
atgagacttctcatcctggccctccttggcatctgctctctcactgcatacattgtggaaggtgtagggagtgaagtctcacataggaggacctgtgtgagcctcactacccagcgactgccagttagcagaatcaagacctacaccatcacggaaggctccttgagagcagtaatttttattaccaaacgtggcctaaaagtctgtgctgatccacaagccacgtgggtgagagacgtggtcaggagcatggacaggaaatccaacaccagaaataacatgatccagaccaagccaacaggaacccagcaatcgaccaatacagctgtgaccctgactggc

SEQ ID NO: 37 - protein, 154 aa, Homo sapiens
MAPVRSLNCTLRDSQQKSLVMSGPYELKALHLQGQDMEQQVVFSMSFVQGEESNDKIPVALGLKEKNLYLSCVLKDDKPTLQLESVDPKNYPKKKMEKRFVFNKIEINNKLEFESAQFPNWYISTSQAENMPVFLGGTKGGQDITDFTMQFVSS

SEQ ID NO: 38 - DNA, 462 bp, Homo sapiens
  source 1..462: mol_type = genomic DNA
atggcacctgtacgatcactgaactgcacgctccgggactcacagcaaaaaagcttggtgatgtctggtccatatgaactgaaagctctccacctccagggacaggatatggagcaacaagtggtgttctccatgtcctttgtacaaggagaagaaagtaatgacaaaatacctgtggccttgggcctcaaggaaaagaatctgtacctgtcctgcgtgttgaaagatgataagcccactctacagctggagagtgtagatcccaaaaattacccaaagaagaagatggaaaagcgatttgtcttcaacaagatagaaatcaataacaagctggaatttgagtctgcccagttccccaactggtacatcagcacctctcaagcagaaaacatgcccgtcttcctgggagggaccaaaggcggccaggatataactgacttcaccatgcaatttgtgtcttcc

SEQ ID NO: 39 - RNA, 462 nt, Homo sapiens
  source 1..462: mol_type = transcribed RNA
atggcacctgtacgatcactgaactgcacgctccgggactcacagcaaaaaagcttggtgatgtctggtccatatgaactgaaagctctccacctccagggacaggatatggagcaacaagtggtgttctccatgtcctttgtacaaggagaagaaagtaatgacaaaatacctgtggccttgggcctcaaggaaaagaatctgtacctgtcctgcgtgttgaaagatgataagcccactctacagctggagagtgtagatcccaaaaattacccaaagaagaagatggaaaagcgatttgtcttcaacaagatagaaatcaataacaagctggaatttgagtctgcccagttccccaactggtacatcagcacctctcaagcagaaaacatgcccgtcttcctgggagggaccaaaggcggccaggatataactgacttcaccatgcaatttgtgtcttcc

SEQ ID NO: 40 - protein, 173 aa, Homo sapiens
MDRAKLLLLLLLLLLPQAQAAPVRSLNCTLRDSQQKSLVMSGPYELKALHLQGQDMEQQVVFSMSFVQGEESNDKIPVALGLKEKNLYLSCVLKDDKPTLQLESVDPKNYPKKKMEKRFVFNKIEINNKLEFESAQFPNWYISTSQAENMPVFLGGTKGGQDITDFTMQFVSS

SEQ ID NO: 41 - DNA, 519 bp, Homo sapiens
  source 1..519: mol_type = genomic DNA
atggacagagccaagctgctgctgttgctgctccttctgctgctgcctcaggctcaagctgcacctgtacgatcactgaactgcacgctccgggactcacagcaaaaaagcttggtgatgtctggtccatatgaactgaaagctctccacctccagggacaggatatggagcaacaagtggtgttctccatgtcctttgtacaaggagaagaaagtaatgacaaaatacctgtggccttgggcctcaaggaaaagaatctgtacctgtcctgcgtgttgaaagatgataagcccactctacagctggagagtgtagatcccaaaaattacccaaagaagaagatggaaaagcgatttgtcttcaacaagatagaaatcaataacaagctggaatttgagtctgcccagttccccaactggtacatcagcacctctcaagcagaaaacatgcccgtcttcctgggagggaccaaaggcggccaggatataactgacttcaccatgcaatttgtgtcttcc

SEQ ID NO: 42 - RNA, 519 nt, Homo sapiens
  source 1..519: mol_type = transcribed RNA
atggacagagccaagctgctgctgttgctgctccttctgctgctgcctcaggctcaagctgcacctgtacgatcactgaactgcacgctccgggactcacagcaaaaaagcttggtgatgtctggtccatatgaactgaaagctctccacctccagggacaggatatggagcaacaagtggtgttctccatgtcctttgtacaaggagaagaaagtaatgacaaaatacctgtggccttgggcctcaaggaaaagaatctgtacctgtcctgcgtgttgaaagatgataagcccactctacagctggagagtgtagatcccaaaaattacccaaagaagaagatggaaaagcgatttgtcttcaacaagatagaaatcaataacaagctggaatttgagtctgcccagttccccaactggtacatcagcacctctcaagcagaaaacatgcccgtcttcctgggagggaccaaaggcggccaggatataactgacttcaccatgcaatttgtgtcttcc

SEQ ID NO: 43 - protein, 153 aa, Homo sapiens
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 44 - DNA, 459 bp, Homo sapiens
  source 1..459: mol_type = genomic DNA
atgtacaggatgcaactcctgtcttgcattgcactaagtcttgcacttgtcacaaacagtgcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgact

SEQ ID NO: 45 - RNA, 459 nt, Homo sapiens
  source 1..459: mol_type = transcribed RNA
atgtacaggatgcaactcctgtcttgcattgcactaagtcttgcacttgtcacaaacagtgcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgact

SEQ ID NO: 46 - protein, 158 aa, Homo sapiens
MYFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 47 - DNA, 474 bp, Homo sapiens
  source 1..474: mol_type = genomic DNA
atgtactttggcaagcttgaatctaaattatcagtcataagaaatttgaatgaccaagttctcttcattgaccaaggaaatcggcctctatttgaagatatgactgattctgactgtagagataatgcaccccggaccatatttattataagtatgtataaagatagccagcctagaggtatggctgtaactatctctgtgaagtgtgagaaaatttcaactctctcctgtgagaacaaaattatttcctttaaggaaatgaatcctcctgataacatcaaggatacaaaaagtgacatcatattctttcagagaagtgtcccaggacatgataataagatgcaatttgaatcttcatcatacgaaggatactttctagcttgtgaaaaagagagagacctttttaaactcattttgaaaaaagaggatgaattgggggatagatctataatgttcactgttcaaaacgaagac

SEQ ID NO: 48 - RNA, 474 nt, Homo sapiens
  source 1..474: mol_type = transcribed RNA
atgtactttggcaagcttgaatctaaattatcagtcataagaaatttgaatgaccaagttctcttcattgaccaaggaaatcggcctctatttgaagatatgactgattctgactgtagagataatgcaccccggaccatatttattataagtatgtataaagatagccagcctagaggtatggctgtaactatctctgtgaagtgtgagaaaatttcaactctctcctgtgagaacaaaattatttcctttaaggaaatgaatcctcctgataacatcaaggatacaaaaagtgacatcatattctttcagagaagtgtcccaggacatgataataagatgcaatttgaatcttcatcatacgaaggatactttctagcttgtgaaaaagagagagacctttttaaactcattttgaaaaaagaggatgaattgggggatagatctataatgttcactgttcaaaacgaagac

SEQ ID NO: 49 - protein, 177 aa, Homo sapiens
MDRAKLLLLLLLLLLPQAQAYFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 50 - DNA, 531 bp, Homo sapiens
  source 1..531: mol_type = genomic DNA
atggacagagccaagctgctgctgttgctgctccttctgctgctgcctcaggctcaagcttactttggcaagcttgaatctaaattatcagtcataagaaatttgaatgaccaagttctcttcattgaccaaggaaatcggcctctatttgaagatatgactgattctgactgtagagataatgcaccccggaccatatttattataagtatgtataaagatagccagcctagaggtatggctgtaactatctctgtgaagtgtgagaaaatttcaactctctcctgtgagaacaaaattatttcctttaaggaaatgaatcctcctgataacatcaaggatacaaaaagtgacatcatattctttcagagaagtgtcccaggacatgataataagatgcaatttgaatcttcatcatacgaaggatactttctagcttgtgaaaaagagagagacctttttaaactcattttgaaaaaagaggatgaattgggggatagatctataatgttcactgttcaaaacgaagac

SEQ ID NO: 51 - RNA, 531 nt, Homo sapiens
  source 1..531: mol_type = transcribed RNA
atggacagagccaagctgctgctgttgctgctccttctgctgctgcctcaggctcaagcttactttggcaagcttgaatctaaattatcagtcataagaaatttgaatgaccaagttctcttcattgaccaaggaaatcggcctctatttgaagatatgactgattctgactgtagagataatgcaccccggaccatatttattataagtatgtataaagatagccagcctagaggtatggctgtaactatctctgtgaagtgtgagaaaatttcaactctctcctgtgagaacaaaattatttcctttaaggaaatgaatcctcctgataacatcaaggatacaaaaagtgacatcatattctttcagagaagtgtcccaggacatgataataagatgcaatttgaatcttcatcatacgaaggatactttctagcttgtgaaaaagagagagacctttttaaactcattttgaaaaaagaggatgaattgggggatagatctataatgttcactgttcaaaacgaagac

SEQ ID NO: 52 - protein, 177 aa, Homo sapiens
MKLQCVSLWLLGTILILCSVDNHGLRRCLISTDMHHIEESFQEIKRAIQAKDTFPNVTILSTLETLQIIKPLDVCCVTKNLLAFYVDRVFKDHQEPNPKILRKISSIANSFLYMQKTLRQCQEQRQCHCRQEATNATRVIHDNYDQLEVHAAAIKSLGELDVFLAWINKNHEVMFSA

SEQ ID NO: 53 - DNA, 531 bp, Homo sapiens
  source 1..531: mol_type = genomic DNA
atgaagttacagtgtgtttccctttggctcctgggtacaatactgatattgtgctcagtagacaaccacggtctcaggagatgtctgatttccacagacatgcaccatatagaagagagtttccaagaaatcaaaagagccatccaagctaaggacaccttcccaaatgtcactatcctgtccacattggagactctgcagatcattaagcccttagatgtgtgctgcgtgaccaagaacctcctggcgttctacgtggacagggtgttcaaggatcatcaggagccaaaccccaaaatcttgagaaaaatcagcagcattgccaactctttcctctacatgcagaaaactctgcggcaatgtcaggaacagaggcagtgtcactgcaggcaggaagccaccaatgccaccagagtcatccatgacaactatgatcagctggaggtccacgctgctgccattaaatccctgggagagctcgacgtctttctagcctggattaataagaatcatgaagtaatgttctcagct

SEQ ID NO: 54 - RNA, 531 nt, Homo sapiens
  source 1..531: mol_type = transcribed RNA
atgaagttacagtgtgtttccctttggctcctgggtacaatactgatattgtgctcagtagacaaccacggtctcaggagatgtctgatttccacagacatgcaccatatagaagagagtttccaagaaatcaaaagagccatccaagctaaggacaccttcccaaatgtcactatcctgtccacattggagactctgcagatcattaagcccttagatgtgtgctgcgtgaccaagaacctcctggcgttctacgtggacagggtgttcaaggatcatcaggagccaaaccccaaaatcttgagaaaaatcagcagcattgccaactctttcctctacatgcagaaaactctgcggcaatgtcaggaacagaggcagtgtcactgcaggcaggaagccaccaatgccaccagagtcatccatgacaactatgatcagctggaggtccacgctgctgccattaaatccctgggagagctcgacgtctttctagcctggattaataagaatcatgaagtaatgttctcagct

SEQ ID NO: 55 - protein, 176 aa, Homo sapiens
MKASSLAFSLLSAAFYLLWTPSTGLKTLNLGSCVIATNLQEIRNGFSEIRGSVQAKDGNIDIRILRRTESLQDTKPANRCCLLRHLLRLYLDRVFKNYQTPDHYTLRKISSLANSFLTIKKDLRLCHAHMTCHCGEEAMKKYSQILSHFEKLEPQAAVVKALGELDILLQWMEETE

SEQ ID NO: 56 - DNA, 528 bp, Homo sapiens
  source 1..528: mol_type = genomic DNA
atgaaagcctctagtcttgccttcagccttctctctgctgcgttttatctcctatggactccttccactggactgaagacactcaatttgggaagctgtgtgatcgccacaaaccttcaggaaatacgaaatggattttctgagatacggggcagtgtgcaagccaaagatggaaacattgacatcagaatcttaaggaggactgagtctttgcaagacacaaagcctgcgaatcgatgctgcctcctgcgccatttgctaagactctatctggacagggtatttaaaaactaccagacccctgaccattatactctccggaagatcagcagcctcgccaattcctttcttaccatcaagaaggacctccggctctgtcatgcccacatgacatgccattgtggggaggaagcaatgaagaaatacagccagattctgagtcactttgaaaagctggaacctcaggcagcagttgtgaaggctttgggggaactagacattcttctgcaatggatggaggagacagaa

SEQ ID NO: 57 - RNA, 528 nt, Homo sapiens
  source 1..528: mol_type = transcribed RNA
atgaaagcctctagtcttgccttcagccttctctctgctgcgttttatctcctatggactccttccactggactgaagacactcaatttgggaagctgtgtgatcgccacaaaccttcaggaaatacgaaatggattttctgagatacggggcagtgtgcaagccaaagatggaaacattgacatcagaatcttaaggaggactgagtctttgcaagacacaaagcctgcgaatcgatgctgcctcctgcgccatttgctaagactctatctggacagggtatttaaaaactaccagacccctgaccattatactctccggaagatcagcagcctcgccaattcctttcttaccatcaagaaggacctccggctctgtcatgcccacatgacatgccattgtggggaggaagcaatgaagaaatacagccagattctgagtcactttgaaaagctggaacctcaggcagcagttgtgaaggctttgggggaactagacattcttctgcaatggatggaggagacagaa

SEQ ID NO: 58 - protein, 162 aa, Homo sapiens
MRSSPGNMERIVICLMVIFLGTLVHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 59 - DNA, 486 bp, Homo sapiens
  source 1..486: mol_type = genomic DNA
atgagatccagtcctggcaacatggagaggattgtcatctgtctgatggtcatcttcttggggacactggtccacaaatcaagctcccaaggtcaagatcgccacatgattagaatgcgtcaacttatagatattgttgatcagctgaaaaattatgtgaatgacttggtccctgaatttctgccagctccagaagatgtagagacaaactgtgagtggtcagctttttcctgctttcagaaggcccaactaaagtcagcaaatacaggaaacaatgaaaggataatcaatgtatcaattaaaaagctgaagaggaaaccaccttccacaaatgcagggagaagacagaaacacagactaacatgcccttcatgtgattcttatgagaaaaaaccacccaaagaattcctagaaagattcaaatcacttctccaaaagatgattcatcagcatctgtcctctagaacacacggaagtgaagattcc

SEQ ID NO: 60 - RNA, 486 nt, Homo sapiens
  source 1..486: mol_type = transcribed RNA
atgagatccagtcctggcaacatggagaggattgtcatctgtctgatggtcatcttcttggggacactggtccacaaatcaagctcccaaggtcaagatcgccacatgattagaatgcgtcaacttatagatattgttgatcagctgaaaaattatgtgaatgacttggtccctgaatttctgccagctccagaagatgtagagacaaactgtgagtggtcagctttttcctgctttcagaaggcccaactaaagtcagcaaatacaggaaacaatgaaaggataatcaatgtatcaattaaaaagctgaagaggaaaccaccttccacaaatgcagggagaagacagaaacacagactaacatgcccttcatgtgattcttatgagaaaaaaccacccaaagaattcctagaaagattcaaatcacttctccaaaagatgattcatcagcatctgtcctctagaacacacggaagtgaagattcc

SEQ ID NO: 61 - protein, 179 aa, Homo sapiens
MAALQKSVSSFLMGTLATSCLLLLALLVQGGAAAPISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACI

SEQ ID NO: 62 - DNA, 537 bp, Homo sapiens
  source 1..537: mol_type = genomic DNA
atggccgccctgcagaaatctgtgagctctttccttatggggaccctggccaccagctgcctccttctcttggccctcttggtacagggaggagcagctgcgcccatcagctcccactgcaggcttgacaagtccaacttccagcagccctatatcaccaaccgcaccttcatgctggctaaggaggctagcttggctgataacaacacagacgttcgtctcattggggagaaactgttccacggagtcagtatgagtgagcgctgctatctgatgaagcaggtgctgaacttcacccttgaagaagtgctgttccctcaatctgataggttccagccttatatgcaggaggtggtgcccttcctggccaggctcagcaacaggctaagcacatgtcatattgaaggtgatgacctgcatatccagaggaatgtgcaaaagctgaaggacacagtgaaaaagcttggagagagtggagagatcaaagcaattggagaactggatttgctgtttatgtctctgagaaatgcctgcatt

SEQ ID NO: 63 - RNA, 537 nt, Homo sapiens
  source 1..537: mol_type = transcribed RNA
atggccgccctgcagaaatctgtgagctctttccttatggggaccctggccaccagctgcctccttctcttggccctcttggtacagggaggagcagctgcgcccatcagctcccactgcaggcttgacaagtccaacttccagcagccctatatcaccaaccgcaccttcatgctggctaaggaggctagcttggctgataacaacacagacgttcgtctcattggggagaaactgttccacggagtcagtatgagtgagcgctgctatctgatgaagcaggtgctgaacttcacccttgaagaagtgctgttccctcaatctgataggttccagccttatatgcaggaggtggtgcccttcctggccaggctcagcaacaggctaagcacatgtcatattgaaggtgatgacctgcatatccagaggaatgtgcaaaagctgaaggacacagtgaaaaagcttggagagagtggagagatcaaagcaattggagaactggatttgctgtttatgtctctgagaaatgcctgcatt

SEQ ID NO: 64 - protein, 196 aa, Homo sapiens
MDRAKLLLLLLLLLLPQAQAAFGISGVQKYTRALHDSSITGISPITEYLASLSTYNDQSITFALEDESYEIYVEDLKKDEKKDKVLLSYYESQHPSNESGDGVDGKMLMVTLSPTKDFWLHANNKEHSVELHKCEKPLPDQAFFVLHNMHSNCVSFECKTDPGVFIGVKDNHLALIKVDSSENLCTENILFKLSET

SEQ ID NO: 65 - DNA, 588 bp, Homo sapiens
  source 1..588: mol_type = genomic DNA
atggacagagccaagctgctgctgttgctgctccttctgctgctgcctcaggctcaagctgcctttggtatatcaggggtccagaaatatactagagcacttcatgattcaagtatcacaggaatttcacctattacagagtatcttgcttctctaagcacatacaatgatcaatccattacttttgctttggaggatgaaagttatgagatatatgttgaagacttgaaaaaagatgaaaagaaagataaggtgttactgagttactatgagtctcaacacccctcaaatgaatcaggtgacggtgttgatggtaagatgttaatggtaaccctgagtcctacaaaagacttctggttgcatgccaacaacaaggaacactctgtggagctccataagtgtgaaaaaccactgccagaccaggccttctttgtccttcataatatgcactccaactgtgtttcatttgaatgcaagactgatcctggagtgtttataggtgtaaaggataatcatcttgctctgattaaagtagactcttctgagaatttgtgtactgaaaatatcttgtttaagctctctgaaact

SEQ ID NO: 66 - RNA, 588 nt, Homo sapiens
  source 1..588: mol_type = transcribed RNA
atggacagagccaagctgctgctgttgctgctccttctgctgctgcctcaggctcaagctgcctttggtatatcaggggtccagaaatatactagagcacttcatgattcaagtatcacaggaatttcacctattacagagtatcttgcttctctaagcacatacaatgatcaatccattacttttgctttggaggatgaaagttatgagatatatgttgaagacttgaaaaaagatgaaaagaaagataaggtgttactgagttactatgagtctcaacacccctcaaatgaatcaggtgacggtgttgatggtaagatgttaatggtaaccctgagtcctacaaaagacttctggttgcatgccaacaacaaggaacactctgtggagctccataagtgtgaaaaaccactgccagaccaggccttctttgtccttcataatatgcactccaactgtgtttcatttgaatgcaagactgatcctggagtgtttataggtgtaaaggataatcatcttgctctgattaaagtagactcttctgagaatttgtgtactgaaaatatcttgtttaagctctctgaaact

SEQ ID NO: 67 - protein, 173 aa, Homo sapiens
MDRAKLLLLLLLLLLPQAQAKIDTPQQGSIQDINHRVWVLQDQTLIAVPRKDRMSPVTIALISCRHVETLEKDRGNPIYLGLNGLNLCLMCAKVGDQPTLQLKEKDIMDLYNQPEPVKSFLFYHSQSGRNSTFESVAFPGWFIAVSSEGGCPLILTQELGKANTTDFGLTMLF

SEQ ID NO: 68 - DNA, 519 bp, Homo sapiens
  source 1..519: mol_type = genomic DNA
atggacagagccaagctgctgctgttgctgctccttctgctgctgcctcaggctcaagctaaaattgacacacctcagcaggggagcattcaggatatcaatcatcgggtgtgggttcttcaggaccagacgctcatagcagtcccgaggaaggaccgtatgtctccagtcactattgccttaatctcatgccgacatgtggagacccttgagaaagacagagggaaccccatctacctgggcctgaatggactcaatctctgcctgatgtgtgctaaagtcggggaccagcccacactgcagctgaaggaaaaggatataatggatttgtacaaccaacccgagcctgtgaagtcctttctcttctaccacagccagagtggcaggaactccaccttcgagtctgtggctttccctggctggttcatcgctgtcagctctgaaggaggctgtcctctcatccttacccaagaactggggaaagccaacactactgactttgggttaactatgctgttt

SEQ ID NO: 69 - RNA, 519 nt, Homo sapiens
  source 1..519: mol_type = transcribed RNA
atggacagagccaagctgctgctgttgctgctccttctgctgctgcctcaggctcaagctaaaattgacacacctcagcaggggagcattcaggatatcaatcatcgggtgtgggttcttcaggaccagacgctcatagcagtcccgaggaaggaccgtatgtctccagtcactattgccttaatctcatgccgacatgtggagacccttgagaaagacagagggaaccccatctacctgggcctgaatggactcaatctctgcctgatgtgtgctaaagtcggggaccagcccacactgcagctgaaggaaaaggatataatggatttgtacaaccaacccgagcctgtgaagtcctttctcttctaccacagccagagtggcaggaactccaccttcgagtctgtggctttccctggctggttcatcgctgtcagctctgaaggaggctgtcctctcatccttacccaagaactggggaaagccaacactactgactttgggttaactatgctgttt

SEQ ID NO: 70 - protein, 233 aa, Homo sapiens
MSTESMIRDVELAEEALPKKTGGPQGSRRCLFLSLFSFLIVAGATTLFCLLHFGVIGPQREEFPRDLSLISPLAQAVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 71 - DNA, 699 bp, Homo sapiens
  source 1..699: mol_type = genomic DNA
atgagcactgaaagcatgatccgggacgtggagctggccgaggaggcgctccccaagaagacaggggggccccagggctccaggcggtgcttgttcctcagcctcttctccttcctgatcgtggcaggcgccaccacgctcttctgcctgctgcactttggagtgatcggcccccagagggaagagttccccagggacctctctctaatcagccctctggcccaggcagtcagatcatcttctcgaaccccgagtgacaagcctgtagcccatgttgtagcaaaccctcaagctgaggggcagctccagtggctgaaccgccgggccaatgccctcctggccaatggcgtggagctgagagataaccagctggtggtgccatcagagggcctgtacctcatctactcccaggtcctcttcaagggccaaggctgcccctccacccatgtgctcctcacccacaccatcagccgcatcgccgtctcctaccagaccaaggtcaacctcctctctgccatcaagagcccctgccagagggagaccccagagggggctgaggccaagccctggtatgagcccatctatctgggaggggtcttccagctggagaagggtgaccgactcagcgctgagatcaatcggcccgactatctcgactttgccgagtctgggcaggtctactttgggatcattgccctg

SEQ ID NO: 72 - RNA, 699 nt, Homo sapiens
  source 1..699: mol_type = transcribed RNA
atgagcactgaaagcatgatccgggacgtggagctggccgaggaggcgctccccaagaagacaggggggccccagggctccaggcggtgcttgttcctcagcctcttctccttcctgatcgtggcaggcgccaccacgctcttctgcctgctgcactttggagtgatcggcccccagagggaagagttccccagggacctctctctaatcagccctctggcccaggcagtcagatcatcttctcgaaccccgagtgacaagcctgtagcccatgttgtagcaaaccctcaagctgaggggcagctccagtggctgaaccgccgggccaatgccctcctggccaatggcgtggagctgagagataaccagctggtggtgccatcagagggcctgtacctcatctactcccaggtcctcttcaagggccaaggctgcccctccacccatgtgctcctcacccacaccatcagccgcatcgccgtctcctaccagaccaaggtcaacctcctctctgccatcaagagcccctgccagagggagaccccagagggggctgaggccaagccctggtatgagcccatctatctgggaggggtcttccagctggagaagggtgaccgactcagcgctgagatcaatcggcccgactatctcgactttgccgagtctgggcaggtctactttgggatcattgccctg

SEQ ID NO: 73 - protein, 177 aa, Homo sapiens
MDRAKLLLLLLLLLLPQAQAVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 74 - DNA, 531 bp, Homo sapiens
  source 1..531: mol_type = genomic DNA
atggacagagccaagctgctgctgttgctgctccttctgctgctgcctcaggctcaagctgtcagatcatcttctcgaaccccgagtgacaagcctgtagcccatgttgtagcaaaccctcaagctgaggggcagctccagtggctgaaccgccgggccaatgccctcctggccaatggcgtggagctgagagataaccagctggtggtgccatcagagggcctgtacctcatctactcccaggtcctcttcaagggccaaggctgcccctccacccatgtgctcctcacccacaccatcagccgcatcgccgtctcctaccagaccaaggtcaacctcctctctgccatcaagagcccctgccagagggagaccccagagggggctgaggccaagccctggtatgagcccatctatctgggaggggtcttccagctggagaagggtgaccgactcagcgctgagatcaatcggcccgactatctcgactttgccgagtctgggcaggtctactttgggatcattgccctg

SEQ ID NO: 75 - RNA, 531 nt, Homo sapiens
  source 1..531: mol_type = transcribed RNA
atggacagagccaagctgctgctgttgctgctccttctgctgctgcctcaggctcaagctgtcagatcatcttctcgaaccccgagtgacaagcctgtagcccatgttgtagcaaaccctcaagctgaggggcagctccagtggctgaaccgccgggccaatgccctcctggccaatggcgtggagctgagagataaccagctggtggtgccatcagagggcctgtacctcatctactcccaggtcctcttcaagggccaaggctgcccctccacccatgtgctcctcacccacaccatcagccgcatcgccgtctcctaccagaccaaggtcaacctcctctctgccatcaagagcccctgccagagggagaccccagagggggctgaggccaagccctggtatgagcccatctatctgggaggggtcttccagctggagaagggtgaccgactcagcgctgagatcaatcggcccgactatctcgactttgccgagtctgggcaggtctactttgggatcattgccctg

SEQ ID NO: 76 - protein, 285 aa, Homo sapiens
MDDSTEREQSRLTSCLKKREEMKLKECVSILPRKESPSVRSSKDGKLLAATLLLALLSCCLTVVSFYQVAALQGDLASLRAELQGHHAEKLPAGAGAPKAGLEEAPAVTAGLKIFEPPAPGEGNSSQNSRNKRAVQGPEETVTQDCLQLIADSETPTIQKGSYTFVPWLLSFKRGSALEEKENKILVKETGYFFIYGQVLYTDKTYAMGHLIQRKKVHVFGDELSLVTLFRCIQNMPETLPNNSCYSAGIAKLEEGDELQLAIPRENAQISLDGDVTFFGALKLL

SEQ ID NO: 77 - DNA, 855 bp, Homo sapiens
  source 1..855: mol_type = genomic DNA
atggatgactccacagaaagggagcagtcacgccttacttcttgccttaagaaaagagaagaaatgaaactgaaggagtgtgtttccatcctcccacggaaggaaagcccctctgtccgatcctccaaagacggaaagctgctggctgcaaccttgctgctggcactgctgtcttgctgcctcacggtggtgtctttctaccaggtggccgccctgcaaggggacctggccagcctccgggcagagctgcagggccaccacgcggagaagctgccagcaggagcaggagcccccaaggccggcctggaggaagctccagctgtcaccgcgggactgaaaatctttgaaccaccagctccaggagaaggcaactccagtcagaacagcagaaataagcgtgccgttcagggtccagaagaaacagtcactcaagactgcttgcaactgattgcagacagtgaaacaccaactatacaaaaaggatcttacacatttgttccatggcttctcagctttaaaaggggaagtgccctagaagaaaaagagaataaaatattggtcaaagaaactggttacttttttatatatggtcaggttttatatactgataagacctacgccatgggacatctaattcagaggaagaaggtccatgtctttggggatgaattgagtctggtgactttgtttcgatgtattcaaaatatgcctgaaacactacccaataattcctgctattcagctggcattgcaaaactggaagaaggagatgaactccaacttgcaataccaagagaaaatgcacaaatatcactggatggagatgtcacattttttggtgcattgaaactgctg

SEQ ID NO: 78 - RNA, 855 nt, Homo sapiens
  source 1..855: mol_type = transcribed RNA
atggatgactccacagaaagggagcagtcacgccttacttcttgccttaagaaaagagaagaaatgaaactgaaggagtgtgtttccatcctcccacggaaggaaagcccctctgtccgatcctccaaagacggaaagctgctggctgcaaccttgctgctggcactgctgtcttgctgcctcacggtggtgtctttctaccaggtggccgccctgcaaggggacctggccagcctccgggcagagctgcagggccaccacgcggagaagctgccagcaggagcaggagcccccaaggccggcctggaggaagctccagctgtcaccgcgggactgaaaatctttgaaccaccagctccaggagaaggcaactccagtcagaacagcagaaataagcgtgccgttcagggtccagaagaaacagtcactcaagactgcttgcaactgattgcagacagtgaaacaccaactatacaaaaaggatcttacacatttgttccatggcttctcagctttaaaaggggaagtgccctagaagaaaaagagaataaaatattggtcaaagaaactggttacttttttatatatggtcaggttttatatactgataagacctacgccatgggacatctaattcagaggaagaaggtccatgtctttggggatgaattgagtctggtgactttgtttcgatgtattcaaaatatgcctgaaacactacccaataattcctgctattcagctggcattgcaaaactggaagaaggagatgaactccaacttgcaataccaagagaaaatgcacaaatatcactggatggagatgtcacattttttggtgcattgaaactgctg

SEQ ID NO: 79 - protein, 172 aa, Homo sapiens
MDRAKLLLLLLLLLLPQAQAAVQGPEETVTQDCLQLIADSETPTIQKGSYTFVPWLLSFKRGSALEEKENKILVKETGYFFIYGQVLYTDKTYAMGHLIQRKKVHVFGDELSLVTLFRCIQNMPETLPNNSCYSAGIAKLEEGDELQLAIPRENAQISLDGDVTFFGALKLL

SEQ ID NO: 80 - DNA, 516 bp, Homo sapiens
  source 1..516: mol_type = genomic DNA
atggacagagccaagctgctgctgttgctgctccttctgctgctgcctcaggctcaagctgccgttcagggtccagaagaaacagtcactcaagactgcttgcaactgattgcagacagtgaaacaccaactatacaaaaaggatcttacacatttgttccatggcttctcagctttaaaaggggaagtgccctagaagaaaaagagaataaaatattggtcaaagaaactggttacttttttatatatggtcaggttttatatactgataagacctacgccatgggacatctaattcagaggaagaaggtccatgtctttggggatgaattgagtctggtgactttgtttcgatgtattcaaaatatgcctgaaacactacccaataattcctgctattcagctggcattgcaaaactggaagaaggagatgaactccaacttgcaataccaagagaaaatgcacaaatatcactggatggagatgtcacattttttggtgcattgaaactgctg

SEQ ID NO: 81 - RNA, 516 nt, Homo sapiens
  source 1..516: mol_type = transcribed RNA
atggacagagccaagctgctgctgttgctgctccttctgctgctgcctcaggctcaagctgccgttcagggtccagaagaaacagtcactcaagactgcttgcaactgattgcagacagtgaaacaccaactatacaaaaaggatcttacacatttgttccatggcttctcagctttaaaaggggaagtgccctagaagaaaaagagaataaaatattggtcaaagaaactggttacttttttatatatggtcaggttttatatactgataagacctacgccatgggacatctaattcagaggaagaaggtccatgtctttggggatgaattgagtctggtgactttgtttcgatgtattcaaaatatgcctgaaacactacccaataattcctgctattcagctggcattgcaaaactggaagaaggagatgaactccaacttgcaataccaagagaaaatgcacaaatatcactggatggagatgtcacattttttggtgcattgaaactgctg

SEQ ID NO: 82 - protein, 234 aa, Homo sapiens
MDPGLQQALNGMAPPGDTAMHVPAGSVASHLGTTSRSYFYLTTATLALCLVFTVATIMVLVVQRTDSIPNSPDNVPLKGGNCSEDLLCILKRAPFKKSWAYLQVAKHLNKTKLSWNKDGILHGVRYQDGNLVIQFPGLYFIICQLQFLVQCPNNSVDLKLELLINKHIKKQALVTVCESGMQTKHVYQNLSQFLLDYLQVNTTISVNVDTFQYIDTSTFPLENVLSIFLYSNSD

SEQ ID NO: 83 - DNA, 705 bp, Homo sapiens
  source 1..705: mol_type = genomic DNA
atggacccagggctgcagcaagcactcaacggaatggcccctcctggagacacagccatgcatgtgccggcgggctccgtggccagccacctggggaccacgagccgcagctatttctatttgaccacagccactctggctctgtgccttgtcttcacggtggccactattatggtgttggtcgttcagaggacggactccattcccaactcacctgacaacgtccccctcaaaggaggaaattgctcagaagacctcttatgtatcctgaaaagggctccattcaagaagtcatgggcctacctccaagtggcaaagcatctaaacaaaaccaagttgtcttggaacaaagatggcattctccatggagtcagatatcaggatgggaatctggtgatccaattccctggtttgtacttcatcatttgccaactgcagtttcttgtacaatgcccaaataattctgtcgatctgaagttggagcttctcatcaacaagcatatcaaaaaacaggccctggtgacagtgtgtgagtctggaatgcaaacgaaacacgtataccagaatctctctcaattcttgctggattacctgcaggtcaacaccaccatatcagtcaatgtggatacattccagtacatagatacaagcacctttcctcttgagaatgtgttgtccatcttcttatacagtaattcagactga

SEQ ID NO: 84 - RNA, 705 nt, Homo sapiens
  source 1..705: mol_type = transcribed RNA
atggacccagggctgcagcaagcactcaacggaatggcccctcctggagacacagccatgcatgtgccggcgggctccgtggccagccacctggggaccacgagccgcagctatttctatttgaccacagccactctggctctgtgccttgtcttcacggtggccactattatggtgttggtcgttcagaggacggactccattcccaactcacctgacaacgtccccctcaaaggaggaaattgctcagaagacctcttatgtatcctgaaaagggctccattcaagaagtcatgggcctacctccaagtggcaaagcatctaaacaaaaccaagttgtcttggaacaaagatggcattctccatggagtcagatatcaggatgggaatctggtgatccaattccctggtttgtacttcatcatttgccaactgcagtttcttgtacaatgcccaaataattctgtcgatctgaagttggagcttctcatcaacaagcatatcaaaaaacaggccctggtgacagtgtgtgagtctggaatgcaaacgaaacacgtataccagaatctctctcaattcttgctggattacctgcaggtcaacaccaccatatcagtcaatgtggatacattccagtacatagatacaagcacctttcctcttgagaatgtgttgtccatcttcttatacagtaattcagactga

SEQ ID NO: 85 - protein, 261 aa, Homo sapiens
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 86 - DNA, 786 bp, Homo sapiens
  source 1..786: mol_type = genomic DNA
atgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacagctttgaaatgcaaaaaggtgatcagaatcctcaaattgcggcacatgtcataagtgaggccagcagtaaaacaacatctgtgttacagtgggctgaaaaaggatactacaccatgagcaacaacttggtaaccctggaaaatgggaaacagctgaccgttaaaagacaaggactctattatatctatgcccaagtcaccttctgttccaatcgggaagcttcgagtcaagctccatttatagccagcctctgcctaaagtcccccggtagattcgagagaatcttactcagagctgcaaatacccacagttccgccaaaccttgcgggcaacaatccattcacttgggaggagtatttgaattgcaaccaggtgcttcggtgtttgtcaatgtgactgatccaagccaagtgagccatggcactggcttcacgtcctttggcttactcaaactctga

SEQ ID NO: 87 - RNA, 786 nt, Homo sapiens
  source 1..786: mol_type = transcribed RNA
atgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacagctttgaaatgcaaaaaggtgatcagaatcctcaaattgcggcacatgtcataagtgaggccagcagtaaaacaacatctgtgttacagtgggctgaaaaaggatactacaccatgagcaacaacttggtaaccctggaaaatgggaaacagctgaccgttaaaagacaaggactctattatatctatgcccaagtcaccttctgttccaatcgggaagcttcgagtcaagctccatttatagccagcctctgcctaaagtcccccggtagattcgagagaatcttactcagagctgcaaatacccacagttccgccaaaccttgcgggcaacaatccattcacttgggaggagtatttgaattgcaaccaggtgcttcggtgtttgtcaatgtgactgatccaagccaagtgagccatggcactggcttcacgtcctttggcttactcaaactctga

SEQ ID NO: 88 - protein, 193 aa, Homo sapiens
MPEEGSGCSVRRRPYGCVLRAALVPLVAGLVICLVVCIQRFAQAQQQLPLESLGWDVAELQLNHTGPQQDPRLYWQGGPALGRSFLHGPELDKGQLRIHRDGIYMVHIQVTLAICSSTTASRHHPTTLAVGICSPASRSISLLRLSFHQGCTIASQRLTPLARGDTLCTNLTGTLLPSRNTDETFFGVQWVRP

SEQ ID NO: 89 - DNA, 582 bp, Homo sapiens
  source 1..582: mol_type = genomic DNA
atgccggaggagggttcgggctgctcggtgcggcgcaggccctatgggtgcgtcctgcgggctgctttggtcccattggtcgcgggcttggtgatctgcctcgtggtgtgcatccagcgcttcgcacaggctcagcagcagctgccgctcgagtcacttgggtgggacgtagctgagctgcagctgaatcacacaggacctcagcaggaccccaggctatactggcaggggggcccagcactgggccgctccttcctgcatggaccagagctggacaaggggcagctacgtatccatcgtgatggcatctacatggtacacatccaggtgacgctggccatctgctcctccacgacggcctccaggcaccaccccaccaccctggccgtgggaatctgctctcccgcctcccgtagcatcagcctgctgcgtctcagcttccaccaaggttgtaccattgcctcccagcgcctgacgcccctggcccgaggggacacactctgcaccaacctcactgggacacttttgccttcccgaaacactgatgagaccttctttggagtgcagtgggtgcgcccctga

SEQ ID NO: 90 - RNA, 582 nt, Homo sapiens
  source 1..582: mol_type = transcribed RNA
atgccggaggagggttcgggctgctcggtgcggcgcaggccctatgggtgcgtcctgcgggctgctttggtcccattggtcgcgggcttggtgatctgcctcgtggtgtgcatccagcgcttcgcacaggctcagcagcagctgccgctcgagtcacttgggtgggacgtagctgagctgcagctgaatcacacaggacctcagcaggaccccaggctatactggcaggggggcccagcactgggccgctccttcctgcatggaccagagctggacaaggggcagctacgtatccatcgtgatggcatctacatggtacacatccaggtgacgctggccatctgctcctccacgacggcctccaggcaccaccccaccaccctggccgtgggaatctgctctcccgcctcccgtagcatcagcctgctgcgtctcagcttccaccaaggttgtaccattgcctcccagcgcctgacgcccctggcccgaggggacacactctgcaccaacctcactgggacacttttgccttcccgaaacactgatgagaccttctttggagtgcagtgggtgcgcccctga

SEQ ID NO: 91 - protein, 205 aa, Homo sapiens
MTPPERLFLPRVCGTTLHLLLLGLLLVLLPGAQGLPGVGLTPSAAQTARQHPKMHLAHSTLKPAAHLIGDPSKQNSLLWRANTDRAFLQDGFSLSNNSLLVPTSGIYFVYSQVVFSGKAYSPKATSSPLYLAHEVQLFSSQYPFHVPLLSSQKMVYPGLQEPWLHSMYHGAAFQLTQGDQLSTHTDGIPHLVLSPSTVFFGAFAL

SEQ ID NO: 92 - DNA, 615 bp, Homo sapiens
  source 1..615: mol_type = genomic DNA
atgacaccacctgaacgtctcttcctcccaagggtgtgtggcaccaccctacacctcctccttctggggctgctgctggttctgctgcctggggcccaggggctccctggtgttggcctcacaccttcagctgcccagactgcccgtcagcaccccaagatgcatcttgcccacagcaccctcaaacctgctgctcacctcattggagaccccagcaagcagaactcactgctctggagagcaaacacggaccgtgccttcctccaggatggtttctccttgagcaacaattctctcctggtccccaccagtggcatctacttcgtctactcccaggtggtcttctctgggaaagcctactctcccaaggccacctcctccccactctacctggcccatgaggtccagctcttctcctcccagtaccccttccatgtgcctctcctcagctcccagaagatggtgtatccagggctgcaggaaccctggctgcactcgatgtaccacggggctgcgttccagctcacccagggagaccagctatccacccacacagatggcatcccccacctagtcctcagccctagtactgtcttctttggagccttcgctctg

SEQ ID NO: 93 - RNA, 615 nt, Homo sapiens
  source 1..615: mol_type = transcribed RNA
atgacaccacctgaacgtctcttcctcccaagggtgtgtggcaccaccctacacctcctccttctggggctgctgctggttctgctgcctggggcccaggggctccctggtgttggcctcacaccttcagctgcccagactgcccgtcagcaccccaagatgcatcttgcccacagcaccctcaaacctgctgctcacctcattggagaccccagcaagcagaactcactgctctggagagcaaacacggaccgtgccttcctccaggatggtttctccttgagcaacaattctctcctggtccccaccagtggcatctacttcgtctactcccaggtggtcttctctgggaaagcctactctcccaaggccacctcctccccactctacctggcccatgaggtccagctcttctcctcccagtaccccttccatgtgcctctcctcagctcccagaagatggtgtatccagggctgcaggaaccctggctgcactcgatgtaccacggggctgcgttccagctcacccagggagaccagctatccacccacacagatggcatcccccacctagtcctcagccctagtactgtcttctttggagccttcgctctg

SEQ ID NO: 94 - protein, 233 aa, Homo sapiens; identical to SEQ ID NO: 70

SEQ ID NO: 95 - DNA, 699 bp, Homo sapiens; identical to SEQ ID NO: 71
  source 1..699: mol_type = genomic DNA

SEQ ID NO: 96 - RNA, 699 nt, Homo sapiens; identical to SEQ ID NO: 72
  source 1..699: mol_type = transcribed RNA

SEQ ID NO: 97 - protein, 281 aa, Homo sapiens [sequence omitted]

SEQ ID NO: 98 - DNA, 843 bp, Homo sapiens
  source 1..843: mol_type = genomic DNA
atggctatgatggaggtccaggggggacccagcctgggacagacctgcgtgctgatcgtgatcttcacagtgctcctgcagtctctctgtgtggctgtaacttacgtgtactttaccaacgagctgaagcagatgcaggacaagtactccaaaagtggcattgcttgtttcttaaaagaagatgacagttattgggaccccaatgacgaagagagtatgaacagcccctgctggcaagtcaagtggcaactccgtcagctcgttagaaagatgattttgagaacctctgaggaaaccatttctacagttcaagaaaagcaacaaaatatttctcccctagtgagagaaagaggtcctcagagagtagcagctcacataactgggaccagaggaagaagcaacacattgtcttctccaaactccaagaatgaaaaggctctgggccgcaaaataaactcctgggaatcatcaaggagtgggcattcattcctgagcaacttgcacttgaggaatggtgaactggtcatccatgaaaaagggttttactacatctattcccaaacatactttcgatttcaggaggaaataaaagaaaacacaaagaacgacaaacaaatggtccaatatatttacaaatacacaagttatcctgaccctatattgttgatgaaaagtgctagaaatagttgttggtctaaagatgcagaatatggactctattccatctatcaagggggaatatttgagcttaaggaaaatgacagaatttttgtttctgtaacaaatgagcacttgatagacatggaccatgaagccagtttttttggggcctttttagttggc

SEQ ID NO: 99 - RNA, 843 nt, Homo sapiens
  source 1..843: mol_type = transcribed RNA
atggctatgatggaggtccaggggggacccagcctgggacagacctgcgtgctgatcgtgatcttcacagtgctcctgcagtctctctgtgtggctgtaacttacgtgtactttaccaacgagctgaagcagatgcaggacaagtactccaaaagtggcattgcttgtttcttaaaagaagatgacagttattgggaccccaatgacgaagagagtatgaacagcccctgctggcaagtcaagtggcaactccgtcagctcgttagaaagatgattttgagaacctctgaggaaaccatttctacagttcaagaaaagcaacaaaatatttctcccctagtgagagaaagaggtcctcagagagtagcagctcacataactgggaccagaggaagaagcaacacattgtcttctccaaactccaagaatgaaaaggctctgggccgcaaaataaactcctgggaatcatcaaggagtgggcattcattcctgagcaacttgcacttgaggaatggtgaactggtcatccatgaaaaagggttttactacatctattcccaaacatactttcgatttcaggaggaaataaaagaaaacacaaagaacgacaaacaaatggtccaatatatttacaaatacacaagttatcctgaccctatattgttgatgaaaagtgctagaaatagttgttggtctaaagatgcagaatatggactctattccatctatcaagggggaatatttgagcttaaggaaaatgacagaatttttgtttctgtaacaaatgagcacttgatagacatggaccatgaagccagtttttttggggcctttttagttggc

SEQ ID NO: 100 - protein, 160 aa, Homo sapiens
MVPSAGQLALFALGIVLAACQALENSTSPLSADPPVAAAVVSHFNDCPDSHTQFCFHGTCRFLVQEDKPACVCHSGYVGARCEHADLLAVVAASQKKQAITALVVVSIVALAVLIITCVLIHCCQVRKHCEWCRALICRHEKPSALLKGRTACCHSETVV

SEQ ID NO: 101 - DNA, 358 bp, Homo sapiens
  source 1..358: mol_type = genomic DNA
atggtcccctcggctggacagctcgccctgttcgctctgggtattgtgttggctgcgtgccaggccttggagaacagcacgtccccgctgagtgcagacccgcccgtggctgcagcagtggtgtcccattttaatgactgcccagattcccacactcagttctgcttccatggaacctgcaggtttttggtgcaggaggacaagccagcatgtgtctgccattctgggtacgttggtgcacgctgtgagcatgcggacctcctggccgtggtggctgccagccagaagaagcaggccatcaccgccttggtggtggtctccatcgtggccctggctgtccttatcatcacatgtgtgc

SEQ ID NO: 102 - RNA, 358 nt, Homo sapiens
  source 1..358: mol_type = transcribed RNA
atggtcccctcggctggacagctcgccctgttcgctctgggtattgtgttggctgcgtgccaggccttggagaacagcacgtccccgctgagtgcagacccgcccgtggctgcagcagtggtgtcccattttaatgactgcccagattcccacactcagttctgcttccatggaacctgcaggtttttggtgcaggaggacaagccagcatgtgtctgccattctgggtacgttggtgcacgctgtgagcatgcggacctcctggccgtggtggctgccagccagaagaagcaggccatcaccgccttggtggtggtctccatcgtggccctggctgtccttatcatcacatgtgtgc

SEQ ID NO: 103 - protein, 70 aa, Homo sapiens
MDRAKLLLLLLLLLLPQAQAVVSHFNDCPDSHTQFCFHGTCRFLVQEDKPACVCHSGYVGARCEHADLLA

SEQ ID NO: 104 - DNA, 210 bp, Homo sapiens
  source 1..210: mol_type = genomic DNA
atggacagagccaagctgctgctgttgctgctccttctgctgctgcctcaggctcaagctgtggtgtcccattttaatgactgcccagattcccacactcagttctgcttccatggaacctgcaggtttttggtgcaggaggacaagccagcatgtgtctgccattctgggtacgttggtgcacgctgtgagcatgcggacctcctggcc

SEQ ID NO: 105 - RNA, 210 nt, Homo sapiens
  source 1..210: mol_type = transcribed RNA
atggacagagccaagctgctgctgttgctgctccttctgctgctgcctcaggctcaagctgtggtgtcccattttaatgactgcccagattcccacactcagttctgcttccatggaacctgcaggtttttggtgcaggaggacaagccagcatgtgtctgccattctgggtacgttggtgcacgctgtgagcatgcggacctcctggcc

SEQ ID NO: 106 - protein, 132 aa, Homo sapiens
MDRAKLLLLLLLLLLPQAQAALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 107 - DNA, 396 bp, Homo sapiens
  source 1..396: mol_type = genomic DNA
atggacagagccaagctgctgctgttgctgctccttctgctgctgcctcaggctcaagctgccctggacaccaactattgcttcagctccacggagaagaactgctgcgtgcggcagctgtacattgacttccgcaaggacctcggctggaagtggatccacgagcccaagggctaccatgccaacttctgcctcgggccctgcccctacatttggagcctggacacgcagtacagcaaggtcctggccctgtacaaccagcataacccgggcgcctcggcggcgccgtgctgcgtgccgcaggcgctggagccgctgcccatcgtgtactacgtgggccgcaagcccaaggtggagcagctgtccaacatgatcgtgcgctcctgcaagtgcagc

SEQ ID NO: 108 - RNA, 396 nt, Homo sapiens
  source 1..396: mol_type = transcribed RNA
atggacagagccaagctgctgctgttgctgctccttctgctgctgcctcaggctcaagctgccctggacaccaactattgcttcagctccacggagaagaactgctgcgtgcggcagctgtacattgacttccgcaaggacctcggctggaagtggatccacgagcccaagggctaccatgccaacttctgcctcgggccctgcccctacatttggagcctggacacgcagtacagcaaggtcctggccctgtacaaccagcataacccgggcgcctcggcggcgccgtgctgcgtgccgcaggcgctggagccgctgcccatcgtgtactacgtgggccgcaagcccaaggtggagcagctgtccaacatgatcgtgcgctcctgcaagtgcagc

SEQ ID NO: 109 - protein, 554 aa, Homo sapiens
MTAPGAAGRCPPTTWLGSLLLLVCLLASRSITEEVSEYCSHMIGSGHLQSLQRLIDSQMETSCQITFEFVDQEQLKDPVCYLKKAFLLVQDIMEDTMRFRDNTPNAIAIVQLQELSLRLKSCFTKDYEEHDKACVRTFYETPLQLLEKVKNVFNETKNLLDKDWNIFSKNCNNSFAECSSQDVVTKPDCNCLYPKAIPSSDPASVSPHQPLAPSMAPVAGLTWEDSEGTEGSSLLPGEQPLHTVDPGSAKQRPPRSTCQSFEPPETPVVKDSTIGGSPQPRPSVGAFNPGMEDILDSAMGTNWVPEEASGEASEIPVPQGTELSPSRPGGGSMQTEPARPSNFLSASSPLPASAKGQQPADVTGTALPRVGPVRPTGQDWNHTPQKTDHPSALLRDPPEPGSPRISSLRPQGLSNPSTLSAQPQLSRSHSSGSVLPLGELEGRRSTRDRRSPAEPEGGPASEGAARPLPRFNSVPLTDTGHERQSEGSFSPQLQESVFHLLVPSVILVLLAVGGLLFYRWRRRSHQEPQRADSPLEQPEGSPLTQDDRQVELPV

SEQ ID NO: 110 - DNA, 1662 bp, Homo sapiens
  source 1..1662: mol_type = genomic DNA
atgaccgcgccgggcgccgccgggcgctgccctcccacgacatggctgggctccctgctgttgttggtctgtctcctggcgagcaggagtatcaccgaggaggtgtcggagtactgtagccacatgattgggagtggacacctgcagtctctgcagcggctgattgacagtcagatggagacctcgtgccaaattacatttgagtttgtagaccaggaacagttgaaagatccagtgtgctaccttaagaaggcatttctcctggtacaagacataatggaggacaccatgcgcttcagagataacacccccaatgccatcgccattgtgcagctgcaggaactctctttgaggctgaagagctgcttcaccaaggattatgaagagcatgacaaggcctgcgtccgaactttctatgagacacctctccagttgctggagaaggtcaagaatgtctttaatgaaacaaagaatctccttgacaaggactggaatattttcagcaagaactgcaacaacagctttgctgaatgctccagccaagatgtggtgaccaagcctgattgcaactgcctgtaccccaaagccatccctagcagtgacccggcctctgtctcccctcatcagcccctcgccccctccatggcccctgtggctggcttgacctgggaggactctgagggaactgagggcagctccctcttgcctggtgagcagcccctgcacacagtggatccaggcagtgccaagcagcggccacccaggagcacctgccagagctttgagccgccagagaccccagttgtcaaggacagcaccatcggtggctcaccacagcctcgcccctctgtcggggccttcaaccccgggatggaggatattcttgactctgcaatgggcactaattgggtcccagaagaagcctctggagaggccagtgagattcccgtaccccaagggacagagctttccccctccaggccaggagggggcagcatgcagacagagcccgccagacccagcaacttcctctcagcatcttctccactccctgcatcagcaaagggccaacagccggcagatgtaactggtaccgccttgcccagggtgggccccgtgaggcccactggccaggactggaatcacaccccccagaagacagaccatccatctgccctgctcagagaccccccggagccaggctctcccaggatctcatcactgcgcccccagggcctcagcaacccctccaccctctctgctcagccacagctttccagaagccactcctcgggcagcgtgctgccccttggggagctggagggcaggaggagcaccagggatcggaggagccccgcagagccagaaggaggaccagcaagtgaaggggcagccaggcccctgccccgttttaactccgttcctttgactgacacaggccatgagaggcagtccgagggatccttcagcccgcagctccaggagtctgtcttccacctgctggtgcccagtgtcatcctggtcttgctggccgtcggaggcctcttgttctacaggtggaggcggcggagccatcaagagcctcagagagcggattctcccttggagcaaccagagggcagccccctgactcaggatgacagacaggtggaactgccagtg

SEQ ID NO: 111 - RNA, 1662 nt, Homo sapiens
  source 1..1662: mol_type = transcribed RNA
atgaccgcgccgggcgccgccgggcgctgccctcccacgacatggctgggctccctgctgttgttggtctgtctcctggcgagcaggagtatcaccgaggaggtgtcggagtactgtagccacatgattgggagtggacacctgcagtctctgcagcggctgattgacagtcagatggagacctcgtgccaaattacatttgagtttgtagaccaggaacagttgaaagatccagtgtgctaccttaagaaggcatttctcctggtacaagacataatggaggacaccatgcgcttcagagataacacccccaatgccatcgccattgtgcagctgcaggaactctctttgaggctgaagagctgcttcaccaaggattatgaagagcatgacaaggcctgcgtccgaactttctatgagacacctctccagttgctggagaaggtcaagaatgtctttaatgaaacaaagaatctccttgacaaggactggaatattttcagcaagaactgcaacaacagctttgctgaatgctccagccaagatgtggtgaccaagcctgattgcaactgcctgtaccccaaagccatccctagcagtgacccggcctctgtctcccctcatcagcccctcgccccctccatggcccctgtggctggcttgacctgggaggactctgagggaactgagggcagctccctcttgcctggtgagcagcccctgcacacagtggatccaggcagtgccaagcagcggccacccaggagcacctgccagagctttgagccgccagagaccccagttgtcaaggacagcaccatcggtggctcaccacagcctcgcccctctgtcggggccttcaaccccgggatggaggatattcttgactctgcaatgggcactaattgggtcccagaagaagcctctggagaggccagtgagattcccgtaccccaagggacagagctttccccctccaggccaggagggggcagcatgcagacagagcccgccagacccagcaacttcctctcagcatcttctccactccctgcatcagcaaagggccaacagccggcagatgtaactggtaccgccttgcccagggtgggccccgtgaggcccactggccaggactggaatcacaccccccagaagacagaccatccatctgccctgctcagagaccccccggagccaggctctcccaggatctcatcactgcgcccccagggcctcagcaacccctccaccctctctgctcagccacagctttccagaagccactcctcgggcagcgtgctgccccttggggagctggagggcaggaggagcaccagggatcggaggagccccgcagagccagaaggaggaccagcaagtgaaggggcagccaggcccctgccccgttttaactccgttcctttgactgacacaggccatgagaggcagtccgagggatccttcagcccgcagctccaggagtctgtcttccacctgctggtgcccagtgtcatcctggtcttgctggccgtcggaggcctcttgttctacaggtggaggcggcggagccatcaagagcctcagagagcggattctcccttggagcaaccagagggcagccccctgactcaggatgacagacaggtggaactgccagtg

SEQ ID NO: 112 - protein, 144 aa, Homo sapiens
MWLQSLLLLGTVACSISAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQE

SEQ ID NO: 113 - DNA, 432 bp, Homo sapiens
  source 1..432: mol_type = genomic DNA
atgtggctgcagagcctgctgctcttgggcactgtggcctgcagcatctctgcacccgcccgctcgcccagccccagcacgcagccctgggagcatgtgaatgccatccaggaggcccggcgtctcctgaacctgagtagagacactgctgctgagatgaatgaaacagtagaagtcatctcagaaatgtttgacctccaggagccgacctgcctacagacccgcctggagctgtacaagcagggcctgcggggcagcctcaccaagctcaagggccccttgaccatgatggccagccactacaagcagcactgccctccaaccccggaaacttcctgtgcaacccagattatcacctttgaaagtttcaaagagaacctgaaggactttctgcttgtcatcccctttgactgctgggagccagtccaggag

SEQ ID NO: 114 - RNA, 432 nt, Homo sapiens
  source 1..432: mol_type = transcribed RNA
atgtggctgcagagcctgctgctcttgggcactgtggcctgcagcatctctgcacccgcccgctcgcccagccccagcacgcagccctgggagcatgtgaatgccatccaggaggcccggcgtctcctgaacctgagtagagacactgctgctgagatgaatgaaacagtagaagtcatctcagaaatgtttgacctccaggagccgacctgcctacagacccgcctggagctgtacaagcagggcctgcggggcagcctcaccaagctcaagggccccttgaccatgatggccagccactacaagcagcactgccctccaaccccggaaacttcctgtgcaacccagattatcacctttgaaagtttcaaagagaacctgaaggactttctgcttgtcatcccctttgactgctgggagccagtccaggag

SEQ ID NO: 115 - protein, 207 aa, Homo sapiens
MAGPATQSPMKLMALQLLLWHSALWTVQEATPLGPASSLPQSFLLKCLEQVRKIQGDGAALQEKLVSECATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQP

SEQ ID NO: 116 - DNA, 621 bp, Homo sapiens
  source 1..621: mol_type = genomic DNA
atggctggacctgccacccagagccccatgaagctgatggccctgcagctgctgctgtggcacagtgcactctggacagtgcaggaagccacccccctgggccctgccagctccctgccccagagcttcctgctcaagtgcttagagcaagtgaggaagatccagggcgatggcgcagcgctccaggagaagctggtgagtgagtgtgccacctacaagctgtgccaccccgaggagctggtgctgctcggacactctctgggcatcccctgggctcccctgagcagctgccccagccaggccctgcagctggcaggctgcttgagccaactccatagcggccttttcctctaccaggggctcctgcaggccctggaagggatctcccccgagttgggtcccaccttggacacactgcagctggacgtcgccgactttgccaccaccatctggcagcagatggaagaactgggaatggcccctgccctgcagcccacccagggtgccatgccggccttcgcctctgctttccagcgccgggcaggaggggtcctggttgcctcccatctgcagagcttcctggaggtgtcgtaccgcgttctacgccaccttgcccagccc

SEQ ID NO: 117 - RNA, 621 nt, Homo sapiens
  source 1..621: mol_type = transcribed RNA
atggctggacctgccacccagagccccatgaagctgatggccctgcagctgctgctgtggcacagtgcactctggacagtgcaggaagccacccccctgggccctgccagctccctgccccagagcttcctgctcaagtgcttagagcaagtgaggaagatccagggcgatggcgcagcgctccaggagaagctggtgagtgagtgtgccacctacaagctgtgccaccccgaggagctggtgctgctcggacactctctgggcatcccctgggctcccctgagcagctgccccagccaggccctgcagctggcaggctgcttgagccaactccatagcggccttttcctctaccaggggctcctgcaggccctggaagggatctcccccgagttgggtcccaccttggacacactgcagctggacgtcgccgactttgccaccaccatctggcagcagatggaagaactgggaatggcccctgccctgcagcccacccagggtgccatgccggccttcgcctctgctttccagcgccgggcaggaggggtcctggttgcctcccatctgcagagcttcctggaggtgtcgtaccgcgttctacgccaccttgcccagccc

SEQ ID NO: 118 - protein, 110 aa, Homo sapiens
MSDAAVDTSSEITTKDLKEKKEVVEEAENGRDAPANGNANEENGEQEADNEVDEEEEEGGEEEEEEEEGDGEEEDGDEDEEAESATGKRAAEDDEDDDVDTKKQKTDEDD

SEQ ID NO: 119 - DNA, 330 bp, Homo sapiens
  source 1..330: mol_type = genomic DNA
atgtcagacgcagccgtagacaccagctccgaaatcaccaccaaggacttaaaggagaagaaggaagttgtggaagaggcagaaaatggaagagacgcccctgctaacgggaatgctaatgaggaaaatggggagcaggaggctgacaatgaggtagacgaagaagaggaagaaggtggggaggaagaggaggaggaagaagaaggtgatggtgaggaagaggatggagatgaagatgaggaagctgagtcagctacgggcaagcgggcagctgaagatgatgaggatgacgatgtcgataccaagaagcagaagaccgacgaggatgac

SEQ ID NO: 120 - RNA, 330 nt, Homo sapiens
  source 1..330: mol_type = transcribed RNA
atgtcagacgcagccgtagacaccagctccgaaatcaccaccaaggacttaaaggagaagaaggaagttgtggaagaggcagaaaatggaagagacgcccctgctaacgggaatgctaatgaggaaaatggggagcaggaggctgacaatgaggtagacgaagaagaggaagaaggtggggaggaagaggaggaggaagaagaaggtgatggtgaggaagaggatggagatgaagatgaggaagctgagtcagctacgggcaagcgggcagctgaagatgatgaggatgacgatgtcgataccaagaagcagaagaccgacgaggatgac

SEQ ID NO: 121 - protein, 129 aa, Homo sapiens
MDRAKLLLLLLLLLLPQAQASDAAVDTSSEITTKDLKEKKEVVEEAENGRDAPANGNANEENGEQEADNEVDEEEEEGGEEEEEEEEGDGEEEDGDEDEEAESATGKRAAEDDEDDDVDTKKQKTDEDD

SEQ ID NO: 122 - DNA, 387 bp, Homo sapiens
  source 1..387: mol_type = genomic DNA
atggacagagccaagctgctgctgttgctgctccttctgctgctgcctcaggctcaagcttcagacgcagccgtagacaccagctccgaaatcaccaccaaggacttaaaggagaagaaggaagttgtggaagaggcagaaaatggaagagacgcccctgctaacgggaatgctaatgaggaaaatggggagcaggaggctgacaatgaggtagacgaagaagaggaagaaggtggggaggaagaggaggaggaagaagaaggtgatggtgaggaagaggatggagatgaagatgaggaagctgagtcagctacgggcaagcgggcagctgaagatgatgaggatgacgatgtcgataccaagaagcagaagaccgacgaggatgac

SEQ ID NO: 123 - RNA, 387 nt, Homo sapiens
  source 1..387: mol_type = transcribed RNA
atggacagagccaagctgctgctgttgctgctccttctgctgctgcctcaggctcaagcttcagacgcagccgtagacaccagctccgaaatcaccaccaaggacttaaaggagaagaaggaagttgtggaagaggcagaaaatggaagagacgcccctgctaacgggaatgctaatgaggaaaatggggagcaggaggctgacaatgaggtagacgaagaagaggaagaaggtggggaggaagaggaggaggaagaagaaggtgatggtgaggaagaggatggagatgaagatgaggaagctgagtcagctacgggcaagcgggcagctgaagatgatgaggatgacgatgtcgataccaagaagcagaagaccgacgaggatgac

SEQ ID NO: 124 - protein, 30 aa, Homo sapiens
MDRAKLLLLLLLLLLPQAQATKKQKTDEDD

SEQ ID NO: 125 - DNA, 90 bp, Homo sapiens
  source 1..90: mol_type = genomic DNA
atggacagagccaagctgctgctgttgctgctccttctgctgctgcctcaggctcaagctaccaagaagcagaagaccgacgaggatgac

SEQ ID NO: 126 - RNA, 90 nt, Homo sapiens
  source 1..90: mol_type = transcribed RNA
atggacagagccaagctgctgctgttgctgctccttctgctgctgcctcaggctcaagctaccaagaagcagaagaccgacgaggatgac

SEQ ID NO: 127 - protein, 48 aa, Homo sapiens
MDRAKLLLLLLLLLLPQAQADAAVDTSSEITTKDLKEKKEVVEEAENG

SEQ ID NO: 128 - DNA, 144 bp, Homo sapiens
  source 1..144: mol_type = genomic DNA
atggacagagccaagctgctgctgttgctgctccttctgctgctgcctcaggctcaagctgacgcagccgtagacaccagctccgaaatcaccaccaaggacttaaaggagaagaaggaagttgtggaagaggcagaaaatgga

SEQ ID NO: 129 - RNA, 144 nt, Homo sapiens
  source 1..144: mol_type = transcribed RNA
atggacagagccaagctgctgctgttgctgctccttctgctgctgcctcaggctcaagctgacgcagccgtagacaccagctccgaaatcaccaccaaggacttaaaggagaagaaggaagttgtggaagaggcagaaaatgga

SEQ ID NO: 130 - protein, 235 aa, Homo sapiens
MTVLAPAWSPTTYLLLLLLLSSGLSGTQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAPTAPQPPLLLLLLLPVGLLLLAAAWCLHWQRTRRRTPRPGEQVPPVPSPQDLLLVEH

SEQ ID NO: 131 - DNA, 705 bp, Homo sapiens
  source 1..705: mol_type = genomic DNA
atgacagtgctggcgccagcctggagcccaacaacctatctcctcctgctgctgctgctgagctcgggactcagtgggacccaggactgctccttccaacacagccccatctcctccgacttcgctgtcaaaatccgtgagctgtctgactacctgcttcaagattacccagtcaccgtggcctccaacctgcaggacgaggagctctgcgggggcctctggcggctggtcctggcacagcgctggatggagcggctcaagactgtcgctgggtccaagatgcaaggcttgctggagcgcgtgaacacggagatacactttgtcaccaaatgtgcctttcagcccccccccagctgtcttcgcttcgtccagaccaacatctcccgcctcctgcaggagacctccgagcagctggtggcgctgaagccctggatcactcgccagaacttctcccggtgcctggagctgcagtgtcagcccgactcctcaaccctgccacccccatggagtccccggcccctggaggccacagccccgacagccccgcagccccctctgctcctcctactgctgctgcccgtgggcctcctgctgctggccgctgcctggtgcctgcactggcagaggacgcggcggaggacaccccgccctggggagcaggtgccccccgtccccagtccccaggacctgctgcttgtggagcac

SEQ ID NO: 132 - RNA, 705 nt, Homo sapiens
  source 1..705: mol_type = transcribed RNA
atgacagtgctggcgccagcctggagcccaacaacctatctcctcctgctgctgctgctgagctcgggactcagtgggacccaggactgctccttccaacacagccccatctcctccgacttcgctgtcaaaatccgtgagctgtctgactacctgcttcaagattacccagtcaccgtggcctccaacctgcaggacgaggagctctgcgggggcctctggcggctggtcctggcacagcgctggatggagcggctcaagactgtcgctgggtccaagatgcaaggcttgctggagcgcgtgaacacggagatacactttgtcaccaaatgtgcctttcagcccccccccagctgtcttcgcttcgtccagaccaacatctcccgcctcctgcaggagacctccgagcagctggtggcgctgaagccctggatcactcgccagaacttctcccggtgcctggagctgcagtgtcagcccgactcctcaaccctgccacccccatggagtccccggcccctggaggccacagccccgacagccccgcagccccctctgctcctcctactgctgctgcccgtgggcctcctgctgctggccgctgcctggtgcctgcactggcagaggacgcggcggaggacaccccgccctggggagcaggtgccccccgtccccagtccccaggacctgctgcttgtggagcac

SEQ ID NO: 133 - protein, 178 aa, Homo sapiens
MDRAKLLLLLLLLLLPQAQATQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAPTAPQP

SEQ ID NO: 134 - DNA, 534 bp, Homo sapiens
  source 1..534: mol_type = genomic DNA
atggacagagccaagctgctgctgttgctgctccttctgctgctgcctcaggctcaagctacccaggactgctccttccaacacagccccatctcctccgacttcgctgtcaaaatccgtgagctgtctgactacctgcttcaagattacccagtcaccgtggcctccaacctgcaggacgaggagctctgcgggggcctctggcggctggtcctggcacagcgctggatggagcggctcaagactgtcgctgggtccaagatgcaaggcttgctggagcgcgtgaacacggagatacactttgtcaccaaatgtgcctttcagcccccccccagctgtcttcgcttcgtccagaccaacatctcccgcctcctgcaggagacctccgagcagctggtggcgctgaagccctggatcactcgccagaacttctcccggtgcctggagctgcagtgtcagcccgactcctcaaccctgccacccccatggagtccccggcccctggaggccacagccccgacagccccgcagccc

SEQ ID NO: 135 - RNA, 534 nt, Homo sapiens
  source 1..534: mol_type = transcribed RNA
atggacagagccaagctgctgctgttgctgctccttctgctgctgcctcaggctcaagctacccaggactgctccttccaacacagccccatctcctccgacttcgctgtcaaaatccgtgagctgtctgactacctgcttcaagattacccagtcaccgtggcctccaacctgcaggacgaggagctctgcgggggcctctggcggctggtcctggcacagcgctggatggagcggctcaagactgtcgctgggtccaagatgcaaggcttgctggagcgcgtgaacacggagatacactttgtcaccaaatgtgcctttcagcccccccccagctgtcttcgcttcgtccagaccaacatctcccgcctcctgcaggagacctccgagcagctggtggcgctgaagccctggatcactcgccagaacttctcccggtgcctggagctgcagtgtcagcccgactcctcaaccctgccacccccatggagtccccggcccctggaggccacagccccgacagccccgcagccc

SEQ ID NO: 136 - protein, 19 aa, Porcine teschovirus-1
ATNFSLLKQAGDVEENPGP

SEQ ID NO: 137 - protein, 20 aa, Thosea asigna virus
QCTNYALLKLAGDVESNPGP

SEQ ID NO: 138 - protein, 22 aa, Foot and mouth disease virus
VKQTLNFDLLKLAGDVESNPGP

SEQ ID NO: 139 - protein, 6 aa, unidentified
  VARIANT 2: note = X can be any amino acid
  VARIANT 5: note = X can be any amino acid
RXRKXR

SEQ ID NO: 140 - protein, 6 aa, unidentified
RRRRRR

SEQ ID NO: 141 - protein, 27 aa, unidentified
RRRRRRGSGEGRGSLLTCGDVEENPGP

SEQ ID NO: 142 - DNA, 461 bp, Foot and mouth disease virus
  source 1..461: mol_type = genomic DNA
agcaggtttccccaactgacacaaaacgtgcaacttgaaactccgcctggtctttccaggtctagaggggtaacactttgtactgcgtttggctccacgctcgatccactggcgagtgttagtaacagcactgttgcttcgtagcggagcatgacggccgtgggaactcctccttggtaacaaggacccacggggccaaaagccacgcccacacgggcccgtcatgtgtgcaaccccagcacggcgactttactgcgaaacccactttaaagtgacattgaaactggtacccacacactggtgacaggctaaggatgcccttcaggtaccccgaggtaacacgcgacactcgggatctgagaaggggactggggcttctataaaagcgctcggtttaaaaagcttctatgcctgaataggtgaccggaggtcggcacctttcctttgcaattactgaccac

SEQ ID NO: 143 - DNA, 551 bp, Encephalomyocarditis virus
  source 1..551: mol_type = genomic DNA
cgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctcccctcaagcgtattcaacaaggggctgaaggatgcccagaaggtaccccattgtatgggatctgatctggggcctcggtgcacatgcttttcatgtgtttagtcgaggttaaaaaacgtctaggccccccgaaccacggggacgtggttttcctttgaaaaacacgatgataata

SEQ ID NO: 144 - DNA, 45 bp, Synthetic construct
  source 1..45: mol_type = other DNA
ggaggtggcgggtccgggggcgggggtagcggtggcgggggctcc

SEQ ID NO: 145 - protein, 15 aa, Synthetic construct
GGGGSGGGGSGGGGS

SEQ ID NO: 146 - protein, 535 aa, unidentified
MEKVHVDIEEDSPFLRALQRSFPQFEVEAKQVTDNDHANARAFSHLASKLIETEVDPSDTILDIGSAPARRMYSKHKYHCICPMRCAEDPDRLYKYATKLKKNCKEITDKELDKKMKELAAVMSDPDLETETMCLHDDESCRYEGQVAVYQDVYAVDGPTSLYHQANKGVRVAYWIGFDTTPFMFKNLAGAYPSYSTNWADETVLTARNIGLCSSDVMERSRRGMSILRKKYLKPSNNVLFSVGSTIYHEKRDLLRSWHLPSVFHLRGKQNYTCRCETIVSCDGYVVKRIAISPGLYGKPSGYAATMHREGFLCCKVTDTLNGERVSFPVCTYVPATLCDQMTGILATDVSADDAQKLLVGLNQRIVVNGRTQRNTNTMKNYLLPVVAQAFARWAKEYKEDQEDERPLGLRDRQLVMGCCWAFRRHKITSIYKRPDTQTIIKVNSDFHSFVLPRIGSNTLEIGLRTRIRKMLEEHKEPSPLITAEDVQEAKCAADEAKEVREAEELRAALPPLAADVEEPTLEADVDLMLQEAGA

SEQ ID NO: 147 - DNA, 1605 bp, unidentified
  source 1..1605: mol_type = other DNA
atggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggcc

SEQ ID NO: 148 - RNA, 1605 nt, unidentified
  source 1..1605: mol_type = other RNA
atggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggcc

SEQ ID NO: 149 - protein, 794 aa, unidentified
GSVETPRGLIKVTSYDGEDKIGSYAVLSPQAVLKSEKLSCIHPLAEQVIVITHSGRKGRYAVEPYHGKVVVPEGHAIPVQDFQALSESATIVYNEREFVNRYLHHIATHGGALNTDEEYYKTVKPSEHDGEYLYDIDRKQCVKKELVTGLGLTGELVDPPFHEFAYESLRTRPAAPYQVPTIGVYGVPGSGKSGIIKSAVTKKDLVVSAKKENCAEIIRDVKKMKGLDVNARTVDSVLLNGCKHPVETLYIDEAFACHAGTLRALIAIIRPKKAVLCGDPKQCGFFNMMCLKVHFNHEICTQVFHKSISRRCTKSVTSVVSTLFYDKKMRTTNPKETKIVIDTTGSTKPKQDDLILTCFRGWVKQLQIDYKGNEIMTAAASQGLTRKGVYAVRYKVNENPLYAPTSEHVNVLLTRTEDRIVWKTLAGDPWIKTLTAKYPGNFTATIEEWQAEHDAIMRHILERPDPTDVFQNKANVCWAKALVPVLKTAGIDMTTEQWNTVDYFETDKAHSAEIVLNQLCVRFFGLDLDSGLFSAPTVPLSIRNNHWDNSPSPNMYGLNKEVVRQLSRRYPQLPRAVATGRVYDMNTGTLRNYDPRINLVPVNRRLPHALVLHHNEHPQSDFSSFVSKLKGRTVLVVGEKLSVPGKMVDWLSDRPEATFRARLDLGIPGDVPKYDIIFVNVRTPYKYHHYQQCEDHAIKLSMLTKKACLHLNPGGTCVSIGYGYADRASESIIGAIARQFKFSRVCKPKSSLEETEVLFVFIGYDRKARTHNSYKLSSTLTNIYTGSRLHEAGC

SEQ ID NO: 150 - DNA, 2382 bp, unidentified
  source 1..2382: mol_type = other DNA
ggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaattcttacaagctttcatcaaccttgaccaacatttatacaggttccagactccacgaagccggatgt

SEQ ID NO: 151 - RNA, 2382 nt, unidentified
  source 1..2382: mol_type = other RNA
ggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaattcttacaagctttcatcaaccttgaccaacatttatacaggttccagactccacgaagccggatgt

SEQ ID NO: 152 - protein, 556 aa, unidentified
APSYHVVRGDIATATEGVIINAANSKGQPGGGVCGALYKKFPESFDLQPIEVGKARLVKGAAKHIIHAVGPNFNKVSEVEGDKQLAEAYESIAKIVNDNNYKSVAIPLLSTGIFSGNKDRLTQSLNHLLTALDTTDADVAIYCRDKKWEMTLKEAVARREAVEEICISDDSSVTEPDAELVRVHPKSSLAGRKGYSTSDGKTFSYLEGTKFHQAAKDIAEINAMWPVATEANEQVCMYILGESMSSIRSKCPVEESEASTPPSTLPCLCIHAMTPERVQRLKASRPEQITVCSSFPLPKYRITGVQKIQCSQPILFSPKVPAYIHPRKYLVETPPVDETPEPSAENQSTEGTPEQPPLITEDETRTRTPEPIIIEEEEEDSISLLSDGPTHQVLQVEADIHGPPSVSSSSWSIPHASDFDVDSLSILDTLEGASVTSGATSAETNSYFAKSMEFLARPVPAPRTVFRNPPHPAPRTRTPSLAPSRACSRTSLVSTPPGVNRVITREELEALTPSRTPSRSVSRTSLVSNPPGVNRVITREEFEAFVAQQQRFDAGA

SEQ ID NO: 153 - DNA, 1671 bp, unidentified
  source 1..1671: mol_type = other DNA
gcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgca

SEQ ID NO: 154 - RNA, 1671 nt, unidentified
  source 1..1671: mol_type = other RNA
gcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgca

SEQ ID NO: 155 - protein, 607 aa, unidentified
YIFSSDTGQGHLQQKSVRQTVLSEVVLERTELEISYAPRLDQEKEELLRKKLQLNPTPANRSRYQSRKVENMKAITARRILQGLGHYLKAEGKVECYRTLHPVPLYSSSVNRAFSSPKVAVEACNAMLKENFPTVASYCIIPEYDAYLDMVDGASCCLDTASFCPAKLRSFPKKHSYLEPTIRSAVPSAIQNTLQNVLAAATKRNCNVTQMRELPVLDSAAFNVECFKKYACNNEYWETFKENPIRLTEENVVNYITKLKGPKAAALFAKTHNLNMLQDIPMDRFVMDLKRDVKVTPGTKHTEERPKVQVIQAADPLATAYLCGIHRELVRRLNAVLLPNIHTLFDMSAEDFDAIIAEHFQPGDCVLETDIASFDKSEDDAMALTALMILEDLGVDAELLTLIEAAFGEISSIHLPTKTKFKFGAMMKSGMFLTLFVNTVINIVIASRVLRERLTGSPCAAFIGDDNIVKGVKSDKLMADRCATWLNMEVKIIDAVVGEKAPYFCGGFILCDSVTGTACRVADPLKRLFKLGKPLAADDEHDDDRRRALHEESTRWNRVGILSELCKAVESRYETVGTSIIVMAMTTLASSVKSFSYLRGAPITLYG

SEQ ID NO: 156 - DNA, 1821 bp, unidentified
  source 1..1821: mol_type = other DNA
tacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggc

SEQ ID NO: 157 - RNA, 1821 nt, unidentified
  source 1..1821: mol_type = other RNA
tacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggc

SEQ ID NO: 158 - RNA, 44 nt, unidentified
  source 1..44: mol_type = other RNA
atgggcggcgcatgagagaagcccagaccaattacctacccaaa

SEQ ID NO: 159 - RNA, 142 nt, unidentified
  source 1..142: mol_type = other RNA
aattggcaagctgcttacatagaactcgcggcgattggcatgccgccttaaaatttttattttatttttcttttcttttccgaatcggattttgtttttaatatttcaaaaaaaaaaaaaaaaaaaaaaaaaaaaaaaaaaa

SEQ ID NO: 160 - RNA, 7578 nt, Synthetic construct
  source 1..7578: mol_type = other RNA
atgggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaattcttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagtctagcatatggccacc

SEQ ID NO: 161 - DNA, 7578 bp, Synthetic construct
  source 1..7578: mol_type = other DNA
atgggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaattcttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagtctagcatatggccacc

SEQ ID NO: 162 - DNA, 7596 bp, Synthetic construct
  source 1..7596: mol_type = other DNA
taatacgactcactatagatgggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaattcttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagtctagcatatggccacc

SEQ ID NO: 163 - protein, 99 aa, Homo sapiens
MKVSAALLCLLLIAATFIPQGLAQPDAINAPVTCCYNFTNRKISVQRLASYRRITSSKCPKEAVIFKTIVAKEICADPKQKWVQDSMDHLDKQTQTPKT

SEQ ID NO: 164 - DNA, 297 bp, Homo sapiens
  source 1..297: mol_type = genomic DNA
atgaaagtctctgccgcccttctgtgcctgctgctcatagcagccaccttcattccccaagggctcgctcagccagatgcaatcaatgccccagtcacctgctgttataacttcaccaataggaagatctcagtgcagaggctcgcgagctatagaagaatcaccagcagcaagtgtcccaaagaagctgtgatcttcaagaccattgtggccaaggagatctgtgctgaccccaagcagaagtgggttcaggattccatggaccacctggacaagcaaacccaaactccgaagact

SEQ ID NO: 165 - RNA, 297 nt, Homo sapiens
  source 1..297: mol_type = transcribed RNA
atgaaagtctctgccgcccttctgtgcctgctgctcatagcagccaccttcattccccaagggctcgctcagccagatgcaatcaatgccccagtcacctgctgttataacttcaccaataggaagatctcagtgcagaggctcgcgagctatagaagaatcaccagcagcaagtgtcccaaagaagctgtgatcttcaagaccattgtggccaaggagatctgtgctgaccccaagcagaagtgggttcaggattccatggaccacctggacaagcaaacccaaactccgaagact

SEQ ID NO: 166 - protein, 92 aa, Homo sapiens
MQVSTAALAVLLCTMALCNQFSASLAADTPTACCFSYTSRQIPQNFIADYFETSSQCSKPGVIFLTKRSRQVCADPSEEWVQKYVSDLELSA

SEQ ID NO: 167 - DNA, 276 bp, Homo sapiens
  source 1..276: mol_type = genomic DNA
atgcaggtctccactgctgcccttgctgtcctcctctgcaccatggctctctgcaaccagttctctgcatcacttgctgctgacacgccgaccgcctgctgcttcagctacacctcccggcagattccacagaatttcatagctgactactttgagacgagcagccagtgctccaagcccggtgtcatcttcctaaccaagcgaagccggcaggtctgtgctgaccccagtgaggagtgggtccagaaatatgtcagcgacctggagctgagtgcc

SEQ ID NO: 168 - RNA, 276 nt, Homo sapiens
  source 1..276: mol_type = transcribed RNA
atgcaggtctccactgctgcccttgctgtcctcctctgcaccatggctctctgcaaccagttctctgcatcacttgctgctgacacgccgaccgcctgctgcttcagctacacctcccggcagattccacagaatttcatagctgactactttgagacgagcagccagtgctccaagcccggtgtcatcttcctaaccaagcgaagccggcaggtctgtgctgaccccagtgaggagtgggtccagaaatatgtcagcgacctggagctgagtgcc

SEQ ID NO: 169 - protein, 177 aa, Homo sapiens
MFHVSFRYIFGLPPLILVLLPVASSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 170 - DNA, 531 bp, Homo sapiens
  source 1..531: mol_type = genomic DNA
atgttccatgtttcttttaggtatatctttggacttcctcccctgatccttgttctgttgccagtagcatcatctgattgtgatattgaaggtaaagatggcaaacaatatgagagtgttctaatggtcagcatcgatcaattattggacagcatgaaagaaattggtagcaattgcctgaataatgaatttaacttttttaaaagacatatctgtgatgctaataaggaaggtatgtttttattccgtgctgctcgcaagttgaggcaatttcttaaaatgaatagcactggtgattttgatctccacttattaaaagtttcagaaggcacaacaatactgttgaactgcactggccaggttaaaggaagaaaaccagctgccctgggtgaagcccaaccaacaaagagtttggaagaaaataaatctttaaaggaacagaaaaaactgaatgacttgtgtttcctaaagagactattacaagagataaaaacttgttggaataaaattttgatgggcactaaagaacac

SEQ ID NO: 171 - RNA, 531 nt, Homo sapiens
  source 1..531: mol_type = transcribed RNA
atgttccatgtttcttttaggtatatctttggacttcctcccctgatccttgttctgttgccagtagcatcatctgattgtgatattgaaggtaaagatggcaaacaatatgagagtgttctaatggtcagcatcgatcaattattggacagcatgaaagaaattggtagcaattgcctgaataatgaatttaacttttttaaaagacatatctgtgatgctaataaggaaggtatgtttttattccgtgctgctcgcaagttgaggcaatttcttaaaatgaatagcactggtgattttgatctccacttattaaaagtttcagaaggcacaacaatactgttgaactgcactggccaggttaaaggaagaaaaccagctgccctgggtgaagcccaaccaacaaagagtttggaagaaaataaatctttaaaggaacagaaaaaactgaatgacttgtgtttcctaaagagactattacaagagataaaaacttgttggaataaaattttgatgggcactaaagaacac

SEQ ID NO: 172 - protein, 190 aa, Vaccinia virus
MSKIYIDERSDAEIVCAAIKNIGIEGATAAQLTRQLNMEKREVNKALYDLQRSAMVYSSDDIPPRWFMTTEADKPDADAMADVIIDDVSREKSMREDHKSFDDVIPAKKIIDWKDANPVTIINEYCQITKRDWSFRIESVGPSNSPTFYACVDIDGRVFDKADGKSKRDAKNNAAKLAVDKLLGYVIIRF

SEQ ID NO: 173 - DNA, 570 bp, Vaccinia virus
  source 1..570: mol_type = genomic DNA
atgtctaagatctatatcgacgagcgttctgacgcagagattgtgtgtgcggctattaaaaacattggaatcgaaggagctactgctgcacaactaactagacaacttaatatggagaagcgagaagttaataaagctctgtacgatcttcaacgtagtgctatggtgtacagctccgacgatattcctcctcgttggtttatgacaacggaggcggataagccggatgctgatgctatggctgacgtcataatagatgatgtatcccgcgaaaaatcaatgagagaggatcataagtcttttgatgatgttattccggctaaaaaaattattgattggaaagatgctaaccctgtcaccattattaatgagtactgccaaataactaagagagattggtcttttcgtattgaatcagttgggcctagtaactctcctacattttatgcctgtgtagatatcgacggaagagtattcgataaggccgatggaaaatctaaacgagatgctaaaaataatgcagctaaattggctgtagataaacttcttgggtacgtcatcattagattc

SEQ ID NO: 174 - RNA, 570 nt, Vaccinia virus
  source 1..570: mol_type = transcribed RNA
atgtctaagatctatatcgacgagcgttctgacgcagagattgtgtgtgcggctattaaaaacattggaatcgaaggagctactgctgcacaactaactagacaacttaatatggagaagcgagaagttaataaagctctgtacgatcttcaacgtagtgctatggtgtacagctccgacgatattcctcctcgttggtttatgacaacggaggcggataagccggatgctgatgctatggctgacgtcataatagatgatgtatcccgcgaaaaatcaatgagagaggatcataagtcttttgatgatgttattccggctaaaaaaattattgattggaaagatgctaaccctgtcaccattattaatgagtactgccaaataactaagagagattggtcttttcgtattgaatcagttgggcctagtaactctcctacattttatgcctgtgtagatatcgacggaagagtattcgataaggccgatggaaaatctaaacgagatgctaaaaataatgcagctaaattggctgtagataaacttcttgggtacgtcatcattagattc

SEQ ID NO: 175 - protein, 230 aa, Influenza A
MDPNTVSSFQVDCFLWHVRKRVADQELGDAPFLDRLRRDQKSLRGRGSTLGLDIETATRAGKQIVERILKEESDEALKMTMASVPASRYLTDMTLEEMSRDWSMLIPKQKVAGPLCIRMDQAIMDKNIILKANFSVIFDRLETLILLRAFTEEGAIVGEISPLPSLPGHTAEDVKNAVGVLIGGLEWNDNTVRVSETLQRFAWRSSNENGRPPLTPKQKREMAGTIRSEV

SEQ ID NO: 176 - DNA, 690 bp, Influenza A
  source 1..690: mol_type = genomic DNA
atggatccaaacactgtgtcaagctttcaggtagattgctttctttggcatgtccgcaaacgagttgcagaccaagaactaggtgatgccccattccttgatcggcttcgccgagatcagaaatccctaagaggaaggggcagcactcttggtctggacatcgagacagccacacgtgctggaaagcagatagtggagcggattctgaaagaagaatccgatgaggcacttaaaatgaccatggcctctgtacctgcgtcgcgttacctaaccgacatgactcttgaggaaatgtcaagggaatggtccatgctcatacccaagcagaaagtggcaggccctctttgtatcagaatggaccaggcgatcatggataaaaacatcatactgaaagcgaacttcagtgtgatttttgaccggctggagactctaatattgctaagggctttcaccgaagagggagcaattgttggcgaaatttcaccattgccttctcttccaggacatactgctgaggatgtcaaaaatgcagttggagtcctcatcggaggacttgaatggaatgataacacagttcgagtctctgaaactctacagagattcgcttggagaagcagtaatgagaatgggagacctccactcactccaaaacagaaacgagaaatggcgggaacaattaggtcagaagtt

SEQ ID NO: 177 - RNA, 690 nt, Influenza A
  source 1..690: mol_type = transcribed RNA
atggatccaaacactgtgtcaagctttcaggtagattgctttctttggcatgtccgcaaacgagttgcagaccaagaactaggtgatgccccattccttgatcggcttcgccgagatcagaaatccctaagaggaaggggcagcactcttggtctggacatcgagacagccacacgtgctggaaagcagatagtggagcggattctgaaagaagaatccgatgaggcacttaaaatgaccatggcctctgtacctgcgtcgcgttacctaaccgacatgactcttgaggaaatgtcaagggaatggtccatgctcatacccaagcagaaagtggcaggccctctttgtatcagaatggaccaggcgatcatggataaaaacatcatactgaaagcgaacttcagtgtgatttttgaccggctggagactctaatattgctaagggctttcaccgaagagggagcaattgttggcgaaatttcaccattgccttctcttccaggacatactgctgaggatgtcaaaaatgcagttggagtcctcatcggaggacttgaatggaatgataacacagttcgagtctctgaaactctacagagattcgcttggagaagcagtaatgagaatgggagacctccactcactccaaaacagaaacgagaaatggcgggaacaattaggtcagaagtt

SEQ ID NO: 178 - protein, 164 aa, Homo sapiens
MPITRMRMRPWLEMQINSNQIPGLIWINKEEMIFQIPWKHAAKHGWDINKDACLFRSWAIHTGRYKAGEKEPDPKTWKANFRCAMNSLPDIEEVKDQSRNKGSSAVRVYRMLPPLTKNQRKERKSKSSRDAKSKAKRKSCGDSSPDTFSDGLSSSTLPDDHSSY

SEQ ID NO: 179 - DNA, 492 bp, Homo sapiens
  source 1..492: mol_type = genomic DNA
atgcccatcactcggatgcgcatgagaccctggctagagatgcagattaattccaaccaaatcccggggctcatctggattaataaagaggagatgatcttccagatcccatggaagcatgctgccaagcatggctgggacatcaacaaggatgcctgtttgttccggagctgggccattcacacaggccgatacaaagcaggggaaaaggagccagatcccaagacgtggaaggccaactttcgctgtgccatgaactccctgccagatatcgaggaggtgaaagaccagagcaggaacaagggcagctcagctgtgcgagtgtaccggatgcttccacctctcaccaagaaccagagaaaagaaagaaagtcgaagtccagccgagatgctaagagcaaggccaagaggaagtcatgtggggattccagccctgataccttctctgatggactcagcagctccactctgcctgatgaccacagcagctac

SEQ ID NO: 180 - RNA, 492 nt, Homo sapiens
  source 1..492: mol_type = transcribed RNA
atgcccatcactcggatgcgcatgagaccctggctagagatgcagattaattccaaccaaatcccggggctcatctggattaataaagaggagatgatcttccagatcccatggaagcatgctgccaagcatggctgggacatcaacaaggatgcctgtttgttccggagctgggccattcacacaggccgatacaaagcaggggaaaaggagccagatcccaagacgtggaaggccaactttcgctgtgccatgaactccctgccagatatcgaggaggtgaaagaccagagcaggaacaagggcagctcagctgtgcgagtgtaccggatgcttccacctctcaccaagaaccagagaaaagaaagaaagtcgaagtccagccgagatgctaagagcaaggccaagaggaagtcatgtggggattccagccctgataccttctctgatggactcagcagctccactctgcctgatgaccacagcagctac

SEQ ID NO: 181 - RNA, 559 nt, Synthetic construct
  source 1..559: mol_type = other RNA
cggagacggcgcagaagaagaggatctggcgaaggcagaggcagcctgcttacatgtggcgacgtggaagagaaccccggacctatggcccgcgctgctctctccgccgcccccagcaatccccggctcctgcgagtggcactgctgctcctgctcctggtagccgctggccggcgcgcagcaggagcgtccgtggccactgaactgcgctgccagtgcttgcagaccctgcagggaattcaccccaagaacatccaaagtgtgaacgtgaagtcccccggaccccactgcgcccaaaccgaagtcatagccacactcaagaatgggcggaaagcttgcctcaatcctgcatcccccatagttaagaaaatcatcgaaaagatgctgaacagtgacaaatccaactgagcggccgcgaattggcaagctgcttacatagaactcgcggcgattggcatgccgccttaaaatttttattttatttttcttttcttttccgaatcggattttgtttttaatatttcaaaaaaaaaaaaaaaaaaaaaaaaaaaaaaaaaaa

SEQ ID NO: 182 - DNA, 559 bp, Synthetic construct
  source 1..559: mol_type = other DNA
cggagacggcgcagaagaagaggatctggcgaaggcagaggcagcctgcttacatgtggcgacgtggaagagaaccccggacctatggcccgcgctgctctctccgccgcccccagcaatccccggctcctgcgagtggcactgctgctcctgctcctggtagccgctggccggcgcgcagcaggagcgtccgtggccactgaactgcgctgccagtgcttgcagaccctgcagggaattcaccccaagaacatccaaagtgtgaacgtgaagtcccccggaccccactgcgcccaaaccgaagtcatagccacactcaagaatgggcggaaagcttgcctcaatcctgcatcccccatagttaagaaaatcatcgaaaagatgctgaacagtgacaaatccaactgagcggccgcgaattggcaagctgcttacatagaactcgcggcgattggcatgccgccttaaaatttttattttatttttcttttcttttccgaatcggattttgtttttaatatttcaaaaaaaaaaaaaaaaaaaaaaaaaaaaaaaaaaa

SEQ ID NO: 183 - DNA, 2367 bp, Synthetic construct
  source 1..2367: mol_type = other DNA
cggagacggcgcagaagaagaggatctggcgaaggcagaggcagcctgcttacatgtggcgacgtggaagagaaccccggacctatggcccgcgctgctctctccgccgcccccagcaatccccggctcctgcgagtggcactgctgctcctgctcctggtagccgctggccggcgcgcagcaggagcgtccgtggccactgaactgcgctgccagtgcttgcagaccctgcagggaattcaccccaagaacatccaaagtgtgaacgtgaagtcccccggaccccactgcgcccaaaccgaagtcatagccacactcaagaatgggcggaaagcttgcctcaatcctgcatcccccatagttaagaaaatcatcgaaaagatgctgaacagtgacaaatccaactgagcggccgcgaattggcaagctgcttacatagaactcgcggcgattggcatgccgccttaaaatttttattttatttttcttttcttttccgaatcggattttgtttttaatatttcaaaaaaaaaaaaaaaaaaaaaaaaaaaaaaaaaaacgcgtcgaggggaattaattcttgaagacgaaagggccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcgagctc

SEQ ID NO: 184 - DNA, 45 bp, unidentified
  source 1..45: mol_type = other DNA
gggcccctataactctctacggctaacctgaatggactacgacat

(19) United States
(12) Patent Application Publication
(10) Pub. No.: US 2025/0051798 A1
(43) Pub. Date: Feb. 13, 2025

(54) RNA CONSTRUCT

(71) Applicants: Imperial College Innovations Limited, South Kensington (GB); VaxEquity Ltd, Little Chesterford (GB)

(72) Inventors: Paul McKay, London, Greater London (GB); Robin Shattock, London, Greater London (GB)

(73) Assignees: Imperial College Innovations Limited, South Kensington, LN (GB); VaxEquity Ltd, Little Chesterford, ES (GB)

(21) Appl. No.: 18/721,163

(22) PCT Filed: Dec. 16, 2022
(86) PCT No.: PCT/GB2022/053275

§ 371 Date: Jun. 17, 2024

(30) Foreign Application Priority Data
Dec. 17, 2021 (GB) 2118464.3

(51) Int. Cl. C12N 15/86; C07K 14/005; C07K 14/495; C07K 14/52; C07K 14/525; C07K 14/53; C07K 14/535; C07K 14/54; C07K 14/545; C07K 14/55; C07K 14/705

(52) U.S. Cl. CPC C12N 15/86; C07K 14/005; C07K 14/495; C07K 14/521; C07K 14/522; C07K 14/523; C07K 14/525; C07K 14/53; C07K 14/535; C07K 14/54; C07K 14/545; C07K 14/55; C07K 14/70575; C12N 2770/36122; C12N 2770/36143

(57) ABSTRACT

The invention relates to RNA constructs, and particularly, although not exclusively, to mRNA constructs and saRNA replicons and to nucleic acids and expression vectors encoding such RNA constructs. The invention extends to the use of such RNA constructs in therapy, for example in treating diseases and/or in vaccine delivery. The invention extends to pharmaceutical compositions comprising such RNA constructs, and methods and uses thereof.

The present invention relates to RNA constructs, and particularly, although not exclusively, to mRNA constructs and saRNA replicons and to nucleic acids and expression vectors encoding such RNA constructs. The invention extends to the use of such RNA constructs in therapy, for example in treating diseases and/or in vaccine delivery. The invention extends to pharmaceutical compositions comprising such RNA constructs, and methods and uses thereof.

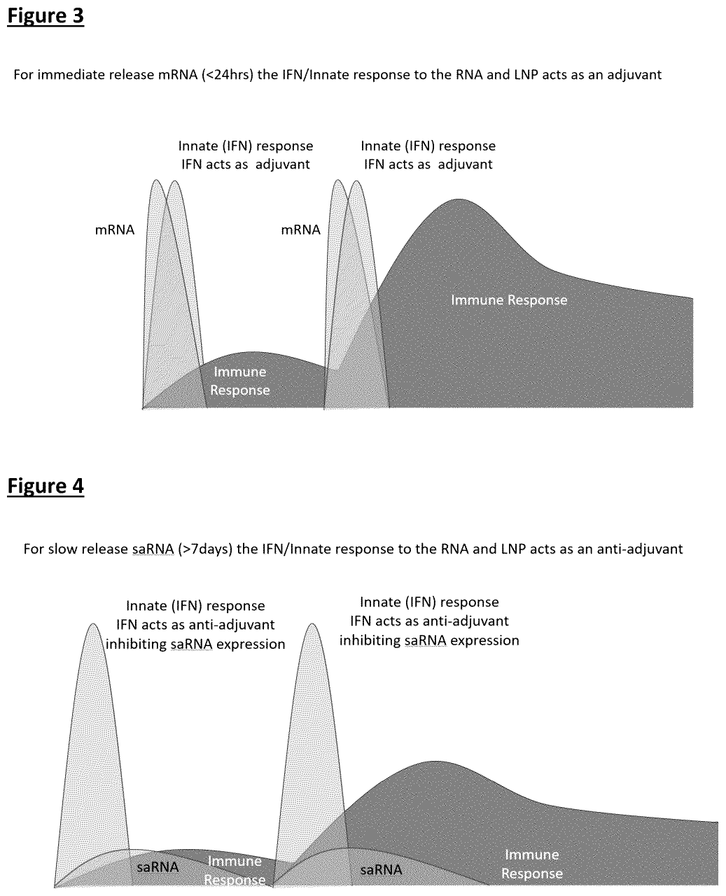

Messenger RNA (mRNA) and self-amplifying (saRNA) constructs are increasingly being tested for use in vaccine formulations. However, as shown in FIGS. 3 and 4, a problem with using standard mRNA and saRNA vaccines is that adverse events associated with the induction of antiviral (i.e. interferon) responses have been rate-limiting with respect to the increased doses of RNA likely to be effective in humans. The reason for this is unclear, but the inventors hypothesize that inherent differences in human innate sensing pose a barrier to the translation of RNA therapeutics from the lab to the clinic. Furthermore, innate sensing of RNA has been associated with the inhibition of protein expression.

If it is possible to block this innate immune response in the host, then the encoded protein may be expressed well, which is good for a biotherapeutic. However, a problem remains that it still may not stimulate a robust immune response.

Accordingly, there is a need in the art to produce new means by which RNA therapeutics, be they mRNA- or saRNA-based, may be delivered and expressed in patients, such that they are able to stimulate a robust immune response in the host.

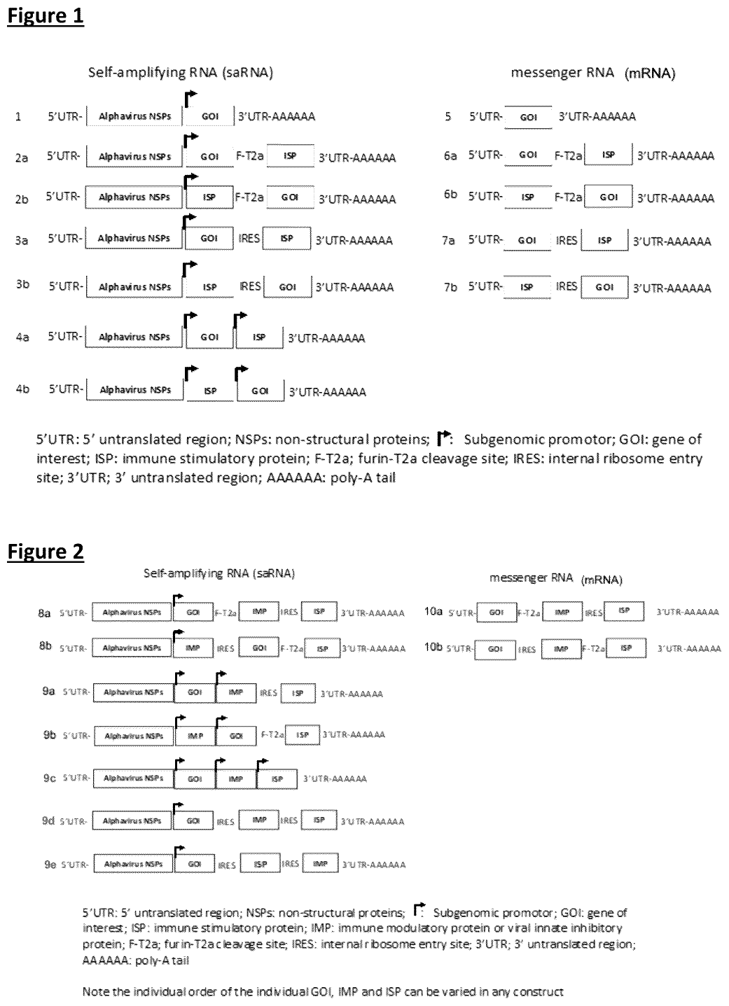

The inventors have investigated the inclusion of a molecular adjuvant referred to herein as an immune stimulatory protein (ISP), which is incorporated into the RNA vaccine, and believe that it will result in stimulating a greater immune response in patients. As described in the examples and as shown in FIGS. 1 and 2, the inventors have produced numerous embodiments of RNA construct, which encode a therapeutic biomolecule (i.e. gene of interest, GOI) and an ISP, and in some embodiments, an additional human innate modulatory protein (IMP) or a viral immune inhibitor protein (IIP). The ISP drives or steers the induction of an immune response to the expressed GOI or immunogen or where the co-encoded IMP or IIP blocks innate signalling pathways leading to interferon production and stimulation of ISRE or interferon signalling. In essence, the ISP is a molecular adjuvant, which is used either alone with the GOI, or in conjunction with an IMP or IIP and the GOI.

The skilled person will appreciate that an adjuvant is a pharmacological or immunological agent that improves the immune response of a vaccine, including RNA vaccines. Adjuvants may be added to a vaccine to boost the immune response to produce more antibodies and longer-lasting immunity, thereby minimizing the dose of specific antigen needed, for example a viral, bacterial or fungal coat protein etc. Molecular adjuvants may also be used to enhance the efficacy of a vaccine by helping to modify the immune response in particular types of immune system cells, for example, by activating T cells instead of antibody-secreting B cells depending on the purpose of the vaccine.

Accordingly, in a first aspect of the invention, there is provided a recombinant RNA construct encoding:
(a) (i) at least one therapeutic biomolecule;
   (ii) at least one immune stimulatory protein (ISP), and optionally (iii) at least one human innate modulatory protein (IMP) and/or at least one viral immune inhibitor protein (IIP); or
(b) (i) at least one immune stimulatory protein (ISP); and/or
   (ii) at least one human innate modulatory protein (IMP) and/or at least one viral immune inhibitor protein (IIP).

Advantageously, and preferably, the immune stimulatory proteins (ISPs), described herein, are designed to potentiate and/or modulate the immune responses to the therapeutic biomolecule (e.g. an antigen) that is encoded by a gene of interest (GOI) on the RNA construct. Increased understanding of molecular events driving adaptive immune responses has assisted the design of ISPs for use in RNA vaccines and the corresponding RNA constructs of the invention. Such ISP adjuvants may comprise RNA-encoded signalling molecules, such as cytokines, chemokines, immune costimulatory molecules, components of innate immunity or toll-like receptor agonists. In order to be effective in the context of RNA vaccination, the encoded ISPs aim to avoid any negative impact on RNA expression itself in the host. Thus, there should preferably be an optimal balance between the expression of the therapeutic biomolecule (e.g. an antigen) encoded by the GOI, potentiation of the immune response, while simultaneously avoiding or circumventing any innate suppression of RNA translation and/or replication (as in the case of saRNA, for example). This may be achieved by the careful selection of ISPs that do not have or actively suppress the expression of pathways that restrict or reduce RNA expression. In this respect, the GOI encoding the therapeutic biomolecule may be combined with a ISP in a manner that potentiates both expression and immunogenicity.

However, the additional inclusion of factors that directly block innate suppression of RNA expression (i.e. the IMPs and/or IIPs) may provide additional advantages. Furthermore, in certain configurations there may be a benefit from the use of an ISP that provides advantageous modulation of the adaptive immune response, but also trigger intracellular pathways that restrict RNA expression. Here, the combination of the ISP with a human IMP and/or viral IIP may provide an additional advantage, ensuring the positive modulatory function of the ISP is maintained while reducing the impact of any negative innate signalling pathway which leads to reduced RNA GOI expression.

In one preferred embodiment, the recombinant RNA construct comprises a nucleotide sequence encoding (i) at least one therapeutic biomolecule; (ii) at least one immune stimulatory protein (ISP), and optionally (iii) a human innate modulatory protein (IMP) and/or a viral immune inhibitor protein (IIP). More preferably, the recombinant RNA construct comprises a nucleotide sequence encoding (i) at least one therapeutic biomolecule; (ii) at least one immune stimulatory protein (ISP), and (iii) a human innate modulatory protein (IMP) and/or a viral immune inhibitor protein (IIP).

In another embodiment, however, the recombinant RNA construct comprises a nucleotide sequence encoding (i) at least one immune stimulatory protein (ISP) and/or (ii) a human innate modulatory protein (IMP) and/or a viral immune inhibitor protein (IIP). Thus, the recombinant RNA construct may comprise a nucleotide sequence encoding (i) at least one immune stimulatory protein (ISP); or (ii) a human innate modulatory protein (IMP) and/or a viral immune inhibitor protein (IIP). Preferably, however, the recombinant RNA construct comprises a nucleotide sequence encoding (i) at least one immune stimulatory protein (ISP); and (ii) a human innate modulatory protein (IMP) and/or a viral immune inhibitor protein (IIP). Preferably, in this embodiment, the construct does not encode, or comprise a nucleotide sequence encoding, a therapeutic biomolecule.

RNA constructs, such as mRNA and saRNA replicons, have been postulated to be potential tools for the delivery and expression of genes of interest for vaccines and therapeutics. However, single stranded mRNA (ssRNA) and double stranded RNA (dsRNA) is detected intracellularly by innate sensing mechanisms that trigger responses, which inhibit protein translation. As a consequence, expression of genes of interest encoded by the RNA construct is significantly impaired and thus the immunogenic or therapeutic potential of RNA constructs, including saRNA and mRNA is limited. Advantageously, the RNA constructs of the invention overcome this problem because they encode, in various embodiments, (i) one or more immune stimulatory proteins (ISP), which enhance or modify the immune response to the encoded therapeutic biomolecule and/or (ii) one or more human innate modulatory protein (IMP) or a viral immune inhibitor protein (IIP), which reduce or ablate the downstream innate inhibition of the transgene expression within the host cell.

Preferably, the at least one IMP and/or IIP is capable of inhibiting the innate immune response to RNA in a subject treated with the RNA construct of the invention. The IMP can, therefore, be described as a modulator of innate immunity, and the IIP can be described as an inhibitor of innate immunity. Advantageously, the presence, in the RNA construct of the first aspect, of one or more IMP or IIP, enables dual protein expression with the biotherapeutic molecule, i.e. a peptide or protein of interest.

As described in the examples, the RNA constructs of the invention (also known as “Stealthicons”) encoding a GOI and ISP have surprisingly been shown to increase GOI expression and/or immunogenicity compared to a conventional VEEV RNA replicon or a conventional RNA. In addition, when IIP or IMP are expressed as part of the same saRNA or RNA, together with an ISP, they have been shown to increase GOI and ISP expression in vitro resulting in recruitment of immune cells. Furthermore, the GOI and/or immunogenicity of these saRNA or RNA was further increased compared to RNA expressing ISP alone with the GOI.

The RNA construct of any aspect may be single-stranded RNA or double-stranded RNA.

The RNA constructs described herein are “recombinant”, meaning that they are not naturally occurring and have been synthesised using molecular biology techniques, such as genetic recombination, and molecular cloning to thereby create sequences, and combinations of sequences, which do not occur in nature.

The RNA construct of any aspect may comprise an mRNA or a saRNA system.

In one embodiment, the RNA construct comprises mRNA. FIG. 1 (right hand side) illustrates various embodiments of the RNA construct as a mRNA molecule.

In a preferred embodiment, the RNA construct comprises self-amplifying RNA (saRNA). FIG. 1 (left hand side) illustrates various embodiments of the RNA construct as a saRNA molecule. The skilled person would understand that such an RNA construct can also be referred to as a self-replicating RNA virus vector, or an RNA replicon.

Preferably, the saRNA construct comprises or is derived from a positive stranded RNA virus selected from the group of genus consisting of: alphavirus; picornavirus; flavivirus; rubivirus; pestivirus; hepacivirus; calicivirus and coronavirus.

Preferably, the RNA construct comprises or is derived from an alphavirus. Suitable wild-type alphavirus sequences are well-known. Representative examples of suitable alphaviruses include Aura, Bebaru virus, Cabassou, Chikungunya virus, Eastern equine encephalomyelitis virus, Fort Morgan, Getah virus, Kyzylagach, Mayaro, Mayaro virus, Middleburg, Mucambo virus, Ndumu, Pixuna virus, Ross River virus, Semliki Forest, Sindbis virus, Tonate, Triniti, Una, Venezuelan equine encephalomyelitis, Western equine encephalomyelitis, Whataroa, and Y-62-33. Preferably, therefore, the RNA construct comprises or is derived from any of these alphaviruses.

Preferably, the RNA construct comprises or is derived from a virus selected from the group of species consisting of: Venezuelan Equine Encephalitis Virus (VEEV); enterovirus 71; Encephalomyocarditis virus; Kunjin virus; and Middle East respiratory syndrome virus. In one preferred embodiment, the RNA construct comprises or is derived from Kunjin virus. Preferably, the RNA construct comprises or is derived from VEEV.

The immune stimulatory protein (ISP) is preferably a mammalian ISP. More preferably, the ISP is a primate ISP. Most preferably, the ISP is a human ISP. It will be appreciated that the at least one ISP may be a molecular adjuvant.

The ISP may be cytokine. The ISP may be a chemokine, which may be an alpha chemokine, a beta chemokine, a delta chemokine, or a gamma chemokine.

In a preferred embodiment, the ISP is an alpha chemokine. In one embodiment, therefore, the at least one ISP may be CXCL1 (NCBI Reference Sequence: NM_001511.4; UniProtKB—P09341 (GROA_HUMAN), or an orthologue thereof. One embodiment of the polypeptide sequence of CXCL1 (with the signal peptide spanning residues 1-34) is represented herein as SEQ ID No: 1, as follows:

[SEQ ID No: 1]
MARAALSAAPSNPRLLRVALLLLLLVAAGRRAAGASVATELRCQC
LQTLQGIHPKNIQSVNVKSPGPHCAQTEVIATLKNGRKACLNPAS
PIVKKIIEKMLNSDKSN

Therefore, preferably the RNA construct of any aspect comprises a nucleotide sequence which encodes an amino acid sequence substantially as set out in SEQ ID No: 1 or a variant or fragment thereof.

In one embodiment, the CXCL1 polypeptide is encoded by the DNA nucleotide sequence of SEQ ID No: 2, as follows:

[SEQ ID No: 2]
ATGGCCCGCGCTGCTCTCTCCGCCGCCCCCAGCAATCCCCGGCTC
CTGCGAGTGGCACTGCTGCTCCTGCTCCTGGTAGCCGCTGGCCGG
CGCGCAGCAGGAGCGTCCGTGGCCACTGAACTGCGCTGCCAGTGC
TTGCAGACCCTGCAGGGAATTCACCCCAAGAACATCCAAAGTGTG
AACGTGAAGTCCCCCGGACCCCACTGCGCCCAAACCGAAGTCATA
GCCACACTCAAGAATGGGCGGAAAGCTTGCCTCAATCCTGCATCC
CCCATAGTTAAGAAAATCATCGAAAAGATGCTGAACAGTGACAAA
TCCAAC

Accordingly, preferably the CXCL1 polypeptide is encoded by the DNA nucleotide sequence substantially as set out in SEQ ID NO: 2, or a variant or fragment thereof.

Thus, the RNA construct may comprise an RNA nucleotide sequence of SEQ ID No: as follows:

[SEQ ID No: 3]
AUGGCCCGCGCUGCUCUCUCCGCCGCCCCCAGCAAUCCCCGGCUC
CUGCGAGUGGCACUGCUGCUCCUGCUCCUGGUAGCCGCUGGCCGG
CGCGCAGCAGGAGCGUCCGUGGCCACUGAACUGCGCUGCCAGUGC
UUGCAGACCCUGCAGGGAAUUCACCCCAAGAACAUCCAAAGUGUG
AACGUGAAGUCCCCCGGACCCCACUGCGCCCAAACCGAAGUCAUA
GCCACACUCAAGAAUGGGCGGAAAGCUUGCCUCAAUCCUGCAUCC
CCCAUAGUUAAGAAAAUCAUCGAAAAGAUGCUGAACAGUGACAAA
UCCAAC

Furthermore, preferably the RNA construct comprises an RNA nucleotide sequence substantially as set out in SEQ ID No: 3, or a variant or fragment thereof.

In one embodiment, the at least one ISP may be an CXCL8 (IL8) (NCBI Reference Sequence: NM_000584.4; UniProtKB—P10145 (IL8_HUMAN)), or an orthologue thereof. One embodiment of the polypeptide sequence of CXCL8 (with the signal peptide spanning residues 1-20) is represented herein as SEQ ID No: 4, as follows:

[SEQ ID No: 4]
MTSKLAVALLAAFLISAALCEGAVLPRSAKELRCQCIKTYSKPFH
PKFIKELRVIESGPHCANTEIIVKLSDGRELCLDPKENWVQRVVE
KFLKRAENS

Therefore, preferably the RNA construct of any aspect comprises a nucleotide sequence which encodes an amino acid sequence substantially as set out in SEQ ID No: 4, or a variant or fragment thereof.

In one embodiment, the CXCL8 polypeptide is encoded by the DNA nucleotide sequence of SEQ ID No: 5, as follows:

[SEQ ID No: 5]
ATGACTTCCAAGCTGGCCGTGGCTCTCTTGGCAGCCTTCCTGATT
TCTGCAGCTCTGTGTGAAGGTGCAGTTTTGCCAAGGAGTGCTAAA
GAACTTAGATGTCAGTGCATAAAGACATACTCCAAACCTTTCCAC
CCCAAATTTATCAAAGAACTGAGAGTGATTGAGAGTGGACCACAC
TGCGCCAACACAGAAATTATTGTAAAGCTTTCTGATGGAAGAGAG
CTCTGTCTGGACCCCAAGGAAAACTGGGTGCAGAGGGTTGTGGAG
AAGTTTTTGAAGAGGGCTGAGAATTCA

Accordingly, preferably the CXCL8 polypeptide is encoded by the DNA nucleotide sequence substantially as set out in SEQ ID NO: 5, or a variant or fragment thereof.

Thus, the RNA construct may comprise an RNA nucleotide sequence of SEQ ID No: 6, as follows:

[SEQ ID No: 6]
AUGACUUCCAAGCUGGCCGUGGCUCUCUUGGCAGCCUUCCUGAUU
UCUGCAGCUCUGUGUGAAGGUGCAGUUUUGCCAAGGAGUGCUAAA
GAACUUAGAUGUCAGUGCAUAAAGACAUACUCCAAACCUUUCCAC
CCCAAAUUUAUCAAAGAACUGAGAGUGAUUGAGAGUGGACCACAC
UGCGCCAACACAGAAAUUAUUGUAAAGCUUUCUGAUGGAAGAGAG
CUCUGUCUGGACCCCAAGGAAAACUGGGUGCAGAGGGUUGUGGAG
AAGUUUUUGAAGAGGGCUGAGAAUUCA

Furthermore, preferably the RNA construct comprises an RNA nucleotide sequence substantially as set out in SEQ ID No: 6, or a variant or fragment thereof.

In one embodiment, the at least one ISP may be CXCL10 (NCBI Reference Sequence: NM_001565.4; UniProtKB—P02778 (CXL10_HUMAN)), or an orthologue thereof. One embodiment of the polypeptide sequence of CXCL10 (signal peptide is residues 1-21) is represented herein as SEQ ID No: 7, as follows:

[SEQ ID No: 7]
MNQTAILICCLIFLTLSGIQGVPLSRTVRCTCISISNQPVNPRSL
EKLEIIPASQFCPRVEIIATMKKKGEKRCLNPESKAIKNLLKAVS
KERSKRSP

Therefore, preferably the RNA construct of any aspect comprises a nucleotide sequence which encodes an amino acid sequence substantially as set out in SEQ ID No: 7, or a variant or fragment thereof.

In one embodiment, the CXCL10 polypeptide is encoded by the DNA nucleotide sequence of SEQ ID No: 8, as follows:

[SEQ ID No: 8]
ATGAATCAAACTGCCATTCTGATTTGCTGCCTTATCTTTCTGACT
CTAAGTGGCATTCAAGGAGTACCTCTCTCTAGAACTGTACGCTGT
ACCTGCATCAGCATTAGTAATCAACCTGTTAATCCAAGGTCTTTA
GAAAAACTTGAAATTATTCCTGCAAGCCAATTTTGTCCACGTGTT
GAGATCATTGCTACAATGAAAAAGAAGGGTGAGAAGAGATGTCTG
AATCCAGAATCGAAGGCCATCAAGAATTTACTGAAAGCAGTTAGC
AAGGAAAGGTCTAAAAGATCTCCT

Accordingly, preferably the CXCL10 polypeptide is encoded by the DNA nucleotide sequence substantially as set out in SEQ ID NO: 8, or a variant or fragment thereof.

Thus, the RNA construct may comprise an RNA nucleotide sequence of SEQ ID No: 9, as follows:

[SEQ ID No: 9]
AUGAAUCAAACUGCCAUUCUGAUUUGCUGCCUUAUCUUUCUGACU
CUAAGUGGCAUUCAAGGAGUACCUCUCUCUAGAACUGUACGCUGU
ACCUGCAUCAGCAUUAGUAAUCAACCUGUUAAUCCAAGGUCUUUA
GAAAAACUUGAAAUUAUUCCUGCAAGCCAAUUUUGUCCACGUGUU
GAGAUCAUUGCUACAAUGAAAAAGAAGGGUGAGAAGAGAUGUCUG
AAUCCAGAAUCGAAGGCCAUCAAGAAUUUACUGAAAGCAGUUAGC
AAGGAAAGGUCUAAAAGAUCUCCU

Furthermore, preferably the RNA construct comprises an RNA nucleotide sequence substantially as set out in SEQ ID No: 9, or a variant or fragment thereof.

In one embodiment, the at least one ISP may be CXCL11 (NCBI Reference Sequence: NM_005409.5; UniProtKB—O14625 (CXL11_HUMAN)), or an orthologue thereof. One embodiment of the polypeptide sequence of CXCL11 (signal peptide is residues 1-21) is represented herein as SEQ ID No: 10, as follows:

[SEQ ID No: 10]
MSVKGMAIALAVILCATVVQGFPMFKRGRCLCIGPGVKAVKVADI
EKASIMYPSNNCDKIEVIITLKENKGQRCLNPKSKQARLIIKKVE
RKNF

Therefore, preferably the RNA construct of any aspect comprises a nucleotide sequence which encodes an amino acid sequence substantially as set out in SEQ ID No: 10, or a variant or fragment thereof.

In one embodiment, the CXCL11 polypeptide is encoded by the DNA nucleotide sequence of SEQ ID No: 11, as follows:

[SEQ ID No: 11]
ATGAGTGTGAAGGGCATGGCTATAGCCTTGGCTGTGATATTGTGT
GCTACAGTTGTTCAAGGCTTCCCCATGTTCAAAAGAGGACGCTGT
CTTTGCATAGGCCCTGGGGTAAAAGCAGTGAAAGTGGCAGATATT
GAGAAAGCCTCCATAATGTACCCAAGTAACAACTGTGACAAAATA
GAAGTGATTATTACCCTGAAAGAAAATAAAGGACAACGATGCCTA
AATCCCAAATCGAAGCAAGCAAGGCTTATAATCAAAAAAGTTGAA
AGAAAGAATTTT

Accordingly, preferably the CXCL11 polypeptide is encoded by the DNA nucleotide sequence substantially as set out in SEQ ID NO: 11, or a variant or fragment thereof.

Thus, the RNA construct may comprise an RNA nucleotide sequence of SEQ ID No: 12, as follows:

[SEQ ID No: 12]
AUGAGUGUGAAGGGCAUGGCUAUAGCCUUGGCUGUGAUAUUGUGU
GCUACAGUUGUUCAAGGCUUCCCCAUGUUCAAAAGAGGACGCUGU
CUUUGCAUAGGCCCUGGGGUAAAAGCAGUGAAAGUGGCAGAUAUU
GAGAAAGCCUCCAUAAUGUACCCAAGUAACAACUGUGACAAAAUA
GAAGUGAUUAUUACCCUGAAAGAAAAUAAAGGACAACGAUGCCUA
AAUCCCAAAUCGAAGCAAGCAAGGCUUAUAAUCAAAAAAGUUGAA
AGAAAGAAUUUU

Furthermore, preferably the RNA construct comprises an RNA nucleotide sequence substantially as set out in SEQ ID No: 12, or a variant or fragment thereof.

In one embodiment, the at least one ISP may be CXCL12 (NCBI Reference Sequence: NM_000609.7; UniProtKB—P48061 (SDF1_HUMAN)), or an orthologue thereof. One embodiment of the polypeptide sequence of CXCL12 (signal peptide is residues 1-21) is represented herein as SEQ ID No: 13, as follows:

[SEQ ID No: 13]
MNAKVVVVLVLVLTALCLSDGKPVSLSYRCPCRFFESHVARANVK
HLKILNTPNCALQIVARLKNNNRQVCIDPKLKWIQEYLEKALNKR
FKM

Therefore, preferably the RNA construct of any aspect comprises a nucleotide sequence which encodes an amino acid sequence substantially as set out in SEQ ID No: 13, or a variant or fragment thereof.

In one embodiment, the CXCL12 polypeptide is encoded by the DNA nucleotide sequence of SEQ ID No: 14 as follows:

[SEQ ID No: 14]
ATGAACGCCAAGGTCGTGGTCGTGCTGGTCCTCGTGCTGACCGCG
CTCTGCCTCAGCGACGGGAAGCCCGTCAGCCTGAGCTACAGATGC
CCATGCCGATTCTTCGAAAGCCATGTTGCCAGAGCCAACGTCAAG
CATCTCAAAATTCTCAACACTCCAAACTGTGCCCTTCAGATTGTA
GCCCGGCTGAAGAACAACAACAGACAAGTGTGCATTGACCCGAAG
CTAAAGTGGATTCAGGAGTACCTGGAGAAAGCTTTAAACAAGAGG
TTCAAGATG

Accordingly, preferably the CXCL12 polypeptide is encoded by the DNA nucleotide sequence substantially as set out in SEQ ID NO: 14, or a variant or fragment thereof.

Thus, the RNA construct may comprise an RNA nucleotide sequence of SEQ ID No: 15, as follows:

[SEQ ID No: 15]
AUGAACGCCAAGGUCGUGGUCGUGCUGGUCCUCGUGCUGACCGCG
CUCUGCCUCAGCGACGGGAAGCCCGUCAGCCUGAGCUACAGAUGC
CCAUGCCGAUUCUUCGAAAGCCAUGUUGCCAGAGCCAACGUCAAG
CAUCUCAAAAUUCUCAACACUCCAAACUGUGCCCUUCAGAUUGUA
GCCCGGCUGAAGAACAACAACAGACAAGUGUGCAUUGACCCGAAG
CUAAAGUGGAUUCAGGAGUACCUGGAGAAAGCUUUAAACAAGAGG
UUCAAGAUG

Furthermore, preferably the RNA construct comprises an RNA nucleotide sequence substantially as set out in SEQ ID No: 15, or a variant or fragment thereof.

In a preferred embodiment, the ISP is a beta chemokine, preferably any one of CCL1-18, or an orthologue thereof. Hence, in one embodiment, the at least one ISP may be CCL1 (NCBI Reference Sequence: NM_002981.2; UniProtKB—P22362 (CCL1_HUMAN)), or an orthologue thereof. One embodiment of the polypeptide sequence of CCL1 (signal peptide spanning residues 1-23) is represented herein as SEQ ID No: 16, as follows:

[SEQ ID No: 16]
MQIITTALVCLLLAGMWPEDVDSKSMQVPFSRCCESFAEQEIPLR
AILCYRNTSSICSNEGLIFKLKRGKEACALDTVGWVQRHRKMLRH
CPSKRK

Therefore, preferably the RNA construct of any aspect comprises a nucleotide sequence which encodes an amino acid sequence substantially as set out in SEQ ID No: 16, or a variant or fragment thereof.

In one embodiment, the CCL1 polypeptide is encoded by the DNA nucleotide sequence of SEQ ID No: 17, as follows:

[SEQ ID No: 17]
ATGCAGATCATCACCACAGCCCTGGTGTGCTTGCTGCTAGCTGGG
ATGTGGCCGGAAGATGTGGACAGCAAGAGCATGCAGGTACCCTTC
TCCAGATGTTGCTTCTCATTTGCGGAGCAAGAGATTCCCCTGAGG
GCAATCCTGTGTTACAGAAATACCAGCTCCATCTGCTCCAATGAG
GGCTTAATATTCAAGCTGAAGAGAGGCAAAGAGGCCTGCGCCTTG
GACACAGTTGGATGGGTTCAGAGGCACAGAAAAATGCTGAGGCAC
TGCCCGTCAAAAAGAAAA

Accordingly, preferably the CCL1 polypeptide is encoded by the DNA nucleotide sequence substantially as set out in SEQ ID NO: 17, or a variant or fragment thereof.

Thus, the RNA construct may comprise an RNA nucleotide sequence of SEQ ID No: 18, as follows:

[SEQ ID No: 18]
AUGCAGAUCAUCACCACAGCCCUGGUGUGCUUGCUGCUAGCUGGG
AUGUGGCCGGAAGAUGUGGACAGCAAGAGCAUGCAGGUACCCUUC
UCCAGAUGUUGCUUCUCAUUUGCGGAGCAAGAGAUUCCCCUGAGG
GCAAUCCUGUGUUACAGAAAUACCAGCUCCAUCUGCUCCAAUGAG
GGCUUAAUAUUCAAGCUGAAGAGAGGCAAAGAGGCCUGCGCCUUG
GACACAGUUGGAUGGGUUCAGAGGCACAGAAAAAUGCUGAGGCAC
UGCCCGUCAAAAAGAAAA

Furthermore, preferably the RNA construct comprises an RNA nucleotide sequence substantially as set out in SEQ ID No: 18, or a variant or fragment thereof.

In one embodiment, the at least one ISP may be CCL2 (NCBI Reference Sequence: NM_002982.3; UniProtKB—P13500 (CCL2_HUMAN)), or an orthologue thereof. One embodiment of the polypeptide sequence of CCL2 (signal peptide is 1-23) is represented herein as SEQ ID No: 163, as follows:

[SEQ ID No: 163]
MKVSAALLCLLLIAATFIPQGLAQPDAINAPVTCCYNFTNRKISV
QRLASYRRITSSKCPKEAVIFKTIVAKEICADPKQKWVQDSMDHL
DKQTQTPKT

Therefore, preferably the RNA construct of any aspect comprises a nucleotide sequence which encodes an amino acid sequence substantially as set out in SEQ ID No: 163, or a variant or fragment thereof.

In one embodiment, the CCL2 polypeptide is encoded by the DNA nucleotide sequence of SEQ ID No: 164, as follows:

[SEQ ID No: 164]
ATGAAAGTCTCTGCCGCCCTTCTGTGCCTGCTGCTCATAGCAGCC
ACCTTCATTCCCCAAGGGCTCGCTCAGCCAGATGCAATCAATGCC
CCAGTCACCTGCTGTTATAACTTCACCAATAGGAAGATCTCAGTG
CAGAGGCTCGCGAGCTATAGAAGAATCACCAGCAGCAAGTGTCCC
AAAGAAGCTGTGATCTTCAAGACCATTGTGGCCAAGGAGATCTGT
GCTGACCCCAAGCAGAAGTGGGTTCAGGATTCCATGGACCACCTG
GACAAGCAAACCCAAACTCCGAAGACT

Accordingly, preferably the CCL2 polypeptide is encoded by the DNA nucleotide sequence substantially as set out in SEQ ID NO: 164, or a variant or fragment thereof.

Thus, the RNA construct may comprise an RNA nucleotide sequence of SEQ ID No: 165, as follows:

[SEQ ID No: 165]
AUGAAAGUCUCUGCCGCCCUUCUGUGCCUGCUGCUCAUAGCAGCC
ACCUUCAUUCCCCAAGGGCUCGCUCAGCCAGAUGCAAUCAAUGCC
CCAGUCACCUGCUGUUAUAACUUCACCAAUAGGAAGAUCUCAGUG
CAGAGGCUCGCGAGCUAUAGAAGAAUCACCAGCAGCAAGUGUCCC
AAAGAAGCUGUGAUCUUCAAGACCAUUGUGGCCAAGGAGAUCUGU
GCUGACCCCAAGCAGAAGUGGGUUCAGGAUUCCAUGGACCACCUG
GACAAGCAAACCCAAACUCCGAAGACU

Furthermore, preferably the RNA construct comprises an RNA nucleotide sequence substantially as set out in SEQ ID No: 165, or a variant or fragment thereof.

In one embodiment, the at least one ISP may be CCL3 (NCBI Reference Sequence: NM_002983.2; UniProtKB—P10147 (CCL3_HUMAN)), or an orthologue thereof. One embodiment of the polypeptide sequence of CCL3 (signal peptide is 1-23) is represented herein as SEQ ID No: 166, as follows:

[SEQ ID No: 166]
MQVSTAALAVLLCTMALCNQFSASLAADTPTACCFSYTSRQIPQN
FIADYFETSSQCSKPGVIFLTKRSRQVCADPSEEWVQKYVSDLEL
SA

Therefore, preferably the RNA construct of any aspect comprises a nucleotide sequence which encodes an amino acid sequence substantially as set out in SEQ ID No: 166, or a variant or fragment thereof.

In one embodiment, the CCL3 polypeptide is encoded by the DNA nucleotide sequence of SEQ ID No: 167, as follows:

[SEQ ID No: 167]
ATGCAGGTCTCCACTGCTGCCCTTGCTGTCCTCCTCTGCACCATG
GCTCTCTGCAACCAGTTCTCTGCATCACTTGCTGCTGACACGCCG
ACCGCCTGCTGCTTCAGCTACACCTCCCGGCAGATTCCACAGAAT
TTCATAGCTGACTACTTTGAGACGAGCAGCCAGTGCTCCAAGCCC
GGTGTCATCTTCCTAACCAAGCGAAGCCGGCAGGTCTGTGCTGAC
CCCAGTGAGGAGTGGGTCCAGAAATATGTCAGCGACCTGGAGCTG
AGTGCC

Accordingly, preferably the CCL2 polypeptide is encoded by the DNA nucleotide sequence substantially as set out in SEQ ID NO: 167, or a variant or fragment thereof.

Thus, the RNA construct may comprise an RNA nucleotide sequence of SEQ ID No: 168, as follows:

[SEQ ID No: 168]
AUGCAGGUCUCCACUGCUGCCCUUGCUGUCCUCCUCUGCACCAUG
GCUCUCUGCAACCAGUUCUCUGCAUCACUUGCUGCUGACACGCCG
ACCGCCUGCUGCUUCAGCUACACCUCCCGGCAGAUUCCACAGAAU
UUCAUAGCUGACUACUUUGAGACGAGCAGCCAGUGCUCCAAGCCC
GGUGUCAUCUUCCUAACCAAGCGAAGCCGGCAGGUCUGUGCUGAC
CCCAGUGAGGAGUGGGUCCAGAAAUAUGUCAGCGACCUGGAGCUG
AGUGCC

Furthermore, preferably the RNA construct comprises an RNA nucleotide sequence substantially as set out in SEQ ID No: 168, or a variant or fragment thereof.

In one embodiment, the at least one ISP may be CCL20 (NCBI Reference Sequence: NM_004591.3; UniProtKB—P78556 (CCL20_HUMAN)), or an orthologue thereof. One embodiment of the polypeptide sequence of CCL20 (signal peptide is 1-26 is represented herein as SE ID No: 19, as follows:

[SEQ ID No: 19]
MCCTKSLLLAALMSVLLLHLCGESEAASNFDCCLGYTDRILHPKF
IVGFTRQLANEGCDINAIIFHTKKKLSVCANPKQTWVKYIVRLLS
KKVKNM

Therefore, preferably the RNA construct of any aspect comprises a nucleotide sequence which encodes an amino acid sequence substantially as set out in SEQ ID No: 19, or a variant or fragment thereof.

In one embodiment, the CCL20 polypeptide is encoded by the DNA nucleotide sequence of SEQ ID No: 20, as follows:

[SEQ ID No: 20]
ATGTGCTGTACCAAGAGTTTGCTCCTGGCTGCTTTGATGTCAGTG
CTGCTACTCCACCTCTGCGGCGAATCAGAAGCAGCAAGCAACTTT
GACTGCTGTCTTGGATACACAGACCGTATTCTTCATCCTAAATTT
ATTGTGGGCTTCACACGGCAGCTGGCCAATGAAGGCTGTGACATC
AATGCTATCATCTTTCACACAAAGAAAAAGTTGTCTGTGTGCGCA
AATCCAAAACAGACTTGGGTGAAATATATTGTGCGTCTCCTCAGT
AAAAAAGTCAAGAACATG

Accordingly, preferably the CCL20 polypeptide is encoded by the DNA nucleotide sequence substantially as set out in SEQ ID NO: 20, or a variant or fragment thereof.

Thus, the RNA construct may comprise an RNA nucleotide sequence of SEQ ID No: 21, as follows:

[SEQ ID No: 21]
AUGUGCUGUACCAAGAGUUUGCUCCUGGCUGCUUUGAUGUCAGUG
CUGCUACUCCACCUCUGCGGCGAAUCAGAAGCAGCAAGCAACUUU
GACUGCUGUCUUGGAUACACAGACCGUAUUCUUCAUCCUAAAUUU
AUUGUGGGCUUCACACGGCAGCUGGCCAAUGAAGGCUGUGACAUC
AAUGCUAUCAUCUUUCACACAAAGAAAAAGUUGUCUGUGUGCGCA
AAUCCAAAACAGACUUGGGUGAAAUAUAUUGUGCGUCUCCUCAGU
AAAAAAGUCAAGAACAUG

Furthermore, preferably the RNA construct comprises an RNA nucleotide sequence substantially as set out in SEQ ID No: 21, or a variant or fragment thereof.

In one embodiment, the at least one ISP may be CCL21 (NCBI Reference Sequence: NM_002989.4; UniProtKB—O00585 (CCL21_HUMAN)), or an orthologue thereof. One embodiment of the polypeptide sequence of CCL21 (signal peptide is 1-23) is represented herein as SEQ ID No: 22, as follows:

[SEQ ID No: 22]
MAQSLALSLLILVLAFGIPRTQGSDGGAQDCCLKYSQRKIPAKVV
RSYRKQEPSLGCSIPAILFLPRKRSQAELCADPKELWVQQLMQHL
DKTPSPQKPAQGCRKDRGASKTGKKGKGSKGCKRTERSQTPKGP

Therefore, preferably the RNA construct of any aspect comprises a nucleotide sequence which encodes an amino acid sequence substantially as set out in SEQ ID No: 22, or a variant or fragment thereof.

In one embodiment, the CCL21 polypeptide is encoded by the DNA nucleotide sequence of SEQ ID No: 23, as follows:

[SEQ ID No: 23]
ATGGCTCAGTCACTGGCTCTGAGCCTCCTTATCCTGGTTCTGGCC
TTTGGCATCCCCAGGACCCAAGGCAGTGATGGAGGGGCTCAGGAC
TGTTGCCTCAAGTACAGCCAAAGGAAGATTCCCGCCAAGGTTGTC
CGCAGCTACCGGAAGCAGGAACCAAGCTTAGGCTGCTCCATCCCA
GCTATCCTGTTCTTGCCCCGCAAGCGCTCTCAGGCAGAGCTATGT
GCAGACCCAAAGGAGCTCTGGGTGCAGCAGCTGATGCAGCATCTG
GACAAGACACCATCCCCACAGAAACCAGCCCAGGGCTGCAGGAAG
GACAGGGGGGCCTCCAAGACTGGCAAGAAAGGAAAGGGCTCCAAA
GGCTGCAAGAGGACTGAGCGGTCACAGACCCCTAAAGGGCCA

Accordingly, preferably the CCL21 polypeptide is encoded by the DNA nucleotide sequence substantially as set out in SEQ ID NO: 23, or a variant or fragment thereof.

Thus, the RNA construct may comprise an RNA nucleotide sequence of SEQ ID No: 24, as follows:

[SEQ ID No: 24]
AUGGCUCAGUCACUGGCUCUGAGCCUCCUUAUCCUGGUUCUGGCC
UUUGGCAUCCCCAGGACCCAAGGCAGUGAUGGAGGGGCUCAGGAC
UGUUGCCUCAAGUACAGCCAAAGGAAGAUUCCCGCCAAGGUUGUC
CGCAGCUACCGGAAGCAGGAACCAAGCUUAGGCUGCUCCAUCCCA
GCUAUCCUGUUCUUGCCCCGCAAGCGCUCUCAGGCAGAGCUAUGU
GCAGACCCAAAGGAGCUCUGGGUGCAGCAGCUGAUGCAGCAUCUG
GACAAGACACCAUCCCCACAGAAACCAGCCCAGGGCUGCAGGAAG
GACAGGGGGGCCUCCAAGACUGGCAAGAAAGGAAAGGGCUCCAAA
GGCUGCAAGAGGACUGAGCGGUCACAGACCCCUAAAGGGCCA

Furthermore, preferably the RNA construct comprises an RNA nucleotide sequence substantially as set out in SEQ ID No: 24, or a variant or fragment thereof.

In a preferred embodiment, the ISP is a delta chemokine. Hence, in one embodiment, the at least one ISP may be CX3CL1 or Fractaikine (NCBI Reference Sequence: NM_002996.6; UniProtKB—P78423 (X3CL1_HUMAN)), or an orthologue thereof. One embodiment of the polypeptide sequence of CX3CL1 (signal peptide is 1-24) is represented herein as SEQ ID No: 25, as follows:

[SEQ ID No: 25]
MAPISLSWLLRLATFCHLTVLLAGQHHGVTKCNITCSKMTSKIPV
ALLIHYQQNQASCGKRAIILETRQHRLFCADPKEQWVKDAMQHLD
RQAAALTRNGGTFEKQIGEVKPRTTPAAGGMDESVVLEPEATGES
SSLEPTPSSQEAQRALGTSPELPTGVTGSSGTRLPPTPKAQDGGP
VGTELFRVPPVSTAATWQSSAPHQPGPSLWAEAKTSEAPSTQDPS
TQASTASSPAPEENAPSEGQRVWGQGQSPRPENSLEREEMGPVPA
HTDAFQDWGPGSMAHVSVVPVSSEGTPSREPVASGSWTPKAEEPI
HATMDPQRLGVLITPVPDAQAATRRQAVGLLAFLGLLFCLGVAMF
TYQSLQGCPRKMAGEMAEGLRYIPRSCGSNSYVLVPV

Therefore, preferably the RNA construct of any aspect comprises a nucleotide sequence which encodes an amino acid sequence substantially as set out in SEQ ID No: 25, or a variant or fragment thereof.

In one embodiment, the CX3CL1 polypeptide is encoded by the DNA nucleotide sequence of SEQ ID No: 26, as follows:

[SEQ ID No: 26]
ATGGCTCCGATATCTCTGTCGTGGCTGCTCCGCTTGGCCACCTTC
TGCCATCTGACTGTCCTGCTGGCTGGACAGCACCACGGTGTGACG
AAATGCAACATCACGTGCAGCAAGATGACATCAAAGATACCTGTA
GCTTTGCTCATCCACTATCAACAGAACCAGGCATCATGCGGCAAA
CGCGCAATCATCTTGGAGACGAGACAGCACAGGCTGTTCTGTGCC
GACCCGAAGGAGCAATGGGTCAAGGACGCGATGCAGCATCTGGAC
CGCCAGGCTGCTGCCCTAACTCGAAATGGCGGCACCTTCGAGAAG
CAGATCGGCGAGGTGAAGCCCAGGACCACCCCTGCCGCCGGGGGA
ATGGACGAGTCTGTGGTCCTGGAGCCCGAAGCCACAGGCGAAAGC
AGTAGCCTGGAGCCGACTCCTTCTTCCCAGGAAGCACAGAGGGCC
CTGGGGACCTCCCCAGAGCTGCCGACGGGCGTGACTGGTTCCTCA
GGGACCAGGCTCCCCCCGACGCCAAAGGCTCAGGATGGAGGGCCT
GTGGGCACGGAGCTTTTCCGAGTGCCTCCCGTCTCCACTGCCGCC
ACGTGGCAGAGTTCTGCTCCCCACCAACCTGGGCCCAGCCTCTGG
GCTGAGGCAAAGACCTCTGAGGCCCCGTCCACCCAGGACCCCTCC
ACCCAGGCCTCCACTGCGTCCTCCCCAGCCCCAGAGGAGAATGCT
CCGTCTGAAGGCCAGCGTGTGTGGGGTCAGGGACAGAGCCCCAGG
CCAGAGAACTCTCTGGAGCGGGAGGAGATGGGTCCCGTGCCAGCG
CACACGGATGCCTTCCAGGACTGGGGGCCTGGCAGCATGGCCCAC
GTCTCTGTGGTCCCTGTCTCCTCAGAAGGGACCCCCAGCAGGGAG
CCAGTGGCTTCAGGCAGCTGGACCCCTAAGGCTGAGGAACCCATC
CATGCCACCATGGACCCCCAGAGGCTGGGCGTCCTTATCACTCCT
GTCCCTGACGCCCAGGCTGCCACCCGGAGGCAGGCGGTGGGGCTG
CTGGCCTTCCTTGGCCTCCTCTTCTGCCTGGGGGTGGCCATGTTC
ACCTACCAGAGCCTCCAGGGCTGCCCTCGAAAGATGGCAGGAGAG
ATGGCGGAGGGCCTTCGCTACATCCCCCGGAGCTGTGGTAGTAAT
TCATATGTCCTGGTGCCCGTG

Accordingly, preferably the CX3CL1 polypeptide is encoded by the DNA nucleotide sequence substantially as set out in SEQ ID NO: 26, or a variant or fragment thereof.

Thus, the RNA construct may comprise an RNA nucleotide sequence of SEQ ID No: 27, as follows:

[SEQ ID No: 27]
AUGGCUCCGAUAUCUCUGUCGUGGCUGCUCCGCUUGGCCACCUUC
UGCCAUCUGACUGUCCUGCUGGCUGGACAGCACCACGGUGUGACG
AAAUGCAACAUCACGUGCAGCAAGAUGACAUCAAAGAUACCUGUA
GCUUUGCUCAUCCACUAUCAACAGAACCAGGCAUCAUGCGGCAAA
CGCGCAAUCAUCUUGGAGACGAGACAGCACAGGCUGUUCUGUGCC
GACCCGAAGGAGCAAUGGGUCAAGGACGCGAUGCAGCAUCUGGAC
CGCCAGGCUGCUGCCCUAACUCGAAAUGGCGGCACCUUCGAGAAG
CAGAUCGGCGAGGUGAAGCCCAGGACCACCCCUGCCGCCGGGGGA
AUGGACGAGUCUGUGGUCCUGGAGCCCGAAGCCACAGGCGAAAGC
AGUAGCCUGGAGCCGACUCCUUCUUCCCAGGAAGCACAGAGGGCC
CUGGGGACCUCCCCAGAGCUGCCGACGGGCGUGACUGGUUCCUCA
GGGACCAGGCUCCCCCCGACGCCAAAGGCUCAGGAUGGAGGGCCU
GUGGGCACGGAGCUUUUCCGAGUGCCUCCCGUCUCCACUGCCGCC
ACGUGGCAGAGUUCUGCUCCCCACCAACCUGGGCCCAGCCUCUGG
GCUGAGGCAAAGACCUCUGAGGCCCCGUCCACCCAGGACCCCUCC
ACCCAGGCCUCCACUGCGUCCUCCCCAGCCCCAGAGGAGAAUGCU
CCGUCUGAAGGCCAGCGUGUGUGGGGUCAGGGACAGAGCCCCAGG
CCAGAGAACUCUCUGGAGCGGGAGGAGAUGGGUCCCGUGCCAGCG
CACACGGAUGCCUUCCAGGACUGGGGGCCUGGCAGCAUGGCCCAC
GUCUCUGUGGUCCCUGUCUCCUCAGAAGGGACCCCCAGCAGGGAG
CCAGUGGCUUCAGGCAGCUGGACCCCUAAGGCUGAGGAACCCAUC
CAUGCCACCAUGGACCCCCAGAGGCUGGGCGUCCUUAUCACUCCU
GUCCCUGACGCCCAGGCUGCCACCCGGAGGCAGGCGGUGGGGCUG
CUGGCCUUCCUUGGCCUCCUCUUCUGCCUGGGGGUGGCCAUGUUC
ACCUACCAGAGCCUCCAGGGCUGCCCUCGAAAGAUGGCAGGAGAG
AUGGCGGAGGGCCUUCGCUACAUCCCCCGGAGCUGUGGUAGUAAU
UCAUAUGUCCUGGUGCCCGUG

Furthermore, preferably the RNA construct comprises an RNA nucleotide sequence substantially as set out in SEQ ID No: 27, or a variant or fragment thereof.

In one embodiment, the at least one ISP may be CX3CL1 or Fractalkine (Secreted Form; NCBI Reference Sequence: NM_002996.6; UniProtKB—P78423 (X3CL1_HUMAN)), or an orthologue thereof. One embodiment of the polypeptide sequence of secreted CX3CL1 (signal is 1-20) is represented herein as SEQ ID No: 28, as follows:

[SEQ ID No: 28]
MDRAKLLLLLLLLLLPQAQAQHHGVTKCNITCSKMTSKIPVALLI
HYQQNQASCGKRAIILETRQHRLFCADPKEQWVKDAMQHLDRQAA
ALTRNGGTFEKQIGEVKPRTTPAAGGMDESVVLEPEATGESSSLE
PTPSSQEAQRALGTSPELPTGVTGSSGTRLPPTPKAQDGGPVGTE
LFRVPPVSTAATWQSSAPHQPGPSLWAEAKTSEAPSTQDPSTQAS
TASSPAPEENAPSEGQRVWGQGQSPRPENSLEREEMGPVPAHTDA
FQDWGPGSMAHVSVVPVSSEGTPSREPVASGSWTPKAEEPIHATM
DPQRLGVLITPVPDAQAATRRQ

Therefore, preferably the RNA construct of any aspect comprises a nucleotide sequence which encodes an amino acid sequence substantially as set out in SEQ ID No: 28, or a variant or fragment thereof.

In one embodiment, the secreted CX3CL1 polypeptide is encoded by the DNA nucleotide sequence of SEQ ID No: 29, as follows:

[SEQ ID No: 29]
ATGGACAGAGCCAAGCTGCTGCTGTTGCTGCTCCTTCTGCTGCTG
CCTCAGGCTCAAGCTCAGCACCACGGTGTGACGAAATGCAACATC
ACGTGCAGCAAGATGACATCAAAGATACCTGTAGCTTTGCTCATC
CACTATCAACAGAACCAGGCATCATGCGGCAAACGCGCAATCATC
TTGGAGACGAGACAGCACAGGCTGTTCTGTGCCGACCCGAAGGAG
CAATGGGTCAAGGACGCGATGCAGCATCTGGACCGCCAGGCTGCT
GCCCTAACTCGAAATGGCGGCACCTTCGAGAAGCAGATCGGCGAG
GTGAAGCCCAGGACCACCCCTGCCGCCGGGGGAATGGACGAGTCT
GTGGTCCTGGAGCCCGAAGCCACAGGCGAAAGCAGTAGCCTGGAG
CCGACTCCTTCTTCCCAGGAAGCACAGAGGGCCCTGGGGACCTCC
CCAGAGCTGCCGACGGGCGTGACTGGTTCCTCAGGGACCAGGCTC
CCCCCGACGCCAAAGGCTCAGGATGGAGGGCCTGTGGGCACGGAG
CTTTTCCGAGTGCCTCCCGTCTCCACTGCCGCCACGTGGCAGAGT
TCTGCTCCCCACCAACCTGGGCCCAGCCTCTGGGCTGAGGCAAAG
ACCTCTGAGGCCCCGTCCACCCAGGACCCCTCCACCCAGGCCTCC
ACTGCGTCCTCCCCAGCCCCAGAGGAGAATGCTCCGTCTGAAGGC
CAGCGTGTGTGGGGTCAGGGACAGAGCCCCAGGCCAGAGAACTCT
CTGGAGCGGGAGGAGATGGGTCCCGTGCCAGCGCACACGGATGCC
TTCCAGGACTGGGGGCCTGGCAGCATGGCCCACGTCTCTGTGGTC
CCTGTCTCCTCAGAAGGGACCCCCAGCAGGGAGCCAGTGGCTTCA
GGCAGCTGGACCCCTAAGGCTGAGGAACCCATCCATGCCACCATG
GACCCCCAGAGGCTGGGCGTCCTTATCACTCCTGTCCCTGACGCC
CAGGCTGCCACCCGGAGGCAG

Accordingly, preferably the secreted CX3CL1 polypeptide is encoded by the DNA nucleotide sequence substantially as set out in SEQ ID NO: 29, or a variant or fragment thereof.

Thus, the RNA construct may comprise an RNA nucleotide sequence of SEQ ID No: 30, as follows:

[SEQ ID No: 30]
AUGGACAGAGCCAAGCUGCUGCUGUUGCUGCUCCUUCUGCUGCUG
CCUCAGGCUCAAGCUCAGCACCACGGUGUGACGAAAUGCAACAUC
ACGUGCAGCAAGAUGACAUCAAAGAUACCUGUAGCUUUGCUCAUC
CACUAUCAACAGAACCAGGCAUCAUGCGGCAAACGCGCAAUCAUC
UUGGAGACGAGACAGCACAGGCUGUUCUGUGCCGACCCGAAGGAG
CAAUGGGUCAAGGACGCGAUGCAGCAUCUGGACCGCCAGGCUGCU
GCCCUAACUCGAAAUGGCGGCACCUUCGAGAAGCAGAUCGGCGAG
GUGAAGCCCAGGACCACCCCUGCCGCCGGGGGAAUGGACGAGUCU
GUGGUCCUGGAGCCCGAAGCCACAGGCGAAAGCAGUAGCCUGGAG
CCGACUCCUUCUUCCCAGGAAGCACAGAGGGCCCUGGGGACCUCC
CCAGAGCUGCCGACGGGCGUGACUGGUUCCUCAGGGACCAGGCUC
CCCCCGACGCCAAAGGCUCAGGAUGGAGGGCCUGUGGGCACGGAG
CUUUUCCGAGUGCCUCCCGUCUCCACUGCCGCCACGUGGCAGAGU
UCUGCUCCCCACCAACCUGGGCCCAGCCUCUGGGCUGAGGCAAAG
ACCUCUGAGGCCCCGUCCACCCAGGACCCCUCCACCCAGGCCUCC
ACUGCGUCCUCCCCAGCCCCAGAGGAGAAUGCUCCGUCUGAAGGC
CAGCGUGUGUGGGGUCAGGGACAGAGCCCCAGGCCAGAGAACUCU
CUGGAGCGGGAGGAGAUGGGUCCCGUGCCAGCGCACACGGAUGCC
UUCCAGGACUGGGGGCCUGGCAGCAUGGCCCACGUCUCUGUGGUC
CCUGUCUCCUCAGAAGGGACCCCCAGCAGGGAGCCAGUGGCUUCA
GGCAGCUGGACCCCUAAGGCUGAGGAACCCAUCCAUGCCACCAUG
GACCCCCAGAGGCUGGGCGUCCUUAUCACUCCUGUCCCUGACGCC
CAGGCUGCCACCCGGAGGCAG

Furthermore, preferably the RNA construct comprises an RNA nucleotide sequence substantially as set out in SEQ ID No: 30, or a variant or fragment thereof.

In a preferred embodiment, the ISP is a gamma chemokine. In one embodiment, the at least one ISP may be XCL1 (NCBI Reference Sequence: NM_002995.3; UniProtKB—P47992 (XCL1_HUMAN)), or an orthologue thereof. One embodiment of the polypeptide sequence of the XCL1 (signal is 1-21) is represented herein as SEQ ID No: 31, as follows:

[SEQ ID No: 31]
MRLLILALLGICSLTAYIVEGVGSEVSDKRTCVSLTTQRLPVSRI
KTYTITEGSLRAVIFITKRGLKVCADPQATWVRDVVRSMDRKSNT
RNNMIQTKPTGTQQSTNTAVTLTG

Therefore, preferably the RNA construct of any aspect comprises a nucleotide sequence which encodes an amino acid sequence substantially as set out in SEQ ID No: 31, or a variant or fragment thereof.

In one embodiment, the XCL1 polypeptide is encoded by the DNA nucleotide sequence of SEQ ID No: 32, as follows:

[SEQ ID No: 32]
ATGAGACTTCTCATCCTGGCCCTCCTTGGCATCTGCTCTCTCACT
GCATACATTGTGGAAGGTGTAGGGAGTGAAGTCTCAGATAAGAGG
ACCTGTGTGAGCCTCACTACCCAGCGACTGCCGGTTAGCAGAATC
AAGACCTACACCATCACGGAAGGCTCCTTGAGAGCAGTAATTTTT
ATTACCAAACGTGGCCTAAAAGTCTGTGCTGATCCACAAGCCACA
TGGGTGAGAGACGTGGTCAGGAGCATGGACAGGAAATCCAACACC
AGAAATAACATGATCCAGACCAAGCCAACAGGAACCCAGCAATCG
ACCAATACAGCTGTGACTCTGACTGGC

Accordingly, preferably the XCL1 polypeptide is encoded by the DNA nucleotide sequence substantially as set out in SEQ ID NO: 32, or a variant or fragment thereof.

Thus, the RNA construct may comprise an RNA nucleotide sequence of SEQ ID No: 33, as follows:

[SEQ ID NO: 33]
AUGAGACUUCUCAUCCUGGCCCUCCUUGGCAUCUGCUCUCUCACU
GCAUACAUUGUGGAAGGUGUAGGGAGUGAAGUCUCAGAUAAGAGG
ACCUGUGUGAGCCUCACUACCCAGCGACUGCCGGUUAGCAGAAUC
AAGACCUACACCAUCACGGAAGGCUCCUUGAGAGCAGUAAUUUUU
AUUACCAAACGUGGCCUAAAAGUCUGUGCUGAUCCACAAGCCACA
UGGGUGAGAGACGUGGUCAGGAGCAUGGACAGGAAAUCCAACACC
AGAAAUAACAUGAUCCAGACCAAGCCAACAGGAACCCAGCAAUCG
ACCAAUACAGCUGUGACUCUGACUGGC

Furthermore, preferably the RNA construct comprises an RNA nucleotide sequence substantially as set out in SEQ ID No: 33, or a variant or fragment thereof.

In one embodiment, the at least one ISP may be XCL2 (NCBI Reference Sequence: NM_003175.4; UniProtKB—Q9UBD3 (XCL2_HUMAN)), or an orthologue thereof. One embodiment of the polypeptide sequence of XCL2 (signal 1-21) is represented herein as SEQ ID No: 34, as follows:

[SEQ ID NO: 34]
MRLLILALLGICSLTAYIVEGVGSEVSHRRTCVSLTTQRLPVSRI
KTYTITEGSLRAVIFITKRGLKVCADPQATWVRDVVRSMDRKSNT
RNNMIQTKPTGTQQSTNTAVTLTG

Therefore, preferably the RNA construct of any aspect comprises a nucleotide sequence which encodes an amino acid sequence substantially as set out in SEQ ID No: 34, or a variant or fragment thereof.

In one embodiment, the XCL2 polypeptide is encoded by the DNA nucleotide sequence of SEQ ID No: 35, as follows:

[SEQ ID NO: 35]
ATGAGACTTCTCATCCTGGCCCTCCTTGGCATCTGCTCTCTCACT
GCATACATTGTGGAAGGTGTAGGGAGTGAAGTCTCACATAGGAGG
ACCTGTGTGAGCCTCACTACCCAGCGACTGCCAGTTAGCAGAATC
AAGACCTACACCATCACGGAAGGCTCCTTGAGAGCAGTAATTTTT
ATTACCAAACGTGGCCTAAAAGTCTGTGCTGATCCACAAGCCACG
TGGGTGAGAGACGTGGTCAGGAGCATGGACAGGAAATCCAACACC
AGAAATAACATGATCCAGACCAAGCCAACAGGAACCCAGCAATCG
ACCAATACAGCTGTGACCCTGACTGGC

Accordingly, preferably the XCL2 polypeptide is encoded by the DNA nucleotide sequence substantially as set out in SEQ ID NO: 35, or a variant or fragment thereof.

Thus, the RNA construct may comprise an RNA nucleotide sequence of SEQ ID No: 36, as follows:

[SEQ ID NO: 36]
AUGAGACUUCUCAUCCUGGCCCUCCUUGGCAUCUGCUCUCUCACU
GCAUACAUUGUGGAAGGUGUAGGGAGUGAAGUCUCACAUAGGAGG
ACCUGUGUGAGCCUCACUACCCAGCGACUGCCAGUUAGCAGAAUC
AAGACCUACACCAUCACGGAAGGCUCCUUGAGAGCAGUAAUUUUU
AUUACCAAACGUGGCCUAAAAGUCUGUGCUGAUCCACAAGCCACG
UGGGUGAGAGACGUGGUCAGGAGCAUGGACAGGAAAUCCAACACC
AGAAAUAACAUGAUCCAGACCAAGCCAACAGGAACCCAGCAAUCG
ACCAAUACAGCUGUGACCCUGACUGGC

Furthermore, preferably the RNA construct comprises an RNA nucleotide sequence substantially as set out in SEQ ID No: 36, or a variant or fragment thereof.

Alternatively, the ISP may be an interleukin. The interleukin may be any of IL1-38, or an orthologue thereof. In one embodiment, the at least one ISP may be IL-1b (NCBI Reference Sequence: NM_000576.3; UniProtKB—P01584 (IL1B_HUMAN)), or an orthologue thereof. One embodiment of the polypeptide sequence of IL-1b (the active form, which may benefit from the addition of a secretion signal) is represented herein as SEQ ID No: 37, as follows:

[SEQ ID NO: 37]
MAPVRSLNCTLRDSQQKSLVMSGPYELKALHLQGQDMEQQVVFSM
SFVQGEESNDKIPVALGLKEKNLYLSCVLKDDKPTLQLESVDPKN
YPKKKMEKRFVENKIEINNKLEFESAQFPNWYISTSQAENMPVFL
GGTKGGQDITDFTMQFVSS

Therefore, preferably the RNA construct of any aspect comprises a nucleotide sequence which encodes an amino acid sequence substantially as set out in SEQ ID No: 37, or a variant or fragment thereof.

In one embodiment, the IL-1b polypeptide is encoded by the DNA nucleotide sequence of SEQ ID No: 38, as follows:

[SEQ ID NO: 38]
ATGGCACCTGTACGATCACTGAACTGCACGCTCCGGGACTCACAG
CAAAAAAGCTTGGTGATGTCTGGTCCATATGAACTGAAAGCTCTC
CACCTCCAGGGACAGGATATGGAGCAACAAGTGGTGTTCTCCATG
TCCTTTGTACAAGGAGAAGAAAGTAATGACAAAATACCTGTGGCC
TTGGGCCTCAAGGAAAAGAATCTGTACCTGTCCTGCGTGTTGAAA
GATGATAAGCCCACTCTACAGCTGGAGAGTGTAGATCCCAAAAAT
TACCCAAAGAAGAAGATGGAAAAGCGATTTGTCTTCAACAAGATA
GAAATCAATAACAAGCTGGAATTTGAGTCTGCCCAGTTCCCCAAC
TGGTACATCAGCACCTCTCAAGCAGAAAACATGCCCGTCTTCCTG
GGAGGGACCAAAGGCGGCCAGGATATAACTGACTTCACCATGCAA
TTTGTGTCTTCC

Accordingly, preferably the IL-1b polypeptide is encoded by the DNA nucleotide sequence substantially as set out in SEQ ID NO: 38, or a variant or fragment thereof.

Thus, the RNA construct may comprise an RNA nucleotide sequence of SEQ ID No: 39, as follows:

[SEQ ID NO: 39]
AUGGCACCUGUACGAUCACUGAACUGCACGCUCCGGGACUCACAG
CAAAAAAGCUUGGUGAUGUCUGGUCCAUAUGAACUGAAAGCUCUC
CACCUCCAGGGACAGGAUAUGGAGCAACAAGUGGUGUUCUCCAUG
UCCUUUGUACAAGGAGAAGAAAGUAAUGACAAAAUACCUGUGGCC
UUGGGCCUCAAGGAAAAGAAUCUGUACCUGUCCUGCGUGUUGAAA
GAUGAUAAGCCCACUCUACAGCUGGAGAGUGUAGAUCCCAAAAAU
UACCCAAAGAAGAAGAUGGAAAAGCGAUUUGUCUUCAACAAGAUA
GAAAUCAAUAACAAGCUGGAAUUUGAGUCUGCCCAGUUCCCCAAC
UGGUACAUCAGCACCUCUCAAGCAGAAAACAUGCCCGUCUUCCUG
GGAGGGACCAAAGGCGGCCAGGAUAUAACUGACUUCACCAUGCAA
UUUGUGUCUUCC

Furthermore, preferably the RNA construct comprises an RNA nucleotide sequence substantially as set out in SEQ ID No: 39, or a variant or fragment thereof.

In one embodiment, the at least one ISP may be IL-1b with an enhanced secretion signal (NCBI Reference Sequence: NM_000576.3; UniProtKB—P01584 (IL1B_HUMAN)), or an orthologue thereof. One embodiment of the polypeptide sequence of IL-1b (with an added secretion signal) is represented herein as SEQ ID No: 40, as follows:

[SEQ ID NO: 40]
MDRAKLLLLLLLLLLPQAQAAPVRSLNCTLRDSQQKSLVMSGPYE
LKALHLQGQDMEQQVVFSMSFVQGEESNDKIPVALGLKEKNLYLS
CVLKDDKPTLQLESVDPKNYPKKKMEKRFVFNKIEINNKLEFESA
QFPNWYISTSQAENMPVFLGGTKGGQDITDFTMQFVSS

Therefore, preferably the RNA construct of any aspect comprises a nucleotide sequence which encodes an amino acid sequence substantially as set out in SEQ ID No: 40, or a variant or fragment thereof.

In one embodiment, the IL-1b polypeptide with an enhanced secretion signal is encoded by the DNA nucleotide sequence of SEQ ID No: 41, as follows:

[SEQ ID NO: 41]
ATGGACAGAGCCAAGCTGCTGCTGTTGCTGCTCCTTCTGCTGCTG
CCTCAGGCTCAAGCTGCACCTGTACGATCACTGAACTGCACGCTC
CGGGACTCACAGCAAAAAAGCTTGGTGATGTCTGGTCCATATGAA
CTGAAAGCTCTCCACCTCCAGGGACAGGATATGGAGCAACAAGTG
GTGTTCTCCATGTCCTTTGTACAAGGAGAAGAAAGTAATGACAAA
ATACCTGTGGCCTTGGGCCTCAAGGAAAAGAATCTGTACCTGTCC
TGCGTGTTGAAAGATGATAAGCCCACTCTACAGCTGGAGAGTGTA
GATCCCAAAAATTACCCAAAGAAGAAGATGGAAAAGCGATTTGTC
TTCAACAAGATAGAAATCAATAACAAGCTGGAATTTGAGTCTGCC
CAGTTCCCCAACTGGTACATCAGCACCTCTCAAGCAGAAAACATG
CCCGTCTTCCTGGGAGGGACCAAAGGCGGCCAGGATATAACTGAC
TTCACCATGCAATTTGTGTCTTCC

Accordingly, preferably the IL-1b polypeptide with an enhanced secretion signal is encoded by the DNA nucleotide sequence substantially as set out in SEQ ID NO: 41, or a variant or fragment thereof.

Thus, the RNA construct may comprise an RNA nucleotide sequence of SEQ ID No: 42, as follows:

[SEQ ID NO: 42]
AUGGACAGAGCCAAGCUGCUGCUGUUGCUGCUCCUUCUGCUGCUG
CCUCAGGCUCAAGCUGCACCUGUACGAUCACUGAACUGCACGCUC
CGGGACUCACAGCAAAAAAGCUUGGUGAUGUCUGGUCCAUAUGAA
CUGAAAGCUCUCCACCUCCAGGGACAGGAUAUGGAGCAACAAGUG
GUGUUCUCCAUGUCCUUUGUACAAGGAGAAGAAAGUAAUGACAAA
AUACCUGUGGCCUUGGGCCUCAAGGAAAAGAAUCUGUACCUGUCC
UGCGUGUUGAAAGAUGAUAAGCCCACUCUACAGCUGGAGAGUGUA
GAUCCCAAAAAUUACCCAAAGAAGAAGAUGGAAAAGCGAUUUGUC
UUCAACAAGAUAGAAAUCAAUAACAAGCUGGAAUUUGAGUCUGCC
CAGUUCCCCAACUGGUACAUCAGCACCUCUCAAGCAGAAAACAUG
CCCGUCUUCCUGGGAGGGACCAAAGGCGGCCAGGAUAUAACUGAC
UUCACCAUGCAAUUUGUGUCUUCC

Furthermore, preferably the RNA construct comprises an RNA nucleotide sequence substantially as set out in SEQ ID No: 42, or a variant or fragment thereof.

In one embodiment, the at least one ISP may be IL-2 (NCBI Reference Sequence: NM_000586.4; UniProtKB—P60568 (IL2_HUMAN)), or an orthologue thereof. One embodiment of the polypeptide sequence of IL-2 (signal peptide 1-20) is represented herein as SEQ ID No: 43, as follows:

[SEQ ID NO: 43]
MYRMQLLSCIALSLALVINSAPTSSSTKKTQLQLEHLLLDLQMIL
NGINNYKNPKLTRMLTFKFYMPKKATELKHLQCLEEELKPLEEVL
NLAQSKNFHLRPRDLISNINVIVLELKGSETTFMCEYADETATIV
EFLNRWITFCQSIISTLT

Therefore, preferably the RNA construct of any aspect comprises a nucleotide sequence which encodes an amino acid sequence substantially as set out in SEQ ID No: 43, or a variant or fragment thereof.

In one embodiment, the IL-2 polypeptide is encoded by the DNA nucleotide sequence of SEQ ID No: 44, as follows:

[SEQ ID NO: 44]
ATGTACAGGATGCAACTCCTGTCTTGCATTGCACTAAGTCTTGCA
CTTGTCACAAACAGTGCACCTACTTCAAGTTCTACAAAGAAAACA
CAGCTACAACTGGAGCATTTACTGCTGGATTTACAGATGATTTTG
AATGGAATTAATAATTACAAGAATCCCAAACTCACCAGGATGCTC
ACATTTAAGTTTTACATGCCCAAGAAGGCCACAGAACTGAAACAT
CTTCAGTGTCTAGAAGAAGAACTCAAACCTCTGGAGGAAGTGCTA
AATTTAGCTCAAAGCAAAAACTTTCACTTAAGACCCAGGGACTTA
ATCAGCAATATCAACGTAATAGTTCTGGAACTAAAGGGATCTGAA
ACAACATTCATGTGTGAATATGCTGATGAGACAGCAACCATTGTA
GAATTTCTGAACAGATGGATTACCTTTTGTCAAAGCATCATCTCA
ACACTGACT

Accordingly, preferably the IL-2 polypeptide is encoded by the DNA nucleotide sequence substantially as set out in SEQ ID NO: 44, or a variant or fragment thereof.

Thus, the RNA construct may comprise an RNA nucleotide sequence of SEQ ID No: 45, as follows:

[SEQ ID NO: 45]
AUGUACAGGAUGCAACUCCUGUCUUGCAUUGCACUAAGUCUUGCA
CUUGUCACAAACAGUGCACCUACUUCAAGUUCUACAAAGAAAACA
CAGCUACAACUGGAGCAUUUACUGCUGGAUUUACAGAUGAUUUUG
AAUGGAAUUAAUAAUUACAAGAAUCCCAAACUCACCAGGAUGCUC
ACAUUUAAGUUUUACAUGCCCAAGAAGGCCACAGAACUGAAACAU
CUUCAGUGUCUAGAAGAAGAACUCAAACCUCUGGAGGAAGUGCUA
AAUUUAGCUCAAAGCAAAAACUUUCACUUAAGACCCAGGGACUUA
AUCAGCAAUAUCAACGUAAUAGUUCUGGAACUAAAGGGAUCUGAA
ACAACAUUCAUGUGUGAAUAUGCUGAUGAGACAGCAACCAUUGUA
GAAUUUCUGAACAGAUGGAUUACCUUUUGUCAAAGCAUCAUCUCA
ACACUGACU

Furthermore, preferably the RNA construct comprises an RNA nucleotide sequence substantially as set out in SEQ ID No: 45, or a variant or fragment thereof.

In one embodiment, the at least one ISP may be IL-7 (NCBI Reference Sequence: NM_000880.3; UniProtKB—P13232 (IL7_HUMAN)), or an orthologue thereof. One embodiment of the polypeptide sequence of IL-7 (signal peptide is 1-25) is represented herein as SEQ ID No: 169, as follows:

[SEQ ID NO: 169]
MFHVSFRYIFGLPPLILVLLPVASSDCDIEGKDGKQYESVLMVSI
DQLLDSMKEIGSNCLNNEFNFFKRHICDANKEGMFLFRAARKLRQ
FLKMNSTGDFDLHLLKVSEGTTILLNCTGQVKGRKPAALGEAQPT
KSLEENKSLKEQKKLNDLCFLKRLLQEIKTCWNKILMGTKEH

Therefore, preferably the RNA construct of any aspect comprises a nucleotide sequence which encodes an amino acid sequence substantially as set out in SEQ ID No: 169, or a variant or fragment thereof.

In one embodiment, the IL-7 polypeptide is encoded by the DNA nucleotide sequence of SEQ ID No: 170, as follows:

[SEQ ID NO: 170]
ATGTTCCATGTTTCTTTTAGGTATATCTTTGGACTTCCTCCCCTG
ATCCTTGTTCTGTTGCCAGTAGCATCATCTGATTGTGATATTGAA
GGTAAAGATGGCAAACAATATGAGAGTGTTCTAATGGTCAGCATC
GATCAATTATTGGACAGCATGAAAGAAATTGGTAGCAATTGCCTG
AATAATGAATTTAACTTTTTTAAAAGACATATCTGTGATGCTAAT
AAGGAAGGTATGTTTTTATTCCGTGCTGCTCGCAAGTTGAGGCAA
TTTCTTAAAATGAATAGCACTGGTGATTTTGATCTCCACTTATTA
AAAGTTTCAGAAGGCACAACAATACTGTTGAACTGCACTGGCCAG
GTTAAAGGAAGAAAACCAGCTGCCCTGGGTGAAGCCCAACCAACA
AAGAGTTTGGAAGAAAATAAATCTTTAAAGGAACAGAAAAAACTG
AATGACTTGTGTTTCCTAAAGAGACTATTACAAGAGATAAAAACT
TGTTGGAATAAAATTTTGATGGGCACTAAAGAACAC

Accordingly, preferably the IL-7 polypeptide is encoded by the DNA nucleotide sequence substantially as set out in SEQ ID NO: 170, or a variant or fragment thereof.

Thus, the RNA construct may comprise an RNA nucleotide sequence of SEQ ID No: 171, as follows:

[SEQ ID No: 171]
AUGUUCCAUGUUUCUUUUAGGUAUAUCUUUGGACUUCCUCCCCUG
AUCCUUGUUCUGUUGCCAGUAGCAUCAUCUGAUUGUGAUAUUGAA
GGUAAAGAUGGCAAACAAUAUGAGAGUGUUCUAAUGGUCAGCAUC
GAUCAAUUAUUGGACAGCAUGAAAGAAAUUGGUAGCAAUUGCCUG
AAUAAUGAAUUUAACUUUUUUAAAAGACAUAUCUGUGAUGCUAAU
AAGGAAGGUAUGUUUUUAUUCCGUGCUGCUCGCAAGUUGAGGCAA
UUUCUUAAAAUGAAUAGCACUGGUGAUUUUGAUCUCCACUUAUUA
AAAGUUUCAGAAGGCACAACAAUACUGUUGAACUGCACUGGCCAG
GUUAAAGGAAGAAAACCAGCUGCCCUGGGUGAAGCCCAACCAACA
AAGAGUUUGGAAGAAAAUAAAUCUUUAAAGGAACAGAAAAAACUG
AAUGACUUGUGUUUCCUAAAGAGACUAUUACAAGAGAUAAAAACU
UGUUGGAAUAAAAUUUUGAUGGGCACUAAAGAACAC

Furthermore, preferably the RNA construct comprises an RNA nucleotide sequence substantially as set out in SEQ ID No: 171, or a variant or fragment thereof.

In one embodiment, the at least one ISP may be IL-18 (37-93) (NCBI Reference Sequence: NM_001562.4; UniProtKB—Q14116 (IL18_HUMAN)), or an orthologue thereof. One embodiment of the polypeptide sequence of IL-18 (37-93) (which may benefit from the addition of a signal peptide) is represented herein as SEQ ID No: 46, as follows:

[SEQ ID No: 46]
MYFGKLESKLSVIRNLNDQVLFIDQGNRPLFEDMTDSDCRDNAPR
TIFIISMYKDSQPRGMAVTISVKCEKISTLSCENKIISFKEMNPP
DNIKDTKSDIIFFQRSVPGHDNKMQFESSSYEGYFLACEKERDLF
KLILKKEDELGDRSIMFTVQNED

Therefore, preferably the RNA construct of any aspect comprises a nucleotide sequence which encodes an amino acid sequence substantially as set out in SEQ ID No: 46, or a variant or fragment thereof.

In one embodiment, the IL-18 (37-93) polypeptide is encoded by the DNA nucleotide sequence of SEQ ID No: 47, as follows:

[SEQ ID No: 47]
ATGTACTTTGGCAAGCTTGAATCTAAATTATCAGTCATAAGAAAT
TTGAATGACCAAGTTCTCTTCATTGACCAAGGAAATCGGCCTCTA
TTTGAAGATATGACTGATTCTGACTGTAGAGATAATGCACCCCGG
ACCATATTTATTATAAGTATGTATAAAGATAGCCAGCCTAGAGGT
ATGGCTGTAACTATCTCTGTGAAGTGTGAGAAAATTTCAACTCTC
TCCTGTGAGAACAAAATTATTTCCTTTAAGGAAATGAATCCTCCT
GATAACATCAAGGATACAAAAAGTGACATCATATTCTTTCAGAGA
AGTGTCCCAGGACATGATAATAAGATGCAATTTGAATCTTCATCA
TACGAAGGATACTTTCTAGCTTGTGAAAAAGAGAGAGACCTTTTT
AAACTCATTTTGAAAAAAGAGGATGAATTGGGGGATAGATCTATA
ATGTTCACTGTTCAAAACGAAGAC

Accordingly, preferably the IL-18 (37-93) polypeptide is encoded by the DNA nucleotide sequence substantially as set out in SEQ ID NO: 47, or a variant or fragment thereof.

Thus, the RNA construct may comprise an RNA nucleotide sequence of SEQ ID No: 48, as follows:

[SEQ ID No: 48]
AUGUACUUUGGCAAGCUUGAAUCUAAAUUAUCAGUCAUAAGAAAU
UUGAAUGACCAAGUUCUCUUCAUUGACCAAGGAAAUCGGCCUCUA
UUUGAAGAUAUGACUGAUUCUGACUGUAGAGAUAAUGCACCCCGG
ACCAUAUUUAUUAUAAGUAUGUAUAAAGAUAGCCAGCCUAGAGGU
AUGGCUGUAACUAUCUCUGUGAAGUGUGAGAAAAUUUCAACUCUC
UCCUGUGAGAACAAAAUUAUUUCCUUUAAGGAAAUGAAUCCUCCU
GAUAACAUCAAGGAUACAAAAAGUGACAUCAUAUUCUUUCAGAGA
AGUGUCCCAGGACAUGAUAAUAAGAUGCAAUUUGAAUCUUCAUCA
UACGAAGGAUACUUUCUAGCUUGUGAAAAAGAGAGAGACCUUUUU
AAACUCAUUUUGAAAAAAGAGGAUGAAUUGGGGGAUAGAUCUAUA
AUGUUCACUGUUCAAAACGAAGAC

Furthermore, preferably the RNA construct comprises an RNA nucleotide sequence substantially as set out in SEQ ID No: 48, or a variant or fragment thereof.

In one embodiment, the at least one ISP may be IL-18 (37-93) with an enhanced signal sequence (NCBI Reference Sequence: NM_001562.4; UniProtKB—Q14116 (IL18_HUMAN)), or an orthologue thereof. One embodiment of the polypeptide sequence of IL-18 (37-93) (with an added secretion signal) is represented herein as SEQ ID No: 49, as follows:

[SEQ ID No: 49]
MDRAKLLLLLLLLLLPQAQAYFGKLESKLSVIRNLNDQVLFIDQG
NRPLFEDMTDSDCRDNAPRTIFIISMYKDSQPRGMAVTISVKCEK
ISTLSCENKIISFKEMNPPDNIKDTKSDIIFFQRSVPGHDNKMQF
ESSSYEGYFLACEKERDLFKLILKKEDELGDRSIMFTVQNED

Therefore, preferably the RNA construct of any aspect comprises a nucleotide sequence which encodes an amino acid sequence substantially as set out in SEQ ID No: 49, or a variant or fragment thereof.

In one embodiment, the IL-18 (37-93) polypeptide with an enhanced signal sequence is encoded by the DNA nucleotide sequence of SEQ ID No: 50, as follows:

[SEQ ID No: 50]
ATGGACAGAGCCAAGCTGCTGCTGTTGCTGCTCCTTCTGCTGCTG
CCTCAGGCTCAAGCTTACTTTGGCAAGCTTGAATCTAAATTATCA
GTCATAAGAAATTTGAATGACCAAGTTCTCTTCATTGACCAAGGA
AATCGGCCTCTATTTGAAGATATGACTGATTCTGACTGTAGAGAT
AATGCACCCCGGACCATATTTATTATAAGTATGTATAAAGATAGC
CAGCCTAGAGGTATGGCTGTAACTATCTCTGTGAAGTGTGAGAAA
ATTTCAACTCTCTCCTGTGAGAACAAAATTATTTCCTTTAAGGAA
ATGAATCCTCCTGATAACATCAAGGATACAAAAAGTGACATCATA
TTCTTTCAGAGAAGTGTCCCAGGACATGATAATAAGATGCAATTT
GAATCTTCATCATACGAAGGATACTTTCTAGCTTGTGAAAAAGAG
AGAGACCTTTTTAAACTCATTTTGAAAAAAGAGGATGAATTGGGG
GATAGATCTATAATGTTCACTGTTCAAAACGAAGAC

Accordingly, preferably the IL-18 (37-93) polypeptide with an enhanced signal sequence polypeptide is encoded by the DNA nucleotide sequence substantially as set out in SEQ ID NO: 50, or a variant or fragment thereof.

Thus, the RNA construct may comprise an RNA nucleotide sequence of SEQ ID No: 51, as follows:

[SEQ ID No: 51]
AUGGACAGAGCCAAGCUGCUGCUGUUGCUGCUCCUUCUGCUGCUG
CCUCAGGCUCAAGCUUACUUUGGCAAGCUUGAAUCUAAAUUAUCA
GUCAUAAGAAAUUUGAAUGACCAAGUUCUCUUCAUUGACCAAGGA
AAUCGGCCUCUAUUUGAAGAUAUGACUGAUUCUGACUGUAGAGAU
AAUGCACCCCGGACCAUAUUUAUUAUAAGUAUGUAUAAAGAUAGC
CAGCCUAGAGGUAUGGCUGUAACUAUCUCUGUGAAGUGUGAGAAA
AUUUCAACUCUCUCCUGUGAGAACAAAAUUAUUUCCUUUAAGGAA
AUGAAUCCUCCUGAUAACAUCAAGGAUACAAAAAGUGACAUCAUA
UUCUUUCAGAGAAGUGUCCCAGGACAUGAUAAUAAGAUGCAAUUU
GAAUCUUCAUCAUACGAAGGAUACUUUCUAGCUUGUGAAAAAGAG
AGAGACCUUUUUAAACUCAUUUUGAAAAAAGAGGAUGAAUUGGGG
GAUAGAUCUAUAAUGUUCACUGUUCAAAACGAAGAC

Furthermore, preferably the RNA construct comprises an RNA nucleotide sequence substantially as set out in SEQ ID No: 51, or a variant or fragment thereof.

In one embodiment, the at least one ISP may be IL-19 (NCBI Reference Sequence: NM_013371.4; UniProtKB—Q9UHD0 (IL19_HUMAN)), or an orthologue thereof. One embodiment of the polypeptide sequence of IL-19 (signal peptide 1-24) is represented herein as SEQ ID No: 52, as follows:

[SEQ ID No: 52]
MKLQCVSLWLLGTILILCSVDNHGLRRCLISTDMHHIEESFQEIK
RAIQAKDTFPNVTILSTLETLQIIKPLDVCCVTKNLLAFYVDRVF
KDHQEPNPKILRKISSIANSFLYMQKTLRQCQEQRQCHCRQEATN
ATRVIHDNYDQLEVHAAAIKSLGELDVFLAWINKNHEVMESA

Therefore, preferably the RNA construct of any aspect comprises a nucleotide sequence which encodes an amino acid sequence substantially as set out in SEQ ID No: 52, or a variant or fragment thereof.

In one embodiment, the IL-19 polypeptide is encoded by the DNA nucleotide sequence of SEQ ID No: 53, as follows:

[SEQ ID No: 53]
ATGAAGTTACAGTGTGTTTCCCTTTGGCTCCTGGGTACAATACTG
ATATTGTGCTCAGTAGACAACCACGGTCTCAGGAGATGTCTGATT
TCCACAGACATGCACCATATAGAAGAGAGTTTCCAAGAAATCAAA
AGAGCCATCCAAGCTAAGGACACCTTCCCAAATGTCACTATCCTG
TCCACATTGGAGACTCTGCAGATCATTAAGCCCTTAGATGTGTGC
TGCGTGACCAAGAACCTCCTGGCGTTCTACGTGGACAGGGTGTTC
AAGGATCATCAGGAGCCAAACCCCAAAATCTTGAGAAAAATCAGC
AGCATTGCCAACTCTTTCCTCTACATGCAGAAAACTCTGCGGCAA
TGTCAGGAACAGAGGCAGTGTCACTGCAGGCAGGAAGCCACCAAT
GCCACCAGAGTCATCCATGACAACTATGATCAGCTGGAGGTCCAC
GCTGCTGCCATTAAATCCCTGGGAGAGCTCGACGTCTTTCTAGCC
TGGATTAATAAGAATCATGAAGTAATGTTCTCAGCT

Accordingly, preferably the IL-19 polypeptide is encoded by the DNA nucleotide sequence substantially as set out in SEQ ID NO: 53, or a variant or fragment thereof.

Thus, the RNA construct may comprise an RNA nucleotide sequence of SEQ ID No: 54, as follows:

[SEQ ID No: 54]
AUGAAGUUACAGUGUGUUUCCCUUUGGCUCCUGGGUACAAUACUG
AUAUUGUGCUCAGUAGACAACCACGGUCUCAGGAGAUGUCUGAUU
UCCACAGACAUGCACCAUAUAGAAGAGAGUUUCCAAGAAAUCAAA
AGAGCCAUCCAAGCUAAGGACACCUUCCCAAAUGUCACUAUCCUG
UCCACAUUGGAGACUCUGCAGAUCAUUAAGCCCUUAGAUGUGUGC
UGCGUGACCAAGAACCUCCUGGCGUUCUACGUGGACAGGGUGUUC
AAGGAUCAUCAGGAGCCAAACCCCAAAAUCUUGAGAAAAAUCAGC
AGCAUUGCCAACUCUUUCCUCUACAUGCAGAAAACUCUGCGGCAA
UGUCAGGAACAGAGGCAGUGUCACUGCAGGCAGGAAGCCACCAAU
GCCACCAGAGUCAUCCAUGACAACUAUGAUCAGCUGGAGGUCCAC
GCUGCUGCCAUUAAAUCCCUGGGAGAGCUCGACGUCUUUCUAGCC
UGGAUUAAUAAGAAUCAUGAAGUAAUGUUCUCAGCU

Furthermore, preferably the RNA construct comprises an RNA nucleotide sequence substantially as set out in SEQ ID No: 54, or a variant or fragment thereof.

In one embodiment, the at least one ISP may be IL-20 (NCBI Reference Sequence: NM_018724.4; UniProtKB—Q9NYY1 (IL20_HUMAN)), or an orthologue thereof. One embodiment of the polypeptide sequence of IL-20 (signal peptide 1-24) is represented herein as SEQ ID No: 55, as follows:

[SEQ ID No: 55]
MKASSLAFSLLSAAFYLLWTPSTGLKTLNLGSCVIATNLQEIRNG
FSEIRGSVQAKDGNIDIRILRRTESLQDTKPANRCCLLRHLLRLY
LDRVFKNYQTPDHYTLRKISSLANSELTIKKDLRLCHAHMTCHCG
EEAMKKYSQILSHFEKLEPQAAVVKALGELDILLQWMEETE

Therefore, preferably the RNA construct of any aspect comprises a nucleotide sequence which encodes an amino acid sequence substantially as set out in SEQ ID No: 55, or a variant or fragment thereof.

In one embodiment, the IL-20 polypeptide is encoded by the DNA nucleotide sequence of SEQ ID No: 56, as follows:

[SEQ ID No: 56]
ATGAAAGCCTCTAGTCTTGCCTTCAGCCTTCTCTCTGCTGCGTTT
TATCTCCTATGGACTCCTTCCACTGGACTGAAGACACTCAATTTG
GGAAGCTGTGTGATCGCCACAAACCTTCAGGAAATACGAAATGGA
TTTTCTGAGATACGGGGCAGTGTGCAAGCCAAAGATGGAAACATT
GACATCAGAATCTTAAGGAGGACTGAGTCTTTGCAAGACACAAAG
CCTGCGAATCGATGCTGCCTCCTGCGCCATTTGCTAAGACTCTAT
CTGGACAGGGTATTTAAAAACTACCAGACCCCTGACCATTATACT
CTCCGGAAGATCAGCAGCCTCGCCAATTCCTTTCTTACCATCAAG
AAGGACCTCCGGCTCTGTCATGCCCACATGACATGCCATTGTGGG
GAGGAAGCAATGAAGAAATACAGCCAGATTCTGAGTCACTTTGAA
AAGCTGGAACCTCAGGCAGCAGTTGTGAAGGCTTTGGGGGAACTA
GACATTCTTCTGCAATGGATGGAGGAGACAGAA

Accordingly, preferably the IL-20 polypeptide is encoded by the DNA nucleotide sequence substantially as set out in SEQ ID NO: 56, or a variant or fragment thereof.

Thus, the RNA construct may comprise an RNA nucleotide sequence of SEQ ID No: 57, as follows:

[SEQ ID No: 57]
AUGAAAGCCUCUAGUCUUGCCUUCAGCCUUCUCUCUGCUGCGUUU
UAUCUCCUAUGGACUCCUUCCACUGGACUGAAGACACUCAAUUUG
GGAAGCUGUGUGAUCGCCACAAACCUUCAGGAAAUACGAAAUGGA
UUUUCUGAGAUACGGGGCAGUGUGCAAGCCAAAGAUGGAAACAUU
GACAUCAGAAUCUUAAGGAGGACUGAGUCUUUGCAAGACACAAAG
CCUGCGAAUCGAUGCUGCCUCCUGCGCCAUUUGCUAAGACUCUAU
CUGGACAGGGUAUUUAAAAACUACCAGACCCCUGACCAUUAUACU
CUCCGGAAGAUCAGCAGCCUCGCCAAUUCCUUUCUUACCAUCAAG
AAGGACCUCCGGCUCUGUCAUGCCCACAUGACAUGCCAUUGUGGG
GAGGAAGCAAUGAAGAAAUACAGCCAGAUUCUGAGUCACUUUGAA
AAGCUGGAACCUCAGGCAGCAGUUGUGAAGGCUUUGGGGGAACUA
GACAUUCUUCUGCAAUGGAUGGAGGAGACAGAA

Furthermore, preferably the RNA construct comprises an RNA nucleotide sequence substantially as set out in SEQ ID No: 57, or a variant or fragment thereof.

In one embodiment, the at least one ISP may be IL-21 (NCBI Reference Sequence: NM_021803.4; UniProtKB—Q9HBE4 (IL21_HUMAN)), or an orthologue thereof. One embodiment of the polypeptide sequence of IL-21 (signal peptide 1-24) is represented herein as SEQ ID No: 58, as follows:

[SEQ ID No: 58]
MRSSPGNMERIVICLMVIFLGTLVHKSSSQGQDRHMIRMRQLIDI
VDQLKNYVNDLVPEFLPAPEDVETNCEWSAFSCFQKAQLKSANTG
NNERIINVSIKKLKRKPPSTNAGRRQKHRLTCPSCDSYEKKPPKE
FLERFKSLLQKMIHQHLSSRTHGSEDS

Therefore, preferably the RNA construct of any aspect comprises a nucleotide sequence which encodes an amino acid sequence substantially as set out in SEQ ID No: 58, or a variant or fragment thereof.

In one embodiment, the IL-21 polypeptide is encoded by the DNA nucleotide sequence of SEQ ID No: 59, as follows:

[SEQ ID No: 59]
ATGAGATCCAGTCCTGGCAACATGGAGAGGATTGTCATCTGTCTG
ATGGTCATCTTCTTGGGGACACTGGTCCACAAATCAAGCTCCCAA
GGTCAAGATCGCCACATGATTAGAATGCGTCAACTTATAGATATT
GTTGATCAGCTGAAAAATTATGTGAATGACTTGGTCCCTGAATTT
CTGCCAGCTCCAGAAGATGTAGAGACAAACTGTGAGTGGTCAGCT
TTTTCCTGCTTTCAGAAGGCCCAACTAAAGTCAGCAAATACAGGA
AACAATGAAAGGATAATCAATGTATCAATTAAAAAGCTGAAGAGG
AAACCACCTTCCACAAATGCAGGGAGAAGACAGAAACACAGACTA
ACATGCCCTTCATGTGATTCTTATGAGAAAAAACCACCCAAAGAA
TTCCTAGAAAGATTCAAATCACTTCTCCAAAAGATGATTCATCAG
CATCTGTCCTCTAGAACACACGGAAGTGAAGATTCC

Accordingly, preferably the IL-21 polypeptide is encoded by the DNA nucleotide sequence substantially as set out in SEQ ID NO: 59, or a variant or fragment thereof.

Thus, the RNA construct may comprise an RNA nucleotide sequence of SEQ ID No: 60, as follows:

[SEQ ID No: 60]
AUGAGAUCCAGUCCUGGCAACAUGGAGAGGAUUGUCAUCUGUCUG
AUGGUCAUCUUCUUGGGGACACUGGUCCACAAAUCAAGCUCCCAA
GGUCAAGAUCGCCACAUGAUUAGAAUGCGUCAACUUAUAGAUAUU
GUUGAUCAGCUGAAAAAUUAUGUGAAUGACUUGGUCCCUGAAUUU
CUGCCAGCUCCAGAAGAUGUAGAGACAAACUGUGAGUGGUCAGCU
UUUUCCUGCUUUCAGAAGGCCCAACUAAAGUCAGCAAAUACAGGA
AACAAUGAAAGGAUAAUCAAUGUAUCAAUUAAAAAGCUGAAGAGG
AAACCACCUUCCACAAAUGCAGGGAGAAGACAGAAACACAGACUA
ACAUGCCCUUCAUGUGAUUCUUAUGAGAAAAAACCACCCAAAGAA
UUCCUAGAAAGAUUCAAAUCACUUCUCCAAAAGAUGAUUCAUCAG
CAUCUGUCCUCUAGAACACACGGAAGUGAAGAUUCC

Furthermore, preferably the RNA construct comprises an RNA nucleotide sequence substantially as set out in SEQ ID No: 60, or a variant or fragment thereof.

In one embodiment, the at least one ISP may be IL-22 (NCBI Reference Sequence: NM_020525.5; UniProtKB—Q9GZX6 (IL22_HUMAN)), or an orthologue thereof. One embodiment of the polypeptide sequence of IL-22 (signal peptide 1-33) is represented herein as SEQ ID No: 61, as follows:

[SEQ ID No: 61]
MAALQKSVSSFLMGTLATSCLLLLALLVQGGAAAPISSHCRLDKS
NFQQPYITNRTFMLAKEASLADNNTDVRLIGEKLFHGVSMSERCY
LMKQVLNFTLEEVLFPQSDRFQPYMQEVVPFLARLSNRLSTCHIE
GDDLHIQRNVQKLKDTVKKLGESGEIKAIGELDLLEMSLRNACI

Therefore, preferably the RNA construct of any aspect comprises a nucleotide sequence which encodes an amino acid sequence substantially as set out in SEQ ID No: 61, or a variant or fragment thereof.

In one embodiment, the IL-22 polypeptide is encoded by the DNA nucleotide sequence of SEQ ID No: 62, as follows:

[SEQ ID No: 62]
ATGGCCGCCCTGCAGAAATCTGTGAGCTCTTTCCTTATGGGGACC
CTGGCCACCAGCTGCCTCCTTCTCTTGGCCCTCTTGGTACAGGGA
GGAGCAGCTGCGCCCATCAGCTCCCACTGCAGGCTTGACAAGTCC
AACTTCCAGCAGCCCTATATCACCAACCGCACCTTCATGCTGGCT
AAGGAGGCTAGCTTGGCTGATAACAACACAGACGTTCGTCTCATT
GGGGAGAAACTGTTCCACGGAGTCAGTATGAGTGAGCGCTGCTAT
CTGATGAAGCAGGTGCTGAACTTCACCCTTGAAGAAGTGCTGTTC
CCTCAATCTGATAGGTTCCAGCCTTATATGCAGGAGGTGGTGCCC
TTCCTGGCCAGGCTCAGCAACAGGCTAAGCACATGTCATATTGAA
GGTGATGACCTGCATATCCAGAGGAATGTGCAAAAGCTGAAGGAC
ACAGTGAAAAAGCTTGGAGAGAGTGGAGAGATCAAAGCAATTGGA
GAACTGGATTTGCTGTTTATGTCTCTGAGAAATGCCTGCATT

Accordingly, preferably the IL-22 polypeptide is encoded by the DNA nucleotide sequence substantially as set out in SEQ ID NO: 62, or a variant or fragment thereof.

Thus, the RNA construct may comprise an RNA nucleotide sequence of SEQ ID No: 63, as follows:

[SEQ ID No: 63]
AUGGCCGCCCUGCAGAAAUCUGUGAGCUCUUUCCUUAUGGGGACC
CUGGCCACCAGCUGCCUCCUUCUCUUGGCCCUCUUGGUACAGGGA
GGAGCAGCUGCGCCCAUCAGCUCCCACUGCAGGCUUGACAAGUCC
AACUUCCAGCAGCCCUAUAUCACCAACCGCACCUUCAUGCUGGCU
AAGGAGGCUAGCUUGGCUGAUAACAACACAGACGUUCGUCUCAUU
GGGGAGAAACUGUUCCACGGAGUCAGUAUGAGUGAGCGCUGCUAU
CUGAUGAAGCAGGUGCUGAACUUCACCCUUGAAGAAGUGCUGUUC
CCUCAAUCUGAUAGGUUCCAGCCUUAUAUGCAGGAGGUGGUGCCC
UUCCUGGCCAGGCUCAGCAACAGGCUAAGCACAUGUCAUAUUGAA
GGUGAUGACCUGCAUAUCCAGAGGAAUGUGCAAAAGCUGAAGGAC
ACAGUGAAAAAGCUUGGAGAGAGUGGAGAGAUCAAAGCAAUUGGA
GAACUGGAUUUGCUGUUUAUGUCUCUGAGAAAUGCCUGCAUU

Furthermore, preferably the RNA construct comprises an RNA nucleotide sequence substantially as set out in SEQ ID No: 63, or a variant or fragment thereof.

In one embodiment, the at least one ISP may be IL-33 (NCBI Reference Sequence: NM_033439.4; UniProtKB—O95760 (IL33_HUMAN)), or an orthologue thereof. One embodiment of the polypeptide sequence of IL-33 (with an added secretion signal) is represented herein as SEQ ID No: 64, as follows:

[SEQ ID No: 64]
MDRAKLLLLLLLLLLPQAQAAFGISGVQKYTRALHDSSITGISPI
TEYLASLSTYNDQSITFALEDESYEIYVEDLKKDEKKDKVLLSYY
ESQHPSNESGDGVDGKMLMVTLSPTKDEWLHANNKEHSVELHKCE
KPLPDQAFFVLHNMHSNCVSFECKTDPGVFIGVKDNHLALIKVDS
SENLCTENILFKLSET

Therefore, preferably the RNA construct of any aspect comprises a nucleotide sequence which encodes an amino acid sequence substantially as set out in SEQ ID No: 64, or a variant or fragment thereof.

In one embodiment, the IL-33 polypeptide is encoded by the DNA nucleotide sequence of SEQ ID No: 65, as follows:

[SEQ ID No: 65]
ATGGACAGAGCCAAGCTGCTGCTGTTGCTGCTCCTTCTGCTGCTG
CCTCAGGCTCAAGCTGCCTTTGGTATATCAGGGGTCCAGAAATAT
ACTAGAGCACTTCATGATTCAAGTATCACAGGAATTTCACCTATT
ACAGAGTATCTTGCTTCTCTAAGCACATACAATGATCAATCCATT
ACTTTTGCTTTGGAGGATGAAAGTTATGAGATATATGTTGAAGAC
TTGAAAAAAGATGAAAAGAAAGATAAGGTGTTACTGAGTTACTAT
GAGTCTCAACACCCCTCAAATGAATCAGGTGACGGTGTTGATGGT
AAGATGTTAATGGTAACCCTGAGTCCTACAAAAGACTTCTGGTTG
CATGCCAACAACAAGGAACACTCTGTGGAGCTCCATAAGTGTGAA
AAACCACTGCCAGACCAGGCCTTCTTTGTCCTTCATAATATGCAC
TCCAACTGTGTTTCATTTGAATGCAAGACTGATCCTGGAGTGTTT
ATAGGTGTAAAGGATAATCATCTTGCTCTGATTAAAGTAGACTCT
TCTGAGAATTTGTGTACTGAAAATATCTTGTTTAAGCTCTCTGAA
ACT

Accordingly, preferably the IL-33 polypeptide is encoded by the DNA nucleotide sequence substantially as set out in SEQ ID NO: 65, or a variant or fragment thereof.

Thus, the RNA construct may comprise an RNA nucleotide sequence of SEQ ID No: 66, as follows:

[SEQ ID No: 66]
AUGGACAGAGCCAAGCUGCUGCUGUUGCUGCUCCUUCUGCUGCUG
CCUCAGGCUCAAGCUGCCUUUGGUAUAUCAGGGGUCCAGAAAUAU
ACUAGAGCACUUCAUGAUUCAAGUAUCACAGGAAUUUCACCUAUU
ACAGAGUAUCUUGCUUCUCUAAGCACAUACAAUGAUCAAUCCAUU
ACUUUUGCUUUGGAGGAUGAAAGUUAUGAGAUAUAUGUUGAAGAC
UUGAAAAAAGAUGAAAAGAAAGAUAAGGUGUUACUGAGUUACUAU
GAGUCUCAACACCCCUCAAAUGAAUCAGGUGACGGUGUUGAUGGU
AAGAUGUUAAUGGUAACCCUGAGUCCUACAAAAGACUUCUGGUUG
CAUGCCAACAACAAGGAACACUCUGUGGAGCUCCAUAAGUGUGAA
AAACCACUGCCAGACCAGGCCUUCUUUGUCCUUCAUAAUAUGCAC
UCCAACUGUGUUUCAUUUGAAUGCAAGACUGAUCCUGGAGUGUUU
AUAGGUGUAAAGGAUAAUCAUCUUGCUCUGAUUAAAGUAGACUCU
UCUGAGAAUUUGUGUACUGAAAAUAUCUUGUUUAAGCUCUCUGAA
ACU

Furthermore, preferably the RNA construct comprises an RNA nucleotide sequence substantially as set out in SEQ ID No: 66, or a variant or fragment thereof.

In one embodiment, the at least one ISP may be IL-36 alpha (NCBI Reference Sequence: NM_014440.3; UniProtKB—Q9UHA7 (IL36A_HUMAN)), or an orthologue thereof. One embodiment of the polypeptide sequence of IL-36 alpha with an added secretion signal is represented herein as SEQ ID No: 67, as follows:

[SEQ ID No: 67]
MDRAKLLLLLLLLLLPQAQAKIDTPQQGSIQDINHRVWVLQDQTL
IAVPRKDRMSPVTIALISCRHVETLEKDRGNPIYLGLNGLNLCLM
CAKVGDQPTLQLKEKDIMDLYNQPEPVKSFLFYHSQSGRNSTFES
VAFPGWFIAVSSEGGCPLILTQELGKANTTDFGLTMLF

Therefore, preferably the RNA construct of any aspect comprises a nucleotide sequence which encodes an amino acid sequence substantially as set out in SEQ ID No: 67, or a variant or fragment thereof.

In one embodiment, the IL-36 alpha polypeptide is encoded by the DNA nucleotide sequence of SEQ ID No: 68, as follows:

[SEQ ID No: 68]
ATGGACAGAGCCAAGCTGCTGCTGTTGCTGCTCCTTCTGCTGCTG
CCTCAGGCTCAAGCTAAAATTGACACACCTCAGCAGGGGAGCATT
CAGGATATCAATCATCGGGTGTGGGTTCTTCAGGACCAGACGCTC
ATAGCAGTCCCGAGGAAGGACCGTATGTCTCCAGTCACTATTGCC
TTAATCTCATGCCGACATGTGGAGACCCTTGAGAAAGACAGAGGG
AACCCCATCTACCTGGGCCTGAATGGACTCAATCTCTGCCTGATG
TGTGCTAAAGTCGGGGACCAGCCCACACTGCAGCTGAAGGAAAAG
GATATAATGGATTTGTACAACCAACCCGAGCCTGTGAAGTCCTTT
CTCTTCTACCACAGCCAGAGTGGCAGGAACTCCACCTTCGAGTCT
GTGGCTTTCCCTGGCTGGTTCATCGCTGTCAGCTCTGAAGGAGGC
TGTCCTCTCATCCTTACCCAAGAACTGGGGAAAGCCAACACTACT
GACTTTGGGTTAACTATGCTGTTT

Accordingly, preferably the IL-36 alpha polypeptide is encoded by the DNA nucleotide sequence substantially as set out in SEQ ID NO: 68, or a variant or fragment thereof.

Thus, the RNA construct may comprise an RNA nucleotide sequence of SEQ ID No: 69, as follows:

[SEQ ID No: 69]
AUGGACAGAGCCAAGCUGCUGCUGUUGCUGCUCCUUCUGCUGCUG
CCUCAGGCUCAAGCUAAAAUUGACACACCUCAGCAGGGGAGCAUU
CAGGAUAUCAAUCAUCGGGUGUGGGUUCUUCAGGACCAGACGCUC
AUAGCAGUCCCGAGGAAGGACCGUAUGUCUCCAGUCACUAUUGCC
UUAAUCUCAUGCCGACAUGUGGAGACCCUUGAGAAAGACAGAGGG
AACCCCAUCUACCUGGGCCUGAAUGGACUCAAUCUCUGCCUGAUG
UGUGCUAAAGUCGGGGACCAGCCCACACUGCAGCUGAAGGAAAAG
GAUAUAAUGGAUUUGUACAACCAACCCGAGCCUGUGAAGUCCUUU
CUCUUCUACCACAGCCAGAGUGGCAGGAACUCCACCUUCGAGUCU
GUGGCUUUCCCUGGCUGGUUCAUCGCUGUCAGCUCUGAAGGAGGC
UGUCCUCUCAUCCUUACCCAAGAACUGGGGAAAGCCAACACUACU
GACUUUGGGUUAACUAUGCUGUUU

Furthermore, preferably the RNA construct comprises an RNA nucleotide sequence substantially as set out in SEQ ID No: 69, or a variant or fragment thereof.

In one embodiment, the at least one ISP may be Tumour Necrosis Factor, Membrane Form (NCBI Reference Sequence: NM_000594.4; UniProtKB—P01375 (TNFA_HUMAN)), or an orthologue thereof. One embodiment of the polypeptide sequence of the Tumour Necrosis Factor, Membrane Form is represented herein as SEQ ID No: 70, as follows:

[SEQ ID No: 70]
MSTESMIRDVELAEEALPKKTGGPQGSRRCLFLSLESFLIVAGAT
TLFCLLHFGVIGPQREEFPRDLSLISPLAQAVRSSSRTPSDKPVA
HVVANPQAEGQLQWLNRRANALLANGVELRDNQLVVPSEGLYLIY
SQVLFKGQGCPSTHVLLTHTISRIAVSYQTKVNLLSAIKSPCQRE
TPEGAEAKPWYEPIYLGGVFQLEKGDRLSAEINRPDYLDFAESGQ
VYFGIIAL

Therefore, preferably the RNA construct of any aspect comprises a nucleotide sequence which encodes an amino acid sequence substantially as set out in SEQ ID No: 70, or a variant or fragment thereof.

In one embodiment, the Tumour Necrosis Factor, Membrane Form polypeptide is encoded by the DNA nucleotide sequence of SEQ ID No: 71, as follows:

[SEQ ID No: 71]
ATGAGCACTGAAAGCATGATCCGGGACGTGGAGCTGGCCGAGGAG
GCGCTCCCCAAGAAGACAGGGGGGCCCCAGGGCTCCAGGCGGTGC
TTGTTCCTCAGCCTCTTCTCCTTCCTGATCGTGGCAGGCGCCACC
ACGCTCTTCTGCCTGCTGCACTTTGGAGTGATCGGCCCCCAGAGG
GAAGAGTTCCCCAGGGACCTCTCTCTAATCAGCCCTCTGGCCCAG
GCAGTCAGATCATCTTCTCGAACCCCGAGTGACAAGCCTGTAGCC
CATGTTGTAGCAAACCCTCAAGCTGAGGGGCAGCTCCAGTGGCTG
AACCGCCGGGCCAATGCCCTCCTGGCCAATGGCGTGGAGCTGAGA
GATAACCAGCTGGTGGTGCCATCAGAGGGCCTGTACCTCATCTAC
TCCCAGGTCCTCTTCAAGGGCCAAGGCTGCCCCTCCACCCATGTG
CTCCTCACCCACACCATCAGCCGCATCGCCGTCTCCTACCAGACC
AAGGTCAACCTCCTCTCTGCCATCAAGAGCCCCTGCCAGAGGGAG
ACCCCAGAGGGGGCTGAGGCCAAGCCCTGGTATGAGCCCATCTAT
CTGGGAGGGGTCTTCCAGCTGGAGAAGGGTGACCGACTCAGCGCT
GAGATCAATCGGCCCGACTATCTCGACTTTGCCGAGTCTGGGCAG
GTCTACTTTGGGATCATTGCCCTG

Accordingly, preferably the Tumour Necrosis Factor, Membrane Form polypeptide is encoded by the DNA nucleotide sequence substantially as set out in SEQ ID NO: 71, or a variant or fragment thereof.

Thus, the RNA construct may comprise an RNA nucleotide sequence of SEQ ID No: 72, as follows:

[SEQ ID No: 72]
AUGAGCACUGAAAGCAUGAUCCGGGACGUGGAGCUGGCCGAGGAG
GCGCUCCCCAAGAAGACAGGGGGGCCCCAGGGCUCCAGGCGGUGC
UUGUUCCUCAGCCUCUUCUCCUUCCUGAUCGUGGCAGGCGCCACC
ACGCUCUUCUGCCUGCUGCACUUUGGAGUGAUCGGCCCCCAGAGG
GAAGAGUUCCCCAGGGACCUCUCUCUAAUCAGCCCUCUGGCCCAG
GCAGUCAGAUCAUCUUCUCGAACCCCGAGUGACAAGCCUGUAGCC
CAUGUUGUAGCAAACCCUCAAGCUGAGGGGCAGCUCCAGUGGCUG
AACCGCCGGGCCAAUGCCCUCCUGGCCAAUGGCGUGGAGCUGAGA
GAUAACCAGCUGGUGGUGCCAUCAGAGGGCCUGUACCUCAUCUAC
UCCCAGGUCCUCUUCAAGGGCCAAGGCUGCCCCUCCACCCAUGUG
CUCCUCACCCACACCAUCAGCCGCAUCGCCGUCUCCUACCAGACC
AAGGUCAACCUCCUCUCUGCCAUCAAGAGCCCCUGCCAGAGGGAG
ACCCCAGAGGGGGCUGAGGCCAAGCCCUGGUAUGAGCCCAUCUAU
CUGGGAGGGGUCUUCCAGCUGGAGAAGGGUGACCGACUCAGCGCU
GAGAUCAAUCGGCCCGACUAUCUCGACUUUGCCGAGUCUGGGCAG
GUCUACUUUGGGAUCAUUGCCCUG

Furthermore, preferably the RNA construct comprises an RNA nucleotide sequence substantially as set out in SEQ ID No: 72, or a variant or fragment thereof.

In one embodiment, the at least one ISP may be Tumour Necrosis Factor, Soluble Form (NCBI Reference Sequence: NM_000594.4; UniProtKB—P01375 (TNFA_HUMAN)), or an orthologue thereof. One embodiment of the polypeptide sequence of Tumour Necrosis Factor, Soluble Form (with an added secretion signal) is represented herein as SEQ ID No: 73, as follows:

[SEQ ID No: 73]
MDRAKLLLLLLLLLLPQAQAVRSSSRTPSDKPVAHVVANPQAEGQ
LQWLNRRANALLANGVELRDNQLVVPSEGLYLIYSQVLFKGQGCP
STHVLLTHTISRIAVSYQTKVNLLSAIKSPCQRETPEGAEAKPWY
EPIYLGGVFQLEKGDRLSAEINRPDYLDFAESGQVYFGIIAL

Therefore, preferably the RNA construct of any aspect comprises a nucleotide sequence which encodes an amino acid sequence substantially as set out in SEQ ID No: 73, or a variant or fragment thereof.

In one embodiment, the Tumour Necrosis Factor, Soluble Form polypeptide is encoded by the DNA nucleotide sequence of SEQ ID No: 74, as follows:

[SEQ ID No: 74]
ATGGACAGAGCCAAGCTGCTGCTGTTGCTGCTCCTTCTGCTGCTG
CCTCAGGCTCAAGCTGTCAGATCATCTTCTCGAACCCCGAGTGAC
AAGCCTGTAGCCCATGTTGTAGCAAACCCTCAAGCTGAGGGGCAG
CTCCAGTGGCTGAACCGCCGGGCCAATGCCCTCCTGGCCAATGGC
GTGGAGCTGAGAGATAACCAGCTGGTGGTGCCATCAGAGGGCCTG
TACCTCATCTACTCCCAGGTCCTCTTCAAGGGCCAAGGCTGCCCC
TCCACCCATGTGCTCCTCACCCACACCATCAGCCGCATCGCCGTC
TCCTACCAGACCAAGGTCAACCTCCTCTCTGCCATCAAGAGCCCC
TGCCAGAGGGAGACCCCAGAGGGGGCTGAGGCCAAGCCCTGGTAT
GAGCCCATCTATCTGGGAGGGGTCTTCCAGCTGGAGAAGGGTGAC
CGACTCAGCGCTGAGATCAATCGGCCCGACTATCTCGACTTTGCC
GAGTCTGGGCAGGTCTACTTTGGGATCATTGCCCTG

Accordingly, preferably the Tumour Necrosis Factor, Soluble Form polypeptide is encoded by the DNA nucleotide sequence substantially as set out in SEQ ID NO: 74, or a variant or fragment thereof.

Thus, the RNA construct may comprise an RNA nucleotide sequence of SEQ ID No: 75, as follows:

[SEQ ID No: 75]
AUGGACAGAGCCAAGCUGCUGCUGUUGCUGCUCCUUCUGCUGCUG
CCUCAGGCUCAAGCUGUCAGAUCAUCUUCUCGAACCCCGAGUGAC
AAGCCUGUAGCCCAUGUUGUAGCAAACCCUCAAGCUGAGGGGCAG
CUCCAGUGGCUGAACCGCCGGGCCAAUGCCCUCCUGGCCAAUGGC
GUGGAGCUGAGAGAUAACCAGCUGGUGGUGCCAUCAGAGGGCCUG
UACCUCAUCUACUCCCAGGUCCUCUUCAAGGGCCAAGGCUGCCCC
UCCACCCAUGUGCUCCUCACCCACACCAUCAGCCGCAUCGCCGUC
UCCUACCAGACCAAGGUCAACCUCCUCUCUGCCAUCAAGAGCCCC
UGCCAGAGGGAGACCCCAGAGGGGGCUGAGGCCAAGCCCUGGUAU
GAGCCCAUCUAUCUGGGAGGGGUCUUCCAGCUGGAGAAGGGUGAC
CGACUCAGCGCUGAGAUCAAUCGGCCCGACUAUCUCGACUUUGCC
GAGUCUGGGCAGGUCUACUUUGGGAUCAUUGCCCUG

Furthermore, preferably the RNA construct comprises an RNA nucleotide sequence substantially as set out in SEQ ID No: 75, or a variant or fragment thereof.

In one embodiment, the at least one ISP may be Human BAFF, Membrane Form (NCBI Reference Sequence: NM_006573.5; UniProtKB—Q9Y275 (TN13B_HUMAN)), or an orthologue thereof. One embodiment of the polypeptide sequence of Human BAFF, Membrane Form is represented herein as SEQ ID No: 76, as follows:

[SEQ ID No: 76]
MDDSTEREQSRLTSCLKKREEMKLKECVSILPRKESPSVRSSKDG
KLLAATLLLALLSCCLTVVSFYQVAALQGDLASLRAELQGHHAEK
LPAGAGAPKAGLEEAPAVTAGLKIFEPPAPGEGNSSQNSRNKRAV
QGPEETVTQDCLQLIADSETPTIQKGSYTFVPWLLSFKRGSALEE
KENKILVKETGYFFIYGQVLYTDKTYAMGHLIQRKKVHVFGDELS
LVTLFRCIQNMPETLPNNSCYSAGIAKLEEGDELQLAIPRENAQI
SLDGDVTFFGALKLL

Therefore, preferably the RNA construct of any aspect comprises a nucleotide sequence which encodes an amino acid sequence substantially as set out in SEQ ID No: 76, or a variant or fragment thereof.

In one embodiment, the Human BAFF, Membrane Form polypeptide is encoded by the DNA nucleotide sequence of SEQ ID No: 77, as follows:

[SEQ ID No: 77]
ATGGATGACTCCACAGAAAGGGAGCAGTCACGCCTTACTTCTTGC
CTTAAGAAAAGAGAAGAAATGAAACTGAAGGAGTGTGTTTCCATC
CTCCCACGGAAGGAAAGCCCCTCTGTCCGATCCTCCAAAGACGGA
AAGCTGCTGGCTGCAACCTTGCTGCTGGCACTGCTGTCTTGCTGC
CTCACGGTGGTGTCTTTCTACCAGGTGGCCGCCCTGCAAGGGGAC
CTGGCCAGCCTCCGGGCAGAGCTGCAGGGCCACCACGCGGAGAAG
CTGCCAGCAGGAGCAGGAGCCCCCAAGGCCGGCCTGGAGGAAGCT
CCAGCTGTCACCGCGGGACTGAAAATCTTTGAACCACCAGCTCCA
GGAGAAGGCAACTCCAGTCAGAACAGCAGAAATAAGCGTGCCGTT
CAGGGTCCAGAAGAAACAGTCACTCAAGACTGCTTGCAACTGATT
GCAGACAGTGAAACACCAACTATACAAAAAGGATCTTACACATTT
GTTCCATGGCTTCTCAGCTTTAAAAGGGGAAGTGCCCTAGAAGAA
AAAGAGAATAAAATATTGGTCAAAGAAACTGGTTACTTTTTTATA
TATGGTCAGGTTTTATATACTGATAAGACCTACGCCATGGGACAT
CTAATTCAGAGGAAGAAGGTCCATGTCTTTGGGGATGAATTGAGT
CTGGTGACTTTGTTTCGATGTATTCAAAATATGCCTGAAACACTA
CCCAATAATTCCTGCTATTCAGCTGGCATTGCAAAACTGGAAGAA
GGAGATGAACTCCAACTTGCAATACCAAGAGAAAATGCACAAATA
TCACTGGATGGAGATGTCACATTTTTTGGTGCATTGAAACTGCTG

Accordingly, preferably the Human BAFF, Membrane Form polypeptide is encoded by the DNA nucleotide sequence substantially as set out in SEQ ID NO: 77, or a variant or fragment thereof.

Thus, the RNA construct may comprise an RNA nucleotide sequence of SEQ ID No: 78, as follows:

[SEQ ID No: 78]
AUGGAUGACUCCACAGAAAGGGAGCAGUCACGCCUUACUUCUUGC
CUUAAGAAAAGAGAAGAAAUGAAACUGAAGGAGUGUGUUUCCAUC
CUCCCACGGAAGGAAAGCCCCUCUGUCCGAUCCUCCAAAGACGGA
AAGCUGCUGGCUGCAACCUUGCUGCUGGCACUGCUGUCUUGCUGC
CUCACGGUGGUGUCUUUCUACCAGGUGGCCGCCCUGCAAGGGGAC
CUGGCCAGCCUCCGGGCAGAGCUGCAGGGCCACCACGCGGAGAAG
CUGCCAGCAGGAGCAGGAGCCCCCAAGGCCGGCCUGGAGGAAGCU
CCAGCUGUCACCGCGGGACUGAAAAUCUUUGAACCACCAGCUCCA
GGAGAAGGCAACUCCAGUCAGAACAGCAGAAAUAAGCGUGCCGUU
CAGGGUCCAGAAGAAACAGUCACUCAAGACUGCUUGCAACUGAUU
GCAGACAGUGAAACACCAACUAUACAAAAAGGAUCUUACACAUUU
GUUCCAUGGCUUCUCAGCUUUAAAAGGGGAAGUGCCCUAGAAGAA
AAAGAGAAUAAAAUAUUGGUCAAAGAAACUGGUUACUUUUUUAUA
UAUGGUCAGGUUUUAUAUACUGAUAAGACCUACGCCAUGGGACAU
CUAAUUCAGAGGAAGAAGGUCCAUGUCUUUGGGGAUGAAUUGAGU
CUGGUGACUUUGUUUCGAUGUAUUCAAAAUAUGCCUGAAACACUA
CCCAAUAAUUCCUGCUAUUCAGCUGGCAUUGCAAAACUGGAAGAA
GGAGAUGAACUCCAACUUGCAAUACCAAGAGAAAAUGCACAAAUA
UCACUGGAUGGAGAUGUCACAUUUUUUGGUGCAUUGAAACUGCUG

Furthermore, preferably the RNA construct comprises an RNA nucleotide sequence substantially as set out in SEQ ID No: 78, or a variant or fragment thereof.

In one embodiment, the at least one ISP may be Human BAFF, Soluble Form (NCBI Reference Sequence: NM_006573.5; UniProtKB—Q9Y275 (TN13B_HUMAN)), or an orthologue thereof. One embodiment of the polypeptide sequence of Human BAFF, Soluble Form (with an added secretion signal) is represented herein as SEQ ID No: 79, as follows:

[SEQ ID No: 79]
MDRAKLLLLLLLLLLPQAQAAVQGPEETVTQDCLQLIADSETPTI
QKGSYTFVPWLLSFKRGSALEEKENKILVKETGYFFIYGQVLYTD
KTYAMGHLIQRKKVHVFGDELSLVTLERCIQNMPETLPNNSCYSA
GIAKLEEGDELQLAIPRENAQISLDGDVTFFGALKLL

Therefore, preferably the RNA construct of any aspect comprises a nucleotide sequence which encodes an amino acid sequence substantially as set out in SEQ ID No: 79, or a variant or fragment thereof.

In one embodiment, the Human BAFF, Soluble Form polypeptide is encoded by the DNA nucleotide sequence of SEQ ID No:80, as follows:

[SEQ ID No: 80]
ATGGACAGAGCCAAGCTGCTGCTGTTGCTGCTCCTTCTGCTGCTG
CCTCAGGCTCAAGCTGCCGTTCAGGGTCCAGAAGAAACAGTCACT
CAAGACTGCTTGCAACTGATTGCAGACAGTGAAACACCAACTATA
CAAAAAGGATCTTACACATTTGTTCCATGGCTTCTCAGCTTTAAA
AGGGGAAGTGCCCTAGAAGAAAAAGAGAATAAAATATTGGTCAAA
GAAACTGGTTACTTTTTTATATATGGTCAGGTTTTATATACTGAT
AAGACCTACGCCATGGGACATCTAATTCAGAGGAAGAAGGTCCAT
GTCTTTGGGGATGAATTGAGTCTGGTGACTTTGTTTCGATGTATT
CAAAATATGCCTGAAACACTACCCAATAATTCCTGCTATTCAGCT
GGCATTGCAAAACTGGAAGAAGGAGATGAACTCCAACTTGCAATA
CCAAGAGAAAATGCACAAATATCACTGGATGGAGATGTCACATTT
TTTGGTGCATTGAAACTGCTG

Accordingly, preferably the Human BAFF, Soluble Form polypeptide is encoded by the DNA nucleotide sequence substantially as set out in SEQ ID NO: 80, or a variant or fragment thereof.

Thus, the RNA construct may comprise an RNA nucleotide sequence of SEQ ID No: 81, as follows:

[SEQ ID No: 81]
AUGGACAGAGCCAAGCUGCUGCUGUUGCUGCUCCUUCUGCUGCUG
CCUCAGGCUCAAGCUGCCGUUCAGGGUCCAGAAGAAACAGUCACU
CAAGACUGCUUGCAACUGAUUGCAGACAGUGAAACACCAACUAUA
CAAAAAGGAUCUUACACAUUUGUUCCAUGGCUUCUCAGCUUUAAA
AGGGGAAGUGCCCUAGAAGAAAAAGAGAAUAAAAUAUUGGUCAAA
GAAACUGGUUACUUUUUUAUAUAUGGUCAGGUUUUAUAUACUGAU
AAGACCUACGCCAUGGGACAUCUAAUUCAGAGGAAGAAGGUCCAU
GUCUUUGGGGAUGAAUUGAGUCUGGUGACUUUGUUUCGAUGUAUU
CAAAAUAUGCCUGAAACACUACCCAAUAAUUCCUGCUAUUCAGCU
GGCAUUGCAAAACUGGAAGAAGGAGAUGAACUCCAACUUGCAAUA
CCAAGAGAAAAUGCACAAAUAUCACUGGAUGGAGAUGUCACAUUU
UUUGGUGCAUUGAAACUGCUG

Furthermore, preferably the RNA construct comprises an RNA nucleotide sequence substantially as set out in SEQ ID No: 81, or a variant or fragment thereof.

In one embodiment, the at least one ISP may be Human CD30 Ligand (NCBI Reference Sequence: NM_001244.4; UniProtKB—P32971 (TNFL8_HUMAN)), or an orthologue thereof. One embodiment of the polypeptide sequence of Human CD30 Ligand is represented herein as SEQ ID No: 82, as follows:

[SEQ ID No: 82]
MDPGLQQALNGMAPPGDTAMHVPAGSVASHLGTTSRSYFYLTTAT
LALCLVFTVATIMVLVVQRTDSIPNSPDNVPLKGGNCSEDLLCIL
KRAPFKKSWAYLQVAKHLNKTKLSWNKDGILHGVRYQDGNLVIQF
PGLYFIICQLQFLVQCPNNSVDLKLELLINKHIKKQALVTVCESG
MQTKHVYQNLSQFLLDYLQVNTTISVNVDTFQYIDTSTFPLENVL
SIFLYSNSD

Therefore, preferably the RNA construct of any aspect comprises a nucleotide sequence which encodes an amino acid sequence substantially as set out in SEQ ID No: 82, or a variant or fragment thereof.

In one embodiment, the Human CD30 Ligand polypeptide is encoded by the DNA nucleotide sequence of SEQ ID No: 83, as follows:

[SEQ ID No: 831
ATGGACCCAGGGCTGCAGCAAGCACTCAACGGAATGGCCCCTCCT
GGAGACACAGCCATGCATGTGCCGGCGGGCTCCGTGGCCAGCCAC
CTGGGGACCACGAGCCGCAGCTATTTCTATTTGACCACAGCCACT
CTGGCTCTGTGCCTTGTCTTCACGGTGGCCACTATTATGGTGTTG
GTCGTTCAGAGGACGGACTCCATTCCCAACTCACCTGACAACGTC
CCCCTCAAAGGAGGAAATTGCTCAGAAGACCTCTTATGTATCCTG
AAAAGGGCTCCATTCAAGAAGTCATGGGCCTACCTCCAAGTGGCA
AAGCATCTAAACAAAACCAAGTIGTCTTGGAACAAAGATGGCATT
CTCCATGGAGTCAGATATCAGGATGGGAATCTGGTGATCCAATTC
CCTGGTTTGTACTTCATCATTTGCCAACTGCAGTTTCTTGTACAA
TGCCCAAATAATTCTGTCGATCTGAAGTTGGAGCTTCTCATCAAC
AAGCATATCAAAAAACAGGCCCTGGTGACAGTGTGTGAGTCTGGA
ATGCAAACGAAACACGTATACCAGAATCTCTCTCAATTCTTGCTG
GATTACCTGCAGGTCAACACCACCATATCAGTCAATGTGGATACA
TTCCAGTACATAGATACAAGCACCTTTCCTCTTGAGAATGTGTTG
TCCATCTTCTTATACAGTAATTCAGACTGA

Accordingly, preferably the Human CD30 Ligand polypeptide is encoded by the DNA nucleotide sequence substantially as set out in SEQ ID NO: 83, or a variant or fragment thereof.

Thus, the RNA construct may comprise an RNA nucleotide sequence of SEQ ID No: 84, as follows:

[SEQ ID No: 84]
AUGGACCCAGGGCUGCAGCAAGCACUCAACGGAAUGGCCCCUCCU
GGAGACACAGCCAUGCAUGUGCCGGCGGGCUCCGUGGCCAGCCAC
CUGGGGACCACGAGCCGCAGCUAUUUCUAUUUGACCACAGCCACU
CUGGCUCUGUGCCUUGUCUUCACGGUGGCCACUAUUAUGGUGUUG
GUCGUUCAGAGGACGGACUCCAUUCCCAACUCACCUGACAACGUC
CCCCUCAAAGGAGGAAAUUGCUCAGAAGACCUCUUAUGUAUCCUG
AAAAGGGCUCCAUUCAAGAAGUCAUGGGCCUACCUCCAAGUGGCA
AAGCAUCUAAACAAAACCAAGUUGUCUUGGAACAAAGAUGGCAUU
CUCCAUGGAGUCAGAUAUCAGGAUGGGAAUCUGGUGAUCCAAUUC
CCUGGUUUGUACUUCAUCAUUUGCCAACUGCAGUUUCUUGUACAA
UGCCCAAAUAAUUCUGUCGAUCUGAAGUUGGAGCUUCUCAUCAAC
AAGCAUAUCAAAAAACAGGCCCUGGUGACAGUGUGUGAGUCUGGA
AUGCAAACGAAACACGUAUACCAGAAUCUCUCUCAAUUCUUGCUG
GAUUACCUGCAGGUCAACACCACCAUAUCAGUCAAUGUGGAUACA
UUCCAGUACAUAGAUACAAGCACCUUUCCUCUUGAGAAUGUGUUG
UCCAUCUUCUUAUACAGUAAUUCAGACUGA

Furthermore, preferably the RNA construct comprises an RNA nucleotide sequence substantially as set out in SEQ ID No: 84, or a variant or fragment thereof.

In one embodiment, the at least one ISP may be Human CD40 Ligand (NCBI Reference Sequence: NM_000074.3; UniProtKB—P29965 (CD40L_HUMAN)), or an orthologue thereof. One embodiment of the polypeptide sequence of Human CD40 Ligand is represented herein as SEQ ID No: 85, as follows:

[SEQ ID No: 85]
MIETYNQTSPRSAATGLPISMKIFMYLLTVFLITQMIGSALFAVY
LHRRLDKIEDERNLHEDFVFMKTIQRCNTGERSLSLLNCEEIKSQ
FEGFVKDIMLNKEETKKENSFEMQKGDQNPQIAAHVISEASSKTT
SVLQWAEKGYYTMSNNLVTLENGKQLTVKRQGLYYIYAQVTFCSN
REASSQAPFIASLCLKSPGRFERILLRAANTHSSAKPCGQQSIHL
GGVFELQPGASVFVNVTDPSQVSHGTGFTSFGLLKL

Therefore, preferably the RNA construct of any aspect comprises a nucleotide sequence which encodes an amino acid sequence substantially as set out in SEQ ID No: 85, or a variant or fragment thereof.

In one embodiment, the Human CD40 Ligand polypeptide is encoded by the DNA nucleotide sequence of SEQ ID No: 86, as follows:

[SEQ ID No: 86]
ATGATCGAAACATACAACCAAACTTCTCCCCGATCTGCGGCCACT
GGACTGCCCATCAGCATGAAAATTTTTATGTATTTACTTACTGT
TTTTCTTATCACCCAGATGATTGGGTCAGCACTTTTTGCTGTGTA
TCTTCATAGAAGGTTGGACAAGATAGAAGATGAAAGGAATCTTCA
TGAAGATTTTGTATTCATGAAAACGATACAGAGATGCAACACAGG
AGAAAGATCCTTATCCTTACTGAACTGTGAGGAGATTAAAAGCCA
GTTTGAAGGCTTTGTGAAGGATATAATGTTAAACAAAGAGGAGAC
GAAGAAAGAAAACAGCTTTGAAATGCAAAAAGGTGATCAGAATCC
TCAAATTGCGGCACATGTCATAAGTGAGGCCAGCAGTAAAACAAC
ATCTGTGTTACAGTGGGCTGAAAAAGGATACTACACCATGAGCAA
CAACTTGGTAACCCTGGAAAATGGGAAACAGCTGACCGTTAAAAG
ACAAGGACTCTATTATATCTATGCCCAAGTCACCTTCTGTTCCAA
TCGGGAAGCTTCGAGTCAAGCTCCATTTATAGCCAGCCTCTGCCT
AAAGTCCCCCGGTAGATTCGAGAGAATCTTACTCAGAGCTGCAAA
TACCCACAGTTCCGCCAAACCTTGCGGGCAACAATCCATTCACTT
GGGAGGAGTATTTGAATTGCAACCAGGTGCTTCGGTGTTTGTCAA
TGTGACTGATCCAAGCCAAGTGAGCCATGGCACTGGCTTCACGTC
CTTTGGCTTACTCAAACTCTGA

Accordingly, preferably the Human CD40 Ligand polypeptide is encoded by the DNA nucleotide sequence substantially as set out in SEQ ID NO: 86, or a variant or fragment thereof.

Thus, the RNA construct may comprise an RNA nucleotide sequence of SEQ ID No: 87, as follows:

[SEQ ID No: 87]
AUGAUCGAAACAUACAACCAAACUUCUCCCCGAUCUGCGGCCACU
GGACUGCCCAUCAGCAUGAAAAUUUUUAUGUAUUUACUUACUGUU
UUUCUUAUCACCCAGAUGAUUGGGUCAGCACUUUUUGCUGUGUAU
CUUCAUAGAAGGUUGGACAAGAUAGAAGAUGAAAGGAAUCUUCAU
GAAGAUUUUGUAUUCAUGAAAACGAUACAGAGAUGCAACACAGGA
GAAAGAUCCUUAUCCUUACUGAACUGUGAGGAGAUUAAAAGCCAG
UUUGAAGGCUUUGUGAAGGAUAUAAUGUUAAACAAAGAGGAGACG
AAGAAAGAAAACAGCUUUGAAAUGCAAAAAGGUGAUCAGAAUCCU
CAAAUUGCGGCACAUGUCAUAAGUGAGGCCAGCAGUAAAACAACA
UCUGUGUUACAGUGGGCUGAAAAAGGAUACUACACCAUGAGCAAC
AACUUGGUAACCCUGGAAAAUGGGAAACAGCUGACCGUUAAAAGA
CAAGGACUCUAUUAUAUCUAUGCCCAAGUCACCUUCUGUUCCAAU
CGGGAAGCUUCGAGUCAAGCUCCAUUUAUAGCCAGCCUCUGCCUA
AAGUCCCCCGGUAGAUUCGAGAGAAUCUUACUCAGAGCUGCAAAU
ACCCACAGUUCCGCCAAACCUUGCGGGCAACAAUCCAUUCACUUG
GGAGGAGUAUUUGAAUUGCAACCAGGUGCUUCGGUGUUUGUCAAU
GUGACUGAUCCAAGCCAAGUGAGCCAUGGCACUGGCUUCACGUCC
UUUGGCUUACUCAAACUCUGA

Furthermore, preferably the RNA construct comprises an RNA nucleotide sequence substantially as set out in SEQ ID No: 87, or a variant or fragment thereof.

In one embodiment, the at least one ISP may be Human CD27 Ligand (NCBI Reference Sequence: NM_001252.5; UniProtKB—P32970 (CD70_HUMAN)), or an orthologue thereof. One embodiment of the polypeptide sequence of the Human CD27 Ligand is represented herein as SEQ ID No: 88, as follows:

[SEQ ID No: 88]
MPEEGSGCSVRRRPYGCVLRAALVPLVAGLVICLVVCIQRFAQAQ
QQLPLESLGWDVAELQLNHTGPQQDPRLYWQGGPALGRSFLHGPE
LDKGQLRIHRDGIYMVHIQVTLAICSSTTASRHHPTTLAVGICSP
ASRSISLLRLSFHQGCTIASQRLTPLARGDTLCTNLIGTLLPSRN
TDETFFGVQWVRP

Therefore, preferably the RNA construct of any aspect comprises a nucleotide sequence which encodes an amino acid sequence substantially as set out in SEQ ID No: 88, or a variant or fragment thereof.

In one embodiment, the Human CD27 Ligand polypeptide is encoded by the DNA nucleotide sequence of SEQ ID No: 89, as follows:

[SEQ ID No: 89]
ATGCCGGAGGAGGGTTCGGGCTGCTCGGTGCGGCGCAGGCCCTAT
GGGTGCGTCCTGCGGGCTGCTTTGGTCCCATTGGTCGCGGGCTTG
GTGATCTGCCTCGTGGTGTGCATCCAGCGCTTCGCACAGGCTCAG
CAGCAGCTGCCGCTCGAGTCACTTGGGTGGGACGTAGCTGAGCTG
CAGCTGAATCACACAGGACCTCAGCAGGACCCCAGGCTATACTGG
CAGGGGGGCCCAGCACTGGGCCGCTCCTTCCTGCATGGACCAGAG
CTGGACAAGGGGCAGCTACGTATCCATCGTGATGGCATCTACATG
GTACACATCCAGGTGACGCTGGCCATCTGCTCCTCCACGACGGCC
TCCAGGCACCACCCCACCACCCTGGCCGTGGGAATCTGCTCTCCC
GCCTCCCGTAGCATCAGCCTGCTGCGTCTCAGCTTCCACCAAGGT
TGTACCATTGCCTCCCAGCGCCTGACGCCCCTGGCCCGAGGGGAC
ACACTCTGCACCAACCTCACTGGGACACTTTTGCCTTCCCGAAAC
ACTGATGAGACCTTCTTTGGAGTGCAGTGGGTGCGCCCCTGA

Accordingly, preferably the Human CD27 Ligand polypeptide is encoded by the DNA nucleotide sequence substantially as set out in SEQ ID NO: 89, or a variant or fragment thereof.

Thus, the RNA construct may comprise an RNA nucleotide sequence of SEQ ID No: 90, as follows:

[SEQ ID No: 90]
AUGCCGGAGGAGGGUUCGGGCUGCUCGGUGCGGCGCAGGCCCUAU
GGGUGCGUCCUGCGGGCUGCUUUGGUCCCAUUGGUCGCGGGCUUG
GUGAUCUGCCUCGUGGUGUGCAUCCAGCGCUUCGCACAGGCUCAG
CAGCAGCUGCCGCUCGAGUCACUUGGGUGGGACGUAGCUGAGCUG
CAGCUGAAUCACACAGGACCUCAGCAGGACCCCAGGCUAUACUGG
CAGGGGGGCCCAGCACUGGGCCGCUCCUUCCUGCAUGGACCAGAG
CUGGACAAGGGGCAGCUACGUAUCCAUCGUGAUGGCAUCUACAUG
GUACACAUCCAGGUGACGCUGGCCAUCUGCUCCUCCACGACGGCC
UCCAGGCACCACCCCACCACCCUGGCCGUGGGAAUCUGCUCUCCC
GCCUCCCGUAGCAUCAGCCUGCUGCGUCUCAGCUUCCACCAAGGU
UGUACCAUUGCCUCCCAGCGCCUGACGCCCCUGGCCCGAGGGGAC
ACACUCUGCACCAACCUCACUGGGACACUUUUGCCUUCCCGAAAC
ACUGAUGAGACCUUCUUUGGAGUGCAGUGGGUGCGCCCCUGA

Furthermore, preferably the RNA construct comprises an RNA nucleotide sequence substantially as set out in SEQ ID No: 90, or a variant or fragment thereof.

The ISP may be Tumour necrosis factor, which may be either the membrane form or the soluble form. In one embodiment, the at least one ISP may be Human TNF beta (NCBI Reference Sequence: NM_000595.4; UniProtKB—P01374 (TNFB_HUMAN)), or an orthologue thereof. One embodiment of the polypeptide sequence of Human TNF beta is represented herein as SEQ ID No: 91, as follows:

[SEQ ID No: 91]
MTPPERLFLPRVCGTTLHLLLLGLLLVLLPGAQGLPGVGLTPSAA
QTARQHPKMHLAHSTLKPAAHLIGDPSKQNSLLWRANTDRAFLQD
GFSLSNNSLLVPTSGIYFVYSQVVESGKAYSPKATSSPLYLAHEV
QLESSQYPFHVPLLSSQKMVYPGLQEPWLHSMYHGAAFQLTQGDQ
LSTHTDGIPHLVLSPSTVFFGAFAL

Therefore, preferably the RNA construct of any aspect comprises a nucleotide sequence which encodes an amino acid sequence substantially as set out in SEQ ID No: 91, or a variant or fragment thereof.

In one embodiment, the Human TNF beta polypeptide is encoded by the DNA nucleotide sequence of SEQ ID No: 92, as follows:

[SEQ ID No: 92]
ATGACACCACCTGAACGTCTCTTCCTCCCAAGGGTGTGTGGCACC
ACCCTACACCTCCTCCTTCTGGGGCTGCTGCTGGTTCTGCTGCCT
GGGGCCCAGGGGCTCCCTGGTGTTGGCCTCACACCTTCAGCTGCC
CAGACTGCCCGTCAGCACCCCAAGATGCATCTTGCCCACAGCACC
CTCAAACCTGCTGCTCACCTCATTGGAGACCCCAGCAAGCAGAAC
TCACTGCTCTGGAGAGCAAACACGGACCGTGCCTTCCTCCAGGAT
GGTTTCTCCTTGAGCAACAATTCTCTCCTGGTCCCCACCAGTGGC
ATCTACTTCGTCTACTCCCAGGTGGTCTTCTCTGGGAAAGCCTAC
TCTCCCAAGGCCACCTCCTCCCCACTCTACCTGGCCCATGAGGTC
CAGCTCTTCTCCTCCCAGTACCCCTTCCATGTGCCTCTCCTCAGC
TCCCAGAAGATGGTGTATCCAGGGCTGCAGGAACCCTGGCTGCAC
TCGATGTACCACGGGGCTGCGTTCCAGCTCACCCAGGGAGACCAG
CTATCCACCCACACAGATGGCATCCCCCACCTAGTCCTCAGCCCT
AGTACTGTCTTCTTTGGAGCCTTCGCTCTG

Accordingly, preferably the Human TNF beta polypeptide is encoded by the DNA nucleotide sequence substantially as set out in SEQ ID NO: 92, or a variant or fragment thereof.

Thus, the RNA construct may comprise an RNA nucleotide sequence of SEQ ID No: 93, as follows:

[SEQ ID No: 93]
AUGACACCACCUGAACGUCUCUUCCUCCCAAGGGUGUGUGGCACC
ACCCUACACCUCCUCCUUCUGGGGCUGCUGCUGGUUCUGCUGCCU
GGGGCCCAGGGGCUCCCUGGUGUUGGCCUCACACCUUCAGCUGCC
CAGACUGCCCGUCAGCACCCCAAGAUGCAUCUUGCCCACAGCACC
CUCAAACCUGCUGCUCACCUCAUUGGAGACCCCAGCAAGCAGAAC
UCACUGCUCUGGAGAGCAAACACGGACCGUGCCUUCCUCCAGGAU
GGUUUCUCCUUGAGCAACAAUUCUCUCCUGGUCCCCACCAGUGGC
AUCUACUUCGUCUACUCCCAGGUGGUCUUCUCUGGGAAAGCCUAC
UCUCCCAAGGCCACCUCCUCCCCACUCUACCUGGCCCAUGAGGUC
CAGCUCUUCUCCUCCCAGUACCCCUUCCAUGUGCCUCUCCUCAGC
UCCCAGAAGAUGGUGUAUCCAGGGCUGCAGGAACCCUGGCUGCAC
UCGAUGUACCACGGGGCUGCGUUCCAGCUCACCCAGGGAGACCAG
CUAUCCACCCACACAGAUGGCAUCCCCCACCUAGUCCUCAGCCCU
AGUACUGUCUUCUUUGGAGCCUUCGCUCUG

Furthermore, preferably the RNA construct comprises an RNA nucleotide sequence substantially as set out in SEQ ID No: 93, or a variant or fragment thereof.

In one embodiment, the at least one ISP may be Human TNF alpha (NCBI Reference Sequence: NM_000594.4; UniProtKB—P01375 (TNFA_HUMAN)), or an orthologue thereof. One embodiment of the polypeptide sequence of Human TNF alpha is represented herein as SEQ ID No: 94, as follows:

[SEQ ID No: 94]
MSTESMIRDVELAEEALPKKTGGPQGSRRCLFLSLFSFLIVAGAT
TLFCLLHFGVIGPQREEFPRDLSLISPLAQAVRSSSRTPSDKPVA
HVVANPQAEGQLQWLNRRANALLANGVELRDNQLVVPSEGLYLIY
SQVLFKGQGCPSTHVLLTHTISRIAVSYQTKVNLLSAIKSPCQRE
TPEGAEAKPWYEPIYLGGVFQLEKGDRLSAEINRPDYLDFAESGQ
VYFGIIAL

Therefore, preferably the RNA construct of any aspect comprises a nucleotide sequence which encodes an amino acid sequence substantially as set out in SEQ ID No: 94, or a variant or fragment thereof.

In one embodiment, the Human TNF alpha polypeptide is encoded by the DNA nucleotide sequence of SEQ ID No: 95, as follows:

[SEQ ID No: 95]
ATGAGCACTGAAAGCATGATCCGGGACGTGGAGCTGGCCGAGGAG
GCGCTCCCCAAGAAGACAGGGGGGCCCCAGGGCTCCAGGCGGTGC
TTGTTCCTCAGCCTCTTCTCCTTCCTGATCGTGGCAGGCGCCACC
ACGCTCTTCTGCCTGCTGCACTTTGGAGTGATCGGCCCCCAGAGG
GAAGAGTTCCCCAGGGACCTCTCTCTAATCAGCCCTCTGGCCCAG
GCAGTCAGATCATCTTCTCGAACCCCGAGTGACAAGCCTGTAGCC
CATGTTGTAGCAAACCCTCAAGCTGAGGGGCAGCTCCAGTGGCTG
AACCGCCGGGCCAATGCCCTCCTGGCCAATGGCGTGGAGCTGAGA
GATAACCAGCTGGTGGTGCCATCAGAGGGCCTGTACCTCATCTAC
TCCCAGGTCCTCTTCAAGGGCCAAGGCTGCCCCTCCACCCATGTG
CTCCTCACCCACACCATCAGCCGCATCGCCGTCTCCTACCAGACC
AAGGTCAACCTCCTCTCTGCCATCAAGAGCCCCTGCCAGAGGGAG
ACCCCAGAGGGGGCTGAGGCCAAGCCCTGGTATGAGCCCATCTAT
CTGGGAGGGGTCTTCCAGCTGGAGAAGGGTGACCGACTCAGCGCT
GAGATCAATCGGCCCGACTATCTCGACTTTGCCGAGTCTGGGCAG
GTCTACTTTGGGATCATTGCCCTG

Accordingly, preferably the Human TNF alpha polypeptide is encoded by the DNA nucleotide sequence substantially as set out in SEQ ID NO: 95, or a variant or fragment thereof.

Thus, the RNA construct may comprise an RNA nucleotide sequence of SEQ ID No: 96, as follows:

[SEQ ID No: 96]
AUGAGCACUGAAAGCAUGAUCCGGGACGUGGAGCUGGCCGAGGAG
GCGCUCCCCAAGAAGACAGGGGGGCCCCAGGGCUCCAGGCGGUGC
UUGUUCCUCAGCCUCUUCUCCUUCCUGAUCGUGGCAGGCGCCACC
ACGCUCUUCUGCCUGCUGCACUUUGGAGUGAUCGGCCCCCAGAGG
GAAGAGUUCCCCAGGGACCUCUCUCUAAUCAGCCCUCUGGCCCAG
GCAGUCAGAUCAUCUUCUCGAACCCCGAGUGACAAGCCUGUAGCC
CAUGUUGUAGCAAACCCUCAAGCUGAGGGGCAGCUCCAGUGGCUG
AACCGCCGGGCCAAUGCCCUCCUGGCCAAUGGCGUGGAGCUGAGA
GAUAACCAGCUGGUGGUGCCAUCAGAGGGCCUGUACCUCAUCUAC
UCCCAGGUCCUCUUCAAGGGCCAAGGCUGCCCCUCCACCCAUGUG
CUCCUCACCCACACCAUCAGCCGCAUCGCCGUCUCCUACCAGACC
AAGGUCAACCUCCUCUCUGCCAUCAAGAGCCCCUGCCAGAGGGAG
ACCCCAGAGGGGGCUGAGGCCAAGCCCUGGUAUGAGCCCAUCUAU
CUGGGAGGGGUCUUCCAGCUGGAGAAGGGUGACCGACUCAGCGCU
GAGAUCAAUCGGCCCGACUAUCUCGACUUUGCCGAGUCUGGGCAG
GUCUACUUUGGGAUCAUUGCCCUG

Furthermore, preferably the RNA construct comprises an RNA nucleotide sequence substantially as set out in SEQ ID No: 96, or a variant or fragment thereof.

In one embodiment, the at least one ISP may be TNFSF10 (NCBI Reference Sequence: NM_003810.4; UniProtKB—P50591 (TNF10_HUMAN)), or an orthologue thereof. One embodiment of the polypeptide sequence of TNFSF10 is represented herein as SEQ ID No: 97, as follows:

[SEQ ID No: 97]
MAMMEVQGGPSLGQTCVLIVIFTVLLQSLCVAVTYVYFTNELKQM
QDKYSKSGIACFLKEDDSYWDPNDEESMNSPCWQVKWQLRQLVRK
MILRTSEETISTVQEKQQNISPLVRERGPQRVAAHITGTRGRSNT
LSSPNSKNEKALGRKINSWESSRSGHSFLSNLHLRNGELVIHEKG
FYYIYSQTYFRFQEEIKENTKNDKQMVQYIYKYTSYPDPILLMKS
ARNSCWSKDAEYGLYSIYQGGIFELKENDRIFVSVTNEHLIDMDH
EASFFGAFLVG

Therefore, preferably the RNA construct of any aspect comprises a nucleotide sequence which encodes an amino acid sequence substantially as set out in SEQ ID No: 97, or a variant or fragment thereof.

In one embodiment, the TNFSF10 polypeptide is encoded by the DNA nucleotide sequence of SEQ ID No: 98, as follows:

[SEQ ID No: 98]
ATGGCTATGATGGAGGTCCAGGGGGGACCCAGCCTGGGACAGACC
TGCGTGCTGATCGTGATCTTCACAGTGCTCCTGCAGTCTCTCTGT
GTGGCTGTAACTTACGTGTACTTTACCAACGAGCTGAAGCAGATG
CAGGACAAGTACTCCAAAAGTGGCATTGCTTGTTTCTTAAAAGAA
GATGACAGTTATTGGGACCCCAATGACGAAGAGAGTATGAACAGC
CCCTGCTGGCAAGTCAAGTGGCAACTCCGTCAGCTCGTTAGAAAG
ATGATTTTGAGAACCTCTGAGGAAACCATTICTACAGTTCAAGAA
AAGCAACAAAATATTTCTCCCCTAGTGAGAGAAAGAGGTCCTCAG
AGAGTAGCAGCTCACATAACTGGGACCAGAGGAAGAAGCAACACA
TTGTCTTCTCCAAACTCCAAGAATGAAAAGGCTCTGGGCCGCAAA
ATAAACTCCTGGGAATCATCAAGGAGTGGGCATTCATTCCTGAGC
AACTTGCACTTGAGGAATGGTGAACTGGTCATCCATGAAAAAGGG
TTTTACTACATCTATTCCCAAACATACTTTCGATTTCAGGAGGAA
ATAAAAGAAAACACAAAGAACGACAAACAAATGGTCCAATATATT
TACAAATACACAAGTTATCCTGACCCTATATTGTTGATGAAAAGT
GCTAGAAATAGTTGTTGGTCTAAAGATGCAGAATATGGACTCTAT
TCCATCTATCAAGGGGGAATATTTGAGCTTAAGGAAAATGACAGA
ATTTTTGTTTCTGTAACAAATGAGCACTTGATAGACATGGACCAT
GAAGCCAGTTTTTTTGGGGCCTTTTTAGTTGGC

Accordingly, preferably the TNFSF10 polypeptide is encoded by the DNA nucleotide sequence substantially as set out in SEQ ID NO: 98, or a variant or fragment thereof.

Thus, the RNA construct may comprise an RNA nucleotide sequence of SEQ ID No: 99, as follows:

[SEQ ID No: 99]
AUGGCUAUGAUGGAGGUCCAGGGGGGACCCAGCCUGGGACAGACC
UGCGUGCUGAUCGUGAUCUUCACAGUGCUCCUGCAGUCUCUCUGU
GUGGCUGUAACUUACGUGUACUUUACCAACGAGCUGAAGCAGAUG
CAGGACAAGUACUCCAAAAGUGGCAUUGCUUGUUUCUUAAAAGAA
GAUGACAGUUAUUGGGACCCCAAUGACGAAGAGAGUAUGAACAGC
CCCUGCUGGCAAGUCAAGUGGCAACUCCGUCAGCUCGUUAGAAAG
AUGAUUUUGAGAACCUCUGAGGAAACCAUUUCUACAGUUCAAGAA
AAGCAACAAAAUAUUUCUCCCCUAGUGAGAGAAAGAGGUCCUCAG
AGAGUAGCAGCUCACAUAACUGGGACCAGAGGAAGAAGCAACACA
UUGUCUUCUCCAAACUCCAAGAAUGAAAAGGCUCUGGGCCGCAAA
AUAAACUCCUGGGAAUCAUCAAGGAGUGGGCAUUCAUUCCUGAGC
AACUUGCACUUGAGGAAUGGUGAACUGGUCAUCCAUGAAAAAGGG
UUUUACUACAUCUAUUCCCAAACAUACUUUCGAUUUCAGGAGGAA
AUAAAAGAAAACACAAAGAACGACAAACAAAUGGUCCAAUAUAUU
UACAAAUACACAAGUUAUCCUGACCCUAUAUUGUUGAUGAAAAGU
GCUAGAAAUAGUUGUUGGUCUAAAGAUGCAGAAUAUGGACUCUAU
UCCAUCUAUCAAGGGGGAAUAUUUGAGCUUAAGGAAAAUGACAGA
AUUUUUGUUUCUGUAACAAAUGAGCACUUGAUAGACAUGGACCAU
GAAGCCAGUUUUUUUGGGGCCUUUUUAGUUGGC

Furthermore, preferably the RNA construct comprises an RNA nucleotide sequence substantially as set out in SEQ ID No: 99, or a variant or fragment thereof.

Preferably, the ISP is a growth factor. In one embodiment, the at least one ISP may be Transforming Growth Factor α (TGFα) (Membrane Bound; NCBI Reference Sequence: NM_003236.4; UniProtKB—P01135 (TGFA_HUMAN)), or an orthologue thereof. One embodiment of the polypeptide sequence of TGFα is represented herein as SEQ ID No: 100, as follows:

[SEQ ID No: 100]
MVPSAGQLALFALGIVLAACQALENSTSPLSADPPVAAAVVSHEN
DCPDSHTQFCFHGTCRFLVQEDKPACVCHSGYVGARCEHADLLAV
VAASQKKQAITALVVVSIVALAVLIITCVLIHCCQVRKHCEWCRA
LICRHEKPSALLKGRTACCHSETVV

Therefore, preferably the RNA construct of any aspect comprises a nucleotide sequence which encodes an amino acid sequence substantially as set out in SEQ ID No: 100, or a variant or fragment thereof.

In one embodiment, the TGFα polypeptide is encoded by the DNA nucleotide sequence of SEQ ID No: 101, as follows:

[SEQ ID No: 101]
ATGGTCCCCTCGGCTGGACAGCTCGCCCTGTTCGCTCTGGGTATT
GTGTTGGCTGCGTGCCAGGCCTTGGAGAACAGCACGTCCCCGCTG
AGTGCAGACCCGCCCGTGGCTGCAGCAGTGGTGTCCCATTTTAAT
GACTGCCCAGATTCCCACACTCAGTTCTGCTTCCATGGAACCTGC
AGGTTTTTGGTGCAGGAGGACAAGCCAGCATGTGTCTGCCATTCT
GGGTACGTTGGTGCACGCTGTGAGCATGCGGACCTCCTGGCCGTG
GTGGCTGCCAGCCAGAAGAAGCAGGCCATCACCGCCTTGGTGGTG
GTCTCCATCGTGGCCCTGGCTGTCCTTATCATCACATGTGTGC

Accordingly, preferably the TGFα polypeptide is encoded by the DNA nucleotide sequence substantially as set out in SEQ ID NO: 101, or a variant or fragment thereof.

Thus, the RNA construct may comprise an RNA nucleotide sequence of SEQ ID No: 102, as follows:

[SEQ ID No: 102]
AUGGUCCCCUCGGCUGGACAGCUCGCCCUGUUCGCUCUGGGUAUU
GUGUUGGCUGCGUGCCAGGCCUUGGAGAACAGCACGUCCCCGCUG
AGUGCAGACCCGCCCGUGGCUGCAGCAGUGGUGUCCCAUUUUAAU
GACUGCCCAGAUUCCCACACUCAGUUCUGCUUCCAUGGAACCUGC
AGGUUUUUGGUGCAGGAGGACAAGCCAGCAUGUGUCUGCCAUUCU
GGGUACGUUGGUGCACGCUGUGAGCAUGCGGACCUCCUGGCCGUG
GUGGCUGCCAGCCAGAAGAAGCAGGCCAUCACCGCCUUGGUGGUG
GUCUCCAUCGUGGCCCUGGCUGUCCUUAUCAUCACAUGUGUGC

Furthermore, preferably the RNA construct comprises an RNA nucleotide sequence substantially as set out in SEQ ID No: 102, or a variant or fragment thereof.

In one embodiment, the at least one ISP may be Transforming Growth Factor α (TGFα) (Soluble mature form; NCBI Reference Sequence: NM_003236.4; UniProtKB—P01135 (TGFA_HUMAN)), or an orthologue thereof. One embodiment of the polypeptide sequence of soluble TGFα is represented herein as SEQ ID No: 103, as follows:

[SEQ ID No: 103]
MDRAKLLLLLLLLLLPQAQAVVSHENDCPDSHTQFCFHGTCRFLV
QEDKPACVCHSGYVGARCEHADLLA

Therefore, preferably the RNA construct of any aspect comprises a nucleotide sequence which encodes an amino acid sequence substantially as set out in SEQ ID No: 103, or a variant or fragment thereof.

In one embodiment, the soluble TGFα; polypeptide is encoded by the DNA nucleotide sequence of SEQ ID No: 104, as follows:

[SEQ ID No: 104]
ATGGACAGAGCCAAGCTGCTGCTGTTGCTGCTCCTTCTGCTGCTG
CCTCAGGCTCAAGCTGTGGTGTCCCATTTTAATGACTGCCCAGAT
TCCCACACTCAGTTCTGCTTCCATGGAACCTGCAGGTTTTTGGTG
CAGGAGGACAAGCCAGCATGTGTCTGCCATTCTGGGTACGTTGGT
GCACGCTGTGAGCATGCGGACCTCCTGGCC

Accordingly, preferably the soluble TGFα polypeptide is encoded by the DNA nucleotide sequence substantially as set out in SEQ ID NO: 104, or a variant or fragment thereof.

Thus, the RNA construct may comprise an RNA nucleotide sequence of SEQ ID No: 105, as follows:

[SEQ ID No: 105]
AUGGACAGAGCCAAGCUGCUGCUGUUGCUGCUCCUUCUGCUGCUGCCUCA
GGCUCAAGCUGUGGUGUCCCAUUUUAAUGACUGCCCAGAUUCCCACACUC
AGUUCUGCUUCCAUGGAACCUGCAGGUUUUUGGUGCAGGAGGACAAGCCA
GCAUGUGUCUGCCAUUCUGGGUACGUUGGUGCACGCUGUGAGCAUGCGGA
CCUCCUGGCC

Furthermore, preferably the RNA construct comprises an RNA nucleotide sequence substantially as set out in SEQ ID No: 105, or a variant or fragment thereof.

In one embodiment, the at least one ISP may be Transforming Growth Factor β (TGFβ) family (NCBI Reference Sequence: NM_000660.7; UniProtKB—P01137 (TGFB1_HUMAN), or an orthologue thereof. One embodiment of the polypeptide sequence of TGFβ is represented herein as SEQ ID No:106 as follows:

[SEQ ID No: 106]
MDRAKLLLLLLLLLLPQAQAALDTNYCESSTEKNCCVRQLYIDERKDLGW
KWIHEPKGYHANFCLGPCPYIWSLDTQYSKVLALYNQHNPGASAAPCCVP
QALEPLPIVYYVGRKPKVEQLSNMIVRSCKCS

Therefore, preferably the RNA construct of any aspect comprises a nucleotide sequence which encodes an amino acid sequence substantially as set out in SEQ ID No: 106, or a variant or fragment thereof.

In one embodiment, the TGFβ polypeptide is encoded by the DNA nucleotide sequence of SEQ ID No: 107, as follows:

[SEQ ID No: 107]
ATGGACAGAGCCAAGCTGCTGCTGTTGCTGCTCCTTCTGCTGCTGCCTCA
GGCTCAAGCTGCCCTGGACACCAACTATTGCTTCAGCTCCACGGAGAAGA
ACTGCTGCGTGCGGCAGCTGTACATTGACTTCCGCAAGGACCTCGGCTGG
AAGTGGATCCACGAGCCCAAGGGCTACCATGCCAACTTCTGCCTCGGGCC
CTGCCCCTACATTTGGAGCCTGGACACGCAGTACAGCAAGGTCCTGGCCC
TGTACAACCAGCATAACCCGGGCGCCTCGGCGGCGCCGTGCTGCGTGCCG
CAGGCGCTGGAGCCGCTGCCCATCGTGTACTACGTGGGCCGCAAGCCCAA
GGTGGAGCAGCTGTCCAACATGATCGTGCGCTCCTGCAAGTGCAGC

Accordingly, preferably the TGFβ polypeptide is encoded by the DNA nucleotide sequence substantially as set out in SEQ ID NO: 107, or a variant or fragment thereof.

Thus, the RNA construct may comprise an RNA nucleotide sequence of SEQ ID No: 108, as follows:

[SEQ ID No: 108]
AUGGACAGAGCCAAGCUGCUGCUGUUGCUGCUCCUUCUGCUGCUGCCUCA
GGCUCAAGCUGCCCUGGACACCAACUAUUGCUUCAGCUCCACGGAGAAGA
ACUGCUGCGUGCGGCAGCUGUACAUUGACUUCCGCAAGGACCUCGGCUGG
AAGUGGAUCCACGAGCCCAAGGGCUACCAUGCCAACUUCUGCCUCGGGCC
CUGCCCCUACAUUUGGAGCCUGGACACGCAGUACAGCAAGGUCCUGGCCC
UGUACAACCAGCAUAACCCGGGCGCCUCGGCGGCGCCGUGCUGCGUGCCG
CAGGCGCUGGAGCCGCUGCCCAUCGUGUACUACGUGGGCCGCAAGCCCAA
GGUGGAGCAGCUGUCCAACAUGAUCGUGCGCUCCUGCAAGUGCAGC

Furthermore, preferably the RNA construct comprises an RNA nucleotide sequence substantially as set out in SEQ ID No: 108, or a variant or fragment thereof.

In another embodiment, the ISP may be a colony stimulating factor. In one embodiment, the at least one ISP may be CSF1 (macrophage colony-stimulating factor; NCBI Reference Sequence: NM_000757.6; UniProtKB—P09603 (CSF1_HUMAN)), or an orthologue thereof. One embodiment of the polypeptide sequence of CSF1 (signal is 1-32) is represented herein as SEQ ID No: 109, as follows:

[SEQ ID No: 109]
MTAPGAAGRCPPTTWLGSLLLLVCLLASRSITEEVSEYCSHMIGSGHLQS
LQRLIDSQMETSCQITFEFVDQEQLKDPVCYLKKAFLLVQDIMEDTMRFR
DNTPNAIAIVQLQELSLRLKSCFTKDYEEHDKACVRTFYETPLQLLEKVK
NVFNETKNLLDKDWNIFSKNCNNSFAECSSQDVVTKPDCNCLYPKAIPSS
DPASVSPHQPLAPSMAPVAGLTWEDSEGTEGSSLLPGEQPLHTVDPGSAK
QRPPRSTCQSFEPPETPVVKDSTIGGSPQPRPSVGAFNPGMEDILDSAMG
TNWVPEEASGEASEIPVPQGTELSPSRPGGGSMQTEPARPSNFLSASSPL
PASAKGQQPADVTGTALPRVGPVRPTGQDWNHTPQKTDHPSALLRDPPEP
GSPRISSLRPQGLSNPSTLSAQPQLSRSHSSGSVLPLGELEGRRSTRDRR
SPAEPEGGPASEGAARPLPRFNSVPLTDTGHERQSEGSFSPQLQESVFHL
LVPSVILVLLAVGGLLFYRWRRRSHQEPQRADSPLEQPEGSPLTQDDRQV
ELPV

Therefore, preferably the RNA construct of any aspect comprises a nucleotide sequence which encodes an amino acid sequence substantially as set out in SEQ ID No: 109, or a variant or fragment thereof.

In one embodiment, the CSF1 polypeptide is encoded by the DNA nucleotide sequence of SEQ ID No: 110, as follows:

[SEQ ID No: 110]
ATGACCGCGCCGGGCGCCGCCGGGCGCTGCCCTCCCACGACATGGCTGGG
CTCCCTGCTGTTGTTGGTCTGTCTCCTGGCGAGCAGGAGTATCACCGAGG
AGGTGTCGGAGTACTGTAGCCACATGATTGGGAGTGGACACCTGCAGTCT
CTGCAGCGGCTGATTGACAGTCAGATGGAGACCTCGTGCCAAATTACATT
TGAGTTTGTAGACCAGGAACAGTTGAAAGATCCAGTGTGCTACCTTAAGA
AGGCATTTCTCCTGGTACAAGACATAATGGAGGACACCATGCGCTTCAGA
GATAACACCCCCAATGCCATCGCCATTGTGCAGCTGCAGGAACTCTCTTT
GAGGCTGAAGAGCTGCTTCACCAAGGATTATGAAGAGCATGACAAGGCCT
GCGTCCGAACTTTCTATGAGACACCTCTCCAGTTGCTGGAGAAGGTCAAG
AATGTCTTTAATGAAACAAAGAATCTCCTTGACAAGGACTGGAATATTTT
CAGCAAGAACTGCAACAACAGCTTTGCTGAATGCTCCAGCCAAGATGTGG
TGACCAAGCCTGATTGCAACTGCCTGTACCCCAAAGCCATCCCTAGCAGT
GACCCGGCCTCTGTCTCCCCTCATCAGCCCCTCGCCCCCTCCATGGCCCC
TGTGGCTGGCTTGACCTGGGAGGACTCTGAGGGAACTGAGGGCAGCTCCC
TCTTGCCTGGTGAGCAGCCCCTGCACACAGTGGATCCAGGCAGTGCCAAG
CAGCGGCCACCCAGGAGCACCTGCCAGAGCTTTGAGCCGCCAGAGACCCC
AGTTGTCAAGGACAGCACCATCGGTGGCTCACCACAGCCTCGCCCCTCTG
TCGGGGCCTTCAACCCCGGGATGGAGGATATTCTTGACTCTGCAATGGGC
ACTAATTGGGTCCCAGAAGAAGCCTCTGGAGAGGCCAGTGAGATTCCCGT
ACCCCAAGGGACAGAGCTTTCCCCCTCCAGGCCAGGAGGGGGCAGCATGC
AGACAGAGCCCGCCAGACCCAGCAACTTCCTCTCAGCATCTTCTCCACTC
CCTGCATCAGCAAAGGGCCAACAGCCGGCAGATGTAACTGGTACCGCCTT
GCCCAGGGTGGGCCCCGTGAGGCCCACTGGCCAGGACTGGAATCACACCC
CCCAGAAGACAGACCATCCATCTGCCCTGCTCAGAGACCCCCCGGAGCCA
GGCTCTCCCAGGATCTCATCACTGCGCCCCCAGGGCCTCAGCAACCCCTC
CACCCTCTCTGCTCAGCCACAGCTTTCCAGAAGCCACTCCTCGGGCAGCG
TGCTGCCCCTTGGGGAGCTGGAGGGCAGGAGGAGCACCAGGGATCGGAGG
AGCCCCGCAGAGCCAGAAGGAGGACCAGCAAGTGAAGGGGCAGCCAGGCC
CCTGCCCCGTTTTAACTCCGTTCCTTTGACTGACACAGGCCATGAGAGGC
AGTCCGAGGGATCCTTCAGCCCGCAGCTCCAGGAGTCTGTCTTCCACCTG
CTGGTGCCCAGTGTCATCCTGGTCTTGCTGGCCGTCGGAGGCCTCTTGTT
CTACAGGTGGAGGCGGCGGAGCCATCAAGAGCCTCAGAGAGCGGATTCTC
CCTTGGAGCAACCAGAGGGCAGCCCCCTGACTCAGGATGACAGACAGGTG
GAACTGCCAGTG

Accordingly, preferably the CSF1 polypeptide is encoded by the DNA nucleotide sequence substantially as set out in SEQ ID NO: 110, or a variant or fragment thereof.

Thus, the RNA construct may comprise an RNA nucleotide sequence of SEQ ID No: 111 as follows:

[SEQ ID No: 111]
AUGACCGCGCCGGGCGCCGCCGGGCGCUGCCCUCCCACGACAUGGCUGGG
CUCCCUGCUGUUGUUGGUCUGUCUCCUGGCGAGCAGGAGUAUCACCGAGG
AGGUGUCGGAGUACUGUAGCCACAUGAUUGGGAGUGGACACCUGCAGUCU
CUGCAGCGGCUGAUUGACAGUCAGAUGGAGACCUCGUGCCAAAUUACAUU
UGAGUUUGUAGACCAGGAACAGUUGAAAGAUCCAGUGUGCUACCUUAAGA
AGGCAUUUCUCCUGGUACAAGACAUAAUGGAGGACACCAUGCGCUUCAGA
GAUAACACCCCCAAUGCCAUCGCCAUUGUGCAGCUGCAGGAACUCUCUUU
GAGGCUGAAGAGCUGCUUCACCAAGGAUUAUGAAGAGCAUGACAAGGCCU
GCGUCCGAACUUUCUAUGAGACACCUCUCCAGUUGCUGGAGAAGGUCAAG
AAUGUCUUUAAUGAAACAAAGAAUCUCCUUGACAAGGACUGGAAUAUUUU
CAGCAAGAACUGCAACAACAGCUUUGCUGAAUGCUCCAGCCAAGAUGUGG
UGACCAAGCCUGAUUGCAACUGCCUGUACCCCAAAGCCAUCCCUAGCAGU
GACCCGGCCUCUGUCUCCCCUCAUCAGCCCCUCGCCCCCUCCAUGGCCCC
UGUGGCUGGCUUGACCUGGGAGGACUCUGAGGGAACUGAGGGCAGCUCCC
UCUUGCCUGGUGAGCAGCCCCUGCACACAGUGGAUCCAGGCAGUGCCAAG
CAGCGGCCACCCAGGAGCACCUGCCAGAGCUUUGAGCCGCCAGAGACCCC
AGUUGUCAAGGACAGCACCAUCGGUGGCUCACCACAGCCUCGCCCCUCUG
UCGGGGCCUUCAACCCCGGGAUGGAGGAUAUUCUUGACUCUGCAAUGGGC
ACUAAUUGGGUCCCAGAAGAAGCCUCUGGAGAGGCCAGUGAGAUUCCCGU
ACCCCAAGGGACAGAGCUUUCCCCCUCCAGGCCAGGAGGGGGCAGCAUGC
AGACAGAGCCCGCCAGACCCAGCAACUUCCUCUCAGCAUCUUCUCCACUC
CCUGCAUCAGCAAAGGGCCAACAGCCGGCAGAUGUAACUGGUACCGCCUU
GCCCAGGGUGGGCCCCGUGAGGCCCACUGGCCAGGACUGGAAUCACACCC
CCCAGAAGACAGACCAUCCAUCUGCCCUGCUCAGAGACCCCCCGGAGCCA
GGCUCUCCCAGGAUCUCAUCACUGCGCCCCCAGGGCCUCAGCAACCCCUC
CACCCUCUCUGCUCAGCCACAGCUUUCCAGAAGCCACUCCUCGGGCAGCG
UGCUGCCCCUUGGGGAGCUGGAGGGCAGGAGGAGCACCAGGGAUCGGAGG
AGCCCCGCAGAGCCAGAAGGAGGACCAGCAAGUGAAGGGGCAGCCAGGCC
CCUGCCCCGUUUUAACUCCGUUCCUUUGACUGACACAGGCCAUGAGAGGC
AGUCCGAGGGAUCCUUCAGCCCGCAGCUCCAGGAGUCUGUCUUCCACCUG
CUGGUGCCCAGUGUCAUCCUGGUCUUGCUGGCCGUCGGAGGCCUCUUGUU
CUACAGGUGGAGGCGGCGGAGCCAUCAAGAGCCUCAGAGAGCGGAUUCUC
CCUUGGAGCAACCAGAGGGCAGCCCCCUGACUCAGGAUGACAGACAGGUG
GAACUGCCAGUG

Furthermore, preferably the RNA construct comprises an RNA nucleotide sequence substantially as set out in SEQ ID No: 111, or a variant or fragment thereof.

In one embodiment, the at least one ISP may be CFS2 (or GMCSF; NCBI Reference Sequence: NM_000758.4; UniProtKB—P04141 (CSF2_HUMAN) Protein), or an orthologue thereof. One embodiment of the polypeptide sequence of CFS2 (signal is 1-16) is represented herein as SEQ ID No: 112, as follows:

[SEQ ID No: 112]
MWLQSLLLLGTVACSISAPARSPSPSTQPWEHVNAIQEARRLLNLSRDTA
AEMNETVEVISEMFDLQEPTCLQTRLELYKQGLRGSLTKLKGPLTMMASH
YKQHCPPTPETSCATQIITFESFKENLKDELLVIPFDCWEPVQE

Therefore, preferably the RNA construct of any aspect comprises a nucleotide sequence which encodes an amino acid sequence substantially as set out in SEQ ID No: 112, or a variant or fragment thereof.

In one embodiment, the CFS2 polypeptide is encoded by the DNA nucleotide sequence of SEQ ID No: 113, as follows:

[SEQ ID No: 113]
ATGTGGCTGCAGAGCCTGCTGCTCTTGGGCACTGTGGCCTGCAGCATCTC
TGCACCCGCCCGCTCGCCCAGCCCCAGCACGCAGCCCTGGGAGCATGTGA
ATGCCATCCAGGAGGCCCGGCGTCTCCTGAACCTGAGTAGAGACACTGCT
GCTGAGATGAATGAAACAGTAGAAGTCATCTCAGAAATGTTTGACCTCCA
GGAGCCGACCTGCCTACAGACCCGCCTGGAGCTGTACAAGCAGGGCCTGC
GGGGCAGCCTCACCAAGCTCAAGGGCCCCTTGACCATGATGGCCAGCCAC
TACAAGCAGCACTGCCCTCCAACCCCGGAAACTTCCTGTGCAACCCAGAT
TATCACCTTTGAAAGTTTCAAAGAGAACCTGAAGGACTTTCTGCTTGTCA
TCCCCTTTGACTGCTGGGAGCCAGTCCAGGAG

Accordingly, preferably the CFS2 polypeptide is encoded by the DNA nucleotide sequence substantially as set out in SEQ ID NO: 113, or a variant or fragment thereof.

Thus, the RNA construct may comprise an RNA nucleotide sequence of SEQ ID No: 114, as follows:

[SEQ ID No: 114]
AUGUGGCUGCAGAGCCUGCUGCUCUUGGGCACUGUGGCCUGCAGCAUCUC
UGCACCCGCCCGCUCGCCCAGCCCCAGCACGCAGCCCUGGGAGCAUGUGA
AUGCCAUCCAGGAGGCCCGGCGUCUCCUGAACCUGAGUAGAGACACUGCU
GCUGAGAUGAAUGAAACAGUAGAAGUCAUCUCAGAAAUGUUUGACCUCCA
GGAGCCGACCUGCCUACAGACCCGCCUGGAGCUGUACAAGCAGGGCCUGC
GGGGCAGCCUCACCAAGCUCAAGGGCCCCUUGACCAUGAUGGCCAGCCAC
UACAAGCAGCACUGCCCUCCAACCCCGGAAACUUCCUGUGCAACCCAGAU
UAUCACCUUUGAAAGUUUCAAAGAGAACCUGAAGGACUUUCUGCUUGUCA
UCCCCUUUGACUGCUGGGAGCCAGUCCAGGAG

Furthermore, preferably the RNA construct comprises an RNA nucleotide sequence substantially as set out in SEQ ID No: 114, or a variant or fragment thereof.

In one embodiment, the at least one ISP may be CSF3—Granulocyte colony-stimulating factors (also called G-CSF and filgrastim; NCBI Reference Sequence: NM_000759.4; UniProtKB—P09919 (CSF3_HUMAN)), or an orthologue thereof. One embodiment of the polypeptide sequence of the CSF3 is represented herein as SEQ ID No: 115, as follows:

[SEQ ID No: 115]
MAGPATQSPMKLMALQLLLWHSALWTVQEATPLGPASSLPQSFLLKCLEQ
VRKIQGDGAALQEKLVSECATYKLCHPEELVLLGHSLGIPWAPLSSCPSQ
ALQLAGCLSQLHSGLFLYQGLLQALEGISPELGPTLDTLQLDVADFATTI
WQQMEELGMAPALQPTQGAMPAFASAFQRRAGGVLVASHLQSFLEVSYRV
LRHLAQP

Therefore, preferably the RNA construct of any aspect comprises a nucleotide sequence which encodes an amino acid sequence substantially as set out in SEQ ID No: 115, or a variant or fragment thereof.

In one embodiment, the CSF3 polypeptide is encoded by the DNA nucleotide sequence of SEQ ID No: 116, as follows:

[SEQ ID No: 116]
ATGGCTGGACCTGCCACCCAGAGCCCCATGAAGCTGATGGCCCTGCAGCT
GCTGCTGTGGCACAGTGCACTCTGGACAGTGCAGGAAGCCACCCCCCTGG
GCCCTGCCAGCTCCCTGCCCCAGAGCTTCCTGCTCAAGTGCTTAGAGCAA
GTGAGGAAGATCCAGGGCGATGGCGCAGCGCTCCAGGAGAAGCTGGTGAG
TGAGTGTGCCACCTACAAGCTGTGCCACCCCGAGGAGCTGGTGCTGCTCG
GACACTCTCTGGGCATCCCCTGGGCTCCCCTGAGCAGCTGCCCCAGCCAG
GCCCTGCAGCTGGCAGGCTGCTTGAGCCAACTCCATAGCGGCCTTTTCCT
CTACCAGGGGCTCCTGCAGGCCCTGGAAGGGATCTCCCCCGAGTTGGGTC
CCACCTTGGACACACTGCAGCTGGACGTCGCCGACTTTGCCACCACCATC
TGGCAGCAGATGGAAGAACTGGGAATGGCCCCTGCCCTGCAGCCCACCCA
GGGTGCCATGCCGGCCTTCGCCTCTGCTTTCCAGCGCCGGGCAGGAGGGG
TCCTGGTTGCCTCCCATCTGCAGAGCTTCCTGGAGGTGTCGTACCGCGTT
CTACGCCACCTTGCCCAGCCC

Accordingly, preferably the CSF3 polypeptide is encoded by the DNA nucleotide sequence substantially as set out in SEQ ID NO: 116, or a variant or fragment thereof.

Thus, the RNA construct may comprise an RNA nucleotide sequence of SEQ ID No: 117, as follows:

[SEQ ID No: 117]
AUGGCUGGACCUGCCACCCAGAGCCCCAUGAAGCUGAUGGCCCUGCAGCU
GCUGCUGUGGCACAGUGCACUCUGGACAGUGCAGGAAGCCACCCCCCUGG
GCCCUGCCAGCUCCCUGCCCCAGAGCUUCCUGCUCAAGUGCUUAGAGCAA
GUGAGGAAGAUCCAGGGCGAUGGCGCAGCGCUCCAGGAGAAGCUGGUGAG
UGAGUGUGCCACCUACAAGCUGUGCCACCCCGAGGAGCUGGUGCUGCUCG
GACACUCUCUGGGCAUCCCCUGGGCUCCCCUGAGCAGCUGCCCCAGCCAG
GCCCUGCAGCUGGCAGGCUGCUUGAGCCAACUCCAUAGCGGCCUUUUCCU
CUACCAGGGGCUCCUGCAGGCCCUGGAAGGGAUCUCCCCCGAGUUGGGUC
CCACCUUGGACACACUGCAGCUGGACGUCGCCGACUUUGCCACCACCAUC
UGGCAGCAGAUGGAAGAACUGGGAAUGGCCCCUGCCCUGCAGCCCACCCA
GGGUGCCAUGCCGGCCUUCGCCUCUGCUUUCCAGCGCCGGGCAGGAGGGG
UCCUGGUUGCCUCCCAUCUGCAGAGCUUCCUGGAGGUGUCGUACCGCGUU
CUACGCCACCUUGCCCAGCCC

Furthermore, preferably the RNA construct comprises an RNA nucleotide sequence substantially as set out in SEQ ID No: 117, or a variant or fragment thereof.

In one embodiment, the at least one ISP may be Prothymosin alpha (proTα) (1-111; NCBI Reference Sequence: NM_002823.5; UniProtKB—P06454 (PTMA_HUMAN)), or an orthologue thereof. One embodiment of the polypeptide sequence of proTα is represented herein as SEQ ID No: 118, as follows:

[SEQ ID No: 118]
MSDAAVDTSSEITTKDLKEKKEVVEEAENGRDAPANGNANEENGEQEADN
EVDEEEEEGGEEEEEEEEGDGEEEDGDEDEEAESATGKRAAEDDEDDDVD
TKKQKTDEDD

Therefore, preferably the RNA construct of any aspect comprises a nucleotide sequence which encodes an amino acid sequence substantially as set out in SEQ ID No: 118, or a variant or fragment thereof.

In one embodiment, the proTα polypeptide is encoded by the DNA nucleotide sequence of SEQ ID No: 119, as follows:

[SEQ ID No: 119]
ATGTCAGACGCAGCCGTAGACACCAGCTCCGAAATCACCACCAAGGACTT
AAAGGAGAAGAAGGAAGTTGTGGAAGAGGCAGAAAATGGAAGAGACGCCC
CTGCTAACGGGAATGCTAATGAGGAAAATGGGGAGCAGGAGGCTGACAAT
GAGGTAGACGAAGAAGAGGAAGAAGGTGGGGAGGAAGAGGAGGAGGAAGA
AGAAGGTGATGGTGAGGAAGAGGATGGAGATGAAGATGAGGAAGCTGAGT
CAGCTACGGGCAAGCGGGCAGCTGAAGATGATGAGGATGACGATGTCGAT
ACCAAGAAGCAGAAGACCGACGAGGATGAC

Accordingly, preferably the proTα polypeptide is encoded by the DNA nucleotide sequence substantially as set out in SEQ ID NO: 119, or a variant or fragment thereof.

Thus, the RNA construct may comprise an RNA nucleotide sequence of SEQ ID No: 120, as follows:

[SEQ ID No: 120]
AUGUCAGACGCAGCCGUAGACACCAGCUCCGAAAUCACCACCAAGGACU
UAAAGGAGAAGAAGGAAGUUGUGGAAGAGGCAGAAAAUGGAAGAGACGC
CCCUGCUAACGGGAAUGCUAAUGAGGAAAAUGGGGAGCAGGAGGCUGAC
AAUGAGGUAGACGAAGAAGAGGAAGAAGGUGGGGAGGAAGAGGAGGAGG
AAGAAGAAGGUGAUGGUGAGGAAGAGGAUGGAGAUGAAGAUGAGGAAGC
UGAGUCAGCUACGGGCAAGCGGGCAGCUGAAGAUGAUGAGGAUGACGAU
GUCGAUACCAAGAAGCAGAAGACCGACGAGGAUGAC

Furthermore, preferably the RNA construct comprises an RNA nucleotide sequence substantially as set out in SEQ ID No: 120, or a variant or fragment thereof.

In one embodiment, the at least one ISP may be Prothymosin alpha (proTα) (1-11—with secretion signal; NCBI Reference Sequence: NM_002823.5; UniProtKB—P06454 (PTMA_HUMAN)), or an orthologue thereof. One embodiment of the polypeptide sequence of secreted proTα is represented herein as SEQ ID No: 121, as follows:

[SEQ ID No: 121]
MDRAKLLLLLLLLLLPQAQASDAAVDTSSEITTKDLKEKKEVVEEAENG
RDAPANGNANEENGEQEADNEVDEEEEEGGEEEEEEEEGDGEEEDGDED
EEAESATGKRAAEDDEDDDVDTKKQKTDEDD

Therefore, preferably the RNA construct of any aspect comprises a nucleotide sequence which encodes an amino acid sequence substantially as set out in SEQ ID No: 121, or a variant or fragment thereof.

In one embodiment, the secreted proTα polypeptide is encoded by the DNA nucleotide sequence of SEQ ID No: 122, as follows:

[SEQ ID No: 122]
ATGGACAGAGCCAAGCTGCTGCTGTTGCTGCTCCTTCTGCTGCTGCCTC
AGGCTCAAGCTTCAGACGCAGCCGTAGACACCAGCTCCGAAATCACCAC
CAAGGACTTAAAGGAGAAGAAGGAAGTTGTGGAAGAGGCAGAAAATGGA
AGAGACGCCCCTGCTAACGGGAATGCTAATGAGGAAAATGGGGAGCAGG
AGGCTGACAATGAGGTAGACGAAGAAGAGGAAGAAGGTGGGGAGGAAGA
GGAGGAGGAAGAAGAAGGTGATGGTGAGGAAGAGGATGGAGATGAAGAT
GAGGAAGCTGAGTCAGCTACGGGCAAGCGGGCAGCTGAAGATGATGAGG
ATGACGATGTCGATACCAAGAAGCAGAAGACCGACGAGGATGAC

Accordingly, preferably the secreted proTα polypeptide is encoded by the DNA nucleotide sequence substantially as set out in SEQ ID NO: 122, or a variant or fragment thereof.

Thus, the RNA construct may comprise an RNA nucleotide sequence of SEQ ID No: 123, as follows:

[SEQ ID No: 123]
AUGGACAGAGCCAAGCUGCUGCUGUUGCUGCUCCUUCUGCUGCUGCCUC
AGGCUCAAGCUUCAGACGCAGCCGUAGACACCAGCUCCGAAAUCACCAC
CAAGGACUUAAAGGAGAAGAAGGAAGUUGUGGAAGAGGCAGAAAAUGGA
AGAGACGCCCCUGCUAACGGGAAUGCUAAUGAGGAAAAUGGGGAGCAGG
AGGCUGACAAUGAGGUAGACGAAGAAGAGGAAGAAGGUGGGGAGGAAGA
GGAGGAGGAAGAAGAAGGUGAUGGUGAGGAAGAGGAUGGAGAUGAAGAU
GAGGAAGCUGAGUCAGCUACGGGCAAGCGGGCAGCUGAAGAUGAUGAGG
AUGACGAUGUCGAUACCAAGAAGCAGAAGACCGACGAGGAUGAC

Furthermore, preferably the RNA construct comprises an RNA nucleotide sequence substantially as set out in SEQ ID No: 123, or a variant or fragment thereof.

In one embodiment, the at least one ISP may be Prothymosin alpha (proTα) (100-109—with secretion signal; NCBI Reference Sequence: NM_002823.5; UniProtKB—P06454 (PTMA_HUMAN), or an orthologue thereof. One embodiment of the polypeptide sequence of the secreted proTα is represented herein as SEQ ID No: 124, as follows:

[SEQ ID No: 124]
MDRAKLLLLLLLLLLPQAQATKKQKTDEDD

Therefore, preferably the RNA construct of any aspect comprises a nucleotide sequence which encodes an amino acid sequence substantially as set out in SEQ ID No: 124, or a variant or fragment thereof.

In one embodiment, the secreted proTα polypeptide is encoded by the DNA nucleotide sequence of SEQ ID No: 125, as follows:

[SEQ ID No: 125]
ATGGACAGAGCCAAGCTGCTGCTGTTGCTGCTCCTTCTGCTGCTGCCTC
AGGCTCAAGCTACCAAGAAGCAGAAGACCGACGAGGATGAC

Accordingly, preferably the secreted proTα polypeptide is encoded by the DNA nucleotide sequence substantially as set out in SEQ ID NO: 125, or a variant or fragment thereof.

Thus, the RNA construct may comprise an RNA nucleotide sequence of SEQ ID No: 126, as follows:

[SEQ ID No: 126]
AUGGACAGAGCCAAGCUGCUGCUGUUGCUGCUCCUUCUGCUGCUGCCUC
AGGCUCAAGCUACCAAGAAGCAGAAGACCGACGAGGAUGAC

Furthermore, preferably the RNA construct comprises an RNA nucleotide sequence substantially as set out in SEQ ID No: 126, or a variant or fragment thereof.

In one embodiment, the at least one ISP may be Thymosin alpha1 (Talpha1) (NCBI Reference Sequence: NM_001099285.2; UniProtKB—P06454 (PTMA_HUMAN)), or an orthologue thereof. One embodiment of the polypeptide sequence of Talpha1 is represented herein as SEQ ID No: 127, as follows:

[SEQ ID No: 127]
MDRAKLLLLLLLLLLPQAQADAAVDISSEITTKDLKEKKEVVEEAENG

Therefore, preferably the RNA construct of any aspect comprises a nucleotide sequence which encodes an amino acid sequence substantially as set out in SEQ ID No: 127, or a variant or fragment thereof.

In one embodiment, the Talpha1 polypeptide is encoded by the DNA nucleotide sequence of SEQ ID No: 128, as follows:

[SEQ ID No: 128]
ATGGACAGAGCCAAGCTGCTGCTGTTGCTGCTCCTTCTGCTGCTGCCTC
AGGCTCAAGCTGACGCAGCCGTAGACACCAGCTCCGAAATCACCACCAA
GGACTTAAAGGAGAAGAAGGAAGTTGTGGAAGAGGCAGAAAATGGA

Accordingly, preferably the Talpha1 polypeptide is encoded by the DNA nucleotide sequence substantially as set out in SEQ ID NO: 128, or a variant or fragment thereof.

Thus, the RNA construct may comprise an RNA nucleotide sequence of SEQ ID No: 129, as follows:

[SEQ ID No: 129]
AUGGACAGAGCCAAGCUGCUGCUGUUGCUGCUCCUUCUGCUGCUGCCUC
AGGCUCAAGCUGACGCAGCCGUAGACACCAGCUCCGAAAUCACCACCAA
GGACUUAAAGGAGAAGAAGGAAGUUGUGGAAGAGGCAGAAAAUGGA

Furthermore, preferably the RNA construct comprises an RNA nucleotide sequence substantially as set out in SEQ ID No: 129, or a variant or fragment thereof.

In one embodiment, the at least one ISP may be Transmembrane Flt3L (tFlt3L) (NCBI Reference Sequence: NM_001459.4; UniProtKB—P49771 (FLT3L_HUMAN)), or an orthologue thereof. One embodiment of the polypeptide sequence of t Flt3L is represented herein as SEQ ID No: 130, as follows:

[SEQ ID No: 130]
MTVLAPAWSPTTYLLLLLLLSSGLSGTQDCSFQHSPISSDFAVKIRELS
DYLLQDYPVTVASNLQDEELCGGLWRLVLAQRWMERLKTVAGSKMQGLL
ERVNTEIHFVTKCAFQPPPSCLRFVQTNISRLLQETSEQLVALKPWITR
QNFSRCLELQCQPDSSTLPPPWSPRPLEATAPTAPQPPLLLLLLLPVGL
LLLAAAWCLHWQRTRRRTPRPGEQVPPVPSPQDLLLVEH

Therefore, preferably the RNA construct of any aspect comprises a nucleotide sequence which encodes an amino acid sequence substantially as set out in SEQ ID No: 130, or a variant or fragment thereof.

In one embodiment, the tFlt3L polypeptide is encoded by the DNA nucleotide sequence of SEQ ID No: 131, as follows:

[SEQ ID No: 131]
ATGACAGTGCTGGCGCCAGCCTGGAGCCCAACAACCTATCTCCTCCTGCTGCTGCTGCT
GAGCTCGGGACTCAGTGGGACCCAGGACTGCTCCTTCCAACACAGCCCCATCTCCTCCG
ACTTCGCTGTCAAAATCCGTGAGCTGTCTGACTACCTGCTTCAAGATTACCCAGTCACC
GTGGCCTCCAACCTGCAGGACGAGGAGCTCTGCGGGGGCCTCTGGCGGCTGGTCCTGGC
ACAGCGCTGGATGGAGCGGCTCAAGACTGTCGCTGGGTCCAAGATGCAAGGCTTGCTGG
AGCGCGTGAACACGGAGATACACTTTGTCACCAAATGTGCCTTTCAGCCCCCCCCCAGC
TGTCTTCGCTTCGTCCAGACCAACATCTCCCGCCTCCTGCAGGAGACCTCCGAGCAGCT
GGTGGCGCTGAAGCCCTGGATCACTCGCCAGAACTTCTCCCGGTGCCTGGAGCTGCAGT
GTCAGCCCGACTCCTCAACCCTGCCACCCCCATGGAGTCCCCGGCCCCTGGAGGCCACA
GCCCCGACAGCCCCGCAGCCCCCTCTGCTCCTCCTACTGCTGCTGCCCGTGGGCCTCCT
GCTGCTGGCCGCTGCCTGGTGCCTGCACTGGCAGAGGACGCGGCGGAGGACACCCCGCC
CTGGGGAGCAGGTGCCCCCCGTCCCCAGTCCCCAGGACCTGCTGCTTGTGGAGCAC

Accordingly, preferably the tFlt3L polypeptide is encoded by the DNA nucleotide sequence substantially as set out in SEQ ID NO: 131, or a variant or fragment thereof.

Thus, the RNA construct may comprise an RNA nucleotide sequence of SEQ ID No: 132, as follows:

[SEQ ID No: 132]
AUGACAGUGCUGGCGCCAGCCUGGAGCCCAACAACCUAUCUCCUCCUGCUGCUGCUGCU
GAGCUCGGGACUCAGUGGGACCCAGGACUGCUCCUUCCAACACAGCCCCAUCUCCUCCG
ACUUCGCUGUCAAAAUCCGUGAGCUGUCUGACUACCUGCUUCAAGAUUACCCAGUCACC
GUGGCCUCCAACCUGCAGGACGAGGAGCUCUGCGGGGGCCUCUGGCGGCUGGUCCUGGC
ACAGCGCUGGAUGGAGCGGCUCAAGACUGUCGCUGGGUCCAAGAUGCAAGGCUUGCUGG
AGCGCGUGAACACGGAGAUACACUUUGUCACCAAAUGUGCCUUUCAGCCCCCCCCCAGC
UGUCUUCGCUUCGUCCAGACCAACAUCUCCCGCCUCCUGCAGGAGACCUCCGAGCAGCU
GGUGGCGCUGAAGCCCUGGAUCACUCGCCAGAACUUCUCCCGGUGCCUGGAGCUGCAGU
GUCAGCCCGACUCCUCAACCCUGCCACCCCCAUGGAGUCCCCGGCCCCUGGAGGCCACA
GCCCCGACAGCCCCGCAGCCCCCUCUGCUCCUCCUACUGCUGCUGCCCGUGGGCCUCCU
GCUGCUGGCCGCUGCCUGGUGCCUGCACUGGCAGAGGACGCGGCGGAGGACACCCCGCC
CUGGGGAGCAGGUGCCCCCCGUCCCCAGUCCCCAGGACCUGCUGCUUGUGGAGCAC

Furthermore, preferably the RNA construct comprises an RNA nucleotide sequence substantially as set out in SEQ ID No: 132, or a variant or fragment thereof.

In one embodiment, the at least one ISP may be Soluble sFlt3L (NCBI Reference Sequence: NM_001459.4; UniProtKB—P49771 (FLT3L_HUMAN)), or an orthologue thereof. One embodiment of the polypeptide sequence of the sFlt3L is represented herein as SEQ ID No: 133, as follows:

[SEQ ID No: 133]
MDRAKLLLLLLLLLLPQAQATQDCSFQHSPISSDFAVKIRELSDYLLQDYP
VIVASNLQDEELCGGLWRLVLAQRWMERLKTVAGSKMQGLLERVNTEIHFV
TKCAFQPPPSCLRFVQTNISRLLQETSEQLVALKPWITRQNFSRCLELQCQ
PDSSTLPPPWSPRPLEATAPTAPQP

Therefore, preferably the RNA construct of any aspect comprises a nucleotide sequence which encodes an amino acid sequence substantially as set out in SEQ ID No: 133, or a variant or fragment thereof.

In one embodiment, the sFlt3L polypeptide is encoded by the DNA nucleotide sequence of SEQ ID No: 134, as follows:

[SEQ ID No: 134]
ATGGACAGAGCCAAGCTGCTGCTGTTGCTGCTCCTTCTGCTGCTGCCTCAGGCTCAAGC
TACCCAGGACTGCTCCTTCCAACACAGCCCCATCTCCTCCGACTTCGCTGTCAAAATCC
GTGAGCTGTCTGACTACCTGCTTCAAGATTACCCAGTCACCGTGGCCTCCAACCTGCAG
GACGAGGAGCTCTGCGGGGGCCTCTGGCGGCTGGTCCTGGCACAGCGCTGGATGGAGCG
GCTCAAGACTGTCGCTGGGTCCAAGATGCAAGGCTTGCTGGAGCGCGTGAACACGGAGA
TACACTTTGTCACCAAATGTGCCTTTCAGCCCCCCCCCAGCTGTCTTCGCTTCGTCCAG
ACCAACATCTCCCGCCTCCTGCAGGAGACCTCCGAGCAGCTGGTGGCGCTGAAGCCCTG
GATCACTCGCCAGAACTTCTCCCGGTGCCTGGAGCTGCAGTGTCAGCCCGACTCCTCAA
CCCTGCCACCCCCATGGAGTCCCCGGCCCCTGGAGGCCACAGCCCCGACAGCCCCGCAG
CCC

Accordingly, preferably the sFlt3L polypeptide is encoded by the DNA nucleotide sequence substantially as set out in SEQ ID NO: 134, or a variant or fragment thereof.

Thus, the RNA construct may comprise an RNA nucleotide sequence of SEQ ID No: 135, as follows:

[SEQ ID No: 135]
AUGGACAGAGCCAAGCUGCUGCUGUUGCUGCUCCUUCUGCUGCUGCCUC
AGGCUCAAGCUACCCAGGACUGCUCCUUCCAACACAGCCCCAUCUCCUC
CGACUUCGCUGUCAAAAUCCGUGAGCUGUCUGACUACCUGCUUCAAGAU
UACCCAGUCACCGUGGCCUCCAACCUGCAGGACGAGGAGCUCUGCGGGG
GCCUCUGGCGGCUGGUCCUGGCACAGCGCUGGAUGGAGCGGCUCAAGAC
UGUCGCUGGGUCCAAGAUGCAAGGCUUGCUGGAGCGCGUGAACACGGAG
AUACACUUUGUCACCAAAUGUGCCUUUCAGCCCCCCCCCAGCUGUCUUC
GCUUCGUCCAGACCAACAUCUCCCGCCUCCUGCAGGAGACCUCCGAGCA
GCUGGUGGCGCUGAAGCCCUGGAUCACUCGCCAGAACUUCUCCCGGUGC
CUGGAGCUGCAGUGUCAGCCCGACUCCUCAACCCUGCCACCCCCAUGGA
GUCCCCGGCCCCUGGAGGCCACAGCCCCGACAGCCCCGCAGCCC

Furthermore, preferably the RNA construct comprises an RNA nucleotide sequence substantially as set out in SEQ ID No: 135, or a variant or fragment thereof.

The ISP may not be one that is selected from a group consisting of: an interleukin; IL2; IL4; IL6; IL7; IL12; IL15; IL21; colony stimulating factor (CSF); granulocyte colony stimulating factor (G-CSF); granulocyte-macrophage colony stimulating factor (GM-CSF); and tumour necrosis factor (TNF).

The inventors have also developed novel RNA constructs (saRNA and mRNA), which not only include an ISP, which acts as a molecular adjuvant, but which are also able to overcome the host cell's innate immune system which senses RNA. This has been achieved by expressing viral immune inhibitor proteins (IIPs) and/or human innate modulatory proteins (IMPs), which block or reduce the host's innate immune system machinery, resulting in improved translation (in the case of mRNA) and increased self-amplification and subsequent translation (in the case of saRNA), and therefore greater protein expression levels of the gene of interest, such as an antigen, in a host cell.

Thus, the at least one human innate modulatory protein (IMP) described herein may be any of the IMPs listed below. For example, the IMP can be found with the following NCBI and UniProt accession numbers: IRF1 deleted of DBD and/or NLS (141-325), IRF1 DBD (1-164)—NCBI Reference Sequence: NM_002198.3, UniProtKB—P10914 (IRF1_HUMAN); IRF3 deleted of DBD (191-427)—NCBI Reference Sequence: NM_001571.6, UniProtKB—Q14653 (IRF3_HUMAN); IRF7 DN (238-503)—NCBI Reference Sequence: NM_001572.5, UniProtKB—Q92985 (IRF7_HUMAN); IRF9 DN (143-393), IRF9 DN (182-385), IRF9 DN (200-308), IRF9 DBD (1-120)—NCBI Reference Sequence: NM_006084.5, UniProtKB—Q00978 (IRF9_HUMAN); IRF4 DBD (21-129)—NCBI Reference Sequence: NM_002460.4, UniProtKB—Q15306 (IRF4_HUMAN); IRF5 DBD (1-140), IRF5 A68P DBD (1-140)—NCBI Reference Sequence: NM_032643.5, UniProtKB—Q13568 (IRF5_HUMAN); IRF8 DBD (1-140)—NCBI Reference Sequence: NM_002163, UniProtKB—Q02556 (IRF8_HUMAN); STAT1 DN (Y701F)—NCBI Reference Sequence: NM_007315.4, UniProtKB—P42224 (STAT1_HUMAN); STAT2 DN (133-315), STAT2 DN (F175D)—NCBI Reference Sequence: NM_005419.4, UniProtKB—P52630 (STAT2_HUMAN); HSP90 (CDC37) DN (1-232)—NCBI Reference Sequence: NM_007065.4, UniProtKB—Q16543 (CDC37_HUMAN); STING-Beta DN—GenBank: MF360993.1, UniProtKB—AoA3G1PSE3 (AoA3G1PSE3_HUMAN); A20 or TNFAIP3 DN (369-775), A20 or TNFAIP3 (606-790) DN—NCBI Reference Sequence: NM_006290.4, UniProtKB—P21580 (TNAP3_HUMAN); MFN2 DN (370-500), MFN2 DN (400-480)—NCBI Reference Sequence: NM_001127660.2, UniProtKB—O95140 (MFN2_HUMAN); TARBP2 DN (1-234)—NCBI Reference Sequence: NM_134323.2, UniProtKB—Q15633 (TRBP2_HUMAN); Zinc AVP DN (1-200)—NCBI Reference Sequence: NM_020119.4, UniProtKB—Q7Z2W4 (ZCCHV_HUMAN); PKR saRNA BD DN (1-170)—NCBI Reference Sequence: NM_002759.4, UniProtKB—P19525 (E2AK2_HUMAN); PACT PRKRA DN (1-194)—NCBI Reference Sequence: NM_003690.5, UniProtKB—O75569 (PRKRA_HUMAN); RNAL DN (1-330)—NCBI Reference Sequence: NM_021133.4, UniProtKB—Q05823 (RNSA_HUMAN); OAS3 Domain 1 DN (1-343)—NCBI Reference Sequence: NM_006187.4, UniProtKB—Q9Y6K5 (OAS3_HUMAN); FAF1—NCBI Reference Sequence: NM_007051.3, UniProtKB—Q9UNN5 (FAF1_HUMAN); SOCS1—NCBI Reference Sequence: NM_003745.2, UniProtKB—O15524 (SOCS1_HUMAN); SOCS3—NCBI Reference Sequence: NM_003955.5, UniProtKB—O14543 (SOCS3_HUMAN); USP18—NCBI Reference Sequence: NM_017414.4, UniProtKB—Q9UMW8 (UBP18_HUMAN); USP21—NCBI Reference Sequence: NM_012475.5, UniProtKB—Q9UK80 (UBP21_HUMAN); USP27 (1-438)—NCBI Reference Sequence: NM_001145073.3, UniProtKB—A6NNY8 (USP27_HUMAN); CYLD—NCBI Reference Sequence: NM_015247.3, UniProtKB—Q9NQC7 (CYLD_HUMAN); LGP2—NCBI Reference Sequence: NM_024119.3, UniProtKB—Q96C10 (DHX58_HUMAN); RIG splice variant (DDX58_HUMAN_ISOFORM_2), RIG-1 (DDX 58)—NCBI Reference Sequence: NM_014314.4, UniProtKB—O95786 (DDX58_HUMAN) AA 36-80 deletion; DDX-56—NCBI Reference Sequence: NM_019082.4, UniProtKB—Q9NY93 (DDX56_HUMAN); ARL5B—NCBI Reference Sequence: NM_178815.5, UniProtKB—Q96KC2 (ARL5B_HUMAN); ARL16—NCBI Reference Sequence: NM_001040025.3, UniProtKB—QoP5N6 (ARL16_HUMAN).

In one embodiment, the at least one IMP may be IRF1 DBD (1-164)—referred to as IRF1 (NCBI Reference Sequence: NM_002198.3, UniProtKB—P10914 (IRF1_HUMAN)), or an orthologue thereof. One embodiment of the polypeptide sequence of IRF1 DBD is represented herein as SEQ ID No: 178, as follows:

[SEQ ID No: 178]
MPITRMRMRPWLEMQINSNQIPGLIWINKEEMIFQIPWKHAAKHGWDIN
KDACLFRSWAIHTGRYKAGEKEPDPKTWKANFRCAMNSLPDIEEVKDQS
RNKGSSAVRVYRMLPPLTKNQRKERKSKSSRDAKSKAKRKSCGDSSPDT
FSDGLSSSTLPDDHSSY

Therefore, preferably the RNA construct of any aspect comprises a nucleotide sequence which encodes an amino acid sequence substantially as set out in SEQ ID No: 178, or a variant or fragment thereof.

In one embodiment, the IRF1 DBD (1-164) polypeptide is encoded by the DNA nucleotide sequence of SEQ ID No: 179, as follows:

[SEQ ID No: 179]
ATGCCCATCACTCGGATGCGCATGAGACCCTGGCTAGAGATGCAGATTA
ATTCCAACCAAATCCCGGGGCTCATCTGGATTAATAAAGAGGAGATGAT
CTTCCAGATCCCATGGAAGCATGCTGCCAAGCATGGCTGGGACATCAAC
AAGGATGCCTGTTTGTTCCGGAGCTGGGCCATTCACACAGGCCGATACA
AAGCAGGGGAAAAGGAGCCAGATCCCAAGACGTGGAAGGCCAACTTTCG
CTGTGCCATGAACTCCCTGCCAGATATCGAGGAGGTGAAAGACCAGAGC
AGGAACAAGGGCAGCTCAGCTGTGCGAGTGTACCGGATGCTTCCACCTC
TCACCAAGAACCAGAGAAAAGAAAGAAAGTCGAAGTCCAGCCGAGATGC
TAAGAGCAAGGCCAAGAGGAAGTCATGTGGGGATTCCAGCCCTGATACC
TTCTCTGATGGACTCAGCAGCTCCACTCTGCCTGATGACCACAGCAGCT
AC

Accordingly, preferably the IRF1 DBD (1-164) polypeptide is encoded by the DNA nucleotide sequence substantially as set out in SEQ ID NO: 179, or a variant or fragment thereof.

Thus, the RNA construct may comprise an RNA nucleotide sequence of SEQ ID No: 180, as follows:

[SEQ ID No: 180]
AUGCCCAUCACUCGGAUGCGCAUGAGACCCUGGCUAGAGAUGCAGAUUA
AUUCCAACCAAAUCCCGGGGCUCAUCUGGAUUAAUAAAGAGGAGAUGAU
CUUCCAGAUCCCAUGGAAGCAUGCUGCCAAGCAUGGCUGGGACAUCAAC
AAGGAUGCCUGUUUGUUCCGGAGCUGGGCCAUUCACACAGGCCGAUACA
AAGCAGGGGAAAAGGAGCCAGAUCCCAAGACGUGGAAGGCCAACUUUCG
CUGUGCCAUGAACUCCCUGCCAGAUAUCGAGGAGGUGAAAGACCAGAGC
AGGAACAAGGGCAGCUCAGCUGUGCGAGUGUACCGGAUGCUUCCACCUC
UCACCAAGAACCAGAGAAAAGAAAGAAAGUCGAAGUCCAGCCGAGAUGC
UAAGAGCAAGGCCAAGAGGAAGUCAUGUGGGGAUUCCAGCCCUGAUACC
UUCUCUGAUGGACUCAGCAGCUCCACUCUGCCUGAUGACCACAGCAGCU
AC

Furthermore, preferably the RNA construct comprises an RNA nucleotide sequence substantially as set out in SEQ ID No: 180, or a variant or fragment thereof.

Furthermore, the at least one viral immune inhibitor protein (IIP) described herein may be any of the IIPs listed below. For example, the IIP can be found with the following NCBI and UniProt accession numbers: HPV E6 (human papillomavirus E6; NP_041325.1; Accession Number—NCBI Reference Sequence: NC_001526.4; UniProtKB—P03126 (VE6_HPV16)); HSV ICP34.5 (Herpes simplex virus ICP34.5; YP_009137073.1; Accession Number—NCBI Reference Sequence: NC_001806.2; UniProtKB—P36313 (ICP34_HHV11)); HCV E2 (hepatitis C virus E2; NS1 Protein from polyprotein ADC54662.1; Accession Number—Genomic RNA Translation ADC54662.1; UniProtKB—D3W8R2 (D3W8R2_9HEPC)); HCV NS5a (hepatitis C virus NS5a; isolate H—Genomic RNA translation: AAA45534.1; UniProtKB—P27958 (POLG_HCV77)); VACV E3L (vaccinia virus E3L; JN654977.1, AEY72868.1; Accession Number—Genomic DNA Translation: AEY72868.1; UniProtKB—H2DSW3 (H2DSW3-9POXV)); VACV K3L (vaccinia virus K3L; P20639.1; Accession Number—Genomic DNA Translation: AAA48009.1; UniProtKB—P20639 (K3_VACCC)); Vaccinia C6 (vaccinia virus C6; Accession Number—Genomic DNA Translation: AAA69602.1; UniProtKB—P17362 (C6_VACCW)); MERS ORF8b (Middle East Respiratory Syndrome virus ORF8b; Accession Number—GenBank: ANF29170.1; UniProtKB—AoA1W5LGP6 (AoA1W5LGP6_MERS)); KSHV ORF52 (Kaposi's sarcoma-associated herpesvirus ORF52; Accession Number—Genomic DNA Translation: ACY00451.1; UniProtKB—FSHBL8 (FSHBL8_HHV8)); Ebola VP35 (NP_066244.1; Accession Number—NCBI Reference Sequence: NC_002549.1; UniProtKB—Q05127 (VP35_EBOZM)); SARS-CoV-2 ORF3b, ORF3b*57 variant, ORF3b*79 variant, ORF3b*57 Ecuador variant (Accession Number—NCBI Reference Sequence: NC_045512.2).

In one embodiment, the at least one IIP may be VACV E3L—referred to as E3L (NCBI Reference Sequence: JN654977.1, AEY72868.1; Accession Number—Genomic DNA Translation: AEY72868.1; UniProtKB—H2DSW3 (H2DSW3-9POXV)), or an orthologue thereof. One embodiment of the polypeptide sequence of E3L is represented herein as SEQ ID No: 172, as follows:

[SEQ ID No: 172]
MSKIYIDERSDAEIVCAAIKNIGIEGATAAQLTRQLNMEKREVNKALYD
LQRSAMVYSSDDIPPRWEMTTEADKPDADAMADVIIDDVSREKSMREDH
KSEDDVIPAKKIIDWKDANPVTIINEYCQITKRDWSFRIESVGPSNSPT
FYACVDIDGRVFDKADGKSKRDAKNNAAKLAVDKLLGYVIIRF

Therefore, preferably the RNA construct of any aspect comprises a nucleotide sequence which encodes an amino acid sequence substantially as set out in SEQ ID No: 172, or a variant or fragment thereof.

In one embodiment, the E3L polypeptide is encoded by the DNA nucleotide sequence of SEQ ID No: 173, as follows:

[SEQ ID No: 173]
ATGTCTAAGATCTATATCGACGAGCGTTCTGACGCAGAGATTGTGTGTG
CGGCTATTAAAAACATTGGAATCGAAGGAGCTACTGCTGCACAACTAAC
TAGACAACTTAATATGGAGAAGCGAGAAGTTAATAAAGCTCTGTACGAT
CTTCAACGTAGTGCTATGGTGTACAGCTCCGACGATATTCCTCCTCGTT
GGTTTATGACAACGGAGGCGGATAAGCCGGATGCTGATGCTATGGCTGA
CGTCATAATAGATGATGTATCCCGCGAAAAATCAATGAGAGAGGATCAT
AAGTCTTTTGATGATGTTATTCCGGCTAAAAAAATTATTGATTGGAAAG
ATGCTAACCCTGTCACCATTATTAATGAGTACTGCCAAATAACTAAGAG
AGATTGGTCTTTTCGTATTGAATCAGTTGGGCCTAGTAACTCTCCTACA
TTTTATGCCTGTGTAGATATCGACGGAAGAGTATTCGATAAGGCCGATG
GAAAATCTAAACGAGATGCTAAAAATAATGCAGCTAAATTGGCTGTAGA
TAAACTTCTTGGGTACGTCATCATTAGATTC

Accordingly, preferably the E3L polypeptide is encoded by the DNA nucleotide sequence substantially as set out in SEQ ID NO: 173, or a variant or fragment thereof.

Thus, the RNA construct may comprise an RNA nucleotide sequence of SEQ ID No: 174, as follows:

[SEQ ID No: 174]
AUGUCUAAGAUCUAUAUCGACGAGCGUUCUGACGCAGAGAUUGUGUGUG
CGGCUAUUAAAAACAUUGGAAUCGAAGGAGCUACUGCUGCACAACUAAC
UAGACAACUUAAUAUGGAGAAGCGAGAAGUUAAUAAAGCUCUGUACGAU
CUUCAACGUAGUGCUAUGGUGUACAGCUCCGACGAUAUUCCUCCUCGUU
GGUUUAUGACAACGGAGGCGGAUAAGCCGGAUGCUGAUGCUAUGGCUGA
CGUCAUAAUAGAUGAUGUAUCCCGCGAAAAAUCAAUGAGAGAGGAUCAU
AAGUCUUUUGAUGAUGUUAUUCCGGCUAAAAAAAUUAUUGAUUGGAAAG
AUGCUAACCCUGUCACCAUUAUUAAUGAGUACUGCCAAAUAACUAAGAG
AGAUUGGUCUUUUCGUAUUGAAUCAGUUGGGCCUAGUAACUCUCCUACA
UUUUAUGCCUGUGUAGAUAUCGACGGAAGAGUAUUCGAUAAGGCCGAUG
GAAAAUCUAAACGAGAUGCUAAAAAUAAUGCAGCUAAAUUGGCUGUAGA
UAAACUUCUUGGGUACGUCAUCAUUAGAUUC

Furthermore, preferably the RNA construct comprises an RNA nucleotide sequence substantially as set out in SEQ ID No: 174, or a variant or fragment thereof.

In addition, the IIP may also be selected from a group of IIPs consisting of: HSV-2 Us1; HSV-1 Us1; HSV-1Us11; BVDV Npro; Langat NS5; Influenza NS1 (For example from the Influenza H1N1 (A/Puerto Rico/8/1934 (PR34) strain); PIV-5 V; SARS-CoV-2 ORF3b; and MERS-CoV ORF4a, Vaccinia C6, EV71-2Apro, BVDV nPro, SARS-CoV-2 ORF3b*57 variant, SARS-CoV-2 ORF3b*57 Ecuador variant, SARS-CoV-2 Pangolin ORF3b*57, SARS-CoV-2 ORF3b*79 variant and SARS-CoV-2 ORF3b*79 Pangolin variant.

In one embodiment, the at least one IIP may be Influenza NS1 (For example from the Influenza H1N1 (A/Puerto Rico/8/1934 (PR34) strain—referred to as PR34 (NCBI Reference Sequence: NC_002020.1; UniProtKB—P03496 (NS1_I34A1)) or an orthologue thereof. One embodiment of the polypeptide sequence of PR34 is represented herein as SEQ ID No: 175, as follows:

[SEQ ID No: 175]
MDPNTVSSFQVDCFLWHVRKRVADQELGDAPFLDRLRRDQKSLRGRGST
LGLDIETATRAGKQIVERILKEESDEALKMTMASVPASRYLTDMTLEEM
SRDWSMLIPKQKVAGPLCIRMDQAIMDKNIILKANFSVIFDRLETLILL
RAFTEEGAIVGEISPLPSLPGHTAEDVKNAVGVLIGGLEWNDNTVRVSE
TLQRFAWRSSNENGRPPLTPKQKREMAGTIRSEV

Therefore, preferably the RNA construct of any aspect comprises a nucleotide sequence which encodes an amino acid sequence substantially as set out in SEQ ID No: 175, or a variant or fragment thereof.

In one embodiment, the PR34 polypeptide is encoded by the DNA nucleotide sequence of SEQ ID No: 176, as follows:

[SEQ ID No: 176]
ATGGATCCAAACACTGTGTCAAGCTTTCAGGTAGATTGCTTTCTTTGGC
ATGTCCGCAAACGAGTTGCAGACCAAGAACTAGGTGATGCCCCATTCCT
TGATCGGCTTCGCCGAGATCAGAAATCCCTAAGAGGAAGGGGCAGCACT
CTTGGTCTGGACATCGAGACAGCCACACGTGCTGGAAAGCAGATAGTGG
AGCGGATTCTGAAAGAAGAATCCGATGAGGCACTTAAAATGACCATGGC
CTCTGTACCTGCGTCGCGTTACCTAACCGACATGACTCTTGAGGAAATG
TCAAGGGAATGGTCCATGCTCATACCCAAGCAGAAAGTGGCAGGCCCTC
TTTGTATCAGAATGGACCAGGCGATCATGGATAAAAACATCATACTGAA
AGCGAACTTCAGTGTGATTTTTGACCGGCTGGAGACTCTAATATTGCTA
AGGGCTTTCACCGAAGAGGGAGCAATTGTTGGCGAAATTTCACCATTGC
CTTCTCTTCCAGGACATACTGCTGAGGATGTCAAAAATGCAGTTGGAGT
CCTCATCGGAGGACTTGAATGGAATGATAACACAGTTCGAGTCTCTGAA
ACTCTACAGAGATTCGCTTGGAGAAGCAGTAATGAGAATGGGAGACCTC
CACTCACTCCAAAACAGAAACGAGAAATGGCGGGAACAATTAGGTCAGA
AGTT

Accordingly, preferably the PR34 polypeptide is encoded by the DNA nucleotide sequence substantially as set out in SEQ ID NO: 176, or a variant or fragment thereof.

Thus, the RNA construct may comprise an RNA nucleotide sequence of SEQ ID No: 177, as follows:

[SEQ ID No: 177]
AUGGAUCCAAACACUGUGUCAAGCUUUCAGGUAGAUUGCUUUCUUUGGC
AUGUCCGCAAACGAGUUGCAGACCAAGAACUAGGUGAUGCCCCAUUCCU
UGAUCGGCUUCGCCGAGAUCAGAAAUCCCUAAGAGGAAGGGGCAGCACU
CUUGGUCUGGACAUCGAGACAGCCACACGUGCUGGAAAGCAGAUAGUGG
AGCGGAUUCUGAAAGAAGAAUCCGAUGAGGCACUUAAAAUGACCAUGGC
CUCUGUACCUGCGUCGCGUUACCUAACCGACAUGACUCUUGAGGAAAUG
UCAAGGGAAUGGUCCAUGCUCAUACCCAAGCAGAAAGUGGCAGGCCCUC
UUUGUAUCAGAAUGGACCAGGCGAUCAUGGAUAAAAACAUCAUACUGAA
AGCGAACUUCAGUGUGAUUUUUGACCGGCUGGAGACUCUAAUAUUGCUA
AGGGCUUUCACCGAAGAGGGAGCAAUUGUUGGCGAAAUUUCACCAUUGC
CUUCUCUUCCAGGACAUACUGCUGAGGAUGUCAAAAAUGCAGUUGGAGU
CCUCAUCGGAGGACUUGAAUGGAAUGAUAACACAGUUCGAGUCUCUGAA
ACUCUACAGAGAUUCGCUUGGAGAAGCAGUAAUGAGAAUGGGAGACCUC
CACUCACUCCAAAACAGAAACGAGAAAUGGCGGGAACAAUUAGGUCAGA
AGUU

Furthermore, preferably the RNA construct comprises an RNA nucleotide sequence substantially as set out in SEQ ID No: 177, or a variant or fragment thereof.

The RNA construct comprises a nucleotide sequence which encodes the at least one therapeutic biomolecule. This is referred to as the gene of interest (GOI) in FIG. 1.

Thus, the at least one therapeutic biomolecule may comprise a therapeutic protein. The skilled person would understand that therapeutic protein relates to any protein that has therapeutic application, preferably in human. Exemplary therapeutic biomolecules that can be encoded by the RNA molecule include proteins or peptides derived from pathogens, such as bacteria, viruses, fungi, protozoa/or parasites. Preferably, the protein or peptide is an antigen. Hence, in the embodiment in which the at least one therapeutic biomolecule is an antigen, the RNA construct may be regarded as a vaccine.

The protein or peptide derived from a virus may be a viral antigen. The viral antigen may be derived from a virus selected from the group consisting of: Orthomyxoviruses; Paramyxoviridae viruses; Metapneumovirus and Morbilliviruses; Pneumoviruses; Paramyxoviruses; Poxviridae; Metapneumoviruses; Morbilliviruses; Picornaviruses; Enteroviruseses; Bunyaviruses; Phlebovirus; Nairovirus; Heparnaviruses; Togaviruses; Alphavirus; Arterivirus; Flaviviruses; Pestiviruses; Hepadnaviruses; Rhabdoviruses; Caliciviridae; Coronaviruses; Retroviruses; Reoviruses; Parvoviruses; Delta hepatitis virus (HDV); Hepatitis E virus (HEV); Human Herpesviruses and Papovaviruses.

The Orthomyxoviruses may be Influenza A, B and C. The Paramyxoviridae virus may be Pneumoviruses (RSV), Paramyxoviruses (PIV). The Metapneumovirus may be Morbilliviruses (e.g., measles). The Pneumovirus may be Respiratory syncytial virus (RSV), Bovine respiratory syncytial virus, Pneumonia virus of mice, or Turkey rhinotracheitis virus. The Paramyxovirus may be Parainkuenza virus types 1-4 (PIV), Mumps, Sendai viruses, Simian virus 5, Bovine parainkuenza virus, Nipahvirus, Henipavirus or Newcastle disease virus. The Poxviridae may be Variola vera, for example Variola major and Variola minor. The Metapneumovirus may be human metapneumovirus (hMPV) or avian metapneumoviruses (aMPV). The Morbillivirus may be measles. The Picornaviruses may be Enteroviruses, Rhinoviruses, Heparnavirus, Parechovirus, Cardioviruses andAphthoviruses. The Enteroviruses may be Poliovirus types 1, 2 or 3, Coxsackie A virus types 1 to 22 and 24, Coxsackie B virus types 1 to 6, Echovirus (ECHO) virus) types 1 to 9, 11 to 27 and 29 to 34 or Enterovirus 68 to 71. The Bunyavirus may be California encephalitis virus. The Phlebovirus may be Rift Valley Fever virus. The Nairovirus may be Crimean-Congo hemorrhagic fever virus. The Heparnaviruses may be Hepatitis A virus (HAV). The Togaviruses may be Rubivirus. The Flavivirus may be Tick-borne encephalitis (TBE) virus, Dengue (types 1, 2, 3 or 4) virus, Yellow Fever virus, Japanese encephalitis virus, Kyasanur Forest Virus, West Nile encephalitis virus, St. Louis encephalitis virus, Russian spring-summer encephalitis virus or Powassan encephalitis virus. The Pestivirus may be Bovine viral diarrhea (BVDV), Classical swine fever (CSFV) or Border disease (BDV). The Hepadnavirus may be Hepatitis B virus or Hepatitis C virus. The Rhabdovirus may be Lyssavirus (Rabies virus) or Vesiculovirus (VSV). The Caliciviridae may be Norwalk virus, or Norwalk-like Viruses, such as Hawaii Virus and Snow Mountain Virus. The Coronavirus may be SARS CoV-1, SARS-CoV-2, MERS, Human respiratory coronavirus, Avian infectious bronchitis (IBV), Mouse hepatitis virus (MHV), or Porcine transmissible gastroenteritis virus (TGEV). The Retrovirus may be Oncovirus, a Lentivirus or a Spumavirus. The Reovirus may be an Orthoreo virus, a Rotavirus, an Orbivirus, or a Coltivirus. The Parvovirus may be Parvovirus B 19. The Human Herpesvirus may be Herpes Simplex Viruses (HSV), Varicella-zoster virus (VZV), Epstein-Barr virus (EBV), Cytomegalovirus (CMV), Human Herpesvirus 6 (HHV6), Human Herpesvirus 7 (HHV7), or Human Herpesvirus 8 (HHV8). The Papovavirus may be Papilloma viruses, Polyomaviruses, Adenoviruess or Arenaviruses.

The protein or peptide derived from bacteria may be a bacterial antigen.

The bacterial antigen may derived from a bacterium selected from the group consisting of: Neisseria meningitides, Streptococcus pneumoniae, Streptococcus pyogenes, Moraxella catarrhalis, Bordetella pertussis, Burkholderia sp. (e.g., Burkholderia mallei, Burkholderia pseudomallei and Burkholderia cepacia), Staphylococcus aureus, Haemophilus influenzae, Clostridium tetani (Tetanus), Clostridium perfringens, Clostridium botulinums, Cornynebacterium diphtheriae (Diphtheria), Pseudomonas aeruginosa, Legionella pneumophila, Coxiella burnetii, Brucella sp. (e.g., B. abortus, B. canis, B. melitensis, B. neotomae, B. ovis, B. suis and B. pinnipediae, Francisella sp. (e.g., F. novicida, F. philomiragia and F. tularensis), Streptococcus agalactiae, Neiserria gonorrhoeae, Chlamydia trachomatis, Treponema pallidum (Syphilis), Haemophilus ducreyi, Enterococcus faecalis, Enterococcus faecium, Helicobacter pylori, Staphylococcus saprophyticus, Yersinia enter ocolitica, E. coli, Bacillus anthracis (anthrax), Yersinia pestis (plague), Mycobacterium tuberculosis, Rickettsia, Listeria, Chlamydia pneumoniae, Vibrio cholerae, Salmonella typhi (typhoid fever), Borrelia burgdorfer, Porphyromonas s and Klebsiella sp.

The protein or peptide derived from a fungus may be a fungal antigen.

The fungal antigen may be derived from a fungus selected from the group consisting of Dermatophytres, including: Epidermophyton koccusum, Microsporum audouini, Microsporum canis, Microsporum distortum, Microsporum equinum, Microsporum gypsum, Microsporum nanum, Trichophyton concentricum, Trichophyton equinum, Trichophyton gallinae, Trichophyton gypseum, Trichophyton megnini, Trichophyton mentagrophytes, Trichophyton quinckeanum, Trichophyton rubrum, Trichophyton schoenleini, Trichophyton tonsurans, Trichophyton verrucosum, T verrucosum var. album, var. discoides, var. ochraceum, Trichophyton violaceum, and/or Trichophyton faviforme; or from Aspergillus fumigatus, Aspergillus kavus, Aspergillus niger, Aspergillus nidulans, Aspergillus terreus, Aspergillus sydowii, Aspergillus kavatus, Aspergillus glaucus, Blastoschizomyces capitatus, Candida albicans, Candida enolase, Candida tropicalis, Candida glabrata, Candida krusei, Candida parapsilosis, Candida stellatoidea, Candida kusei, Candida parakwsei, Candida lusitaniae, Candida pseudotropicalis, Candida guilliermondi, Cladosporium carrionii, Coccidioides immitis, Blastomyces dermatidis, Cryptococcus neoformans, Geotrichum clavatum, Histoplasma capsulatum, Klebsiella pneumoniae, Microsporidia, Encephalitozoon spp., Septata intestinalis and Enterocytozoon bieneusi; Brachiola spp, Microsporidium spp., Nosema spp., Pleistophora spp., Trachipleistophora spp., Vittaforma spp Paracoccidioides brasiliensis, Pneumocystis carinii, Pythiumn insidiosum, Pityrosporum ovale, Sacharomyces cerevisiae, Saccharomyces boulardii, Saccharomyces pombe, Scedosporium apiosperum, Sporothrix schenckii, Trichosporon beigelii, Toxoplasma gondii, Penicillium marneffei, Malassezia spp., Fonsecaea spp., Wangiella spp., Sporothrix spp., Basidiobolus spp., Conidiobolus spp., Rhizopus spp, Mucor spp, Absidia spp, Mortierella spp, Cunninghamella spp, Saksenaea spp., Alternaria spp, Curvularia spp, Helminthosporium spp, Fusarium spp, Aspergillus spp, Penicillium spp, Monolinia spp, Rhizoctonia spp, Paecilomyces spp, Pithomyces spp, and Cladosporium spp.

The protein or peptide derived from a protozoan may be a protozoan antigen.

The protozoan antigen may be derived from a protozoan selected from the group consisting of: Entamoeba histolytica, Giardia lambli, Cryptosporidium parvum, Cyclospora cayatanensis and Toxoplasma.

The therapeutic biomolecule may be a protein or peptide derived from a plant. Preferably, the protein or peptide is a plant antigen. For example, the plant antigen may be derived from Ricinus communis.

In another embodiment, the therapeutic biomolecule may be an immunogen or an antigen. Preferably, the immunogen or an antigen is a tumour immunogen or antigen, or cancer immunogen or antigen. The tumour immunogens and antigens may be peptide-containing tumour antigens, such as a polypeptide tumour antigen or glycoprotein tumour antigens.

The tumour antigens may be (a) full length molecules associated with cancer cells, (b) homologs and modified forms of the same, including molecules with deleted, added and/or substituted portions, and (c) fragments of the same.

Suitable tumour immunogens include: class I-restricted antigens recognized by CD 8+ lymphocytes or class II-restricted antigens recognized by CD4+ lymphocytes.

The tumour antigen may be an antigen that is associated with a cancer selected from the group consisting of: a testis cancer, melanoma, lung cancer, head and neck cancer, NSCLC, breast cancer, gastrointestinal cancer, bladder cancer, colorectal cancer, pancreatic cancer, lymphoma, leukaemia, renal cancer, hepatoma, ovarian cancer, gastric cancer and prostate cancer.

The tumour antigen may be selected from:
(a) cancer-testis antigens, such as NY-ESO-I, SSX2, SCP-1, as well as RAGE, BAGE, GAGE and MAGE family polypeptides, for example, GAGE-I, GAGE-2, MAGE-I, MAGE-2, MAGE-3, MAGE-4, MAGE-5, MAGE-6, and MAGE-12 (which can be used, for example, to address melanoma, lung, head and neck, NSCLC, breast, gastrointestinal, and bladder tumours);
(b) mutated antigens, for example, p53 (associated with various solid tumours, e.g., colorectal, lung, head and neck cancer), p21/Ras (associated with, e.g., melanoma, pancreatic cancer and colorectal cancer), CDK4 (associated with, e.g., melanoma), MUM-1 (associated with, e.g., melanoma), caspase-8 (associated with, e.g., head and neck cancer), CIA 0205 (associated with, e.g., bladder cancer), HLA-A2-R1701, beta catenin (associated with, e.g., melanoma), TCR (associated with, e.g., T-cell non-Hodgkins lymphoma), BCR-abl (associated with, e.g., chronic myelogenous leukemia), triosephosphate isomerase, KIA 0205, CDC-27, and LDLR-FUT;
(c) over-expressed antigens, for example, Galectin 4 (associated with, e.g., colorectal cancer), Galectin 9 (associated with, e.g., Hodgkin's disease), proteinase 3 (associated with, e.g., chronic myelogenous leukemia), WT 1 (associated with, e.g., various leukaemias), carbonic anhydrase (associated with, e.g., renal cancer), aldolase A (associated with, e.g., lung cancer), PRAME (associated with, e.g., melanoma), HER-2/neu (associated with, e.g., breast, colon, lung and ovarian cancer), alpha-fetoprotein (associated with, e.g., hepatoma), KSA (associated with, e.g., colorectal cancer), gastrin (associated with, e.g., pancreatic and gastric cancer), telomerase catalytic protein, MUC-I (associated with, e.g., breast and ovarian cancer), G-250 (associated with, e.g., renal cell carcinoma), p53 (associated with, e.g., breast, colon cancer), and carcinoembryonic antigen (associated with, e.g., breast cancer, lung cancer, and cancers of the gastrointestinal tract such as colorectal cancer);
(d) shared antigens, for example, melanoma-melanocyte differentiation antigens, such as MART-1/Melan A, gplOO, MClR, melanocyte-stimulating hormone receptor, tyrosinase, tyrosinase related protein-1/TRPl and tyrosinase related protein-2/TRP2 (associated with, e.g., melanoma);
(e) prostate-associated antigens, such as PAP, PSA, PSMA, PSH-Pl, PSM-Pl, PSM-P2, associated with e.g., prostate cancer; and/or
(f) immunoglobulin idiotypes (associated with myeloma and B cell lymphomas, for example).

The therapeutic biomolecule may be a eukaryotic protein or peptide. In one embodiment, the eukaryotic protein or peptide is a mammalian protein or peptide. The mammalian protein or peptide may be selected from the group consisting of: an enzyme; an enzyme inhibitor; a hormone; an immune system protein; a receptor; a binding protein; a transcription factor; translation factor; tumour growth suppressing protein; a structural protein; and a blood protein.

The enzyme may be selected from the group consisting of: chymosin; gastric lipase; tissue plasminogen activator; streptokinase; a cholesterol biosynthetic or degradative steriodogenic enzyme; kinases; phosphodiesterases; methylases; de-methylases; dehydrogenases; cellulases; proteases; lipases; phospholipases; aromatases; cytochromes; adenylate or guanylaste cyclases and neuramidases.

The enzyme inhibitor may be tissue inhibitor of metalloproteinase (TIMP). The hormone may be growth hormone.

The immune system protein may be selected from the group consisting of: a cytokine; a chemokine; a lymphokine; erythropoietin; an integrin; addressin; selectin; homing receptors; T cell receptors and immunoglobulins.

The cytokine may be an interleukin, for example IL-2, IL-4 and/or IL-6, colony stimulating factor (CSF), granulocyte colony stimulating factor (G-CSF), granulocyte-macrophage colony stimulating factor (GM-CSF) or tumour necrosis factor (TNF).

The chemokine may be a macrophage inflammatory protein-2 and/or a plasminogen activator.

The lymphokine may be an interferon.

The immunoglobulin may be a natural, modified or chimeric immunoglobulin or a fragment thereof. Preferably, the immunoglobulin is a chimeric immunoglobulin having dual activity such as antibody enzyme or antibody-toxin chimera.

The hormone may be selected from the group consisting of: insulin, thyroid hormone, catecholamines, gonadotrophines, trophic hormones, prolactin, oxytocin, dopamine, bovine somatotropin, leptins; growth hormones (e.g., human grown hormone), growth factors (e.g., epidermal growth factor, nerve growth factor, insulin-like growth factor and the like).

The receptor may be a steroid hormone receptor or a peptide receptor. Preferably, the receptor is a growth factor receptor.

The binding protein may be a growth factor binding protein.

The tumour growth suppressing protein may be a protein that inhibits angiogenesis.

The structural protein may be selected from the group consisting of: collagen; fibroin; fibrinogen; elastin; tubulin; actin; and myosin.

The blood protein may be selected from the group consisting of thrombin; serum albumin; Factor VII; Factor VIII; insulin; Factor IX; Factor X; tissue plasminogen activator; protein C; von Willebrand factor; antithrombin III; glucocerebrosidase; erythropoietin granulocyte colony stimulating factor (GCSF) or modified Factor VIII; and anticoagulants.

In one preferred embodiment, the therapeutic biomolecule is a cytokine which is capable of regulating lymphoid homeostasis, preferably a cytokine which is involved in and preferably induces or enhances development, priming, expansion, differentiation and/or survival of T cells. Thus, preferably, the cytokine is an interleukin. Most preferably, IL-2, IL-7, IL-12, IL-15, or IL-21. Especially preferred is IL-7, as described in the Examples.

The therapeutic biomolecule may be protein that is capable of enhancing reprogramming of somatic cells to cells having stem cell characteristics. The protein that is capable of enhancing reprogramming of somatic cells to cells having stem cell characteristics may be selected from the group consisting of: OCT4, SOX2, NANOG, LIN28, p53, ART-4, BAGE, ss-catenin/m, Bcr-abL CAMEL, CAP-1, CASP-8, CDC27/m, CD 4/m, CEA, CLAUDIN-12, c-MYC, CT, Cyp-B, DAM, ELF2M, ETV6-AML1, G250, GAGE, GnT-V, GaplOO, HAGE, HER-2/neu, HPV-E7, HPV-E6, HAST-2, hTERT (or hTRT), LAGE, LDLR/FUT, MAGE-A, MAGE-B, MAGE-C, MART-1/Melan-A, MC1R, Myosin/m, MUC1, MUM-1, -2, -3, NA88-A, NF1, NY-ESO-1, NY-BR-1, p190 minor BCR-abL, Plac-1, Pml/RARa, PRAME, proteinase 3, PSA, PSM, RAGE, RU1 or RU2, SAGE, SART-1 or SART-3, SCGB3A2, SCP1, SCP2, SCP3, SSX, SURVIVIN, TEL/AML1, TPI/m, TRP-1, TRP-2, TRP-2/INT2, TPTE and WT, preferably WT-1.

Preferably, MAGE-A is selected from the group consisting of: MAGE-A 1, MAGE-A2, MAGE-A3, MAGE-A4, MAGE-A5, MAGE-A6, MAGE-A7, MAGE-A8, MAGE-A9, MAGE-A 10, MAGE-A 11, or MAGE-A 12.

Preferably, the protein that is capable of enhancing reprogramming of somatic cells to cells having stem cell characteristics is OCT4, SOX2, LF4; c-MYC; NANOG; LIN28.

The therapeutic biomolecule may be a biomolecule that is utilised for the modification of cells ex vivo for cell-therapy indications. Thus, preferably the therapeutic biomolecule may be selected from the group consisting of an immunoglobulin, a T-cell receptor and NK receptor.

The therapeutic biomolecule may be an RNA molecule that is capable of regulating expression of endogenous host genes, for example an interfering RNA, such as small RNA, siRNA or microRNA.

The sequence encoding the at least one ISP may be disposed anywhere within the RNA constructs of the invention, such that the sequence encoding the therapeutic biomolecule (i.e. the GOI in FIG. 1) may be disposed either 5′ or 3′ to the sequence encoding the at least one ISP.

Similarly, the sequence encoding the at least one IMP and/or IIP may be disposed anywhere within the RNA construct, such that the sequence encoding the therapeutic biomolecule (i.e. the GOI in FIG. 1) may be disposed either 5′ or 3′ to the sequence encoding the at least one IMP and/or IIP. Also, the sequence encoding the at least one IMP and/or IIP may be disposed either 5′ or 3′ to the sequence encoding the ISP.

For example, in one embodiment, the sequence encoding the therapeutic biomolecule (GOI) is preferably disposed 5′ to the sequence encoding the ISP. See for example, the saRNA embodiments 2a, 3a, 4a, and the mRNA embodiments 6a and 7a shown in FIG. 1. Also, see the saRNA embodiments 8a, 9a, 9c, and the mRNA embodiments 10a and 10b shown in FIG. 2.

However, in another embodiment, the sequence encoding the therapeutic biomolecule is preferably disposed 3′ to the sequence encoding the at least one ISP. See for example, the saRNA embodiments 2b, 3b, 4b, and the mRNA embodiments 6b and 7b shown in FIG. 1.

Preferably, the RNA construct comprises at least one promotor, which may be either genomic or subgenomic. Preferably, however, the promoter is a subgenomic promoter, as is shown in FIG. 1 (embodiments 1-4b) and FIG. 2 (embodiments 8a-9c). Preferably, therefore, saRNA constructs of the invention comprise a promoter. The skilled person would understand that the subgenomic promotor relates to a promoter that is operably linked to the sequences encoding the at least one therapeutic biomolecule and the ISP, IIP and/or IMP, such that it enables the transcription of the nucleotide sequence encoding the therapeutic biomolecule, the ISP, IIP and/or IMP.

Preferably, the subgenomic promoter is 26S, which is provided herein as SEQ ID No: 184, as follows:

| [SEQ ID No: 184] | |
|---|---|
| | GGGCCCCTATAACTCTCTACGGCTAACCTGAATGGACTACGACAT |

Accordingly, preferably the promoter (which is preferably a subgenomic promoter) is as substantially as set out in SEQ ID NO: 184, or a variant or fragment thereof.

In one embodiment, the same promotor is operably linked to the sequence encoding the at least therapeutic biomolecule and the sequence encoding the at least one ISP, IIP and/or IMP.

The inventor's designs, wherein the therapeutic biomolecule (i.e. GOI) and the ISP, IIP and/or IMP, are encoded by a single strand of RNA, advantageously enables the use of much smaller doses of RNA, because it ensures that the protein is being expressed in the same cell that is sensing the RNA, and can also be replicated, therefore having the additional aspect of expression and amplification of the innate modulatory component.

Thus, in one embodiment of the RNA construct, the promoter is disposed 5′ of the sequence encoding the at least one therapeutic biomolecule and the sequence encoding the ISP, IIP and/or IMP, such that the promoter is operably linked to both sequences, thereby driving expression of both.

In another embodiment, however, a first promotor is operably linked to the sequence encoding the at least one therapeutic biomolecule, and a second promotor is operably linked the sequence encoding the at least one ISP, IIP and/or IMP. This is referred to as a double genomic construct. Preferably, the first and/or second promoter is genomic or subgenomic. Preferably, both promoters are subgenomic promoters, such as 26S.

In some embodiments, a first promotor is operably linked to the sequence encoding the at least one therapeutic biomolecule, a second promotor is operably linked the sequence encoding the at least one ISP, and a third promoter is operably linked to the sequence encoding the at least one IIP and/or IMP. This is referred to as a treble genomic construct. Preferably, the first, second promoter and/or third promoter is genomic or subgenomic. Preferably, the promoters are subgenomic promoters, such as 26S.

The RNA construct may encode at least two, three, four or five ISP, IIP and/or IMP.

In embodiments in which there is more than one sequence encoding an ISP, IIP and/or IMP, a single promotor may be operably linked to all sequences encoding an ISP, IIP and/or IMP. Alternatively, a promotor may be linked to each of the sequences encoding an ISP, IIP and/or IMP, such that each protein is operably linked to a separate promoter. In this embodiment, the separate promoters may comprise the same promotor sequence or different promoter sequences. In another embodiment, different promotors are operably linked to each sequence encoding an ISP, IIP and/or IMP.

The nucleotide sequence encoding the at least one ISP is preferably disposed 3′ of the nucleotide sequence encoding the therapeutic biomolecule. The nucleotide sequence encoding the at least one IIP is preferably disposed 3′ of the nucleotide sequence encoding the therapeutic biomolecule. The nucleotide sequence encoding the at least one IMP is preferably disposed 3′ of the nucleotide sequence encoding the therapeutic biomolecule.

The order in which the nucleotide sequence encoding a plurality of ISPs, IIPs and/or IMPs can vary, and various embodiments are shown in FIG. 2. For example, in an embodiment, the saRNA construct may comprise a nucleotide sequence encoding a IMP, which is disposed 5′ of a nucleotide sequence encoding an ISP (this is shown in FIG. 2, Embodiment 9d). However, in another embodiment, the saRNA construct may comprise a nucleotide sequence encoding a IMP, which is disposed 3′ of a nucleotide sequence encoding an ISP (this is shown in FIG. 2, Embodiment 9e), i.e. the order of the IMP and ISP are reversed. It will be appreciated that the IMP may be replaced by an ISP or IIP, and so on, so that any order of IMP, ISP and IIP is envisaged.

As described in the Examples, in one preferred embodiment, the saRNA construct may comprise a nucleotide sequence encoding an ISP, and a nucleotide sequence encoding an IIP. Preferably, the ISP is selected from a group consisting of CCL2, CCL3, IL-7 and GM-CSF. Preferably, the IIP is selected from a group consisting of IRF-1, E3L and PR34.

Thus, in one preferred embodiment, the saRNA construct may comprise a nucleotide sequence encoding CCL2, and a nucleotide sequence encoding IRF-1. In another preferred embodiment, the saRNA construct may comprise a nucleotide sequence encoding CCL2, and a nucleotide sequence encoding E3L. In another preferred embodiment, the saRNA construct may comprise a nucleotide sequence encoding CCL2, and a nucleotide sequence encoding PR34.

Thus, in one preferred embodiment, the saRNA construct may comprise a nucleotide sequence encoding CCL3, and a nucleotide sequence encoding IRF-1. In another preferred embodiment, the saRNA construct may comprise a nucleotide sequence encoding CCL3, and a nucleotide sequence encoding E3L. In another preferred embodiment, the saRNA construct may comprise a nucleotide sequence encoding CCL3, and a nucleotide sequence encoding PR34.

Thus, in one preferred embodiment, the saRNA construct may comprise a nucleotide sequence encoding IL-7, and a nucleotide sequence encoding IRF-1. In another preferred embodiment, the saRNA construct may comprise a nucleotide sequence encoding IL-7, and a nucleotide sequence encoding E3L. In another preferred embodiment, the saRNA construct may comprise a nucleotide sequence encoding IL-7, and a nucleotide sequence encoding PR34.

Thus, in one preferred embodiment, the saRNA construct may comprise a nucleotide sequence encoding GM-CSF, and a nucleotide sequence encoding IRF-1. In another preferred embodiment, the saRNA construct may comprise a nucleotide sequence encoding GM-CSF, and a nucleotide sequence encoding E3L. In another preferred embodiment, the saRNA construct may comprise a nucleotide sequence encoding GM-CSF, and a nucleotide sequence encoding PR34.

In each of the above embodiments, the ISP may be encoded 5′ or 3′ of the IIP, and the IIP may be encoded 5′ or 3′ of the ISP.

The RNA construct may further comprise a linker sequence disposed between the sequence encoding the at least one therapeutic biomolecule and the sequence encoding the at least one ISP, IIP and/or IMP. This linker sequence is such that it allows the production of the ISP, IIP and/or IMP and the production of the therapeutic molecule from the single promoter. In one embodiment, the linker sequence encodes a peptide linker that is configured to be digested or cleaved following translation, to thereby separate the at least one therapeutic biomolecule and the at least one ISP, IIP and/or IMP in the host cell. As such, the linker sequence is preferably a cleavable peptide, which may form a cleavage site, for example a 2A peptide (Furler S, Paterna J-C, Weibel M and Bueler H Recombinant AAV vectors containing the foot and mouth disease virus 2A sequence confer efficient bicistronic gene expression in cultured cells and rat substantia nigra neurons Gene Ther. 2001, vol. 8, PP: 864-873).

Preferably, the linker sequence encoding the 2A peptide sequence connects the two coding sequence together. This enables the RNA construct to overcome the size restrictions that may occur with expression in various vectors and enables expression and translation of all of the peptides encoded by the RNA construct to occur under control of a single promoter, as a single protein. Thus, following the translation of the single protein comprising the sequences of the ISP, IIP and/or IMP, the 2A peptide, and the therapeutic biomolecule, cleavage occurs in the viral 2A peptide sequence at the terminal glycine-proline link, thereby liberating two polypeptides.

The 2A spacer sequence may be any known variant, which includes those sequences referred to as E2A, F2A, P2A and T2A, as disclosed in Wang Y et al. Scientific Reports 2015, 5, i.e. suitable 2A peptides include the porcine teschovirus-1 2A (P2A)—ATNFSLLKQAGDVEENPGP (SEQ ID No: 136), thosea asigna virus 2A (T2A)—QCTNYALLKLAGDVESNPGP(SEQ ID No: 137), equine rhinitis A virus 2A (E2A), and Foot and mouth disease virus 2A (F2A) VKQTLNFDLLKLAGDVESNPGP (SEQ ID No: 138). Preferably, the 2A peptide is thosea asigna virus 2A (T2A).

In another embodiment, the cleavable peptide is a self-cleaving peptide. In an embodiment, the linker comprises a viral 2A peptide spacer and further comprises a furin cleavage site. Preferably, the self-cleaving peptide is a furin/2A peptide. Insertion of an upstream furin cleavage site allows the removal of 2A residues that would otherwise remain attached to the upstream protein.

The furin sequence may be disposed 3′ or 5′ of the 2A sequence. Preferably, however, the furin sequence is disposed 5′ of the 2A sequence, and preferably with a GSG spacer disposed between the furin and 2A sequence.

The skilled person would appreciate that furin is a ubiquitous calcium-dependent proprotein convertase located in the secretory pathway (mainly in the golgi and trans-golgi network) that cleaves precursor proteins at a specific recognition sequence—canonically R-X-R/K/X-R (SEQ ID No: 139), and cleaving the proprotein after the final R. Thus, in one embodiment the furin sequence is R-X-R/K/X-R. However, preferably, the furin sequence is the optimised sequence RRRRRR (SEQ ID No: 140) a GSG sequence. Preferably, the GSG spacer is disposed 3′ of the furin sequence and 5′ of the 2A sequence.

Thus, preferably, the spacer sequence is the furin/T2A, as provided by NCBI Reference Sequence: GenBank: AAC97195.1, and provided herein as SEQ ID No: 141, as follows:

[SEQ ID No: 141]
RRRRRRGSGEGRGSLLTCGDVEENPGP

Hence, preferably the spacer sequence comprises an amino acid sequence substantially as set out in SEQ ID NO: 141, or a variant or fragment thereof. FIG. 1 shows embodiments 2a, 2b and 6a, 6b in which the GOI and IMP are linked by a nucleotide sequence which encodes the Furin-T2a cleavage site. In one embodiment, shown as either 2a or 6a in FIG. 1, the F-T2a cleavage site separates a 5′ GOI and a 3′ ISP. In one embodiment, shown as either 2b or 6b in FIG. 1, the F-T2a cleavage site separates a 3′ GOI and a 5′ ISP. Similar arrangements are shown in FIG. 2.

In embodiments in which the RNA construct or replicon comprises more than one sequence encoding an ISP, IIP and/or IMP, the construct may comprise linker sequences disposed between each sequence encoding an ISP, IIP and/or IMP, or only between some IMPs.

In one embodiment, the sequence encoding the at least one therapeutic biomolecule and the sequence encoding the at least one ISP, IIP and/or IMP may be separated by a stop codon followed by an internal ribosome entry site (IRES) sequence capable of initiating translation of the downstream sequence, whichever sequence that may be (i.e. GOI or ISP, IIP and/or IMP as shown in the embodiments in FIGS. 1 and 2). Therefore, preferably the IRES sequence is disposed between the sequence encoding the at least one therapeutic biomolecule and the sequence encoding at least one ISP, IIP and/or IMP. Where multiple sequences encoding at least one ISP, IIP and/or IMP are used, linker sequences may include combinations of known cleavage sequences and/or IRES sequences. In one embodiment, shown as either 3a or 7a in FIG. 1, the IRES site separates a 5′ GOI and a 3′ ISP. In one embodiment, shown as either 3b or 7b in FIG. 1, the IRES site separates a 3′ GOI and a 5′ ISP. Similar arrangements are shown in FIG. 2.

In an embodiment, the IRES is a picornavirus IRES. Oher typical IRES sequences include those such as the IRES sequence of encephalomyocarditis virus (EMCV) or vascular endothelial growth factor and type 1 collagen-inducible protein (VCIP), and would be known to those skilled in the Art.

In other embodiments, the IRES may be selected from a rhinovirus IRES, a hepatitis A virus IRES, a hepatitis C virus IRES, a poliovirus IRES, an enterovirus IRES, a cardiovirus IRES, an aphthovirus IRES, flavivirus IRES, a pestivirus IRES, a cripavirus IRES, a rhopalosiphum padi virus IRES, or any suitable IRES. In particular, the IRES may be any IRES described by the “IRESite” which provides a database of experimentally verified IRES structures (http://www.iresite.org/), or as disclosed in “New Messenger RNA Research Communications” (ISBN: 1-60021-488-6).

In a preferred embodiment, the IRES is a foot-and-mouth disease virus (FMDV) IRES, which may be as set out in SEQ ID No:142, or a fragment or variant thereof, as follows:

[SEQ ID NO: 142]
AGCAGGTTTCCCCAACTGACACAAAACGTGCAACTTGAAACTCCGCCTG
GTCTTTCCAGGTCTAGAGGGGTAACACTTTGTACTGCGTTTGGCTCCAC
GCTCGATCCACTGGCGAGTGTTAGTAACAGCACTGTTGCTTCGTAGCGG
AGCATGACGGCCGTGGGAACTCCTCCTTGGTAACAAGGACCCACGGGGC
CAAAAGCCACGCCCACACGGGCCCGTCATGTGTGCAACCCCAGCACGGC
GACTTTACTGCGAAACCCACTTTAAAGTGACATTGAAACTGGTACCCAC
ACACTGGTGACAGGCTAAGGATGCCCTTCAGGTACCCCGAGGTAACACG
CGACACTCGGGATCTGAGAAGGGGACTGGGGCTTCTATAAAAGCGCTCG
GTTTAAAAAGCTTCTATGCCTGAATAGGTGACCGGAGGTCGGCACCTTT
CCTTTGCAATTACTGACCAC

In another preferred embodiment, the IRES is an encephalomyocarditis virus (EMCV) IRES. The EMCV IRES may be as set out in SEQ ID No:143, or a fragment or variant thereof, as follows:

[SEQ ID NO: 143]
CGTTACTGGCCGAAGCCGCTTGGAATAAGGCCGGTGTGCGTTTGTCTAT
ATGTTATTTTCCACCATATTGCCGTCTTTTGGCAATGTGAGGGCCCGGA
AACCTGGCCCTGTCTTCTTGACGAGCATTCCTAGGGGTCTTTCCCCTCT
CGCCAAAGGAATGCAAGGTCTGTTGAATGTCGTGAAGGAAGCAGTTCCT
CTGGAAGCTTCTTGAAGACAAACAACGTCTGTAGCGACCCTTTGCAGGC
AGCGGAACCCCCCACCTGGCGACAGGTGCCTCTGCGGCCAAAAGCCACG
TGTATAAGATACACCTGCAAAGGCGGCACAACCCCAGTGCCACGTTGTG
AGTTGGATAGTTGTGGAAAGAGTCAAATGGCTCCCCTCAAGCGTATTCA
ACAAGGGGCTGAAGGATGCCCAGAAGGTACCCCATTGTATGGGATCTGA
TCTGGGGCCTCGGTGCACATGCTTTTCATGTGTTTAGTCGAGGTTAAAA
AACGTCTAGGCCCCCCGAACCACGGGGACGTGGTTTTCCTTTGAAAAAC
ACGATGATAATA

Therefore, preferably the IRES comprises a nucleotide sequence substantially as set out in SEQ ID No: 142 or 143, or a fragment or variant thereof.

Alternatively, instead of an IRES or a 2A linker, the linker sequence may comprise a sequence encoding a flexible linker, which allows for the expression of both the therapeutic biomolecule and ISP, IIP and/or IMP as a single polypeptide chain, but wherein the therapeutic biomolecule and ISP, IIP and/or IMP act as independent proteins. Hence, the proteins exert their effects in the same manner as if they were singly expressed.

The flexible linker sequence may be as disclosed by WO 2013/061076 A1 (Oxford Biomedica). The flexible linker sequence may be referred to herein as SEQ ID No:144, or a fragment or variant thereof, as follows:

[SEQ ID NO: 144]
GGAGGTGGCGGGTCCGGGGGCGGGGGTAGCGGTGGCGGGGGCTCC

Preferably, therefore, the flexible linker sequence comprises a nucleotide sequence substantially as set out in SEQ ID No: 144, or a fragment or variant thereof.

In one preferred embodiment, the flexible linker sequence comprises a nucleotide sequence encoding an amino acid sequence referred to herein as SEQ ID NO: 145, or a fragment or variant thereof, as set out below:

| [SEQ ID NO: 145] | |
|---|---|
| | GGGGSGGGGSGGGGS |

Preferably, therefore, the flexible linker sequence encodes an amino acid sequence substantially as set out in SEQ ID No: 145, or a fragment or variant thereof.

In yet another embodiment, the sequence encoding the at least one therapeutic biomolecule and the at least one innate inhibitor protein may be separated by a stop codon followed by a second subgenomic promotor sequence capable of initiating transcription of the downstream sequence. Examples of this embodiment are illustrated in FIG. 1, embodiments 4a and 4b.

The RNA construct (preferably when it is a saRNA construct) may encode at least one non-structural protein (NSP), disposed 5′ or 3′ of the sequence encoding the at least one therapeutic biomolecule and the at least one ISP, IIP and/or IMP. Preferably, the sequence encoding the at least one NSP is disposed 5′ of the sequences encoding the therapeutic biomolecule and the at least one ISP, IIP and/or IMP. Thus, preferably the sequence encoding the at least one NSP is disposed at the 5′ end of the RNA construct.

The at least one non-structural protein, which is encoded by the RNA construct, may be the RNA polymerase NSP4. The one or more NSP preferably encodes a replicase. Preferably, the construct encodes NSP1, NSP2, NSP3 and NSP4. The skilled person would understand that nsP1 is the viral capping enzyme and membrane anchor of the replication complex (RC), while NSP2 is an RNA helicase and the protease responsible for the NS polyprotein processing. NSP3 interacts with several host proteins and may modulate protein poly- and mono-ADP-ribosylation, and NSP4 is the core viral RNA-dependent RNA polymerase.

In one embodiment, NSP1 is provided herein as SEQ ID No: 146, as follows:

[SEQ ID No: 146]
MEKVHVDIEEDSPFLRALQRSFPQFEVEAKQVTDNDHANARAFSHLASK
LIETEVDPSDTILDIGSAPARRMYSKHKYHCICPMRCAEDPDRLYKYAT
KLKKNCKEITDKELDKKMKELAAVMSDPDLETETMCLHDDESCRYEGQV
AVYQDVYAVDGPTSLYHQANKGVRVAYWIGFDTTPFMFKNLAGAYPSYS
TNWADETVLTARNIGLCSSDVMERSRRGMSILRKKYLKPSNNVLFSVGS
TIYHEKRDLLRSWHLPSVFHLRGKQNYTCRCETIVSCDGYVVKRIAISP
GLYGKPSGYAATMHREGFLCCKVTDTLNGERVSFPVCTYVPATLCDQMT
GILATDVSADDAQKLLVGLNQRIVVNGRTQRNTNTMKNYLLPVVAQAFA
RWAKEYKEDQEDERPLGLRDRQLVMGCCWAFRRHKITSIYKRPDTQTII
KVNSDFHSFVLPRIGSNTLEIGLRTRIRKMLEEHKEPSPLITAEDVQEA
KCAADEAKEVREAEELRAALPPLAADVEEPTLEADVDLMLQEAGA

Accordingly, NSP1 preferably comprises an amino acid sequence as substantially as set out in SEQ ID No: 146, or a biologically active variant or fragment thereof.

In one embodiment, NSP1 is encoded by a nucleotide sequence a defined in SEQ ID No: 147, as follows:

[SEQ ID No: 147]
ATGGAGAAAGTTCACGTTGACATCGAGGAAGACAGCCCATTCCTCAGAGCTTTGCAGCG
GAGCTTCCCGCAGTTTGAGGTAGAAGCCAAGCAGGTCACTGATAATGACCATGCTAATG
CCAGAGCGTTTTCGCATCTGGCTTCAAAACTGATCGAAACGGAGGTGGACCCATCCGAC
ACGATCCTTGACATTGGAAGTGCGCCCGCCCGCAGAATGTATTCTAAGCACAAGTATCA
TTGTATCTGTCCGATGAGATGTGCGGAAGATCCGGACAGATTGTATAAGTATGCAACTA
AGCTGAAGAAAAACTGTAAGGAAATAACTGATAAGGAATTGGACAAGAAAATGAAGGAG
CTGGCCGCCGTCATGAGCGACCCTGACCTGGAAACTGAGACTATGTGCCTCCACGACGA
CGAGTCGTGTCGCTACGAAGGGCAAGTCGCTGTTTACCAGGATGTATACGCGGTTGACG
GACCGACAAGTCTCTATCACCAAGCCAATAAGGGAGTTAGAGTCGCCTACTGGATAGGC
TTTGACACCACCCCTTTTATGTTTAAGAACTTGGCTGGAGCATATCCATCATACTCTAC
CAACTGGGCCGACGAAACCGTGTTAACGGCTCGTAACATAGGCCTATGCAGCTCTGACG
TTATGGAGCGGTCACGTAGAGGGATGTCCATTCTTAGAAAGAAGTATTTGAAACCATCC
AACAATGTTCTATTCTCTGTTGGCTCGACCATCTACCACGAGAAGAGGGACTTACTGAG
GAGCTGGCACCTGCCGTCTGTATTTCACTTACGTGGCAAGCAAAATTACACATGTCGGT
GTGAGACTATAGTTAGTTGCGACGGGTACGTCGTTAAAAGAATAGCTATCAGTCCAGGC
CTGTATGGGAAGCCTTCAGGCTATGCTGCTACGATGCACCGCGAGGGATTCTTGTGCTG
CAAAGTGACAGACACATTGAACGGGGAGAGGGTCTCTTTTCCCGTGTGCACGTATGTGC
CAGCTACATTGTGTGACCAAATGACTGGCATACTGGCAACAGATGTCAGTGCGGACGAC
GCGCAAAAACTGCTGGTTGGGCTCAACCAGCGTATAGTCGTCAACGGTCGCACCCAGAG
AAACACCAATACCATGAAAAATTACCTTTTGCCCGTAGTGGCCCAGGCATTTGCTAGGT
GGGCAAAGGAATATAAGGAAGATCAAGAAGATGAAAGGCCACTAGGACTACGAGATAGA
CAGTTAGTCATGGGGTGTTGTTGGGCTTTTAGAAGGCACAAGATAACATCTATTTATAA
GCGCCCGGATACCCAAACCATCATCAAAGTGAACAGCGATTTCCACTCATTCGTGCTGC
CCAGGATAGGCAGTAACACATTGGAGATCGGGCTGAGAACAAGAATCAGGAAAATGTTA
GAGGAGCACAAGGAGCCGTCACCTCTCATTACCGCCGAGGACGTACAAGAAGCTAAGTG
CGCAGCCGATGAGGCTAAGGAGGTGCGTGAAGCCGAGGAGTTGCGCGCAGCTCTACCAC
CTTTGGCAGCTGATGTTGAGGAGCCCACTCTGGAAGCCGATGTCGACTTGATGTTACAA
GAGGCTGGGGCC

Accordingly, NSP1 is preferably encoded by a nucleotide sequence as substantially as set out in SEQ ID No: 147, or a variant or fragment thereof.

Accordingly, therefore, preferably the RNA construct comprises an RNA nucleotide sequence substantially as set out as SEQ ID No: 148, or a variant or fragment thereof.

[SEQ ID No: 148]
AUGGAGAAAGUUCACGUUGACAUCGAGGAAGACAGCCCAUUCCUCAGAGCUUUGCAGCG
GAGCUUCCCGCAGUUUGAGGUAGAAGCCAAGCAGGUCACUGAUAAUGACCAUGCUAAUG
CCAGAGCGUUUUCGCAUCUGGCUUCAAAACUGAUCGAAACGGAGGUGGACCCAUCCGAC
ACGAUCCUUGACAUUGGAAGUGCGCCCGCCCGCAGAAUGUAUUCUAAGCACAAGUAUCA
UUGUAUCUGUCCGAUGAGAUGUGCGGAAGAUCCGGACAGAUUGUAUAAGUAUGCAACUA
AGCUGAAGAAAAACUGUAAGGAAAUAACUGAUAAGGAAUUGGACAAGAAAAUGAAGGAG
CUGGCCGCCGUCAUGAGCGACCCUGACCUGGAAACUGAGACUAUGUGCCUCCACGACGA
CGAGUCGUGUCGCUACGAAGGGCAAGUCGCUGUUUACCAGGAUGUAUACGCGGUUGACG
GACCGACAAGUCUCUAUCACCAAGCCAAUAAGGGAGUUAGAGUCGCCUACUGGAUAGGC
UUUGACACCACCCCUUUUAUGUUUAAGAACUUGGCUGGAGCAUAUCCAUCAUACUCUAC
CAACUGGGCCGACGAAACCGUGUUAACGGCUCGUAACAUAGGCCUAUGCAGCUCUGACG
UUAUGGAGCGGUCACGUAGAGGGAUGUCCAUUCUUAGAAAGAAGUAUUUGAAACCAUCC
AACAAUGUUCUAUUCUCUGUUGGCUCGACCAUCUACCACGAGAAGAGGGACUUACUGAG
GAGCUGGCACCUGCCGUCUGUAUUUCACUUACGUGGCAAGCAAAAUUACACAUGUCGGU
GUGAGACUAUAGUUAGUUGCGACGGGUACGUCGUUAAAAGAAUAGCUAUCAGUCCAGGC
CUGUAUGGGAAGCCUUCAGGCUAUGCUGCUACGAUGCACCGCGAGGGAUUCUUGUGCUG
CAAAGUGACAGACACAUUGAACGGGGAGAGGGUCUCUUUUCCCGUGUGCACGUAUGUGC
CAGCUACAUUGUGUGACCAAAUGACUGGCAUACUGGCAACAGAUGUCAGUGCGGACGAC
GCGCAAAAACUGCUGGUUGGGCUCAACCAGCGUAUAGUCGUCAACGGUCGCACCCAGAG
AAACACCAAUACCAUGAAAAAUUACCUUUUGCCCGUAGUGGCCCAGGCAUUUGCUAGGU
GGGCAAAGGAAUAUAAGGAAGAUCAAGAAGAUGAAAGGCCACUAGGACUACGAGAUAGA
CAGUUAGUCAUGGGGUGUUGUUGGGCUUUUAGAAGGCACAAGAUAACAUCUAUUUAUAA
GCGCCCGGAUACCCAAACCAUCAUCAAAGUGAACAGCGAUUUCCACUCAUUCGUGCUGC
CCAGGAUAGGCAGUAACACAUUGGAGAUCGGGCUGAGAACAAGAAUCAGGAAAAUGUUA
GAGGAGCACAAGGAGCCGUCACCUCUCAUUACCGCCGAGGACGUACAAGAAGCUAAGUG
CGCAGCCGAUGAGGCUAAGGAGGUGCGUGAAGCCGAGGAGUUGCGCGCAGCUCUACCAC
CUUUGGCAGCUGAUGUUGAGGAGCCCACUCUGGAAGCCGAUGUCGACUUGAUGUUACAA
GAGGCUGGGGCC

In one embodiment, NSP2 is provided herein as SEQ ID No: 149, as follows:

[SEQ ID No: 149]
GSVETPRGLIKVTSYDGEDKIGSYAVLSPQAVLKSEKLSCIHPLAEQVIVITHSGRKGR
YAVEPYHGKVVVPEGHAIPVQDFQALSESATIVYNEREFVNRYLHHIATHGGALNTDEE
YYKTVKPSEHDGEYLYDIDRKQCVKKELVTGLGLTGELVDPPFHEFAYESLRTRPAAPY
QVPTIGVYGVPGSGKSGIIKSAVTKKDLVVSAKKENCAEIIRDVKKMKGLDVNARTVDS
VLLNGCKHPVETLYIDEAFACHAGTLRALIAIIRPKKAVLCGDPKQCGFFNMMCLKVHF
NHEICTQVFHKSISRRCTKSVTSVVSTLFYDKKMRTTNPKETKIVIDTTGSTKPKQDDL
ILTCFRGWVKQLQIDYKGNEIMTAAASQGLTRKGVYAVRYKVNENPLYAPTSEHVNVLL
TRTEDRIVWKTLAGDPWIKTLTAKYPGNFTATIEEWQAEHDAIMRHILERPDPTDVFON
KANVCWAKALVPVLKTAGIDMTTEQWNTVDYFETDKAHSAEIVLNQLCVRFFGLDLDSG
LFSAPTVPLSIRNNHWDNSPSPNMYGLNKEVVROLSRRYPOLPRAVATGRVYDMNTGTL
RNYDPRINLVPVNRRLPHALVLHHNEHPQSDFSSFVSKLKGRTVLVVGEKLSVPGKMVD
WLSDRPEATFRARLDLGIPGDVPKYDIIFVNVRTPYKYHHYQQCEDHAIKLSMLTKKAC
LHLNPGGTCVSIGYGYADRASESIIGAIARQFKFSRVCKPKSSLEETEVLFVFIGYDRK
ARTHNSYKLSSTLTNIYTGSRLHEAGC

Accordingly, NSP2 preferably comprises an amino acid sequence as substantially as set out in SEQ ID No: 149, or a biologically active variant or fragment thereof.

In one embodiment, NSP2 is encoded by a nucleotide sequence a defined in SEQ ID No: 150, as follows:

[SEQ ID No: 150]
GGCTCAGTGGAGACACCTCGTGGCTTGATAAAGGTTACCAGCTACGATGGCGAGGACAA
GATCGGCTCTTACGCTGTGCTTTCTCCGCAGGCTGTACTCAAGAGTGAAAAATTATCTT
GCATCCACCCTCTCGCTGAACAAGTCATAGTGATAACACACTCTGGCCGAAAAGGGCGT
TATGCCGTGGAACCATACCATGGTAAAGTAGTGGTGCCAGAGGGACATGCAATACCCGT
CCAGGACTTTCAAGCTCTGAGTGAAAGTGCCACCATTGTGTACAACGAACGTGAGTTCG
TAAACAGGTACCTGCACCATATTGCCACACATGGAGGAGCGCTGAACACTGATGAAGAA
TATTACAAAACTGTCAAGCCCAGCGAGCACGACGGCGAATACCTGTACGACATCGACAG
GAAACAGTGCGTCAAGAAAGAACTAGTCACTGGGCTAGGGCTCACAGGCGAGCTGGTGG
ATCCTCCCTTCCATGAATTCGCCTACGAGAGTCTGAGAACACGACCAGCCGCTCCTTAC
CAAGTACCAACCATAGGGGTGTATGGCGTGCCAGGATCAGGCAAGTCTGGCATCATTAA
AAGCGCAGTCACCAAAAAAGATCTAGTGGTGAGCGCCAAGAAAGAAAACTGTGCAGAAA
TTATAAGGGACGTCAAGAAAATGAAAGGGCTGGACGTCAATGCCAGAACTGTGGACTCA
GTGCTCTTGAATGGATGCAAACACCCCGTAGAGACCCTGTATATTGACGAAGCTTTTGC
TTGTCATGCAGGTACTCTCAGAGCGCTCATAGCCATTATAAGACCTAAAAAGGCAGTGC
TCTGCGGGGATCCCAAACAGTGCGGTTTTTTTAACATGATGTGCCTGAAAGTGCATTTT
AACCACGAGATTTGCACACAAGTCTTCCACAAAAGCATCTCTCGCCGTTGCACTAAATC
TGTGACTTCGGTCGTCTCAACCTTGTTTTACGACAAAAAAATGAGAACGACGAATCCGA
AAGAGACTAAGATTGTGATTGACACTACCGGCAGTACCAAACCTAAGCAGGACGATCTC
ATTCTCACTTGTTTCAGAGGGTGGGTGAAGCAGTTGCAAATAGATTACAAAGGCAACGA
AATAATGACGGCAGCTGCCTCTCAAGGGCTGACCCGTAAAGGTGTGTATGCCGTTCGGT
ACAAGGTGAATGAAAATCCTCTGTACGCACCCACCTCAGAACATGTGAACGTCCTACTG
ACCCGCACGGAGGACCGCATCGTGTGGAAAACACTAGCCGGCGACCCATGGATAAAAAC
ACTGACTGCCAAGTACCCTGGGAATTTCACTGCCACGATAGAGGAGTGGCAAGCAGAGC
ATGATGCCATCATGAGGCACATCTTGGAGAGACCGGACCCTACCGACGTCTTCCAGAAT
AAGGCAAACGTGTGTTGGGCCAAGGCTTTAGTGCCGGTGCTGAAGACCGCTGGCATAGA
CATGACCACTGAACAATGGAACACTGTGGATTATTTTGAAACGGACAAAGCTCACTCAG
CAGAGATAGTATTGAACCAACTATGCGTGAGGTTCTTTGGACTCGATCTGGACTCCGGT
CTATTTTCTGCACCCACTGTTCCGTTATCCATTAGGAATAATCACTGGGATAACTCCCC
GTCGCCTAACATGTACGGGCTGAATAAAGAAGTGGTCCGTCAGCTCTCTCGCAGGTACC
CACAACTGCCTCGGGCAGTTGCCACTGGAAGAGTCTATGACATGAACACTGGTACACTG
CGCAATTATGATCCGCGCATAAACCTAGTACCTGTAAACAGAAGACTGCCTCATGCTTT
AGTCCTCCACCATAATGAACACCCACAGAGTGACTTTTCTTCATTCGTCAGCAAATTGA
AGGGCAGAACTGTCCTGGTGGTCGGGGAAAAGTTGTCCGTCCCAGGCAAAATGGTTGAC
TGGTTGTCAGACCGGCCTGAGGCTACCTTCAGAGCTCGGCTGGATTTAGGCATCCCAGG
TGATGTGCCCAAATATGACATAATATTTGTTAATGTGAGGACCCCATATAAATACCATC
ACTATCAGCAGTGTGAAGACCATGCCATTAAGCTTAGCATGTTGACCAAGAAAGCTTGT
CTGCATCTGAATCCCGGCGGAACCTGTGTCAGCATAGGTTATGGTTACGCTGACAGGGC
CAGCGAAAGCATCATTGGTGCTATAGCGCGGCAGTTCAAGTTTTCCCGGGTATGCAAAC
CGAAATCCTCACTTGAAGAGACGGAAGTTCTGTTTGTATTCATTGGGTACGATCGCAAG
GCCCGTACGCACAATTCTTACAAGCTTTCATCAACCTTGACCAACATTTATACAGGTTC
CAGACTCCACGAAGCCGGATGT

Accordingly, preferably NSP2 is encoded by a nucleotide sequence as substantially as set out in SEQ ID No: 150, or a variant or fragment thereof.

Thus, the RNA construct may comprise SEQ ID No: 151, as follows:

[SEQ ID No: 151]
GGCUCAGUGGAGACACCUCGUGGCUUGAUAAAGGUUACCAGCUACGAUGGCGAGGACAA
GAUCGGCUCUUACGCUGUGCUUUCUCCGCAGGCUGUACUCAAGAGUGAAAAAUUAUCUU
GCAUCCACCCUCUCGCUGAACAAGUCAUAGUGAUAACACACUCUGGCCGAAAAGGGCGU
UAUGCCGUGGAACCAUACCAUGGUAAAGUAGUGGUGCCAGAGGGACAUGCAAUACCCGU
CCAGGACUUUCAAGCUCUGAGUGAAAGUGCCACCAUUGUGUACAACGAACGUGAGUUCG
UAAACAGGUACCUGCACCAUAUUGCCACACAUGGAGGAGCGCUGAACACUGAUGAAGAA
UAUUACAAAACUGUCAAGCCCAGCGAGCACGACGGCGAAUACCUGUACGACAUCGACAG
GAAACAGUGCGUCAAGAAAGAACUAGUCACUGGGCUAGGGCUCACAGGCGAGCUGGUGG
AUCCUCCCUUCCAUGAAUUCGCCUACGAGAGUCUGAGAACACGACCAGCCGCUCCUUAC
CAAGUACCAACCAUAGGGGUGUAUGGCGUGCCAGGAUCAGGCAAGUCUGGCAUCAUUAA
AAGCGCAGUCACCAAAAAAGAUCUAGUGGUGAGCGCCAAGAAAGAAAACUGUGCAGAAA
UUAUAAGGGACGUCAAGAAAAUGAAAGGGCUGGACGUCAAUGCCAGAACUGUGGACUCA
GUGCUCUUGAAUGGAUGCAAACACCCCGUAGAGACCCUGUAUAUUGACGAAGCUUUUGC
UUGUCAUGCAGGUACUCUCAGAGCGCUCAUAGCCAUUAUAAGACCUAAAAAGGCAGUGC
UCUGCGGGGAUCCCAAACAGUGCGGUUUUUUUAACAUGAUGUGCCUGAAAGUGCAUUUU
AACCACGAGAUUUGCACACAAGUCUUCCACAAAAGCAUCUCUCGCCGUUGCACUAAAUC
UGUGACUUCGGUCGUCUCAACCUUGUUUUACGACAAAAAAAUGAGAACGACGAAUCCGA
AAGAGACUAAGAUUGUGAUUGACACUACCGGCAGUACCAAACCUAAGCAGGACGAUCUC
AUUCUCACUUGUUUCAGAGGGUGGGUGAAGCAGUUGCAAAUAGAUUACAAAGGCAACGA
AAUAAUGACGGCAGCUGCCUCUCAAGGGCUGACCCGUAAAGGUGUGUAUGCCGUUCGGU
ACAAGGUGAAUGAAAAUCCUCUGUACGCACCCACCUCAGAACAUGUGAACGUCCUACUG
ACCCGCACGGAGGACCGCAUCGUGUGGAAAACACUAGCCGGCGACCCAUGGAUAAAAAC
ACUGACUGCCAAGUACCCUGGGAAUUUCACUGCCACGAUAGAGGAGUGGCAAGCAGAGC
AUGAUGCCAUCAUGAGGCACAUCUUGGAGAGACCGGACCCUACCGACGUCUUCCAGAAU
AAGGCAAACGUGUGUUGGGCCAAGGCUUUAGUGCCGGUGCUGAAGACCGCUGGCAUAGA
CAUGACCACUGAACAAUGGAACACUGUGGAUUAUUUUGAAACGGACAAAGCUCACUCAG
CAGAGAUAGUAUUGAACCAACUAUGCGUGAGGUUCUUUGGACUCGAUCUGGACUCCGGU
CUAUUUUCUGCACCCACUGUUCCGUUAUCCAUUAGGAAUAAUCACUGGGAUAACUCCCC
GUCGCCUAACAUGUACGGGCUGAAUAAAGAAGUGGUCCGUCAGCUCUCUCGCAGGUACC
CACAACUGCCUCGGGCAGUUGCCACUGGAAGAGUCUAUGACAUGAACACUGGUACACUG
CGCAAUUAUGAUCCGCGCAUAAACCUAGUACCUGUAAACAGAAGACUGCCUCAUGCUUU
AGUCCUCCACCAUAAUGAACACCCACAGAGUGACUUUUCUUCAUUCGUCAGCAAAUUGA
AGGGCAGAACUGUCCUGGUGGUCGGGGAAAAGUUGUCCGUCCCAGGCAAAAUGGUUGAC
UGGUUGUCAGACCGGCCUGAGGCUACCUUCAGAGCUCGGCUGGAUUUAGGCAUCCCAGG
UGAUGUGCCCAAAUAUGACAUAAUAUUUGUUAAUGUGAGGACCCCAUAUAAAUACCAUC
ACUAUCAGCAGUGUGAAGACCAUGCCAUUAAGCUUAGCAUGUUGACCAAGAAAGCUUGU
CUGCAUCUGAAUCCCGGCGGAACCUGUGUCAGCAUAGGUUAUGGUUACGCUGACAGGGC
CAGCGAAAGCAUCAUUGGUGCUAUAGCGCGGCAGUUCAAGUUUUCCCGGGUAUGCAAAC
CGAAAUCCUCACUUGAAGAGACGGAAGUUCUGUUUGUAUUCAUUGGGUACGAUCGCAAG
GCCCGUACGCACAAUUCUUACAAGCUUUCAUCAACCUUGACCAACAUUUAUACAGGUUC
CAGACUCCACGAAGCCGGAUGU

Accordingly, therefore, preferably the RNA construct comprises an RNA nucleotide sequence substantially as set out as SEQ ID No: 151, or a variant or fragment thereof.

In one embodiment, nsP3 is provided herein as SEQ ID No: 152, as follows:

[SEQ ID No: 152]
APSYHVVRGDIATATEGVIINAANSKGQPGGGVCGALYKKEPESFDLQPIEVGKARLVK
GAAKHIIHAVGPNFNKVSEVEGDKQLAEAYESIAKIVNDNNYKSVAIPLLSTGIFSGNK
DRLTQSLNHLLTALDTTDADVAIYCRDKKWEMTLKEAVARREAVEEICISDDSSVTEPD
AELVRVHPKSSLAGRKGYSTSDGKTFSYLEGTKFHQAAKDIAEINAMWPVATEANEQVC
MYILGESMSSIRSKCPVEESEASTPPSTLPCLCIHAMTPERVORLKASRPEQITVCSSF
PLPKYRITGVQKIQCSQPILFSPKVPAYIHPRKYLVETPPVDETPEPSAENQSTEGTPE
QPPLITEDETRTRTPEPIIIEEEEEDSISLLSDGPTHQVLQVEADIHGPPSVSSSSWSI
PHASDFDVDSLSILDTLEGASVTSGATSAETNSYFAKSMEFLARPVPAPRTVERNPPHP
APRTRTPSLAPSRACSRTSLVSTPPGVNRVITREELEALTPSRTPSRSVSRTSLVSNPP
GVNRVITREEFEAFVAQQQRFDAGA

Accordingly, preferably NSP3 comprises an amino acid sequence as substantially as set out in SEQ ID No: 152, or a biologically active variant or fragment thereof.

In one embodiment, NSP3 is encoded by a nucleotide sequence a defined in SEQ ID No: 153, as follows:

[SEQ ID No: 153]
GCACCCTCATATCATGTGGTGCGAGGGGATATTGCCACGGCCACCGAAGGAGTGATTAT
AAATGCTGCTAACAGCAAAGGACAACCTGGCGGAGGGGTGTGCGGAGCGCTGTATAAGA
AATTCCCGGAAAGCTTCGATTTACAGCCGATCGAAGTAGGAAAAGCGCGACTGGTCAAA
GGTGCAGCTAAACATATCATTCATGCCGTAGGACCAAACTTCAACAAAGTTTCGGAGGT
TGAAGGTGACAAACAGTTGGCAGAGGCTTATGAGTCCATCGCTAAGATTGTCAACGATA
ACAATTACAAGTCAGTAGCGATTCCACTGTTGTCCACCGGCATCTTTTCCGGGAACAAA
GATCGACTAACCCAATCATTGAACCATTTGCTGACAGCTTTAGACACCACTGATGCAGA
TGTAGCCATATACTGCAGGGACAAGAAATGGGAAATGACTCTCAAGGAAGCAGTGGCTA
GGAGAGAAGCAGTGGAGGAGATATGCATATCCGACGACTCTTCAGTGACAGAACCTGAT
GCAGAGCTGGTGAGGGTGCATCCGAAGAGTTCTTTGGCTGGAAGGAAGGGCTACAGCAC
AAGCGATGGCAAAACTTTCTCATATTTGGAAGGGACCAAGTTTCACCAGGCGGCCAAGG
ATATAGCAGAAATTAATGCCATGTGGCCCGTTGCAACGGAGGCCAATGAGCAGGTATGC
ATGTATATCCTCGGAGAAAGCATGAGCAGTATTAGGTCGAAATGCCCCGTCGAAGAGTC
GGAAGCCTCCACACCACCTAGCACGCTGCCTTGCTTGTGCATCCATGCCATGACTCCAG
AAAGAGTACAGCGCCTAAAAGCCTCACGTCCAGAACAAATTACTGTGTGCTCATCCTTT
CCATTGCCGAAGTATAGAATCACTGGTGTGCAGAAGATCCAATGCTCCCAGCCTATATT
GTTCTCACCGAAAGTGCCTGCGTATATTCATCCAAGGAAGTATCTCGTGGAAACACCAC
CGGTAGACGAGACTCCGGAGCCATCGGCAGAGAACCAATCCACAGAGGGGACACCTGAA
CAACCACCACTTATAACCGAGGATGAGACCAGGACTAGAACGCCTGAGCCGATCATCAT
CGAAGAGGAAGAAGAGGATAGCATAAGTTTGCTGTCAGATGGCCCGACCCACCAGGTGC
TGCAAGTCGAGGCAGACATTCACGGGCCGCCCTCTGTATCTAGCTCATCCTGGTCCATT
CCTCATGCATCCGACTTTGATGTGGACAGTTTATCCATACTTGACACCCTGGAGGGAGC
TAGCGTGACCAGCGGGGCAACGTCAGCCGAGACTAACTCTTACTTCGCAAAGAGTATGG
AGTTTCTGGCGCGACCGGTGCCTGCGCCTCGAACAGTATTCAGGAACCCTCCACATCCC
GCTCCGCGCACAAGAACACCGTCACTTGCACCCAGCAGGGCCTGCTCGAGAACCAGCCT
AGTTTCCACCCCGCCAGGCGTGAATAGGGTGATCACTAGAGAGGAGCTCGAGGCGCTTA
CCCCGTCACGCACTCCTAGCAGGTCGGTCTCGAGAACCAGCCTGGTCTCCAACCCGCCA
GGCGTAAATAGGGTGATTACAAGAGAGGAGTTTGAGGCGTTCGTAGCACAACAACAATG
ACGGTTTGATGCGGGTGCA

Accordingly, preferably NSP3 is encoded by a nucleotide sequence as substantially as set out in SEQ ID No: 153, or a variant or fragment thereof.

Thus, the RNA construct may comprise SEQ ID No:154, as follows:

[SEQ ID No: 154]
GCACCCUCAUAUCAUGUGGUGCGAGGGGAUAUUGCCACGGCCACCGAAGGAGUGAUUAU
AAAUGCUGCUAACAGCAAAGGACAACCUGGCGGAGGGGUGUGCGGAGCGCUGUAUAAGA
AAUUCCCGGAAAGCUUCGAUUUACAGCCGAUCGAAGUAGGAAAAGCGCGACUGGUCAAA
GGUGCAGCUAAACAUAUCAUUCAUGCCGUAGGACCAAACUUCAACAAAGUUUCGGAGGU
UGAAGGUGACAAACAGUUGGCAGAGGCUUAUGAGUCCAUCGCUAAGAUUGUCAACGAUA
ACAAUUACAAGUCAGUAGCGAUUCCACUGUUGUCCACCGGCAUCUUUUCCGGGAACAAA
GAUCGACUAACCCAAUCAUUGAACCAUUUGCUGACAGCUUUAGACACCACUGAUGCAGA
UGUAGCCAUAUACUGCAGGGACAAGAAAUGGGAAAUGACUCUCAAGGAAGCAGUGGCUA
GGAGAGAAGCAGUGGAGGAGAUAUGCAUAUCCGACGACUCUUCAGUGACAGAACCUGAU
GCAGAGCUGGUGAGGGUGCAUCCGAAGAGUUCUUUGGCUGGAAGGAAGGGCUACAGCAC
AAGCGAUGGCAAAACUUUCUCAUAUUUGGAAGGGACCAAGUUUCACCAGGCGGCCAAGG
AUAUAGCAGAAAUUAAUGCCAUGUGGCCCGUUGCAACGGAGGCCAAUGAGCAGGUAUGC
AUGUAUAUCCUCGGAGAAAGCAUGAGCAGUAUUAGGUCGAAAUGCCCCGUCGAAGAGUC
GGAAGCCUCCACACCACCUAGCACGCUGCCUUGCUUGUGCAUCCAUGCCAUGACUCCAG
AAAGAGUACAGCGCCUAAAAGCCUCACGUCCAGAACAAAUUACUGUGUGCUCAUCCUUU
CCAUUGCCGAAGUAUAGAAUCACUGGUGUGCAGAAGAUCCAAUGCUCCCAGCCUAUAUU
GUUCUCACCGAAAGUGCCUGCGUAUAUUCAUCCAAGGAAGUAUCUCGUGGAAACACCAC
CGGUAGACGAGACUCCGGAGCCAUCGGCAGAGAACCAAUCCACAGAGGGGACACCUGAA
CAACCACCACUUAUAACCGAGGAUGAGACCAGGACUAGAACGCCUGAGCCGAUCAUCAU
CGAAGAGGAAGAAGAGGAUAGCAUAAGUUUGCUGUCAGAUGGCCCGACCCACCAGGUGC
UGCAAGUCGAGGCAGACAUUCACGGGCCGCCCUCUGUAUCUAGCUCAUCCUGGUCCAUU
CCUCAUGCAUCCGACUUUGAUGUGGACAGUUUAUCCAUACUUGACACCCUGGAGGGAGC
UAGCGUGACCAGCGGGGCAACGUCAGCCGAGACUAACUCUUACUUCGCAAAGAGUAUGG
AGUUUCUGGCGCGACCGGUGCCUGCGCCUCGAACAGUAUUCAGGAACCCUCCACAUCCC
GCUCCGCGCACAAGAACACCGUCACUUGCACCCAGCAGGGCCUGCUCGAGAACCAGCCU
AGUUUCCACCCCGCCAGGCGUGAAUAGGGUGAUCACUAGAGAGGAGCUCGAGGCGCUUA
CCCCGUCACGCACUCCUAGCAGGUCGGUCUCGAGAACCAGCCUGGUCUCCAACCCGCCA
GGCGUAAAUAGGGUGAUUACAAGAGAGGAGUUUGAGGCGUUCGUAGCACAACAACAAUG
ACGGUUUGAUGCGGGUGCA

Accordingly, therefore, preferably the RNA construct comprises an RNA nucleotide sequence substantially as set out as SEQ ID No: 154 or a variant or fragment thereof.

In one embodiment, NSP4 is provided herein as SEQ ID No: 155, as follows:

[SEQ ID No: 155]
YIFSSDTGQGHLQQKSVRQTVLSEVVLERTELEISYAPRLDQEKEELLRKKLQLNPTPA
NRSRYQSRKVENMKAITARRILOGLGHYLKAEGKVECYRTLHPVPLYSSSVNRAFSSPK
VAVEACNAMLKENFPTVASYCIIPEYDAYLDMVDGASCCLDTASFCPAKLRSFPKKHSY
LEPTIRSAVPSAIQNTLONVLAAATKRNCNVTOMRELPVLDSAAFNVECFKKYACNNEY
WETFKENPIRLTEENVVNYITKLKGPKAAALFAKTHNLNMLQDIPMDRFVMDLKRDVKV
TPGTKHTEERPKVQVIQAADPLATAYLCGIHRELVRRLNAVLLPNIHTLFDMSAEDFDA
IIAEHFQPGDCVLETDIASFDKSEDDAMALTALMILEDLGVDAELLTLIEAAFGEISSI
HLPTKTKFKFGAMMKSGMFLTLFVNTVINIVIASRVLRERLTGSPCAAFIGDDNIVKGV
KSDKLMADRCATWLNMEVKIIDAVVGEKAPYFCGGFILCDSVTGTACRVADPLKRLFKL
GKPLAADDEHDDDRRRALHEESTRWNRVGILSELCKAVESRYETVGTSIIVMAMTTLAS
SVKSFSYLRGAPITLYG

Accordingly, preferably NSP4 comprises an amino acid sequence as substantially as set out in SEQ ID No: 155, or a biologically active variant or fragment thereof.

In one embodiment, NSP4 is encoded by a nucleotide sequence a defined in SEQ ID No: 156, as follows:

[SEQ ID No: 156]
TACATCTTTTCCTCCGACACCGGTCAAGGGCATTTACAACAAAAATCAGTAAGGCAAAC
GGTGCTATCCGAAGTGGTGTTGGAGAGGACCGAATTGGAGATTTCGTATGCCCCGCGCC
TCGACCAAGAAAAAGAAGAATTACTACGCAAGAAATTACAGTTAAATCCCACACCTGCT
AACAGAAGCAGATACCAGTCCAGGAAGGTGGAGAACATGAAAGCCATAACAGCTAGACG
TATTCTGCAAGGCCTAGGGCATTATTTGAAGGCAGAAGGAAAAGTGGAGTGCTACCGAA
CCCTGCATCCTGTTCCTTTGTATTCATCTAGTGTGAACCGTGCCTTTTCAAGCCCCAAG
GTCGCAGTGGAAGCCTGTAACGCCATGTTGAAAGAGAACTTTCCGACTGTGGCTTCTTA
CTGTATTATTCCAGAGTACGATGCCTATTTGGACATGGTTGACGGAGCTTCATGCTGCT
TAGACACTGCCAGTTTTTGCCCTGCAAAGCTGCGCAGCTTTCCAAAGAAACACTCCTAT
TTGGAACCCACAATACGATCGGCAGTGCCTTCAGCGATCCAGAACACGCTCCAGAACGT
CCTGGCAGCTGCCACAAAAAGAAATTGCAATGTCACGCAAATGAGAGAATTGCCCGTAT
TGGATTCGGCGGCCTTTAATGTGGAATGCTTCAAGAAATATGCGTGTAATAATGAATAT
TGGGAAACGTTTAAAGAAAACCCCATCAGGCTTACTGAAGAAAACGTGGTAAATTACAT
TACCAAATTAAAAGGACCAAAAGCTGCTGCTCTTTTTGCGAAGACACATAATTTGAATA
TGTTGCAGGACATACCAATGGACAGGTTTGTAATGGACTTAAAGAGAGACGTGAAAGTG
ACTCCAGGAACAAAACATACTGAAGAACGGCCCAAGGTACAGGTGATCCAGGCTGCCGA
TCCGCTAGCAACAGCGTATCTGTGCGGAATCCACCGAGAGCTGGTTAGGAGATTAAATG
CGGTCCTGCTTCCGAACATTCATACACTGTTTGATATGTCGGCTGAAGACTTTGACGCT
ATTATAGCCGAGCACTTCCAGCCTGGGGATTGTGTTCTGGAAACTGACATCGCGTCGTT
TGATAAAAGTGAGGACGACGCCATGGCTCTGACCGCGTTAATGATTCTGGAAGACTTAG
GTGTGGACGCAGAGCTGTTGACGCTGATTGAGGCGGCTTTCGGCGAAATTTCATCAATA
CATTTGCCCACTAAAACTAAATTTAAATTCGGAGCCATGATGAAATCTGGAATGTTCCT
CACACTGTTTGTGAACACAGTCATTAACATTGTAATCGCAAGCAGAGTGTTGAGAGAAC
GGCTAACCGGATCACCATGTGCAGCATTCATTGGAGATGACAATATCGTGAAAGGAGTC
AAATCGGACAAATTAATGGCAGACAGGTGCGCCACCTGGTTGAATATGGAAGTCAAGAT
TATAGATGCTGTGGTGGGCGAGAAAGCGCCTTATTTCTGTGGAGGGTTTATTTTGTGTG
ACTCCGTGACCGGCACAGCGTGCCGTGTGGCAGACCCCCTAAAAAGGCTGTTTAAGCTT
GGCAAACCTCTGGCAGCAGACGATGAACATGATGATGACAGGAGAAGGGCATTGCATGA
AGAGTCAACACGCTGGAACCGAGTGGGTATTCTTTCAGAGCTGTGCAAGGCAGTAGAAT
CAAGGTATGAAACCGTAGGAACTTCCATCATAGTTATGGCCATGACTACTCTAGCTAGC
AGTGTTAAATCATTCAGCTACCTGAGAGGGGCCCCTATAACTCTCTACGGC

Accordingly, preferably NSP4 is encoded by a nucleotide sequence as substantially as set out in SEQ ID No: 156, or a variant or fragment thereof.

Thus, the RNA construct may comprise SEQ ID No: 157, as follows:

[SEQ ID No: 157]
UACAUCUUUUCCUCCGACACCGGUCAAGGGCAUUUACAACAAAAAUCAGUAAGGCAAAC
GGUGCUAUCCGAAGUGGUGUUGGAGAGGACCGAAUUGGAGAUUUCGUAUGCCCCGCGCC
UCGACCAAGAAAAAGAAGAAUUACUACGCAAGAAAUUACAGUUAAAUCCCACACCUGCU
AACAGAAGCAGAUACCAGUCCAGGAAGGUGGAGAACAUGAAAGCCAUAACAGCUAGACG
UAUUCUGCAAGGCCUAGGGCAUUAUUUGAAGGCAGAAGGAAAAGUGGAGUGCUACCGAA
CCCUGCAUCCUGUUCCUUUGUAUUCAUCUAGUGUGAACCGUGCCUUUUCAAGCCCCAAG
GUCGCAGUGGAAGCCUGUAACGCCAUGUUGAAAGAGAACUUUCCGACUGUGGCUUCUUA
CUGUAUUAUUCCAGAGUACGAUGCCUAUUUGGACAUGGUUGACGGAGCUUCAUGCUGCU
UAGACACUGCCAGUUUUUGCCCUGCAAAGCUGCGCAGCUUUCCAAAGAAACACUCCUAU
UUGGAACCCACAAUACGAUCGGCAGUGCCUUCAGCGAUCCAGAACACGCUCCAGAACGU
CCUGGCAGCUGCCACAAAAAGAAAUUGCAAUGUCACGCAAAUGAGAGAAUUGCCCGUAU
UGGAUUCGGCGGCCUUUAAUGUGGAAUGCUUCAAGAAAUAUGCGUGUAAUAAUGAAUAU
UGGGAAACGUUUAAAGAAAACCCCAUCAGGCUUACUGAAGAAAACGUGGUAAAUUACAU
UACCAAAUUAAAAGGACCAAAAGCUGCUGCUCUUUUUGCGAAGACACAUAAUUUGAAUA
UGUUGCAGGACAUACCAAUGGACAGGUUUGUAAUGGACUUAAAGAGAGACGUGAAAGUG
ACUCCAGGAACAAAACAUACUGAAGAACGGCCCAAGGUACAGGUGAUCCAGGCUGCCGA
UCCGCUAGCAACAGCGUAUCUGUGCGGAAUCCACCGAGAGCUGGUUAGGAGAUUAAAUG
CGGUCCUGCUUCCGAACAUUCAUACACUGUUUGAUAUGUCGGCUGAAGACUUUGACGCU
AUUAUAGCCGAGCACUUCCAGCCUGGGGAUUGUGUUCUGGAAACUGACAUCGCGUCGUU
UGAUAAAAGUGAGGACGACGCCAUGGCUCUGACCGCGUUAAUGAUUCUGGAAGACUUAG
GUGUGGACGCAGAGCUGUUGACGCUGAUUGAGGCGGCUUUCGGCGAAAUUUCAUCAAUA
CAUUUGCCCACUAAAACUAAAUUUAAAUUCGGAGCCAUGAUGAAAUCUGGAAUGUUCCU
CACACUGUUUGUGAACACAGUCAUUAACAUUGUAAUCGCAAGCAGAGUGUUGAGAGAAC
GGCUAACCGGAUCACCAUGUGCAGCAUUCAUUGGAGAUGACAAUAUCGUGAAAGGAGUC
AAAUCGGACAAAUUAAUGGCAGACAGGUGCGCCACCUGGUUGAAUAUGGAAGUCAAGAU
UAUAGAUGCUGUGGUGGGCGAGAAAGCGCCUUAUUUCUGUGGAGGGUUUAUUUUGUGUG
ACUCCGUGACCGGCACAGCGUGCCGUGUGGCAGACCCCCUAAAAAGGCUGUUUAAGCUU
GGCAAACCUCUGGCAGCAGACGAUGAACAUGAUGAUGACAGGAGAAGGGCAUUGCAUGA
AGAGUCAACACGCUGGAACCGAGUGGGUAUUCUUUCAGAGCUGUGCAAGGCAGUAGAAU
CAAGGUAUGAAACCGUAGGAACUUCCAUCAUAGUUAUGGCCAUGACUACUCUAGCUAGC
AGUGUUAAAUCAUUCAGCUACCUGAGAGGGGCCCCUAUAACUCUCUACGGC

Accordingly, therefore, preferably the RNA construct comprises an RNA nucleotide sequence substantially as set out as SEQ ID No: 157, or a variant or fragment thereof.

Preferably, together with proteins present in a host cell, the non-structural proteins encoded by the RNA construct of the invention form an enzyme complex (i.e. a replicase) that is required for genome replication and transcription of the sequences encoding the at least one therapeutic biomolecule and the at least one ISP, IMP and/or IIP. For example, the one or more non-structural protein may encode a polymerase to enable the construct to amplify the nucleotide sequences encoding the at least one peptide or protein of interest (i.e. therapeutic biomolecule) and the at least one ISP, IMP and/or IIP.

The host cell may be a eukaryotic or prokaryotic host cell. Preferably, the host cell is a eukaryotic host cell. More preferably, the host cell is a mammalian host cell.

The RNA construct may further comprise a promoter disposed 5′ of the at least one non-structural protein, such that the promoter is operably linked to the sequence encoding the at least one non-structural protein and enables expression of the at least one non-structural protein in a host cell.

Preferably, the RNA construct comprises a 5′ UTR conserved sequence element, which may be referred to herein as SEQ ID No: 158, as follows:

[SEQ ID No: 158]
AUGGGCGGCGCAUGAGAGAAGCCCAGACCAAUUACCUACCCAAA

Accordingly, preferably the UTR is disposed 5′ of the at least one non-structural protein and comprises a nucleotide sequence substantially as set out in SEQ ID No: 158, or a fragment or variant thereof.

Preferably, the RNA construct comprises a 3′ UTR conserved sequence element, which may be referred to herein as SEQ ID No: 159, as follows:

[SEQ ID No: 159]
AAUUGGCAAGCUGCUUACAUAGAACUCGCGGCGAUUGGCAUGCCGCCUU
AAAAUUUUUAUUUUAUUUUUCUUUUCUUUUCCGAAUCGGAUUUUGUUUU
UAAUAUUUCAAAAAAAAAAAAAAAAAAAAAAAAAAAAAAAAAAA

Accordingly, preferably the 3′ UTR is disposed 3′ of the at least one non-structural protein and comprises a nucleotide sequence substantially as set out in SEQ ID No: 159, or a fragment or variant thereof.

Preferably, the RNA construct comprises a polyA tail. Preferably, the polyA tail is disposed at the 3′ end of the construct. The poly A tail may comprise at least 35 nt, or at least 40 nt, or at least 45 nt, or at least 50 nt, wherein each nt is an adenine. In another embodiment, the polyA tail may comprise at least 55 nt or at least 60 nt, wherein each nt is an adenine. In yet another embodiment, the polyA tail may comprise at least 60 adenines, followed by one or more non-adenine nucleotides (i.e. G, C or T, preferably guanine), and then another at least 35 nt, or at least 40 nt, or at least 45 nt, or at least 50 nt, or at least 55 nt, or at least 60 nt, wherein each nt is an adenine.

The RNA construct may further comprise a 5′ cap. In the context of the present invention, the term “5′-cap” includes a 5′-cap analog that resembles the RNA cap structure and is modified to possess the ability to stabilize RNA and/or enhance translation of RNA if attached thereto, preferably in vivo and/or in a cell.

An RNA with a 5′-cap may be achieved by in vitro transcription of a DNA template in presence of said 5′-cap, wherein said 5′-cap is co-transcriptionally incorporated into the generated RNA strand, or the RNA may be generated, for example, by in vitro transcription, and the 5′-cap may be attached to the RNA post-transcriptionally using capping enzymes, for example, capping enzymes of vaccinia virus. In capped RNA, the 3′ position of the first base of a (capped) RNA molecule is linked to the 5′ position of the subsequent base of the RNA molecule (“second base”) via a phosphodiester bond.

In one embodiment, the RNA construct comprises, preferably 5′ to 3′, a promoter, a sequence encoding at least one therapeutic biomolecule, a linker sequence, and at least one sequence encoding an ISP, IMP and/or IIP. In one embodiment, the RNA construct comprises, preferably 5′ to 3′, a promoter, a sequence encoding at least one ISP, IMP and/or IIP, a linker sequence, and a sequence encoding at least one therapeutic biomolecule. The linker may be F-T2a or IRES in either embodiment.

In another embodiment, the RNA construct comprises, preferably 5′ to 3′, a promoter, a sequence encoding at least one non-structural protein, a sub genomic promoter, a sequence encoding at least one therapeutic biomolecule, a linker sequence, and a sequence encoding at least one ISP, IMP and/or IIP. In another embodiment, the RNA construct comprises, preferably 5′ to 3′, a promoter, a sequence encoding at least one non-structural protein, a sub genomic promoter, a sequence encoding at least one ISP, IMP and/or IIP, a linker sequence, and a sequence encoding at least one therapeutic biomolecule. The linker may be F-T2a or IRES in either embodiment.

In yet another embodiment, the RNA construct comprises, preferably 5′ to 3′, a promoter, a sequence encoding at least one non-structural protein, a sub genomic promoter, a sequence encoding at least one therapeutic biomolecule, a linker sequence, a sequence encoding at least one ISP, IMP and/or IIP, and a polyA tail. In yet another embodiment, the RNA construct comprises, preferably 5′ to 3′, a promoter, a sequence encoding at least one non-structural protein, a sub genomic promoter, a sequence encoding at least one ISP, IMP and/or IIP, a linker sequence, a sequence encoding at least one therapeutic biomolecule, and a polyA tail. The linker may be F-T2a or IRES in either embodiment.

In another embodiment, the RNA construct comprises, preferably 5′ to 3′, a promoter, a sequence encoding at least one non-structural protein, a first sub genomic promoter, a sequence encoding at least one therapeutic biomolecule, a second sub genomic promoter, a sequence encoding at least one ISP, IMP and/or IIP, and a polyA tail. In another embodiment, the RNA construct comprises, preferably 5′ to 3′, a promoter, a sequence encoding at least one non-structural protein, a first sub genomic promoter, a sequence encoding at least one ISP, IMP and/or IIP, a second sub genomic promoter, a sequence encoding at least one therapeutic biomolecule, and a polyA tail.

Most preferably, the RNA construct comprises, 5′ to 3′, a 5′ cap, a promoter, nsP1, nsP2, NSP3v, NSP4, the sub genomic promoter 26S, a sequence encoding a therapeutic biomolecule, a linker sequence, a sequence encoding the ISP, IMP and/or IIP and a polyA tail. Most preferably, the RNA construct comprises, 5′ to 3′, a 5′ cap, a promoter, nsP1, nsP2, nsP3v, nsP4, the sub genomic promoter 265, a sequence encoding an ISP, IMP and/or IIP, a linker sequence, a sequence encoding a therapeutic biomolecule; and a polyA tail.

In one embodiment, the saRNA comprises, preferably 5′ to 3′: (i) a promoter (preferably a sub-genomic promoter) for driving expression of a sequence encoding at least one therapeutic biomolecule; (ii) a linker sequence (preferably an IRES element); (iii) a sequence encoding an ISP (preferably, CCL2, CCL3, IL-7 or GM-CSF); (iv) a linker sequence (preferably an IRES element); and (v) a sequence encoding an IIP (preferably, IRF-1, E3L or PR34).

In one embodiment, therefore, the RNA construct may comprise a T7 Promoter, 5′UTR, NSP1-4, Sub-Genomic Promoter, GOI (gene of interest is the therapeutic biomolecule), Furin T2A, ISP is CXCL11 (which is the first ISP mentioned herein, but it will be appreciated that any of the ISPs or linkers disclosed herein may be used), 3′UTR, and PolyA tail. Thus, the RNA construct may comprise or consist of SEQ ID No: 160, a GOI, and the sequence of SEQ ID No: 181 in a single construct. SEQ ID No: 160 and SEQ ID No: 181 are as follows, where “GOI” represents the position of the therapeutic biomolecule encoding sequence:

[SEQ ID No: 160]
AUGGGCGGCGCAUGAGAGAAGCCCAGACCAAUUACCUACCCAAAAUGGAGAAAGUUCACG
UUGACAUCGAGGAAGACAGCCCAUUCCUCAGAGCUUUGCAGCGGAGCUUCCCGCAGUUUG
AGGUAGAAGCCAAGCAGGUCACUGAUAAUGACCAUGCUAAUGCCAGAGCGUUUUCGCAUC
UGGCUUCAAAACUGAUCGAAACGGAGGUGGACCCAUCCGACACGAUCCUUGACAUUGGAA
GUGCGCCCGCCCGCAGAAUGUAUUCUAAGCACAAGUAUCAUUGUAUCUGUCCGAUGAGAU
GUGCGGAAGAUCCGGACAGAUUGUAUAAGUAUGCAACUAAGCUGAAGAAAAACUGUAAGG
AAAUAACUGAUAAGGAAUUGGACAAGAAAAUGAAGGAGCUGGCCGCCGUCAUGAGCGACC
CUGACCUGGAAACUGAGACUAUGUGCCUCCACGACGACGAGUCGUGUCGCUACGAAGGGC
AAGUCGCUGUUUACCAGGAUGUAUACGCGGUUGACGGACCGACAAGUCUCUAUCACCAAG
CCAAUAAGGGAGUUAGAGUCGCCUACUGGAUAGGCUUUGACACCACCCCUUUUAUGUUUA
AGAACUUGGCUGGAGCAUAUCCAUCAUACUCUACCAACUGGGCCGACGAAACCGUGUUAA
CGGCUCGUAACAUAGGCCUAUGCAGCUCUGACGUUAUGGAGCGGUCACGUAGAGGGAUGU
CCAUUCUUAGAAAGAAGUAUUUGAAACCAUCCAACAAUGUUCUAUUCUCUGUUGGCUCGA
CCAUCUACCACGAGAAGAGGGACUUACUGAGGAGCUGGCACCUGCCGUCUGUAUUUCACU
UACGUGGCAAGCAAAAUUACACAUGUCGGUGUGAGACUAUAGUUAGUUGCGACGGGUACG
UCGUUAAAAGAAUAGCUAUCAGUCCAGGCCUGUAUGGGAAGCCUUCAGGCUAUGCUGCUA
CGAUGCACCGCGAGGGAUUCUUGUGCUGCAAAGUGACAGACACAUUGAACGGGGAGAGGG
UCUCUUUUCCCGUGUGCACGUAUGUGCCAGCUACAUUGUGUGACCAAAUGACUGGCAUAC
UGGCAACAGAUGUCAGUGCGGACGACGCGCAAAAACUGCUGGUUGGGCUCAACCAGCGUA
UAGUCGUCAACGGUCGCACCCAGAGAAACACCAAUACCAUGAAAAAUUACCUUUUGCCCG
UAGUGGCCCAGGCAUUUGCUAGGUGGGCAAAGGAAUAUAAGGAAGAUCAAGAAGAUGAAA
GGCCACUAGGACUACGAGAUAGACAGUUAGUCAUGGGGUGUUGUUGGGCUUUUAGAAGGC
ACAAGAUAACAUCUAUUUAUAAGCGCCCGGAUACCCAAACCAUCAUCAAAGUGAACAGCG
AUUUCCACUCAUUCGUGCUGCCCAGGAUAGGCAGUAACACAUUGGAGAUCGGGCUGAGAA
CAAGAAUCAGGAAAAUGUUAGAGGAGCACAAGGAGCCGUCACCUCUCAUUACCGCCGAGG
ACGUACAAGAAGCUAAGUGCGCAGCCGAUGAGGCUAAGGAGGUGCGUGAAGCCGAGGAGU
UGCGCGCAGCUCUACCACCUUUGGCAGCUGAUGUUGAGGAGCCCACUCUGGAAGCCGAUG
UCGACUUGAUGUUACAAGAGGCUGGGGCCGGCUCAGUGGAGACACCUCGUGGCUUGAUAA
AGGUUACCAGCUACGAUGGCGAGGACAAGAUCGGCUCUUACGCUGUGCUUUCUCCGCAGG
CUGUACUCAAGAGUGAAAAAUUAUCUUGCAUCCACCCUCUCGCUGAACAAGUCAUAGUGA
UAACACACUCUGGCCGAAAAGGGCGUUAUGCCGUGGAACCAUACCAUGGUAAAGUAGUGG
UGCCAGAGGGACAUGCAAUACCCGUCCAGGACUUUCAAGCUCUGAGUGAAAGUGCCACCA
UUGUGUACAACGAACGUGAGUUCGUAAACAGGUACCUGCACCAUAUUGCCACACAUGGAG
GAGCGCUGAACACUGAUGAAGAAUAUUACAAAACUGUCAAGCCCAGCGAGCACGACGGCG
AAUACCUGUACGACAUCGACAGGAAACAGUGCGUCAAGAAAGAACUAGUCACUGGGCUAG
GGCUCACAGGCGAGCUGGUGGAUCCUCCCUUCCAUGAAUUCGCCUACGAGAGUCUGAGAA
CACGACCAGCCGCUCCUUACCAAGUACCAACCAUAGGGGUGUAUGGCGUGCCAGGAUCAG
GCAAGUCUGGCAUCAUUAAAAGCGCAGUCACCAAAAAAGAUCUAGUGGUGAGCGCCAAGA
AAGAAAACUGUGCAGAAAUUAUAAGGGACGUCAAGAAAAUGAAAGGGCUGGACGUCAAUG
CCAGAACUGUGGACUCAGUGCUCUUGAAUGGAUGCAAACACCCCGUAGAGACCCUGUAUA
UUGACGAAGCUUUUGCUUGUCAUGCAGGUACUCUCAGAGCGCUCAUAGCCAUUAUAAGAC
CUAAAAAGGCAGUGCUCUGCGGGGAUCCCAAACAGUGCGGUUUUUUUAACAUGAUGUGCC
UGAAAGUGCAUUUUAACCACGAGAUUUGCACACAAGUCUUCCACAAAAGCAUCUCUCGCC
GUUGCACUAAAUCUGUGACUUCGGUCGUCUCAACCUUGUUUUACGACAAAAAAAUGAGAA
CGACGAAUCCGAAAGAGACUAAGAUUGUGAUUGACACUACCGGCAGUACCAAACCUAAGC
AGGACGAUCUCAUUCUCACUUGUUUCAGAGGGUGGGUGAAGCAGUUGCAAAUAGAUUACA
AAGGCAACGAAAUAAUGACGGCAGCUGCCUCUCAAGGGCUGACCCGUAAAGGUGUGUAUG
CCGUUCGGUACAAGGUGAAUGAAAAUCCUCUGUACGCACCCACCUCAGAACAUGUGAACG
UCCUACUGACCCGCACGGAGGACCGCAUCGUGUGGAAAACACUAGCCGGCGACCCAUGGA
UAAAAACACUGACUGCCAAGUACCCUGGGAAUUUCACUGCCACGAUAGAGGAGUGGCAAG
CAGAGCAUGAUGCCAUCAUGAGGCACAUCUUGGAGAGACCGGACCCUACCGACGUCUUCC
AGAAUAAGGCAAACGUGUGUUGGGCCAAGGCUUUAGUGCCGGUGCUGAAGACCGCUGGCA
UAGACAUGACCACUGAACAAUGGAACACUGUGGAUUAUUUUGAAACGGACAAAGCUCACU
CAGCAGAGAUAGUAUUGAACCAACUAUGCGUGAGGUUCUUUGGACUCGAUCUGGACUCCG
GUCUAUUUUCUGCACCCACUGUUCCGUUAUCCAUUAGGAAUAAUCACUGGGAUAACUCCC
CGUCGCCUAACAUGUACGGGCUGAAUAAAGAAGUGGUCCGUCAGCUCUCUCGCAGGUACC
CACAACUGCCUCGGGCAGUUGCCACUGGAAGAGUCUAUGACAUGAACACUGGUACACUGC
GCAAUUAUGAUCCGCGCAUAAACCUAGUACCUGUAAACAGAAGACUGCCUCAUGCUUUAG
UCCUCCACCAUAAUGAACACCCACAGAGUGACUUUUCUUCAUUCGUCAGCAAAUUGAAGG
GCAGAACUGUCCUGGUGGUCGGGGAAAAGUUGUCCGUCCCAGGCAAAAUGGUUGACUGGU
UGUCAGACCGGCCUGAGGCUACCUUCAGAGCUCGGCUGGAUUUAGGCAUCCCAGGUGAUG
UGCCCAAAUAUGACAUAAUAUUUGUUAAUGUGAGGACCCCAUAUAAAUACCAUCACUAUC
AGCAGUGUGAAGACCAUGCCAUUAAGCUUAGCAUGUUGACCAAGAAAGCUUGUCUGCAUC
UGAAUCCCGGCGGAACCUGUGUCAGCAUAGGUUAUGGUUACGCUGACAGGGCCAGCGAAA
GCAUCAUUGGUGCUAUAGCGCGGCAGUUCAAGUUUUCCCGGGUAUGCAAACCGAAAUCCU
CACUUGAAGAGACGGAAGUUCUGUUUGUAUUCAUUGGGUACGAUCGCAAGGCCCGUACGC
ACAAUUCUUACAAGCUUUCAUCAACCUUGACCAACAUUUAUACAGGUUCCAGACUCCACG
AAGCCGGAUGUGCACCCUCAUAUCAUGUGGUGCGAGGGGAUAUUGCCACGGCCACCGAAG
GAGUGAUUAUAAAUGCUGCUAACAGCAAAGGACAACCUGGCGGAGGGGUGUGCGGAGCGC
UGUAUAAGAAAUUCCCGGAAAGCUUCGAUUUACAGCCGAUCGAAGUAGGAAAAGCGCGAC
UGGUCAAAGGUGCAGCUAAACAUAUCAUUCAUGCCGUAGGACCAAACUUCAACAAAGUUU
CGGAGGUUGAAGGUGACAAACAGUUGGCAGAGGCUUAUGAGUCCAUCGCUAAGAUUGUCA
ACGAUAACAAUUACAAGUCAGUAGCGAUUCCACUGUUGUCCACCGGCAUCUUUUCCGGGA
ACAAAGAUCGACUAACCCAAUCAUUGAACCAUUUGCUGACAGCUUUAGACACCACUGAUG
CAGAUGUAGCCAUAUACUGCAGGGACAAGAAAUGGGAAAUGACUCUCAAGGAAGCAGUGG
CUAGGAGAGAAGCAGUGGAGGAGAUAUGCAUAUCCGACGACUCUUCAGUGACAGAACCUG
AUGCAGAGCUGGUGAGGGUGCAUCCGAAGAGUUCUUUGGCUGGAAGGAAGGGCUACAGCA
CAAGCGAUGGCAAAACUUUCUCAUAUUUGGAAGGGACCAAGUUUCACCAGGCGGCCAAGG
AUAUAGCAGAAAUUAAUGCCAUGUGGCCCGUUGCAACGGAGGCCAAUGAGCAGGUAUGCA
UGUAUAUCCUCGGAGAAAGCAUGAGCAGUAUUAGGUCGAAAUGCCCCGUCGAAGAGUCGG
AAGCCUCCACACCACCUAGCACGCUGCCUUGCUUGUGCAUCCAUGCCAUGACUCCAGAAA
GAGUACAGCGCCUAAAAGCCUCACGUCCAGAACAAAUUACUGUGUGCUCAUCCUUUCCAU
UGCCGAAGUAUAGAAUCACUGGUGUGCAGAAGAUCCAAUGCUCCCAGCCUAUAUUGUUCU
CACCGAAAGUGCCUGCGUAUAUUCAUCCAAGGAAGUAUCUCGUGGAAACACCACCGGUAG
ACGAGACUCCGGAGCCAUCGGCAGAGAACCAAUCCACAGAGGGGACACCUGAACAACCAC
CACUUAUAACCGAGGAUGAGACCAGGACUAGAACGCCUGAGCCGAUCAUCAUCGAAGAGG
AAGAAGAGGAUAGCAUAAGUUUGCUGUCAGAUGGCCCGACCCACCAGGUGCUGCAAGUCG
AGGCAGACAUUCACGGGCCGCCCUCUGUAUCUAGCUCAUCCUGGUCCAUUCCUCAUGCAU
CCGACUUUGAUGUGGACAGUUUAUCCAUACUUGACACCCUGGAGGGAGCUAGCGUGACCA
GCGGGGCAACGUCAGCCGAGACUAACUCUUACUUCGCAAAGAGUAUGGAGUUUCUGGCGC
GACCGGUGCCUGCGCCUCGAACAGUAUUCAGGAACCCUCCACAUCCCGCUCCGCGCACAA
GAACACCGUCACUUGCACCCAGCAGGGCCUGCUCGAGAACCAGCCUAGUUUCCACCCCGC
CAGGCGUGAAUAGGGUGAUCACUAGAGAGGAGCUCGAGGCGCUUACCCCGUCACGCACUC
CUAGCAGGUCGGUCUCGAGAACCAGCCUGGUCUCCAACCCGCCAGGCGUAAAUAGGGUGA
UUACAAGAGAGGAGUUUGAGGCGUUCGUAGCACAACAACAAUGACGGUUUGAUGCGGGUG
CAUACAUCUUUUCCUCCGACACCGGUCAAGGGCAUUUACAACAAAAAUCAGUAAGGCAAA
CGGUGCUAUCCGAAGUGGUGUUGGAGAGGACCGAAUUGGAGAUUUCGUAUGCCCCGCGCC
UCGACCAAGAAAAAGAAGAAUUACUACGCAAGAAAUUACAGUUAAAUCCCACACCUGCUA
ACAGAAGCAGAUACCAGUCCAGGAAGGUGGAGAACAUGAAAGCCAUAACAGCUAGACGUA
UUCUGCAAGGCCUAGGGCAUUAUUUGAAGGCAGAAGGAAAAGUGGAGUGCUACCGAACCC
UGCAUCCUGUUCCUUUGUAUUCAUCUAGUGUGAACCGUGCCUUUUCAAGCCCCAAGGUCG
CAGUGGAAGCCUGUAACGCCAUGUUGAAAGAGAACUUUCCGACUGUGGCUUCUUACUGUA
UUAUUCCAGAGUACGAUGCCUAUUUGGACAUGGUUGACGGAGCUUCAUGCUGCUUAGACA
CUGCCAGUUUUUGCCCUGCAAAGCUGCGCAGCUUUCCAAAGAAACACUCCUAUUUGGAAC
CCACAAUACGAUCGGCAGUGCCUUCAGCGAUCCAGAACACGCUCCAGAACGUCCUGGCAG
CUGCCACAAAAAGAAAUUGCAAUGUCACGCAAAUGAGAGAAUUGCCCGUAUUGGAUUCGG
CGGCCUUUAAUGUGGAAUGCUUCAAGAAAUAUGCGUGUAAUAAUGAAUAUUGGGAAACGU
UUAAAGAAAACCCCAUCAGGCUUACUGAAGAAAACGUGGUAAAUUACAUUACCAAAUUAA
AAGGACCAAAAGCUGCUGCUCUUUUUGCGAAGACACAUAAUUUGAAUAUGUUGCAGGACA
UACCAAUGGACAGGUUUGUAAUGGACUUAAAGAGAGACGUGAAAGUGACUCCAGGAACAA
AACAUACUGAAGAACGGCCCAAGGUACAGGUGAUCCAGGCUGCCGAUCCGCUAGCAACAG
CGUAUCUGUGCGGAAUCCACCGAGAGCUGGUUAGGAGAUUAAAUGCGGUCCUGCUUCCGA
ACAUUCAUACACUGUUUGAUAUGUCGGCUGAAGACUUUGACGCUAUUAUAGCCGAGCACU
UCCAGCCUGGGGAUUGUGUUCUGGAAACUGACAUCGCGUCGUUUGAUAAAAGUGAGGACG
ACGCCAUGGCUCUGACCGCGUUAAUGAUUCUGGAAGACUUAGGUGUGGACGCAGAGCUGU
UGACGCUGAUUGAGGCGGCUUUCGGCGAAAUUUCAUCAAUACAUUUGCCCACUAAAACUA
AAUUUAAAUUCGGAGCCAUGAUGAAAUCUGGAAUGUUCCUCACACUGUUUGUGAACACAG
UCAUUAACAUUGUAAUCGCAAGCAGAGUGUUGAGAGAACGGCUAACCGGAUCACCAUGUG
CAGCAUUCAUUGGAGAUGACAAUAUCGUGAAAGGAGUCAAAUCGGACAAAUUAAUGGCAG
ACAGGUGCGCCACCUGGUUGAAUAUGGAAGUCAAGAUUAUAGAUGCUGUGGUGGGCGAGA
AAGCGCCUUAUUUCUGUGGAGGGUUUAUUUUGUGUGACUCCGUGACCGGCACAGCGUGCC
GUGUGGCAGACCCCCUAAAAAGGCUGUUUAAGCUUGGCAAACCUCUGGCAGCAGACGAUG
AACAUGAUGAUGACAGGAGAAGGGCAUUGCAUGAAGAGUCAACACGCUGGAACCGAGUGG
GUAUUCUUUCAGAGCUGUGCAAGGCAGUAGAAUCAAGGUAUGAAACCGUAGGAACUUCCA
UCAUAGUUAUGGCCAUGACUACUCUAGCUAGCAGUGUUAAAUCAUUCAGCUACCUGAGAG
GGGCCCCUAUAACUCUCUACGGCUAACCUGAAUGGACUACGACAUAGUCUAGUCCGCCAA
GUCUAGCAUAUGGCCACC --- GOI ---
[SEQ ID No: 181]
CGGAGACGGCGCAGAAGAAGAGGAUCUGGCGAAGGCAGAGGCAGCCUGCUUACAUGUGGC
GACGUGGAAGAGAACCCCGGACCUAUGGCCCGCGCUGCUCUCUCCGCCGCCCCCAGCAA
UCCCCGGCUCCUGCGAGUGGCACUGCUGCUCCUGCUCCUGGUAGCCGCUGGCCGGCGCG
CAGCAGGAGCGUCCGUGGCCACUGAACUGCGCUGCCAGUGCUUGCAGACCCUGCAGGGA
AUUCACCCCAAGAACAUCCAAAGUGUGAACGUGAAGUCCCCCGGACCCCACUGCGCCCA
AACCGAAGUCAUAGCCACACUCAAGAAUGGGCGGAAAGCUUGCCUCAAUCCUGCAUCCC
CCAUAGUUAAGAAAAUCAUCGAAAAGAUGCUGAACAGUGACAAAUCCAACUGAGCGGCC
GCGAAUUGGCAAGCUGCUUACAUAGAACUCGCGGCGAUUGGCAUGCCGCCUUAAAAUUUU
UAUUUUAUUUUUCUUUUCUUUUCCGAAUCGGAUUUUGUUUUUAAUAUUUCAAAAAAAAAA
AAAAAAAAAAAAAAAAAAAAAAAAA

Accordingly, preferably the RNA construct comprises a nucleotide sequence substantially as set out above, comprising or consisting of SEQ ID No: 160, a GOI, and SEQ ID No: 181, or a fragment or variant thereof.

In a fifth aspect of the invention, there is provided a nucleic acid sequence encoding the RNA construct as defined in any one of the first to fourth aspect.

In one embodiment, the nucleic acid sequence may comprise a T7 Promoter, 5′UTR, NSP1-4, Sub-Genomic Promoter, GOI (gene of interest is the therapeutic biomolecule), Furin T2A, ISP is CXCL11 (which is the first ISP mentioned herein, but it will be appreciated that any of the ISPs or linkers disclosed herein may be used), 3′UTR, and PolyA tail. Hence, in one embodiment, the nucleic acid sequence may comprise or consist of SEQ ID No: 161, a GOI, and SEQ ID No: 182 in a single construct. SEQ ID No: 161 and SEQ ID No: 182 are, as follows, where “GOI” represents the position of the therapeutic biomolecule encoding sequence:

[SEQ ID No: 161]
ATGGGCGGCGCATGAGAGAAGCCCAGACCAATTACCTACCCAAAATGGAGAAAGTTCACG
TTGACATCGAGGAAGACAGCCCATTCCTCAGAGCTTTGCAGCGGAGCTTCCCGCAGTTTG
AGGTAGAAGCCAAGCAGGTCACTGATAATGACCATGCTAATGCCAGAGCGTTTTCGCATC
TGGCTTCAAAACTGATCGAAACGGAGGTGGACCCATCCGACACGATCCTTGACATTGGAA
GTGCGCCCGCCCGCAGAATGTATTCTAAGCACAAGTATCATTGTATCTGTCCGATGAGAT
GTGCGGAAGATCCGGACAGATTGTATAAGTATGCAACTAAGCTGAAGAAAAACTGTAAGG
AAATAACTGATAAGGAATTGGACAAGAAAATGAAGGAGCTGGCCGCCGTCATGAGCGACC
CTGACCTGGAAACTGAGACTATGTGCCTCCACGACGACGAGTCGTGTCGCTACGAAGGGC
AAGTCGCTGTTTACCAGGATGTATACGCGGTTGACGGACCGACAAGTCTCTATCACCAAG
CCAATAAGGGAGTTAGAGTCGCCTACTGGATAGGCTTTGACACCACCCCTTTTATGTTTA
AGAACTTGGCTGGAGCATATCCATCATACTCTACCAACTGGGCCGACGAAACCGTGTTAA
CGGCTCGTAACATAGGCCTATGCAGCTCTGACGTTATGGAGCGGTCACGTAGAGGGATGT
CCATTCTTAGAAAGAAGTATTTGAAACCATCCAACAATGTTCTATTCTCTGTTGGCTCGA
CCATCTACCACGAGAAGAGGGACTTACTGAGGAGCTGGCACCTGCCGTCTGTATTTCACT
TACGTGGCAAGCAAAATTACACATGTCGGTGTGAGACTATAGTTAGTTGCGACGGGTACG
TCGTTAAAAGAATAGCTATCAGTCCAGGCCTGTATGGGAAGCCTTCAGGCTATGCTGCTA
CGATGCACCGCGAGGGATTCTTGTGCTGCAAAGTGACAGACACATTGAACGGGGAGAGGG
TCTCTTTTCCCGTGTGCACGTATGTGCCAGCTACATTGTGTGACCAAATGACTGGCATAC
TGGCAACAGATGTCAGTGCGGACGACGCGCAAAAACTGCTGGTTGGGCTCAACCAGCGTA
TAGTCGTCAACGGTCGCACCCAGAGAAACACCAATACCATGAAAAATTACCTTTTGCCCG
TAGTGGCCCAGGCATTTGCTAGGTGGGCAAAGGAATATAAGGAAGATCAAGAAGATGAAA
GGCCACTAGGACTACGAGATAGACAGTTAGTCATGGGGTGTTGTTGGGCTTTTAGAAGGC
ACAAGATAACATCTATTTATAAGCGCCCGGATACCCAAACCATCATCAAAGTGAACAGCG
ATTTCCACTCATTCGTGCTGCCCAGGATAGGCAGTAACACATTGGAGATCGGGCTGAGAA
CAAGAATCAGGAAAATGTTAGAGGAGCACAAGGAGCCGTCACCTCTCATTACCGCCGAGG
ACGTACAAGAAGCTAAGTGCGCAGCCGATGAGGCTAAGGAGGTGCGTGAAGCCGAGGAGT
TGCGCGCAGCTCTACCACCTTTGGCAGCTGATGTTGAGGAGCCCACTCTGGAAGCCGATG
TCGACTTGATGTTACAAGAGGCTGGGGCCGGCTCAGTGGAGACACCTCGTGGCTTGATAA
AGGTTACCAGCTACGATGGCGAGGACAAGATCGGCTCTTACGCTGTGCTTTCTCCGCAGG
CTGTACTCAAGAGTGAAAAATTATCTTGCATCCACCCTCTCGCTGAACAAGTCATAGTGA
TAACACACTCTGGCCGAAAAGGGCGTTATGCCGTGGAACCATACCATGGTAAAGTAGTGG
TGCCAGAGGGACATGCAATACCCGTCCAGGACTTTCAAGCTCTGAGTGAAAGTGCCACCA
TTGTGTACAACGAACGTGAGTTCGTAAACAGGTACCTGCACCATATTGCCACACATGGAG
GAGCGCTGAACACTGATGAAGAATATTACAAAACTGTCAAGCCCAGCGAGCACGACGGCG
AATACCTGTACGACATCGACAGGAAACAGTGCGTCAAGAAAGAACTAGTCACTGGGCTAG
GGCTCACAGGCGAGCTGGTGGATCCTCCCTTCCATGAATTCGCCTACGAGAGTCTGAGAA
CACGACCAGCCGCTCCTTACCAAGTACCAACCATAGGGGTGTATGGCGTGCCAGGATCAG
GCAAGTCTGGCATCATTAAAAGCGCAGTCACCAAAAAAGATCTAGTGGTGAGCGCCAAGA
AAGAAAACTGTGCAGAAATTATAAGGGACGTCAAGAAAATGAAAGGGCTGGACGTCAATG
CCAGAACTGTGGACTCAGTGCTCTTGAATGGATGCAAACACCCCGTAGAGACCCTGTATA
TTGACGAAGCTTTTGCTTGTCATGCAGGTACTCTCAGAGCGCTCATAGCCATTATAAGAC
CTAAAAAGGCAGTGCTCTGCGGGGATCCCAAACAGTGCGGTTTTTTTAACATGATGTGCC
TGAAAGTGCATTTTAACCACGAGATTTGCACACAAGTCTTCCACAAAAGCATCTCTCGCC
GTTGCACTAAATCTGTGACTTCGGTCGTCTCAACCTTGTTTTACGACAAAAAAATGAGAA
CGACGAATCCGAAAGAGACTAAGATTGTGATTGACACTACCGGCAGTACCAAACCTAAGC
AGGACGATCTCATTCTCACTTGTTTCAGAGGGTGGGTGAAGCAGTTGCAAATAGATTACA
AAGGCAACGAAATAATGACGGCAGCTGCCTCTCAAGGGCTGACCCGTAAAGGTGTGTATG
CCGTTCGGTACAAGGTGAATGAAAATCCTCTGTACGCACCCACCTCAGAACATGTGAACG
TCCTACTGACCCGCACGGAGGACCGCATCGTGTGGAAAACACTAGCCGGCGACCCATGGA
TAAAAACACTGACTGCCAAGTACCCTGGGAATTTCACTGCCACGATAGAGGAGTGGCAAG
CAGAGCATGATGCCATCATGAGGCACATCTTGGAGAGACCGGACCCTACCGACGTCTTCC
AGAATAAGGCAAACGTGTGTTGGGCCAAGGCTTTAGTGCCGGTGCTGAAGACCGCTGGCA
TAGACATGACCACTGAACAATGGAACACTGTGGATTATTTTGAAACGGACAAAGCTCACT
CAGCAGAGATAGTATTGAACCAACTATGCGTGAGGTTCTTTGGACTCGATCTGGACTCCG
GTCTATTTTCTGCACCCACTGTTCCGTTATCCATTAGGAATAATCACTGGGATAACTCCC
CGTCGCCTAACATGTACGGGCTGAATAAAGAAGTGGTCCGTCAGCTCTCTCGCAGGTACC
CACAACTGCCTCGGGCAGTTGCCACTGGAAGAGTCTATGACATGAACACTGGTACACTGC
GCAATTATGATCCGCGCATAAACCTAGTACCTGTAAACAGAAGACTGCCTCATGCTTTAG
TCCTCCACCATAATGAACACCCACAGAGTGACTTTTCTTCATTCGTCAGCAAATTGAAGG
GCAGAACTGTCCTGGTGGTCGGGGAAAAGTTGTCCGTCCCAGGCAAAATGGTTGACTGGT
TGTCAGACCGGCCTGAGGCTACCTTCAGAGCTCGGCTGGATTTAGGCATCCCAGGTGATG
TGCCCAAATATGACATAATATTTGTTAATGTGAGGACCCCATATAAATACCATCACTATC
AGCAGTGTGAAGACCATGCCATTAAGCTTAGCATGTTGACCAAGAAAGCTTGTCTGCATC
TGAATCCCGGCGGAACCTGTGTCAGCATAGGTTATGGTTACGCTGACAGGGCCAGCGAAA
GCATCATTGGTGCTATAGCGCGGCAGTTCAAGTTTTCCCGGGTATGCAAACCGAAATCCT
CACTTGAAGAGACGGAAGTTCTGTTTGTATTCATTGGGTACGATCGCAAGGCCCGTACGC
ACAATTCTTACAAGCTTTCATCAACCTTGACCAACATTTATACAGGTTCCAGACTCCACG
AAGCCGGATGTGCACCCTCATATCATGTGGTGCGAGGGGATATTGCCACGGCCACCGAAG
GAGTGATTATAAATGCTGCTAACAGCAAAGGACAACCTGGCGGAGGGGTGTGCGGAGCGC
TGTATAAGAAATTCCCGGAAAGCTTCGATTTACAGCCGATCGAAGTAGGAAAAGCGCGAC
TGGTCAAAGGTGCAGCTAAACATATCATTCATGCCGTAGGACCAAACTTCAACAAAGTTT
CGGAGGTTGAAGGTGACAAACAGTTGGCAGAGGCTTATGAGTCCATCGCTAAGATTGTCA
ACGATAACAATTACAAGTCAGTAGCGATTCCACTGTTGTCCACCGGCATCTTTTCCGGGA
ACAAAGATCGACTAACCCAATCATTGAACCATTTGCTGACAGCTTTAGACACCACTGATG
CAGATGTAGCCATATACTGCAGGGACAAGAAATGGGAAATGACTCTCAAGGAAGCAGTGG
CTAGGAGAGAAGCAGTGGAGGAGATATGCATATCCGACGACTCTTCAGTGACAGAACCTG
ATGCAGAGCTGGTGAGGGTGCATCCGAAGAGTTCTTTGGCTGGAAGGAAGGGCTACAGCA
CAAGCGATGGCAAAACTTTCTCATATTTGGAAGGGACCAAGTTTCACCAGGCGGCCAAGG
ATATAGCAGAAATTAATGCCATGTGGCCCGTTGCAACGGAGGCCAATGAGCAGGTATGCA
TGTATATCCTCGGAGAAAGCATGAGCAGTATTAGGTCGAAATGCCCCGTCGAAGAGTCGG
AAGCCTCCACACCACCTAGCACGCTGCCTTGCTTGTGCATCCATGCCATGACTCCAGAAA
GAGTACAGCGCCTAAAAGCCTCACGTCCAGAACAAATTACTGTGTGCTCATCCTTTCCAT
TGCCGAAGTATAGAATCACTGGTGTGCAGAAGATCCAATGCTCCCAGCCTATATTGTTCT
CACCGAAAGTGCCTGCGTATATTCATCCAAGGAAGTATCTCGTGGAAACACCACCGGTAG
ACGAGACTCCGGAGCCATCGGCAGAGAACCAATCCACAGAGGGGACACCTGAACAACCAC
CACTTATAACCGAGGATGAGACCAGGACTAGAACGCCTGAGCCGATCATCATCGAAGAGG
AAGAAGAGGATAGCATAAGTTTGCTGTCAGATGGCCCGACCCACCAGGTGCTGCAAGTCG
AGGCAGACATTCACGGGCCGCCCTCTGTATCTAGCTCATCCTGGTCCATTCCTCATGCAT
CCGACTTTGATGTGGACAGTTTATCCATACTTGACACCCTGGAGGGAGCTAGCGTGACCA
GCGGGGCAACGTCAGCCGAGACTAACTCTTACTTCGCAAAGAGTATGGAGTTTCTGGCGC
GACCGGTGCCTGCGCCTCGAACAGTATTCAGGAACCCTCCACATCCCGCTCCGCGCACAA
GAACACCGTCACTTGCACCCAGCAGGGCCTGCTCGAGAACCAGCCTAGTTTCCACCCCGC
CAGGCGTGAATAGGGTGATCACTAGAGAGGAGCTCGAGGCGCTTACCCCGTCACGCACTC
CTAGCAGGTCGGTCTCGAGAACCAGCCTGGTCTCCAACCCGCCAGGCGTAAATAGGGTGA
TTACAAGAGAGGAGTTTGAGGCGTTCGTAGCACAACAACAATGACGGTTTGATGCGGGTG
CATACATCTTTTCCTCCGACACCGGTCAAGGGCATTTACAACAAAAATCAGTAAGGCAAA
CGGTGCTATCCGAAGTGGTGTTGGAGAGGACCGAATTGGAGATTTCGTATGCCCCGCGCC
TCGACCAAGAAAAAGAAGAATTACTACGCAAGAAATTACAGTTAAATCCCACACCTGCTA
ACAGAAGCAGATACCAGTCCAGGAAGGTGGAGAACATGAAAGCCATAACAGCTAGACGTA
TTCTGCAAGGCCTAGGGCATTATTTGAAGGCAGAAGGAAAAGTGGAGTGCTACCGAACCC
TGCATCCTGTTCCTTTGTATTCATCTAGTGTGAACCGTGCCTTTTCAAGCCCCAAGGTCG
CAGTGGAAGCCTGTAACGCCATGTTGAAAGAGAACTTTCCGACTGTGGCTTCTTACTGTA
TTATTCCAGAGTACGATGCCTATTTGGACATGGTTGACGGAGCTTCATGCTGCTTAGACA
CTGCCAGTTTTTGCCCTGCAAAGCTGCGCAGCTTTCCAAAGAAACACTCCTATTTGGAAC
CCACAATACGATCGGCAGTGCCTTCAGCGATCCAGAACACGCTCCAGAACGTCCTGGCAG
CTGCCACAAAAAGAAATTGCAATGTCACGCAAATGAGAGAATTGCCCGTATTGGATTCGG
CGGCCTTTAATGTGGAATGCTTCAAGAAATATGCGTGTAATAATGAATATTGGGAAACGT
TTAAAGAAAACCCCATCAGGCTTACTGAAGAAAACGTGGTAAATTACATTACCAAATTAA
AAGGACCAAAAGCTGCTGCTCTTTTTGCGAAGACACATAATTTGAATATGTTGCAGGACA
TACCAATGGACAGGTTTGTAATGGACTTAAAGAGAGACGTGAAAGTGACTCCAGGAACAA
AACATACTGAAGAACGGCCCAAGGTACAGGTGATCCAGGCTGCCGATCCGCTAGCAACAG
CGTATCTGTGCGGAATCCACCGAGAGCTGGTTAGGAGATTAAATGCGGTCCTGCTTCCGA
ACATTCATACACTGTTTGATATGTCGGCTGAAGACTTTGACGCTATTATAGCCGAGCACT
TCCAGCCTGGGGATTGTGTTCTGGAAACTGACATCGCGTCGTTTGATAAAAGTGAGGACG
ACGCCATGGCTCTGACCGCGTTAATGATTCTGGAAGACTTAGGTGTGGACGCAGAGCTGT
TGACGCTGATTGAGGCGGCTTTCGGCGAAATTTCATCAATACATTTGCCCACTAAAACTA
AATTTAAATTCGGAGCCATGATGAAATCTGGAATGTTCCTCACACTGTTTGTGAACACAG
TCATTAACATTGTAATCGCAAGCAGAGTGTTGAGAGAACGGCTAACCGGATCACCATGTG
CAGCATTCATTGGAGATGACAATATCGTGAAAGGAGTCAAATCGGACAAATTAATGGCAG
ACAGGTGCGCCACCTGGTTGAATATGGAAGTCAAGATTATAGATGCTGTGGTGGGCGAGA
AAGCGCCTTATTTCTGTGGAGGGTTTATTTTGTGTGACTCCGTGACCGGCACAGCGTGCC
GTGTGGCAGACCCCCTAAAAAGGCTGTTTAAGCTTGGCAAACCTCTGGCAGCAGACGATG
AACATGATGATGACAGGAGAAGGGCATTGCATGAAGAGTCAACACGCTGGAACCGAGTGG
GTATTCTTTCAGAGCTGTGCAAGGCAGTAGAATCAAGGTATGAAACCGTAGGAACTTCCA
TCATAGTTATGGCCATGACTACTCTAGCTAGCAGTGTTAAATCATTCAGCTACCTGAGAG
GGGCCCCTATAACTCTCTACGGCTAACCTGAATGGACTACGACATAGTCTAGTCCGCCAA
GTCTAGCATATGGCCACC ---GOI---
[SEQ ID No: 182]
CGGAGACGGCGCAGAAGAAGAGGATCTGGCGAAGGCAGAGGCAGCCTGCTTACATGTGGC
GACGTGGAAGAGAACCCCGGACCTATGGCCCGCGCTGCTCTCTCCGCCGCCCCCAGCAA
TCCCCGGCTCCTGCGAGTGGCACTGCTGCTCCTGCTCCTGGTAGCCGCTGGCCGGCGCG
CAGCAGGAGCGTCCGTGGCCACTGAACTGCGCTGCCAGTGCTTGCAGACCCTGCAGGGA
ATTCACCCCAAGAACATCCAAAGTGTGAACGTGAAGTCCCCCGGACCCCACTGCGCCCA
AACCGAAGTCATAGCCACACTCAAGAATGGGCGGAAAGCTTGCCTCAATCCTGCATCCC
CCATAGTTAAGAAAATCATCGAAAAGATGCTGAACAGTGACAAATCCAACTGAGCGGCC
GCGAATTGGCAAGCTGCTTACATAGAACTCGCGGCGATTGGCATGCCGCCTTAAAATTTT
TATTTTATTTTTCTTTTCTTTTCCGAATCGGATTTTGTTTTTAATATTTCAAAAAAAAAA
AAAAAAAAAAAAAAAAAAAAAAAAA

Accordingly, preferably the nucleic acid sequence comprises a nucleotide sequence substantially as set out above, comprising or consisting of SEQ ID No: 161, a GOI, and SEQ ID No: 182, or a fragment or variant thereof.

In a sixth aspect, there is provided an expression cassette comprising a nucleic acid sequence according to the fifth aspect.

The nucleic acid sequences of the invention are preferably harboured in a recombinant vector, for example a recombinant vector for delivery into a host cell of interest to enable production of the RNA construct.

Accordingly, in a seventh aspect, there is provided a recombinant vector comprising the expression cassette according to the sixth aspect.

In one embodiment, the vector may comprise or encode a T7 Promoter, 5′UTR, NSP1-4, Sub-Genomic Promoter, GOI (gene of interest is the therapeutic biomolecule), Furin T2A, ISP is CXCL11 (which is the first ISP mentioned herein, but it will be appreciated that any of the ISPs or linkers disclosed herein may be used), 3′UTR, and PolyA tail. Accordingly, the vector may comprise the nucleic acid sequence of SEQ ID No: 162, a GOI, and the sequence of SEQ ID No: 183 in a single construct. SEQ ID No: 162 and SEQ ID No: 183 are, as follows, where “GOI” represents the position of the therapeutic biomolecule encoding sequence:

[SEQ ID No: 162]
TAATACGACTCACTATAGATGGGCGGCGCATGAGAGAAGCCCAGACCAATTACCTACCCA
AAATGGAGAAAGTTCACGTTGACATCGAGGAAGACAGCCCATTCCTCAGAGCTTTGCAGC
GGAGCTTCCCGCAGTTTGAGGTAGAAGCCAAGCAGGTCACTGATAATGACCATGCTAATG
CCAGAGCGTTTTCGCATCTGGCTTCAAAACTGATCGAAACGGAGGTGGACCCATCCGACA
CGATCCTTGACATTGGAAGTGCGCCCGCCCGCAGAATGTATTCTAAGCACAAGTATCATT
GTATCTGTCCGATGAGATGTGCGGAAGATCCGGACAGATTGTATAAGTATGCAACTAAGC
TGAAGAAAAACTGTAAGGAAATAACTGATAAGGAATTGGACAAGAAAATGAAGGAGCTGG
CCGCCGTCATGAGCGACCCTGACCTGGAAACTGAGACTATGTGCCTCCACGACGACGAGT
CGTGTCGCTACGAAGGGCAAGTCGCTGTTTACCAGGATGTATACGCGGTTGACGGACCGA
CAAGTCTCTATCACCAAGCCAATAAGGGAGTTAGAGTCGCCTACTGGATAGGCTTTGACA
CCACCCCTTTTATGTTTAAGAACTTGGCTGGAGCATATCCATCATACTCTACCAACTGGG
CCGACGAAACCGTGTTAACGGCTCGTAACATAGGCCTATGCAGCTCTGACGTTATGGAGC
GGTCACGTAGAGGGATGTCCATTCTTAGAAAGAAGTATTTGAAACCATCCAACAATGTTC
TATTCTCTGTTGGCTCGACCATCTACCACGAGAAGAGGGACTTACTGAGGAGCTGGCACC
TGCCGTCTGTATTTCACTTACGTGGCAAGCAAAATTACACATGTCGGTGTGAGACTATAG
TTAGTTGCGACGGGTACGTCGTTAAAAGAATAGCTATCAGTCCAGGCCTGTATGGGAAGC
CTTCAGGCTATGCTGCTACGATGCACCGCGAGGGATTCTTGTGCTGCAAAGTGACAGACA
CATTGAACGGGGAGAGGGTCTCTTTTCCCGTGTGCACGTATGTGCCAGCTACATTGTGTG
ACCAAATGACTGGCATACTGGCAACAGATGTCAGTGCGGACGACGCGCAAAAACTGCTGG
TTGGGCTCAACCAGCGTATAGTCGTCAACGGTCGCACCCAGAGAAACACCAATACCATGA
AAAATTACCTTTTGCCCGTAGTGGCCCAGGCATTTGCTAGGTGGGCAAAGGAATATAAGG
AAGATCAAGAAGATGAAAGGCCACTAGGACTACGAGATAGACAGTTAGTCATGGGGTGTT
GTTGGGCTTTTAGAAGGCACAAGATAACATCTATTTATAAGCGCCCGGATACCCAAACCA
TCATCAAAGTGAACAGCGATTTCCACTCATTCGTGCTGCCCAGGATAGGCAGTAACACAT
TGGAGATCGGGCTGAGAACAAGAATCAGGAAAATGTTAGAGGAGCACAAGGAGCCGTCAC
CTCTCATTACCGCCGAGGACGTACAAGAAGCTAAGTGCGCAGCCGATGAGGCTAAGGAGG
TGCGTGAAGCCGAGGAGTTGCGCGCAGCTCTACCACCTTTGGCAGCTGATGTTGAGGAGC
CCACTCTGGAAGCCGATGTCGACTTGATGTTACAAGAGGCTGGGGCCGGCTCAGTGGAGA
CACCTCGTGGCTTGATAAAGGTTACCAGCTACGATGGCGAGGACAAGATCGGCTCTTACG
CTGTGCTTTCTCCGCAGGCTGTACTCAAGAGTGAAAAATTATCTTGCATCCACCCTCTCG
CTGAACAAGTCATAGTGATAACACACTCTGGCCGAAAAGGGCGTTATGCCGTGGAACCAT
ACCATGGTAAAGTAGTGGTGCCAGAGGGACATGCAATACCCGTCCAGGACTTTCAAGCTC
TGAGTGAAAGTGCCACCATTGTGTACAACGAACGTGAGTTCGTAAACAGGTACCTGCACC
ATATTGCCACACATGGAGGAGCGCTGAACACTGATGAAGAATATTACAAAACTGTCAAGC
CCAGCGAGCACGACGGCGAATACCTGTACGACATCGACAGGAAACAGTGCGTCAAGAAAG
AACTAGTCACTGGGCTAGGGCTCACAGGCGAGCTGGTGGATCCTCCCTTCCATGAATTCG
CCTACGAGAGTCTGAGAACACGACCAGCCGCTCCTTACCAAGTACCAACCATAGGGGTGT
ATGGCGTGCCAGGATCAGGCAAGTCTGGCATCATTAAAAGCGCAGTCACCAAAAAAGATC
TAGTGGTGAGCGCCAAGAAAGAAAACTGTGCAGAAATTATAAGGGACGTCAAGAAAATGA
AAGGGCTGGACGTCAATGCCAGAACTGTGGACTCAGTGCTCTTGAATGGATGCAAACACC
CCGTAGAGACCCTGTATATTGACGAAGCTTTTGCTTGTCATGCAGGTACTCTCAGAGCGC
TCATAGCCATTATAAGACCTAAAAAGGCAGTGCTCTGCGGGGATCCCAAACAGTGCGGTT
TTTTTAACATGATGTGCCTGAAAGTGCATTTTAACCACGAGATTTGCACACAAGTCTTCC
ACAAAAGCATCTCTCGCCGTTGCACTAAATCTGTGACTTCGGTCGTCTCAACCTTGTTTT
ACGACAAAAAAATGAGAACGACGAATCCGAAAGAGACTAAGATTGTGATTGACACTACCG
GCAGTACCAAACCTAAGCAGGACGATCTCATTCTCACTTGTTTCAGAGGGTGGGTGAAGC
AGTTGCAAATAGATTACAAAGGCAACGAAATAATGACGGCAGCTGCCTCTCAAGGGCTGA
CCCGTAAAGGTGTGTATGCCGTTCGGTACAAGGTGAATGAAAATCCTCTGTACGCACCCA
CCTCAGAACATGTGAACGTCCTACTGACCCGCACGGAGGACCGCATCGTGTGGAAAACAC
TAGCCGGCGACCCATGGATAAAAACACTGACTGCCAAGTACCCTGGGAATTTCACTGCCA
CGATAGAGGAGTGGCAAGCAGAGCATGATGCCATCATGAGGCACATCTTGGAGAGACCGG
ACCCTACCGACGTCTTCCAGAATAAGGCAAACGTGTGTTGGGCCAAGGCTTTAGTGCCGG
TGCTGAAGACCGCTGGCATAGACATGACCACTGAACAATGGAACACTGTGGATTATTTTG
AAACGGACAAAGCTCACTCAGCAGAGATAGTATTGAACCAACTATGCGTGAGGTTCTTTG
GACTCGATCTGGACTCCGGTCTATTTTCTGCACCCACTGTTCCGTTATCCATTAGGAATA
ATCACTGGGATAACTCCCCGTCGCCTAACATGTACGGGCTGAATAAAGAAGTGGTCCGTC
AGCTCTCTCGCAGGTACCCACAACTGCCTCGGGCAGTTGCCACTGGAAGAGTCTATGACA
TGAACACTGGTACACTGCGCAATTATGATCCGCGCATAAACCTAGTACCTGTAAACAGAA
GACTGCCTCATGCTTTAGTCCTCCACCATAATGAACACCCACAGAGTGACTTTTCTTCAT
TCGTCAGCAAATTGAAGGGCAGAACTGTCCTGGTGGTCGGGGAAAAGTTGTCCGTCCCAG
GCAAAATGGTTGACTGGITGTCAGACCGGCCTGAGGCTACCTTCAGAGCTCGGCTGGATT
TAGGCATCCCAGGTGATGTGCCCAAATATGACATAATATTTGTTAATGTGAGGACCCCAT
ATAAATACCATCACTATCAGCAGTGTGAAGACCATGCCATTAAGCTTAGCATGTTGACCA
AGAAAGCTTGTCTGCATCTGAATCCCGGCGGAACCTGTGTCAGCATAGGTTATGGTTACG
CTGACAGGGCCAGCGAAAGCATCATTGGTGCTATAGCGCGGCAGTTCAAGTTTTCCCGGG
TATGCAAACCGAAATCCTCACTTGAAGAGACGGAAGTTCTGTTTGTATTCATTGGGTACG
ATCGCAAGGCCCGTACGCACAATTCTTACAAGCTTTCATCAACCTTGACCAACATTTATA
CAGGTTCCAGACTCCACGAAGCCGGATGTGCACCCTCATATCATGTGGTGCGAGGGGATA
TTGCCACGGCCACCGAAGGAGTGATTATAAATGCTGCTAACAGCAAAGGACAACCTGGCG
GAGGGGTGTGCGGAGCGCTGTATAAGAAATTCCCGGAAAGCTTCGATTTACAGCCGATCG
AAGTAGGAAAAGCGCGACTGGTCAAAGGTGCAGCTAAACATATCATTCATGCCGTAGGAC
CAAACTTCAACAAAGTTTCGGAGGTTGAAGGTGACAAACAGTTGGCAGAGGCTTATGAGT
CCATCGCTAAGATTGTCAACGATAACAATTACAAGTCAGTAGCGATTCCACTGTTGTCCA
CCGGCATCTTTTCCGGGAACAAAGATCGACTAACCCAATCATTGAACCATTTGCTGACAG
CTTTAGACACCACTGATGCAGATGTAGCCATATACTGCAGGGACAAGAAATGGGAAATGA
CTCTCAAGGAAGCAGTGGCTAGGAGAGAAGCAGTGGAGGAGATATGCATATCCGACGACT
CTTCAGTGACAGAACCTGATGCAGAGCTGGTGAGGGTGCATCCGAAGAGTTCTTTGGCTG
GAAGGAAGGGCTACAGCACAAGCGATGGCAAAACTTTCTCATATTTGGAAGGGACCAAGT
TTCACCAGGCGGCCAAGGATATAGCAGAAATTAATGCCATGTGGCCCGTTGCAACGGAGG
CCAATGAGCAGGTATGCATGTATATCCTCGGAGAAAGCATGAGCAGTATTAGGTCGAAAT
GCCCCGTCGAAGAGTCGGAAGCCTCCACACCACCTAGCACGCTGCCTTGCTTGTGCATCC
ATGCCATGACTCCAGAAAGAGTACAGCGCCTAAAAGCCTCACGTCCAGAACAAATTACTG
TGTGCTCATCCTTTCCATTGCCGAAGTATAGAATCACTGGTGTGCAGAAGATCCAATGCT
CCCAGCCTATATTGTTCTCACCGAAAGTGCCTGCGTATATTCATCCAAGGAAGTATCTCG
TGGAAACACCACCGGTAGACGAGACTCCGGAGCCATCGGCAGAGAACCAATCCACAGAGG
GGACACCTGAACAACCACCACTTATAACCGAGGATGAGACCAGGACTAGAACGCCTGAGC
CGATCATCATCGAAGAGGAAGAAGAGGATAGCATAAGTTTGCTGTCAGATGGCCCGACCC
ACCAGGTGCTGCAAGTCGAGGCAGACATTCACGGGCCGCCCTCTGTATCTAGCTCATCCT
GGTCCATTCCTCATGCATCCGACTTTGATGTGGACAGTTTATCCATACTTGACACCCTGG
AGGGAGCTAGCGTGACCAGCGGGGCAACGTCAGCCGAGACTAACTCTTACTTCGCAAAGA
GTATGGAGTTTCTGGCGCGACCGGTGCCTGCGCCTCGAACAGTATTCAGGAACCCTCCAC
ATCCCGCTCCGCGCACAAGAACACCGTCACTTGCACCCAGCAGGGCCTGCTCGAGAACCA
GCCTAGTTTCCACCCCGCCAGGCGTGAATAGGGTGATCACTAGAGAGGAGCTCGAGGCGC
TTACCCCGTCACGCACTCCTAGCAGGTCGGTCTCGAGAACCAGCCTGGTCTCCAACCCGC
CAGGCGTAAATAGGGTGATTACAAGAGAGGAGTTTGAGGCGTTCGTAGCACAACAACAAT
GACGGTTTGATGCGGGTGCATACATCTTTTCCTCCGACACCGGTCAAGGGCATTTACAAC
AAAAATCAGTAAGGCAAACGGTGCTATCCGAAGTGGTGTTGGAGAGGACCGAATTGGAGA
TTTCGTATGCCCCGCGCCTCGACCAAGAAAAAGAAGAATTACTACGCAAGAAATTACAGT
TAAATCCCACACCTGCTAACAGAAGCAGATACCAGTCCAGGAAGGTGGAGAACATGAAAG
CCATAACAGCTAGACGTATTCTGCAAGGCCTAGGGCATTATTTGAAGGCAGAAGGAAAAG
TGGAGTGCTACCGAACCCTGCATCCTGTTCCTTTGTATTCATCTAGTGTGAACCGTGCCT
TTTCAAGCCCCAAGGTCGCAGTGGAAGCCTGTAACGCCATGTTGAAAGAGAACTTTCCGA
CTGTGGCTTCTTACTGTATTATTCCAGAGTACGATGCCTATTTGGACATGGTTGACGGAG
CTTCATGCTGCTTAGACACTGCCAGTTTTTGCCCTGCAAAGCTGCGCAGCTTTCCAAAGA
AACACTCCTATTTGGAACCCACAATACGATCGGCAGTGCCTTCAGCGATCCAGAACACGC
TCCAGAACGTCCTGGCAGCTGCCACAAAAAGAAATTGCAATGTCACGCAAATGAGAGAAT
TGCCCGTATTGGATTCGGCGGCCTTTAATGTGGAATGCTTCAAGAAATATGCGTGTAATA
ATGAATATTGGGAAACGTTTAAAGAAAACCCCATCAGGCTTACTGAAGAAAACGTGGTAA
ATTACATTACCAAATTAAAAGGACCAAAAGCTGCTGCTCTTTTTGCGAAGACACATAATT
TGAATATGTTGCAGGACATACCAATGGACAGGTTTGTAATGGACTTAAAGAGAGACGTGA
AAGTGACTCCAGGAACAAAACATACTGAAGAACGGCCCAAGGTACAGGTGATCCAGGCTG
CCGATCCGCTAGCAACAGCGTATCTGTGCGGAATCCACCGAGAGCTGGTTAGGAGATTAA
ATGCGGTCCTGCTTCCGAACATTCATACACTGTTTGATATGTCGGCTGAAGACTTTGACG
CTATTATAGCCGAGCACTTCCAGCCTGGGGATTGTGTTCTGGAAACTGACATCGCGTCGT
TTGATAAAAGTGAGGACGACGCCATGGCTCTGACCGCGTTAATGATTCTGGAAGACTTAG
GTGTGGACGCAGAGCTGTTGACGCTGATTGAGGCGGCTTTCGGCGAAATTTCATCAATAC
ATTTGCCCACTAAAACTAAATTTAAATTCGGAGCCATGATGAAATCTGGAATGTTCCTCA
CACTGTTTGTGAACACAGTCATTAACATTGTAATCGCAAGCAGAGTGTTGAGAGAACGGC
TAACCGGATCACCATGTGCAGCATTCATTGGAGATGACAATATCGTGAAAGGAGTCAAAT
CGGACAAATTAATGGCAGACAGGTGCGCCACCTGGTTGAATATGGAAGTCAAGATTATAG
ATGCTGTGGTGGGCGAGAAAGCGCCTTATTTCTGTGGAGGGTTTATTTTGTGTGACTCCG
TGACCGGCACAGCGTGCCGTGTGGCAGACCCCCTAAAAAGGCTGTTTAAGCTTGGCAAAC
CTCTGGCAGCAGACGATGAACATGATGATGACAGGAGAAGGGCATTGCATGAAGAGTCAA
CACGCTGGAACCGAGTGGGTATTCTTTCAGAGCTGTGCAAGGCAGTAGAATCAAGGTATG
AAACCGTAGGAACTTCCATCATAGTTATGGCCATGACTACTCTAGCTAGCAGTGTTAAAT
CATTCAGCTACCTGAGAGGGGCCCCTATAACTCTCTACGGCTAACCTGAATGGACTACGA
CATAGTCTAGTCCGCCAAGTCTAGCATATGGCCACC --- GOI ---
[SEQ ID No: 183]
CGGAGACGGCGCAGAAGAAGAGGATCTGGCGAAGGCAGAGGCAGCCTGCTTACATGTGGC
GACGTGGAAGAGAACCCCGGACCTATGGCCCGCGCTGCTCTCTCCGCCGCCCCCAGCAA
TCCCCGGCTCCTGCGAGTGGCACTGCTGCTCCTGCTCCTGGTAGCCGCTGGCCGGCGCG
CAGCAGGAGCGTCCGTGGCCACTGAACTGCGCTGCCAGTGCTTGCAGACCCTGCAGGGA
ATTCACCCCAAGAACATCCAAAGTGTGAACGTGAAGTCCCCCGGACCCCACTGCGCCCA
AACCGAAGTCATAGCCACACTCAAGAATGGGCGGAAAGCTTGCCTCAATCCTGCATCCC
CCATAGTTAAGAAAATCATCGAAAAGATGCTGAACAGTGACAAATCCAACTGAGCGGCC
GCGAATTGGCAAGCTGCTTACATAGAACTCGCGGCGATTGGCATGCCGCCTTAAAATTTT
TATTTTATTTTTCTTTTCTTTTCCGAATCGGATTTTGTTTTTAATATTTCAAAAAAAAAA
AAAAAAAAAAAAAAAAAAAAAAAAACGCGTCGAGGGGAATTAATTCTTGAAGACGAAAGG
GCCAGGTGGCACTTTTCGGGGAAATGTGCGCGGAACCCCTATTTGTTTATTTTTCTAAAT
ACATTCAAATATGTATCCGCTCATGAGACAATAACCCTGATAAATGCTTCAATAATATTG
AAAAAGGAAGAGTATGAGTATTCAACATTTCCGTGTCGCCCTTATTCCCTTTTTTGCGGC
ATTTTGCCTTCCTGTTTTTGCTCACCCAGAAACGCTGGTGAAAGTAAAAGATGCTGAAGA
TCAGTTGGGTGCACGAGTGGGTTACATCGAACTGGATCTCAACAGCGGTAAGATCCTTGA
GAGTTTTCGCCCCGAAGAACGTTTTCCAATGATGAGCACTTTTAAAGTTCTGCTATGTGG
CGCGGTATTATCCCGTGTTGACGCCGGGCAAGAGCAACTCGGTCGCCGCATACACTATTC
TCAGAATGACTTGGTTGAGTACTCACCAGTCACAGAAAAGCATCTTACGGATGGCATGAC
AGTAAGAGAATTATGCAGTGCTGCCATAACCATGAGTGATAACACTGCGGCCAACTTACT
TCTGACAACGATCGGAGGACCGAAGGAGCTAACCGCTTTTTTGCACAACATGGGGGATCA
TGTAACTCGCCTTGATCGTTGGGAACCGGAGCTGAATGAAGCCATACCAAACGACGAGCG
TGACACCACGATGCCTGTAGCAATGGCAACAACGTTGCGCAAACTATTAACTGGCGAACT
ACTTACTCTAGCTTCCCGGCAACAATTAATAGACTGGATGGAGGCGGATAAAGTTGCAGG
ACCACTTCTGCGCTCGGCCCTTCCGGCTGGCTGGTTTATTGCTGATAAATCTGGAGCCGG
TGAGCGTGGGTCTCGCGGTATCATTGCAGCACTGGGGCCAGATGGTAAGCCCTCCCGTAT
CGTAGTTATCTACACGACGGGGAGTCAGGCAACTATGGATGAACGAAATAGACAGATCGC
TGAGATAGGTGCCTCACTGATTAAGCATTGGTAACTGTCAGACCAAGTTTACTCATATAT
ACTTTAGATTGATTTAAAACTTCATTTTTAATTTAAAAGGATCTAGGTGAAGATCCTTTT
TGATAATCTCATGACCAAAATCCCTTAACGTGAGTTTTCGTTCCACTGAGCGTCAGACCC
CGTAGAAAAGATCAAAGGATCTTCTTGAGATCCTTTTTTTCTGCGCGTAATCTGCTGCTT
GCAAACAAAAAAACCACCGCTACCAGCGGTGGTTTGTTTGCCGGATCAAGAGCTACCAAC
TCTTTTTCCGAAGGTAACTGGCTTCAGCAGAGCGCAGATACCAAATACTGTCCTTCTAGT
GTAGCCGTAGTTAGGCCACCACTTCAAGAACTCTGTAGCACCGCCTACATACCTCGCTCT
GCTAATCCTGTTACCAGTGGCTGCTGCCAGTGGCGATAAGTCGTGTCTTACCGGGTTGGA
CTCAAGACGATAGTTACCGGATAAGGCGCAGCGGTCGGGCTGAACGGGGGGTTCGTGCAC
ACAGCCCAGCTTGGAGCGAACGACCTACACCGAACTGAGATACCTACAGCGTGAGCATTG
AGAAAGCGCCACGCTTCCCGAAGGGAGAAAGGCGGACAGGTATCCGGTAAGCGGCAGGGT
CGGAACAGGAGAGCGCACGAGGGAGCTTCCAGGGGGAAACGCCTGGTATCTTTATAGTCC
TGTCGGGTTTCGCCACCTCTGACTTGAGCGTCGATTTTTGTGATGCTCGTCAGGGGGGCG
GAGCCTATGGAAAAACGCCAGCAACGCGAGCTC

Accordingly, preferably the vector comprises the nucleotide sequence substantially as set out above, comprising or consisting of SEQ ID NO: 162, a GOI, and SEQ ID No: 183, or a variant or fragment thereof.

The saRNA constructs of the invention may be made using a DNA plasmid, as a template. RNA copies may then be made by in vitro transcription using a polymerase, such as T7 polymerase, and the T7 promoter may be upstream of the saRNA. Hence, the saRNA constructs of the invention may be made using the DNA plasmid comprising a nucleic acid sequence as set out in any one of SEQ ID No: 161 or 162, or a variant or fragment thereof, as the template, such as the sequence substantially as set out above, comprising or consisting of SEQ ID No: 161, a GOI, and SEQ ID No: 182, or a variant or fragment thereof, or the sequence substantially as set out above, comprising or consisting of SEQ ID No: 162, a GOI, and SEQ ID No: 183, or a variant or fragment thereof, as the template. Of course, it will be appreciated that other RNA polymerases could be used instead of T7 polymerase, for example the SP6 or the T3 polymerase, in which case the saRNA construct may comprise the SP6 or T3 promoter instead.

The vector of the seventh aspect encoding the RNA construct may for example be a plasmid, cosmid or phage and/or be a viral vector. Such recombinant vectors are highly useful in the delivery systems of the invention for transforming cells with the nucleotide sequences. The nucleotide sequences may preferably be a DNA sequence, and it is this DNA sequence which encodes the RNA sequence forming the RNA construct of the invention.

Recombinant vectors encoding the RNA construct may also include other functional elements. For example, they may further comprise a variety of other functional elements including a suitable promoter for initiating transgene expression upon introduction of the vector in a host cell. For instance, the vector is preferably capable of autonomously replicating in the nucleus of the host cell, such as a bacterial cell. In this case, elements which induce or regulate DNA replication may be required in the recombinant vector. Alternatively, the recombinant vector may be designed such that it integrates into the genome of a host cell. In this case, DNA sequences which favour targeted integration (e.g. by homologous recombination) are envisaged. Suitable promoters may include the SV40 promoter, CMV, EF1a, PGK, viral long terminal repeats, as well as inducible promoters, such as the Tetracycline inducible system, as examples. The cassette or vector may also comprise a terminator, such as the Beta globin, SV40 polyadenylation sequences or synthetic polyadenylation sequences. The recombinant vector may also comprise a promoter or regulator or enhancer to control expression of the nucleic acid as required.

The vector may also comprise DNA coding for a gene that may be used as a selectable marker in the cloning process, i.e. to enable selection of cells that have been transfected or transformed, and to enable the selection of cells harbouring vectors incorporating heterologous DNA. For example, ampicillin, neomycin, puromycin or chloramphenicol resistance is envisaged. Alternatively, the selectable marker gene may be in a different vector to be used simultaneously with the vector containing the transgene(s). The cassette or vector may also comprise DNA involved with regulating expression of the nucleotide sequence, or for targeting the expressed polypeptide to a certain part of the host cell.

Purified vector may be inserted directly into a host cell by suitable means, e.g. direct endocytotic uptake. The vector may be introduced directly into a host cell (e.g. a eukaryotic or prokaryotic cell) by transfection, infection, electroporation, microinjection, cell fusion, protoplast fusion or ballistic bombardment. Alternatively, vectors of the invention may be introduced directly into a host cell using a particle gun.

The nucleic acid molecule may (but not necessarily) be one, which becomes incorporated in the DNA of the host cell. Undifferentiated cells may be stably transformed leading to the production of genetically modified daughter cells (in which case regulation of expression in the subject may be required e.g. with specific transcription factors or gene activators). Alternatively, the delivery system may be designed to favour unstable or transient transformation of differentiated cells. When this is the case, regulation of expression may be less important because expression of the DNA molecule will stop when the transformed cells die or stop expressing the protein.

Alternatively, the delivery system may provide the nucleic acid molecule to the host cell without it being incorporated in a vector. For instance, the nucleic acid molecule may be incorporated within a liposome or virus particle. Alternatively a “naked” nucleic acid molecule may be inserted into a host cell by a suitable means e.g. direct endocytotic uptake.

In an eighth aspect, there is provided a pharmaceutical composition comprising the RNA construct of any one of the first to fourth aspect, the nucleic acid sequence of the fifth aspect, the expression cassette of the sixth aspect or the vector of the seventh aspect, and a pharmaceutically acceptable vehicle.

In a ninth aspect, there is provided a process for making the pharmaceutical composition according to the eighth aspect, the method comprising contacting the RNA construct of any one of the first to fourth aspect, the nucleic acid sequence of the fifth aspect, the expression cassette of the sixth aspect or the vector of the seventh aspect, with a pharmaceutically acceptable vehicle.

In a tenth aspect, there is provided a method of preparing the RNA construct of any one of the first to fourth aspect, the method comprising:
a) i) introducing, into a host cell, the vector of the seventh aspect; and
   ii) culturing the host cell under conditions to result in the production of the RNA construct; or
b) transcribing the RNA construct from the vector according to the seventh aspect.

The host cell of step a) may be a eukaryotic or prokaryotic host cell. Preferably, the host cell is a eukaryotic host cell. More preferably, the host cell is a mammalian host cell such as Human embryonic kidney 293 cells or Chinese hamster ovary (CHO) cells. Step (b) may be performed in vitro or in vivo, preferably in vitro.

Suitable methods of in vitro transcription are well known in the art and would be known to those skilled in the art. For example, as described in Molecular Cloning, A Laboratory Manual, 2nd edition. (1989) editor C Nolan, Cold Spring Harbor Laboratory Press.

The RNA constructs of the invention are particularly suitable for therapy.

While the inventors envisaged that the RNA constructs would be generated by in vitro transcription for in vivo use in therapy, those experienced in the art will recognise that the RNA constructs can be generated in vivo in a subject for therapy, by in vivo delivery of the nucleic acid sequence of the fifth aspect, the expression cassette of the sixth aspect or the vector of the seventh aspect.

Hence, according to an eleventh aspect, there is provided the RNA construct of any one of the first to fourth aspect, the nucleic acid sequence of the fifth aspect, the expression cassette of the sixth aspect, the vector of the seventh aspect or the pharmaceutical composition according to the eighth aspect, for use as a medicament or in therapy.

In a twelfth aspect of the invention, there is provided the RNA construct of any one of the first to fourth aspect, the nucleic acid sequence of the fifth aspect, the expression cassette of the sixth aspect, the vector of the seventh aspect or the pharmaceutical composition according to the eighth aspect, for use in the prevention, amelioration or treatment of a protozoan, fungal, bacterial or viral infection.

The protozoan, fungal, bacterial or viral infection may be an infection of a protozoa, fungus, bacterium or virus as defined above.

In a thirteenth aspect of the invention, there is provided the RNA construct of any one of the first to fourth aspect, the nucleic acid sequence of the fifth aspect, the expression cassette of the sixth aspect, the vector of the seventh aspect or the pharmaceutical composition according to the eighth aspect, for use in the prevention, amelioration or treatment of cancer.

The cancer may be as defined above.

In a fourteenth aspect of the invention, there is provided a method for treating a protozoan, fungal, bacterial or viral infection, the method comprising administering, to a subject in need thereof, a therapeutically effective amount of the RNA construct of any one of the first to fourth aspect, the nucleic acid sequence of the fifth aspect, the expression cassette of the sixth aspect, the vector of the seventh aspect or the pharmaceutical composition according to the eighth aspect.

The protozoan, fungal, bacterial or viral infection to be treated may be an infection of a protozoa, fungus, bacterium or virus as defined above.

In a fifteenth aspect of the invention, there is provided a method for treating cancer, the method comprising administering, to a subject in need thereof, a therapeutically effective amount of the RNA construct of any one of the first to fourth aspect, the nucleic acid sequence of the fifth aspect, the expression cassette of the sixth aspect, the vector of the seventh aspect or the pharmaceutical composition according to the eighth aspect.

The cancer to be treated may be as defined above.

The RNA constructs described herein provides an effective means of vaccinating a subject (e.g. against a viral, bacterial or fungal infection) and cancer.

Accordingly, in a sixteenth aspect of the invention, there is provided a vaccine comprising the RNA construct of any one of the first to fourth aspect, the nucleic acid sequence of the fifth aspect, the expression cassette of the sixth aspect, the vector of the seventh aspect or the pharmaceutical composition according to the eighth aspect.

Preferably, the vaccine comprises a suitable adjuvant, which would be in addition to the molecular adjuvant, i.e. the ISP.

The additional adjuvant may be incorporated into a delivery formulation. The adjuvant incorporated into a delivery formulation may be selected form the group consisting of a bacterial lipopeptide, lipoprotein and lipoteichoic acid; mycobacterial lipoglycan; yeast zymosan, porin, Lipopolysaccharide, Lipid A, monophosphoryl lipid A (MPL), Flagellin, CpG DNA, hemozoin, Tomatine, ISCOM, ISCOMATRIX™, squalene based emulsions, polymers such as PEI, Carbopol, lipid nanoparticles and bacterial toxins (CT, LT).

In a seventeenth aspect of the invention, there is provided the RNA construct of any one of the first to fourth aspect, the nucleic acid sequence of the fifth aspect, the expression cassette of the sixth aspect, the vector of the seventh aspect or the pharmaceutical composition according to the eighth aspect, for use in stimulating an immune response in a subject.

The immune response may be stimulated against a protozoa, bacterium, virus, fungus or cancer as per the antigens defined herein above.

According to an eighteenth aspect, there is provided the RNA construct of any one of the first to fourth aspect, the nucleic acid sequence of the fifth aspect, the expression cassette of the sixth aspect, the vector of the seventh aspect or the pharmaceutical composition according to the eighth aspect, for use in stem cell therapy.

Stem cell therapy may relate to the reprogramming somatic cells to cells having stem cell characteristics.

Somatic cells may be reprogrammed by delivering one or more proteins that are capable of enhancing reprogramming of somatic cells to cells having stem cell characteristics as defined above.

According to a nineteenth aspect, there is provided a method of modifying a cell ex vivo or in vitro, comprising delivering, to the cell, the RNA construct of any one of the first to fourth aspect, the nucleic acid sequence of the fifth aspect, the expression cassette of the sixth aspect, the vector of the seventh aspect or the pharmaceutical composition according to the eighth aspect.

Preferably, the method is performed ex vivo.

The cell may be a eukaryotic or prokaryotic cell. Preferably, the cell is a eukaryotic cell. More preferably, the cell is a mammalian host cell. Most preferably, the cell is a human cell.

Preferably, the modified cell is suitable for cell-therapy indications.

In a twentieth aspect, there is provided a modified cell obtained from, or obtainable by, the method of the nineteenth aspect.

In a twenty first aspect, there is provided the modified cell of the twentieth aspect, for use in therapy, optionally cell therapy.

It will be appreciated that the RNA construct of any one of the first to fourth aspect, the nucleic acid sequence of the fifth aspect, the expression cassette of the sixth aspect, the vector of the seventh aspect or the pharmaceutical composition according to the eighth aspect (herein known as the active agents) may be used in a medicament, which may be used as a monotherapy (i.e. use of the active agent), for treating, ameliorating, or preventing disease or vaccination. Alternatively, the active agents according to the invention may be used as an adjunct to, or in combination with, known therapies for treating, ameliorating, or preventing disease.

The RNA construct, nucleic acid sequence, expression cassette, vector or pharmaceutical composition of the invention may be combined in compositions having a number of different forms depending, in particular, on the manner in which the composition is to be used. Thus, for example, the composition may be in the form of a powder, tablet, capsule, liquid, ointment, cream, gel, hydrogel, aerosol, spray, micellar solution, transdermal patch, liposome suspension, polyplex, emulsion, lipid nanoparticles (with RNA on the surface or encapsulated) or any other suitable form that may be administered to a person or animal in need of treatment or vaccination. It will be appreciated that the vehicle of medicaments according to the invention should be one which is well-tolerated by the subject to whom it is given.

The RNA construct, nucleic acid sequence, expression cassette, vector or pharmaceutical composition of the invention may also be incorporated within a slow- or delayed-release device. Such devices may, for example, be inserted on or under the skin, and the medicament may be released over weeks or even months. The device may be located at least adjacent the treatment site. Such devices may be particularly advantageous when long-term treatment with the genetic construct or the recombinant vector is required and which would normally require frequent administration (e.g. at least daily injection).

In a preferred embodiment, however, medicaments according to the invention may be administered to a subject by injection into the blood stream, muscle, skin or directly into a site requiring treatment. Most preferably, the medicaments, including the RNA construct, are injected into muscle. Injections may be intravenous (bolus or infusion) or subcutaneous (bolus or infusion), or intradermal (bolus or infusion), or intramuscular (bolus or infusion).

It will be appreciated that the amount of RNA construct, nucleic acid sequence, expression cassette, vector or pharmaceutical composition that is required is determined by its biological activity and bioavailability, which in turn depends on the mode of administration, the physiochemical properties of the RNA construct, nucleic acid sequence, expression cassette, vector or pharmaceutical composition and whether it is being used as a monotherapy or in a combined therapy. The frequency of administration will also be influenced by the half-life of the active agent within the subject being treated. Optimal dosages to be administered may be determined by those skilled in the art, and will vary with the particular the RNA construct, nucleic acid sequence, expression cassette, vector or pharmaceutical composition in use, the strength of the pharmaceutical composition, the mode of administration, and the type and advancement of the viral infection. Additional factors depending on the particular subject being treated will result in a need to adjust dosages, including subject age, weight, gender, diet, and time of administration.

Generally, a daily dose of between 0.001 μg/kg of body weight and 10 mg/kg of body weight, or between 0.01 μg/kg of body weight and 1 mg/kg of body weight, of the RNA construct, nucleic acid sequence, expression cassette, vector or pharmaceutical composition of the invention may be used for treating, ameliorating, or preventing a disease, depending upon the active agent used.

Daily doses may be given as a single administration (e.g. a single daily injection or inhalation of a nasal spray). Alternatively, the RNA construct, nucleic acid sequence, expression cassette, vector or pharmaceutical composition may require administration twice or more times during a day. As an example, the RNA construct, nucleic acid sequence, expression cassette, vector or pharmaceutical composition may be administered as two (or more depending upon the severity of the disease being treated) daily doses of between 0.07 μg and 700 mg (i.e. assuming a body weight of 70 kg). A patient receiving treatment may take a first dose upon waking and then a second dose in the evening (if on a two dose regime) or at 3- or 4-hourly intervals thereafter. Alternatively, a slow release device may be used to provide optimal doses of the RNA construct, nucleic acid sequence, expression cassette, vector or pharmaceutical composition according to the invention to a patient without the need to administer repeated doses.

Preferably, however, the RNA construct, nucleic acid sequence, expression cassette, vector or pharmaceutical composition according to the invention may be given as a weekly dose, and more preferably a fortnightly dose.

Known procedures, such as those conventionally employed by the pharmaceutical industry (e.g. in vivo experimentation, clinical trials, etc.), may be used to form specific formulations of the RNA construct, nucleic acid sequence, expression cassette or vector according to the invention and precise therapeutic regimes (such as daily doses of the agents and the frequency of administration).

A “subject” may be a vertebrate, mammal, or domestic animal. Hence, compositions and medicaments according to the invention may be used to treat any mammal, for example livestock (e.g. a horse), pets, or may be used in other veterinary applications. Most preferably, however, the subject is a human being.

A “therapeutically effective amount” of the RNA construct, nucleic acid sequence, expression cassette, vector or pharmaceutical composition is any amount which, when administered to a subject, is the amount of the aforementioned that is needed to ameliorate, prevent or treat any given disease.

For example, the RNA construct, nucleic acid sequence, expression cassette, vector or pharmaceutical composition of the invention may be used may be from about 0.0001 mg to about 800 mg, and preferably from about 0.001 mg to about 500 mg. It is preferred that the amount of the replicon, nucleic acid sequence, expression cassette, vector or pharmaceutical composition is an amount from about 0.01 mg to about 250 mg, and most preferably from about 0.01 mg to about 1 mg. Preferably, the RNA construct, nucleic acid sequence, expression cassette, vector or pharmaceutical composition according to the invention is administered at a dose of 1-200 μg.

A “pharmaceutically acceptable vehicle” as referred to herein, is any known compound or combination of known compounds that are known to those skilled in the art to be useful in formulating pharmaceutical compositions.

In one embodiment, the pharmaceutically acceptable vehicle may be a solid, and the composition may be in the form of a powder or tablet. A solid pharmaceutically acceptable vehicle may include one or more substances which may also act as flavouring agents, lubricants, solubilisers, suspending agents, dyes, fillers, glidants, compression aids, inert binders, sweeteners, preservatives, dyes, coatings, or tablet-disintegrating agents. The vehicle may also be an encapsulating material. In powders, the vehicle is a finely divided solid that is in admixture with the finely divided active agents according to the invention. In tablets, the active agent (e.g. RNA construct, nucleic acid sequence, expression cassette, vector or pharmaceutical composition according to the invention) may be mixed with a vehicle having the necessary compression properties in suitable proportions and compacted in the shape and size desired. The powders and tablets preferably contain up to 99% of the active agents. Suitable solid vehicles include, for example calcium phosphate, magnesium stearate, tale, sugars, lactose, dextrin, starch, gelatin, cellulose, polyvinylpyrrolidine, low melting waxes and ion exchange resins. In another embodiment, the pharmaceutical vehicle may be a gel and the composition may be in the form of a cream or the like.

However, the pharmaceutical vehicle may be a liquid, and the pharmaceutical composition is in the form of a solution. Liquid vehicles are used in preparing solutions, suspensions, emulsions, syrups, elixirs and pressurized compositions. The RNA construct, nucleic acid sequence, expression cassette, vector or pharmaceutical composition according to the invention may be dissolved or suspended in a pharmaceutically acceptable liquid vehicle such as water, an organic solvent, a mixture of both or pharmaceutically acceptable oils or fats. The liquid vehicle can contain other suitable pharmaceutical additives such as solubilisers, emulsifiers, buffers, preservatives, sweeteners, flavouring agents, suspending agents, thickening agents, colours, viscosity regulators, stabilizers or osmo-regulators. Suitable examples of liquid vehicles for oral and parenteral administration include water (partially containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil). For parenteral administration, the vehicle can also be an oily ester such as ethyl oleate and isopropyl myristate. Sterile liquid vehicles are useful in sterile liquid form compositions for parenteral administration. The liquid vehicle for pressurized compositions can be a halogenated hydrocarbon or other pharmaceutically acceptable propellant.

Liquid pharmaceutical compositions, which are sterile solutions or suspensions, can be utilized by, for example, subcutaneous, intradermal, intrathecal, epidural, intraperitoneal, intravenous and particularly intramuscular injection. The nucleic acid sequence, or expression cassette of the invention may be prepared as a sterile solid composition that may be dissolved or suspended at the time of administration using sterile water, saline, or other appropriate sterile injectable medium.

The RNA construct, nucleic acid sequence, expression cassette, vector or pharmaceutical composition of the invention may be administered orally in the form of a sterile solution or suspension containing other solutes or suspending agents (for example, enough saline or glucose to make the solution isotonic), bile salts, acacia, gelatin, sorbitan monoleate, polysorbate 80 (oleate esters of sorbitol and its anhydrides copolymerized with ethylene oxide) and the like. The RNA construct, nucleic acid sequence, expression cassette, vector or pharmaceutical composition according to the invention can also be administered orally either in liquid or solid composition form. Compositions suitable for oral administration include solid forms, such as pills, capsules, granules, tablets, and powders, and liquid forms, such as solutions, syrups, elixirs, and suspensions. Forms useful for parenteral administration include sterile solutions, emulsions, and suspensions.

It will be appreciated that the invention extends to any nucleic acid or peptide or variant, derivative or analogue thereof, which comprises substantially the amino acid or nucleic acid sequences of any of the sequences referred to herein, including variants or fragments thereof. The terms “substantially the amino acid/nucleotide/peptide sequence”, “variant” and “fragment”, can be a sequence that has at least 40% sequence identity with the amino acid/nucleotide/peptide sequences of any one of the sequences referred to herein, for example 40% identity with the sequence identified as SEQ ID Nos: 1-162 and so on.

Amino acid/polynucleotide/polypeptide sequences with a sequence identity which is greater than 65%, more preferably greater than 70%, even more preferably greater than 75%, and still more preferably greater than 80% sequence identity to any of the sequences referred to are also envisaged. Preferably, the amino acid/polynucleotide/polypeptide sequence has at least 85% identity with any of the sequences referred to, more preferably at least 90% identity, even more preferably at least 92% identity, even more preferably at least 95% identity, even more preferably at least 97% identity, even more preferably at least 98% identity and, most preferably at least 99% identity with any of the sequences referred to herein.

The skilled technician will appreciate how to calculate the percentage identity between two amino acid/polynucleotide/polypeptide sequences. In order to calculate the percentage identity between two amino acid/polynucleotide/polypeptide sequences, an alignment of the two sequences must first be prepared, followed by calculation of the sequence identity value. The percentage identity for two sequences may take different values depending on:—(i) the method used to align the sequences, for example, ClustalW, BLAST, FASTA, Smith-Waterman (implemented in different programs), or structural alignment from 3D comparison; and (ii) the parameters used by the alignment method, for example, local vs global alignment, the pair-score matrix used (e.g. BLOSUM62, PAM250, Gonnet etc.), and gap-penalty, e.g. functional form and constants.

Having made the alignment, there are many different ways of calculating percentage identity between the two sequences. For example, one may divide the number of identities by: (i) the length of shortest sequence; (ii) the length of alignment; (iii) the mean length of sequence; (iv) the number of non-gap positions; or (v) the number of equivalenced positions excluding overhangs. Furthermore, it will be appreciated that percentage identity is also strongly length dependent. Therefore, the shorter a pair of sequences is, the higher the sequence identity one may expect to occur by chance.

Hence, it will be appreciated that the accurate alignment of protein or DNA sequences is a complex process. The popular multiple alignment program ClustalW (Thompson et al., 1994, Nucleic Acids Research, 22, 4673-4680; Thompson et al., 1997, Nucleic Acids Research, 24, 4876-4882) is a preferred way for generating multiple alignments of proteins or DNA in accordance with the invention. Suitable parameters for ClustalW may be as follows: For DNA alignments: Gap Open Penalty=15.0, Gap Extension Penalty=6.66, and Matrix=Identity. For protein alignments: Gap Open Penalty=10.0, Gap Extension Penalty=0.2, and Matrix=Gonnet. For DNA and Protein alignments: ENDGAP=−1, and GAPDIST=4. Those skilled in the art will be aware that it may be necessary to vary these and other parameters for optimal sequence alignment.

Preferably, calculation of percentage identities between two amino acid/polynucleotide/polypeptide sequences may then be calculated from such an alignment as (N/T)*100, where N is the number of positions at which the sequences share an identical residue, and T is the total number of positions compared including gaps and either including or excluding overhangs. Preferably, overhangs are included in the calculation. Hence, a most preferred method for calculating percentage identity between two sequences comprises (i) preparing a sequence alignment using the ClustalW program using a suitable set of parameters, for example, as set out above; and (ii) inserting the values of N and T into the following formula:—Sequence Identity=(N/T)*100.

Alternative methods for identifying similar sequences will be known to those skilled in the art. For example, a substantially similar nucleotide sequence will be encoded by a sequence which hybridizes to DNA sequences or their complements under stringent conditions. By stringent conditions, the inventors mean the nucleotide hybridises to filter-bound DNA or RNA in 3× sodium chloride/sodium citrate (SSC) at approximately 45° C. followed by at least one wash in 0.2×SSC/0.1% SDS at approximately 20-65° C. Alternatively, a substantially similar polypeptide may differ by at least 1, but less than 5, 10, 20, 50 or 100 amino acids from the sequences shown in, for example, SEQ ID Nos:1 to 162.

Due to the degeneracy of the genetic code, it is clear that any nucleic acid sequence described herein could be varied or changed without substantially affecting the sequence of the protein encoded thereby, to provide a functional variant thereof. Suitable nucleotide variants are those having a sequence altered by the substitution of different codons that encode the same amino acid within the sequence, thus producing a silent (synonymous) change. Other suitable variants are those having homologous nucleotide sequences but comprising all, or portions of, sequence, which are altered by the substitution of different codons that encode an amino acid with a side chain of similar biophysical properties to the amino acid it substitutes, to produce a conservative change. For example, small non-polar, hydrophobic amino acids include glycine, alanine, leucine, isoleucine, valine, proline, and methionine. Large non-polar, hydrophobic amino acids include phenylalanine, tryptophan and tyrosine. The polar neutral amino acids include serine, threonine, cysteine, asparagine and glutamine. The positively charged (basic) amino acids include lysine, arginine and histidine. The negatively charged (acidic) amino acids include aspartic acid and glutamic acid. It will therefore be appreciated which amino acids may be replaced with an amino acid having similar biophysical properties, and the skilled technician will know the nucleotide sequences encoding these amino acids.

All of the features described herein (including any accompanying claims, abstract and drawings), and/or all of the steps of any method or process so disclosed, may be combined with any of the above aspects in any combination, except combinations where at least some of such features and/or steps are mutually exclusive.

For a better understanding of the invention, and to show how embodiments of the same may be carried into effect, reference will now be made, by way of example, to the accompanying Figures, in which:—

FIG. 1 shows a schematic of various embodiments (denoted 1-7) of the RNA construct of the invention (e.g. a saRNA replicon on the left, or an mRNA construct). The saRNA replicon (1-4) is based on an alpha virus backbone. This so-called ‘Stealthicon’ vector includes a 5′ UTR followed by nucleic acid encoding Non-structural Proteins (NSP1-4) from an alphavirus, such as VEEV, a sub-genomic promoter (SGP), a GOI (Gene of Interest), such as a viral, bacterial, fungal or mammalian protein or antigen, an immune stimulatory protein (ISP), a 3′ UTR and a 3′ poly A tail. The mRNA construct (denoted 5-7) includes a 5′ UTR, a GOI (Gene of Interest), such as a viral, bacterial, fungal or mammalian protein or antigen, an immune stimulatory protein (ISP), a 3′ UTR and a 3′ poly A tail. The ISP and GOI are separated by linkers (F-T2a and IRES), and the order of the ISP and GOI can be varied for both saRNA and mRNA as shown in the different illustrated embodiments;

FIG. 2 shows a schematic of various alternative embodiments of the RNA construct of the invention (e.g. a saRNA replicon on the left, or an mRNA construct). The saRNA replicons on the left are based on an alpha virus backbone. This so-called ‘Stealthicon’ vector includes a 5′ UTR followed by nucleic acid encoding Non-structural Proteins (NSP1-4) from an alphavirus, such as VEEV, a sub-genomic promoter (SGP), a GOI (Gene of Interest), such as a viral, bacterial, fungal or mammalian protein or antigen, a viral-derived or a human-derived immune modulatory or inhibitor protein (IMP), an immune stimulatory protein (ISP), a 3′ UTR and a 3′ poly A tail. The GOI, IMP or IIP and ISP are separated by linkers. The mRNA construct includes a 5′ UTR, a GOI (Gene of Interest), such as a viral, bacterial, fungal or mammalian protein or antigen, a viral-derived or a human-derived immune modulatory or inhibitor protein (IMP), an immune stimulatory protein (ISP), a 3′ UTR and a 3′ poly A tail. The GOI, IMP and ISP are separate by linkers (F-T2a and IRES), and the order of the IMP, ISP and GOI can be varied for both saRNA and mRNA as shown in the different illustrated embodiments;

FIG. 3 illustrates the immune response in a subject vaccinated (an initial primer jab followed by a subsequent boost jab) with a messenger RNA (mRNA) vaccine;

FIG. 4 illustrates the immune response in a subject vaccinated (an initial primer jab followed by a boost jab) with a standard self-amplifying (saRNA) vaccine;

FIG. 5 illustrates the immune response in a subject vaccinated (an initial primer jab followed by a boost jab) with one embodiment of the RNA construct of the invention, for example the Stealthicon vector shown in FIG. 1 or 2;

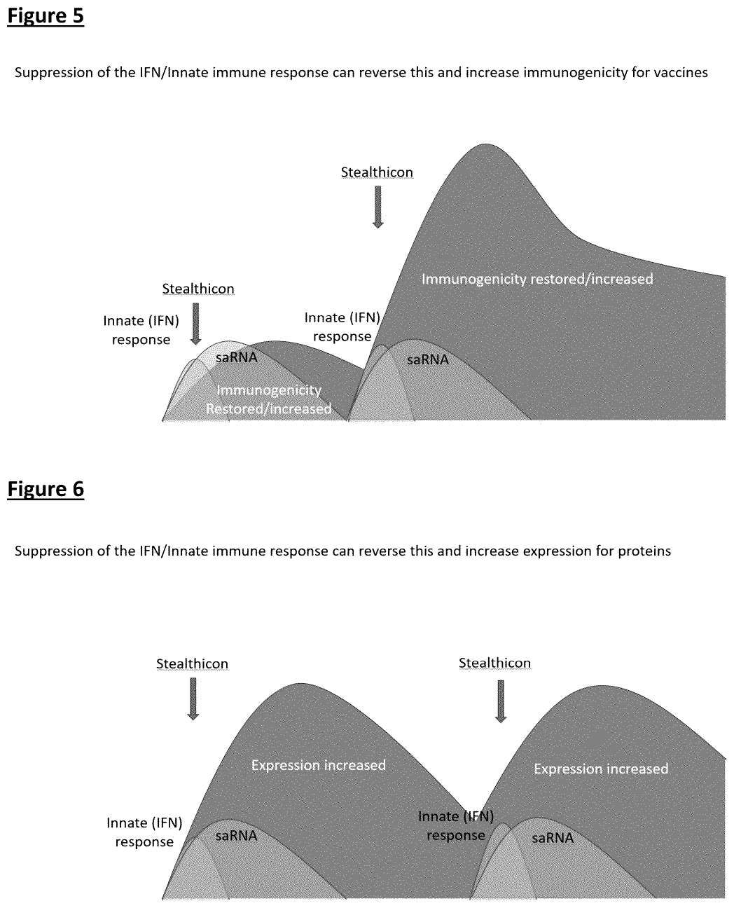

FIG. 6 illustrates the antigen expression level in a subject vaccinated (an initial primer jab followed by a boost jab) with one embodiment of the RNA construct of the invention, i.e. the Stealthicon vector shown in FIG. 1 or 2;

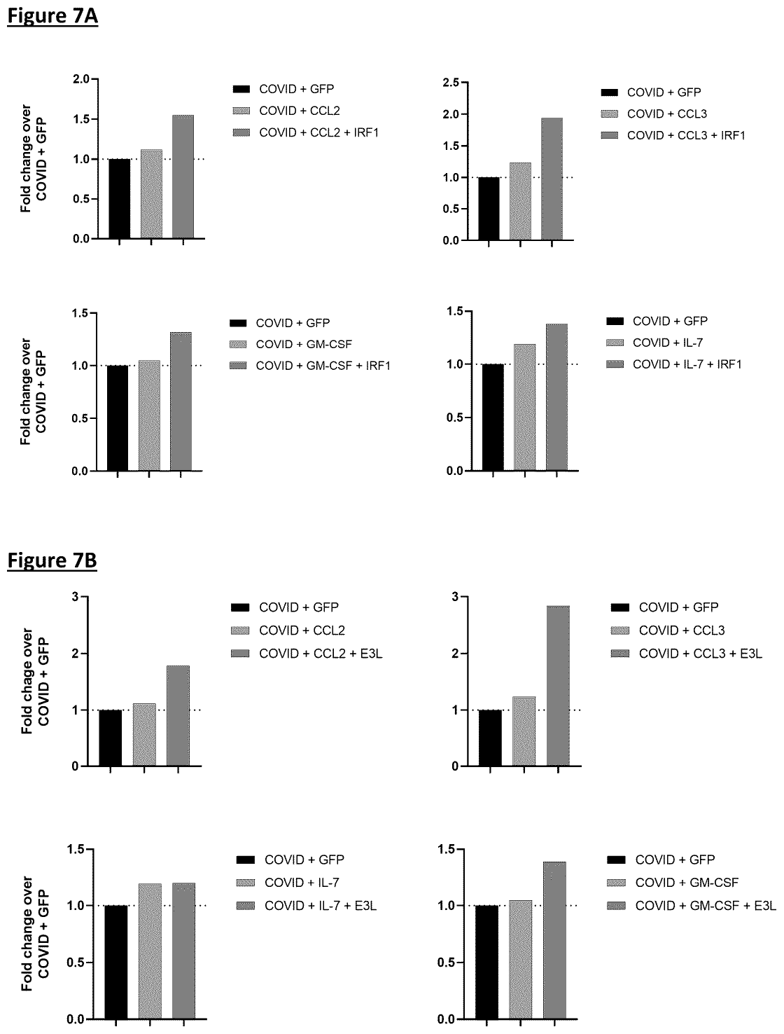

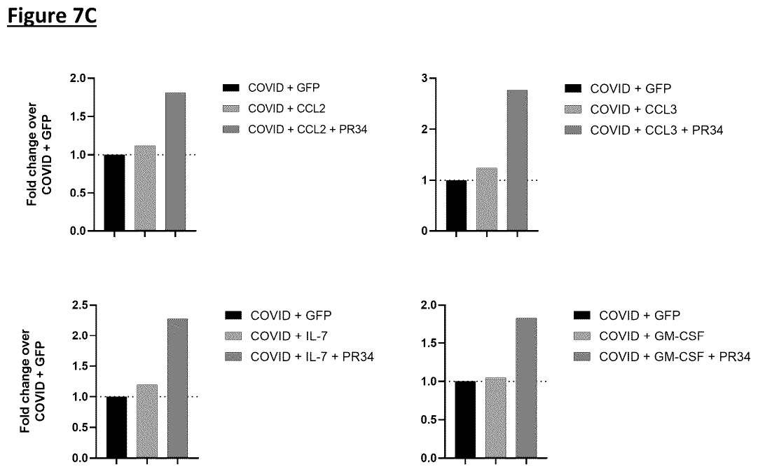

FIG. 7 illustrates in vitro enhancement of GoI expression. saRNA replicon constructs were transfected into Hela cells using lipofectamine and the expression of the Gene of Interest (GoI), in this specific case the COVID spike antigen was used as the model expression antigen. The in vitro expression is presented as a fold enhancement over the control saRNA replicon (saRNA COVID-GFP). The saRNA GOI−ISP (CCL2, CCL3, IL-7 or GM-CSF) replicons are graphed together with the saRNA-GOI-ISP-IIP (in which the IIP is IRF1, E3L or PR34) replicons. FIG. 7A shows GOI+ISP+IRF1 acting as the IIP. FIG. 7B shows GOI+ISP+E3L acting as the IIP. FIG. 7C shows GOI+ISP+PR34 acting as the IIP. As can be seen, there is an enhancement of the COVID expression in the presence of the ISP alone, and there is a marked enhancement by the ISP and the IIP in combination.

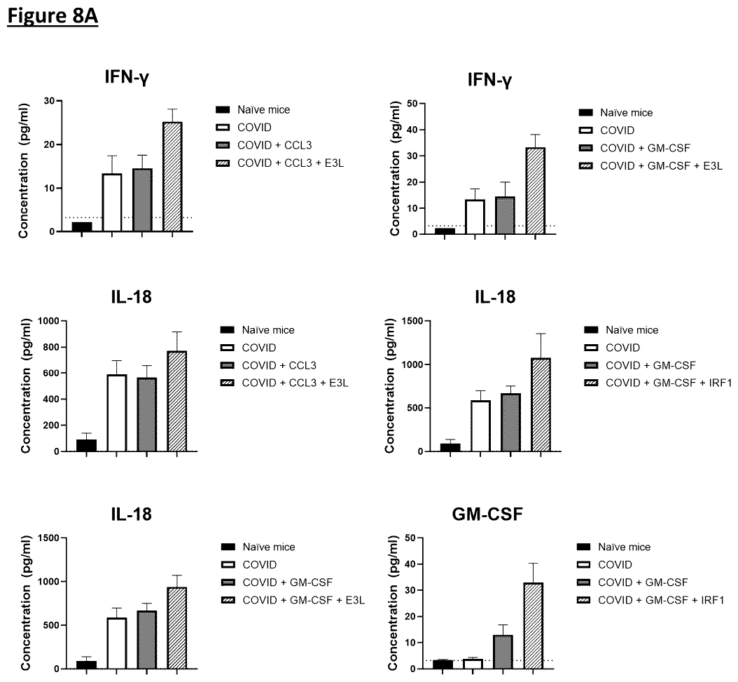

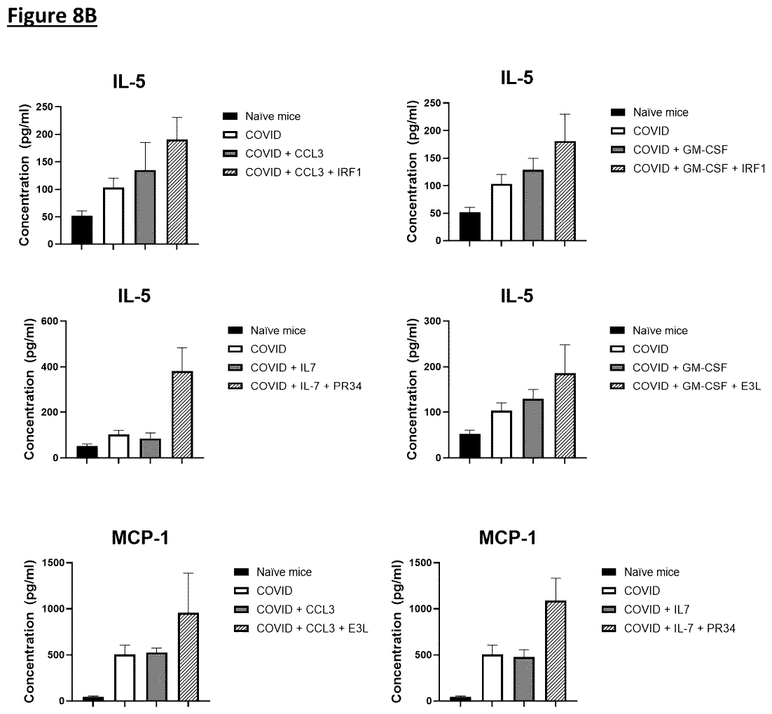

FIGS. 8A and 8B illustrate in vivo enhancement of inflammatory/stimulatory chemokines/cytokines. Groups of mice (n=5) were vaccinated with various lipid nanoparticles (LNP)-formulated saRNA replicons expressing GoI+IIPs alone, GoI+ISPs alone or combinations of GoI+ISPs+IIPs and the murine sera harvested 24 hours post injection. The expressed antigen (COVID spike protein used as the model antigen) and/or the COVID+ISP enhanced the circulating expression levels of various cytokines/chemokines as measured by Luminex; and

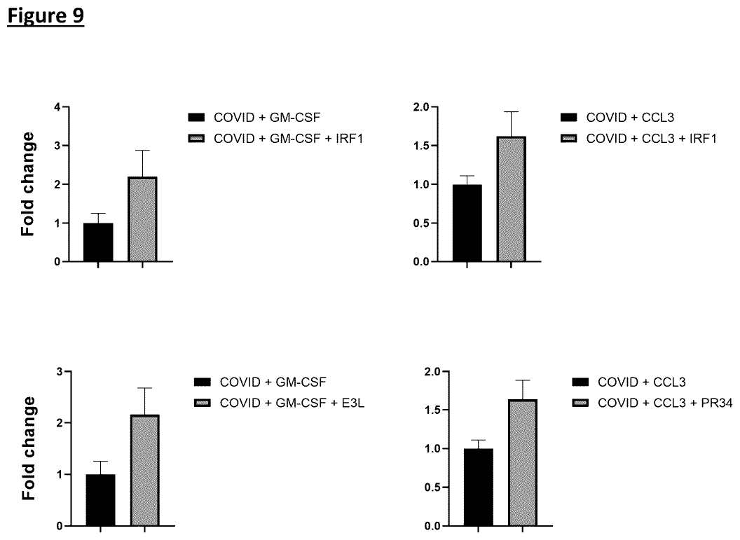

FIG. 9 shows in vivo enhancement of GoI specific antibody immune responses. Groups of mice (n=5) were vaccinated with various LNP-formulated saRNA replicons expressing GoI+IIPs alone, GoI+ISPs alone or combinations of GoI+ISPs+IIPs and the murine sera was tested for antigen-specific antibodies at day 42 post immunisation. These animals received a prime vaccination at day 0 and a boost vaccination at day 28 with 1 μg of LNP formulated saRNA replicon. The saRNA replicons containing ISPs+IIPs were compared to the immune responses in those animals that had received only the ISP clearly demonstrating the enhancement to antibody immunity provided by the activity of the IIPs on the expression of the both the GoI and/or the functionality of the ISPs. Graphs are presented as fold-change of antibody levels when compared to the corresponding ISP alone.

### EXAMPLES

The inventors hypothesized that RNA encoded immune stimulatory proteins (ISP) from humans and other mammals, improve the adjuvant activity of RNA vaccines. Thus, the inventors designed and tested a range of RNA constructs (saRNA and mRNA) containing an immunogen or “gene” of interest (GOI) and an ISP and then characterized whether these constructs had increased expression and/or improved immunogenicity. They also made a further set of constructs that in addition to the GOI and ISP, harboured either an innate inhibitory or modulatory protein (IIP or IMP) known to reduce innate sensing of saRNA or mRNA in the host cell. The inventors then tested whether the GOI protein expression was enhanced in these constructs and whether the immunogenicity of the RNA vaccine was further improved.

### Materials and Methods

### Cloning of Molecular Adjuvant Replicons

Self-amplifying RNA encoding an immune stimulatory protein (ISP), a gene of interest (GOI) and/or and IIP and the replicase derived from the Venezuelan equine encephalitis were cloned into a plasmid vector, as previously described (1). The library of innate immunomodulatory proteins were cloned into the vector backbone as part of a gene of interest (GOI) with a T2A cleavage site (GenBank accession #AAC97195.1), as a second expression cassette separated using an internal ribosome entry site (IRES) or using a double sub-genomic promoter.

(1) A. K. Blakney, P. F. McKay, R. J. Shattock, Structural Components for Amplification of Positive and Negative Strand VEEV Splitzicons. Frontiers in Molecular Biosciences 5, 71 (2018).

Cloning of Molecular Adjuvant mRNA

ISP, IMP or IIP were inserted into a base plasmid using restriction digestion followed by Gibson assembly. The library of innate immunomodulatory proteins were cloned into the vector backbone as part of a gene of interest (GOI) with a T2A cleavage site (GenBank accession #AAC97195.1), as a second expression cassette separated using an internal ribosome entry site (IRES) or using a double sub-genomic promoter

The innate immunomodulatory proteins can be found with the following NCBI and UniProt accession numbers: CXCL1 (NCBI Reference Sequence: NM_001511.4; UniProtKB—P09341 (GROA_HUMAN)); CXCL8 (IL8) (NCBI Reference Sequence: NM_000584.4; UniProtKB—P10145 (IL8_HUMAN)); CXCL10 (NCBI Reference Sequence: NM_001565.4; UniProtKB—P02778 (CXL10_HUMAN)); CXCL11 (NCBI Reference Sequence: NM_005409.5; UniProtKB—O14625 (CXL11_HUMAN)); CXCL12 (NCBI Reference Sequence: NM_000609.7; UniProtKB—P48061 (SDF1_HUMAN)); CCL1 (NCBI Reference Sequence: NM_002981.2; UniProtKB—P22362 (CCL1_HUMAN)); CCL20 (NCBI Reference Sequence: NM_004591.3; UniProtKB—P78556 (CCL20_HUMAN)); CCL21 (NCBI Reference Sequence: NM_002989.4; UniProtKB—O00585 (CCL21_HUMAN)); CX3CL1 or Fractalkine (NCBI Reference Sequence: NM_002996.6; UniProtKB—P78423 (X3CL1_HUMAN)); CX3CL1 or Fractalkine—Secreted Form (NCBI Reference Sequence: NM_002996.6; UniProtKB—P78423 (X3CL1_HUMAN)); XCL1 (NCBI Reference Sequence: NM_002995.3; UniProtKB—P47992 (XCL1_HUMAN)); XCL2 (NCBI Reference Sequence: NM_003175.4; UniProtKB—Q9UBD3 (XCL2_HUMAN)); IL-1b (NCBI Reference Sequence: NM_000576.3; UniProtKB—P01584 (IL1B_HUMAN)); IL-1b—With enhanced secretion signal (NCBI Reference Sequence: NM_000576.3; UniProtKB—P01584 (IL1B_HUMAN)); IL-2 (NCBI Reference Sequence: NM_000586.4; UniProtKB—P60568 (IL2_HUMAN)); IL-18 (37-93) (NCBI Reference Sequence: NM_001562.4; UniProtKB—Q14116 (IL18_HUMAN)); IL-18 (37-93)—With enhanced signal sequence (NCBI Reference Sequence: NM_001562.4; UniProtKB—Q14116 (IL18_HUMAN)); IL-19 (NCBI Reference Sequence: NM_013371.4; UniProtKB—Q9UHD0 (IL19_HUMAN)); IL-20 (NCBI Reference Sequence: NM_018724.4; UniProtKB—Q9NYY1 (IL20_HUMAN)); IL21 (NCBI Reference Sequence: NM_021803.4; UniProtKB—Q9HBE4 (IL21_HUMAN)); IL-22 (NCBI Reference Sequence: NM_020525.5; UniProtKB—Q9GZX6 (IL22_HUMAN)); IL-33 (NCBI Reference Sequence: NM_033439.4; UniProtKB—O95760 (IL33_HUMAN)); IL-36 alpha with signal sequence (NCBI Reference Sequence: NM_014440.3; UniProtKB—Q9UHA7 (IL36A_HUMAN)); Tumour Necrosis Factor—Membrane Form (NCBI Reference Sequence: NM_000594.4; UniProtKB—P01375 (TNFA_HUMAN)); Tumour Necrosis Factor—Soluble Form (NCBI Reference Sequence: NM_000594.4; UniProtKB—P01375 (TNFA_HUMAN)); Human BAFF—Membrane Form (NCBI Reference Sequence: NM_006573.5; UniProtKB—Q9Y275 (TN13B_HUMAN)); Human BAFF—Soluble Form (NCBI Reference Sequence: NM_006573.5; UniProtKB—Q9Y275 (TN13B_HUMAN)); Human CD30 Ligand (NCBI Reference Sequence: NM_001244.4; UniProtKB—P32971 (TNFL8_HUMAN)); Human CD40 Ligand (NCBI Reference Sequence: NM_000074.3; UniProtKB—P29965 (CD40L_HUMAN)); Human CD27 Ligand (NCBI Reference Sequence: NM_001252.5; UniProtKB—P32970 (CD70_HUMAN)); Human TNF beta (NCBI Reference Sequence: NM_000595.4; UniProtKB—P01374 (TNFB_HUMAN)); Human TNF alpha (NCBI Reference Sequence: NM_000594.4; UniProtKB—P01375 (TNFA_HUMAN)); TNFSF10 (NCBI Reference Sequence: NM_003810.4; UniProtKB—P50591 (TNF10_HUMAN)); Transforming Growth Factor α (TGFα)—Membrane Bound (NCBI Reference Sequence: NM_003236.4; UniProtKB—P01135 (TGFA_HUMAN)); Transforming Growth Factor α (TGFβ) Soluble mature form (NCBI Reference Sequence: NM_003236.4; UniProtKB—P01135 (TGFA_HUMAN)); Transforming Growth Factor β (TGFβ) family (NCBI Reference Sequence: NM_000660.7; UniProtKB—P01137 (TGFB1_HUMAN)); CSF1—macrophage colony-stimulating factor (NCBI Reference Sequence: NM_000757.6; UniProtKB—P09603 (CSF1_HUMAN)); CFS2 or GMCSF 144 (NCBI Reference Sequence: NM_000758.4; UniProtKB—P04141 (CSF2_HUMAN)Protein); CSF3—Granulocyte colony-stimulating factors (also called G-CSF and filgrastim) (NCBI Reference Sequence: NM_000759.4; UniProtKB—P09919 (CSF3_HUMAN)); Prothymosin alpha (proTα) 1-111 (NCBI Reference Sequence: NM_002823.5; UniProtKB—P06454 (PTMA_HUMAN)); Prothymosin alpha (proTα) 1-11n—with secretion signal (NCBI Reference Sequence: NM_002823.5; UniProtKB—P06454 (PTMA_HUMAN)); Prothymosin alpha (proTα) 100-109—with secretion signal (NCBI Reference Sequence: NM_002823.5; UniProtKB—P06454 (PTMA_HUMAN)); Thymosin alpha1 (Talpha1) (NCBI Reference Sequence: NM_001099285.2; UniProtKB—P06454 (PTMA_HUMAN)); Transmembrane Flt3L (sFlt3L). (NCBI Reference Sequence: NM_001459.4; UniProtKB—P49771 (FLT3L_HUMAN)); Soluble sFlt3L. (NCBI Reference Sequence: NM_001459.4; UniProtKB—P49771 (FLT3L_HUMAN)).

In Vitro Transcription of saRNA

Plasmid DNA (pDNA) was transformed into Escherichia coli (E. coli) (New England BioLabs, UK) and cultured in 100 mL of Luria Broth (LB) with 100 μg/mL of carbenicillin (Sigma Aldrich, UK). pDNA was isolated using a Plasmid Plus MaxiPrep kit (QIAGEN, UK) and the final concentration measured on a NanoDrop One (ThermoFisher, UK). saRNA was transcribed from the pDNA template using CleanCap Reagent AG (Tebu-bio, France) to produce an RNA transcript with a naturally occurring Cap 1 structure. Briefly, the pDNA template was linearized for 3 h at 37° C., then 1 μg of the linearized pDNA template used in the standard CleanCap Transcription protocol (Tebu-bio, France) according to the manufacturer's protocol. Transcripts were purified by LiCl precipitation at −20° C. for at least 30 min, centrifuged at 20,000 g for 20 min at 4° C. to pellet the RNA, rinsed once with 70% EtOH, centrifuged again at 20,000 g for 5 min at 4° C. and resuspended in UltraPure H₂O (Ambion, UK) and stored at −80° C. until further use.

### In Vitro Transcription of RNA

pDNA was transformed into E. coli (New England BioLabs, UK), cultured in 100 mL of Luria Broth (LB) with 100 μg/mL of carbenicillin (Sigma Aldrich, UK). Plasmid was purified using a Plasmid Plus MaxiPrep kit (QIAGEN, UK) and the concentration and purity measured on a NanoDrop One (ThermoFisher, UK). RNA was transcribed from the plasmid DNA template using the MEGAscript™ T7 Transcription protocol (ThermoFisher, UK) followed by a ScriptCap™ m7G Capping System post translation (Cambio, UK). Briefly, pDNA was linearized for 3 h at 37° C., and 1 μg of the linearized pDNA template used in the standard reaction protocol. After the MEGAscript™ T7 Transcription the transcripts were purified by LiCl precipitation at −20° C. for at least 30 min, then centrifuged at 20,000 g for 20 min at 4° C. to pellet the RNA, rinsed once with 70% EtOH, centrifuged again at 20,000 g for 5 min at 4° C. and resuspended in UltraPure H₂O (Ambion, UK). The transcripts were then post-transcriptionally capped using the ScriptCap™ m7G Capping System standard protocol and finally LiCl precipitated as described above. Purified and Cap 1 capped RNA was then resuspended in UltraPure H₂O (Ambion, UK) and stored at −80° C. until further use.

### Measurement of COVID Expression In Vitro

saRNA replicon constructs containing a GOI and/or an IMPs/IIPs and/or ISPs were tested in HeLa cells where the expression of the COVID Spike protein was used as a model GOI expressed from the sub-genomic 26S promoter.

Cells were cultured in high glucose Dulbecco's Modified Eagle's Medium (cDMEM) (Sigma Aldrich, Merck, UK) containing 10% (v/v) fetal bovine serum (FBS), 5 mg/mL L glutamine (Gibco, ThermoFisher, UK) and 5 mg/mL penicillin/streptomycin (Sigma Aldrich, Merck, UK). HeLa cells were plated at a density of 10000 cells per well into flat clear bottom 96-well plates (Corning Costar) and incubated for 24 hr. 10 uL of OptiMEM (ThermoFisher, UK) containing 0.15 μL lipofectamine MessengerMAX (ThermoFisher, UK) and 100 ng of saRNA constructs or saRNA control (GFP) was added to triplicate wells and after a further 24 hr, cells were harvested and each cell well pellet lysed with 200 μL/well of ice-cold supplemented Pierce™ IP Lysis and incubate the tubes on ice for 5 min. The lysate was then transferred to 1.5 mL Eppendorf tubes and centrifuged to pellet the cell debris at ˜13,000 g for 10 min at 4° C. Supernatant was then transferred to 0.5 mL tubes for further analysis. COVID expression was determined using a Jess Western Blot (BioTechne) instrument, quantitation achieved using protein standards. Anti-COVID primary and secondary chemiluminescent antibodies were first titrated before use.

### In Vivo Expression of Chemokines/Cytokines

Murine chemokines/cytokines were measured using a Luminex ProCartaPlex method from Invitrogen ThermoFisher exactly as described in the product protocol. Mouse sera were prepared form a tail bleed of individual mice and the sera were assessed individually with a group size of n=5, essentially as described by McKay et al., 2020 (2).

### In Vivo Immunogenicity Assessment

Immunogenicity of saRNA and RNA constructs was assessed as described by McKay et al., 2020 (2).

(2) McKay P F, Hu K, Blakeney A K, Samnuan K, Brown J C, Penn R, Zhou J, Bouton C R, Rogers P, Polra K, Lin P J C, Barbosa C, Tam Y K, Barclay W S, Shattock R J (2020) Self amplifying RNA SARS-CoV-2 lipid nanoparticle vaccine candidate induces high neutralising antibody titers in mice. Nat Commun, 11, 3523.

### Example 1—Structural Design of Human Immune Stimulatory Protein (ISP) Constructs

Human immune stimulatory proteins (ISPs) can be incorporated into an RNA construct of the invention, which can be a self-amplifying RNA (saRNA) or a messenger RNA (mRNA) in order to enhance, stimulate or modify the immune responses to the RNA or saRNA encoded gene of interest (GOI), i.e. the protein encoded by a Gene of Interest (GOI), which can be any therapeutic biomolecule, such as an antigen.

Various embodiments of design configurations for the RNA construct of the invention are shown in FIG. 1. SaRNA expression constructs are based on an alphavirus backbone where the non-structural proteins are maintained, but the gene of interest (GOI) is inserted downstream of a subgenomic promotor (SGP) replacing the structural genes of the virus (see Embodiment “1” in FIG. 1). The GOI can be any protein at all, and may include viral, bacterial, fungal or mammalian protein, i.e. a biotherapeutic protein. However, the inventors envisage that the RNA construct of the invention will demonstrate significant utility in the vaccine space, and so the GOI would encode a vaccine antigen, such as a viral, bacterial or fungal protein, such as a coat protein.

saRNA Constructs (Left Hand of FIG. 1)

Any ISP and GOI can be encoded within the saRNA using the following design approaches:
Embodiment “2a” in FIG. 1 shows a saRNA construct encoding a fusion protein including a peptide cleavage motif (e.g. furin-T2a), such that the protein encoded by the GOI (e.g. the antigen of interest) and the ISP are cleaved into separate proteins on translation in the host cell;
In Embodiment “2b” in FIG. 1, the order of the GOI and ISP have been reversed, such that the ISP is 5′ of the GOI, again with a peptide cleavage motif between the ISP and the GOI so that two separate proteins are produced in the host cell following translation of the saRNA construct;
In Embodiment “3a”, the ISP has been inserted downstream of the GOI stop codon. The subgenomic promoter drives translation of the GOI, and expression/translation of the ISP is driven by the inclusion of an internal ribosomal entry site (IRES);
In Embodiment “3b”, the order of the GOI and ISP has been reversed such that translation of the ISP is promoted by the subgenomic promotor and of the GOI by the IRES;
In Embodiment “4a”, the ISP has been inserted downstream of the GOI stop codon. Translation of the GOI is promoted by the first subgenomic promoter and translation of the IIS is driven by the inclusion of a second subgenomic promotor. This is known as the double genomic approach;
In Embodiment “4b”, the position of the ISP and GOI have been swapped around, i.e. with the ISP placed before the GOI. mRNA constructs (right hand of FIG. 1) Referring to FIG. 1, any ISP and GOI can also be encoded within mRNA (see Embodiment “5”) using the following design approaches:
In embodiment “6a”, the mRNA construct encodes a fusion protein including a peptide cleavage motif (e.g. F-T2a) such that the GOI and ISP are cleaved into separate proteins on translation;
In Embodiment “6b”, the order of the GOI and ISP have been reversed such that the ISP is 5′ of the GOI;
In Embodiment “7a”, the ISP has been inserted downstream of the GOI stop codon where translation is driven by the inclusion of an internal ribosomal entry site (IRES);
In Embodiment “7b”, the order of the GOI and ISP has been reversed such that ISP translation is promoted by the 5′ UTR promotor and the GOI by the IRES.

The inventors have tested a large number of human ISPs as described herein in the various embodiments of RNA constructs illustrated in FIG. 1, and believe that they each have potential to modify expression and response to saRNA, mRNA and/or a trans-replicon system.

### Example 2—Structural Design of Human Immune Stimulatory Protein (ISP) Constructs Incorporating a Viral Innate Inhibitor Protein (IIP) and/or a Human Immune Modulatory (IMP)

The inventors have created additional embodiments of the RNA construct of the invention which, in addition to the GOI and ISP, also harbour either a viral innate inhibitory protein (viral IIP) and/or a human innate modulatory protein (IMP). The label/component “IIP” or “IMP” can be used interchangeably in the diagram and in practice. These IIPs and IMPs reduce or ablate the innate recognition and response that may modify or reduce protein expression and translation of the GOI.

saRNA Constructs (Left Hand of FIG. 2)

Any ISP, IMP and GOI can be encoded within the saRNA using the following design approaches:
Embodiment “8a” in FIG. 2 shows a saRNA construct encoding the GOI, IMP and ISP, in which the GOI and IMP are separated by a peptide cleavage motif (e.g. furin-T2a), and IMP and ISP are separated by an IRES, resulting in expression of the GOI (e.g. the antigen of interest) and IMP as a fusion protein which is then cleaved at the cleavage motif into separate proteins on translation in the host cell, and the ISP being separately expressed under the control of the IRES;
In Embodiment “8b” in FIG. 2, the order of the GOI, IMP and ISP have been switched around, such that the IMP is now 5′ of the GOI, which is itself 5′ of the ISP. The IMP is downstream of a subgenomic promoter which drives its expression to produce a protein, and an IRES powers the expression of the GOI and the downstream ISP, as a fusion protein with the GOI. A peptide cleavage motif (e.g. F-T2a) separates the ISP and the GOI such that the single fusion protein of GOI-ISP is cleaved into two separate proteins in the host cell during translation of the saRNA construct;
In Embodiment “9a”, a first subgenomic promoter drives expression of the GOI, and a second 3′ disposed subgenomic promoter drives expression of the IMP. An IRES is disposed 3′ of the IMP and drives expression of the 3′ ISP;
In Embodiment “9b”, the positions of the IMP, ISP and GOI have been swapped around. In this embodiment, a first subgenomic promoter drives expression of the IMP, and a second 3′ disposed subgenomic promoter drives expression of the GOI-ISP fusion protein, which is separated by a cleavage linker (e.g. F-T2a), such that the GOI-ISP is cleaved into two separate proteins in the host cell during translation of the saRNA construct; and
In Embodiment “9c”, three subgenomic promoters are provided, each one of which drives expression of the GOI, IMP and ISP, though clearly these may be in any order.
Embodiment “9d” in FIG. 2 shows a saRNA construct encoding the GOI, IMP and ISP, in which the GOI and IMP are separated by an IRES and the IMP and ISP are separated by an IRES, resulting in expression of the GOI (e.g. the antigen of interest) under the control of the sub-genomic promoter and the IMP and the ISP being separately expressed under the control of each respective IRES.
Embodiment “9e” in FIG. 2 the positions of the IMP, ISP and GOI have been swapped around. This diagram shows a saRNA construct encoding the GOI, IMP and ISP, in which the GOI and ISP are separated by an IRES and the ISP and IMP are separated by an IRES, resulting in expression of the GOI (e.g. the antigen of interest) under the control of the sub-genomic promoter and the ISP and the IMP being separately expressed under the control of each respective IRES. It will be appreciated that further combinations with the GOI or IMP or ISP in each of the three positions are possible.
   mRNA Constructs (Right Hand of FIG. 2)

Referring to FIG. 2, any ISP, IIP and GOI can also be encoded within mRNA (see Embodiment “10”) using the following design approaches:
In embodiment “10a”, the mRNA construct encodes a 5′ GOI linked to a 3′ IMP by a peptide cleavage motif (e.g. F-T2a), such that the GOI and IMP are cleaved into separate proteins on translation. The mRNA construct includes a 3′ IRES followed by an ISP which drives expression of the separate protein; and
In Embodiment “10b”, the order of the GOI, IMP and ISP are the same, but in this embodiment, the GOI and IMP are separated by an IRES and the IMP and ISP are separate by the cleavage site, F-T2a. The order of the GOI, IMP and ISP can be varied in any construct.

### Example 3—Structural Design of Novel saRNA Constructs Comprising ISP/IIP Combinations

Referring to FIG. 2, the inventors have created two further embodiments of an saRNA construct referred to as Embodiment “9d” and “9e”.

In Embodiment “9d”, the saRNA construct encodes a GOI (e.g. the antigen of interest), and an ISP, and also an IMP, in which the GOI and ISP are separated by an IRES element, and the ISP and the IMP are also separated by an IRES element. The IMP is 5′ (upstream) of the ISP which is 3′ (downstream). Expression of the GOI (e.g. the antigen of interest) is under the control of the sub-genomic promoter, and the IMP and the ISP are separately expressed under the control of each respective IRES. This results in expression of the GOI, ISP and IMP as a fusion protein which is then cleaved at the IRES linker elements into separate proteins on translation in the host cell.

In Embodiment “9e”, the positions of the IMP, ISP and GOI have been swapped around in the saRNA construct. The ISP is 5′ (upstream) of the IMP which is 3′ (downstream). The GOI and ISP are separated by an IRES and the ISP and IMP are separated by an IRES, resulting in expression of the GOI (e.g. the antigen of interest) under the control of the sub-genomic promoter and the ISP and the IMP being separately expressed under the control of each respective IRES.

### Example 4—In Vitro Enhancement of GOI Expression

saRNA replicon constructs were transfected into Hela cells using lipofectamine and the expression of the Gene of Interest (GoI), in this specific case the COVID spike antigen was used as the model expression antigen. As shown in FIG. 7, some embodiments of the saRNA constructs encoded a GOI−ISP (CCL2, CCL3, IL-7 or GM-CSF), whereas other embodiments encoded a GOI−ISP (CCL2, CCL3, IL-7 or GM-CSF)-IIP (in which the IIP is IRF1, E3L or PR34). The in vitro expression is presented as a fold enhancement over the control saRNA replicon (saRNA COVID-GFP).

Referring to FIG. 7A, there is shown GOI+ISP (CCL2, CCL3, IL-7 or GM-CSF)+IRF1 (acting as the IIP).

Referring to FIG. 7B, there is shown GOI+ISP (CCL2, CCL3, IL-7 or GM-CSF)+E3L (acting as the IIP).

Referring to FIG. 7C, there is shown GOI+ISP (CCL2, CCL3, IL-7 or GM-CSF)+PR34 (acting as the IIP).

As can be seen in these Figures, there is an enhancement of the COVID expression in the presence of the ISP alone (i.e. CCL2, CCL3, IL-7 or GM-CSF), and there is a marked enhancement by the ISP (CCL2, CCL3, IL-7 or GM-CSF) and the IIP (IRF1, E3L or PR34) in combination.

### Example 5—In Vivo Enhancement of Inflammatory/Stimulatory Chemokines/Cytokines

Mice were vaccinated with various LNP formulated saRNA replicons expressing GoI+IIPs alone, GoI+ISPs alone or combinations of GoI+ISPs+IIPs and the murine sera harvested 24 hours post injection. The expressed antigen (COVID spike protein used as the model antigen) and/or the COVID+ISP enhanced the circulating expression levels of various cytokines/chemokines as measured by Luminex.

Referring to FIGS. 8A and 8B, there are shown in vivo enhancement of inflammatory/stimulatory chemokines/cytokines, i.e. IFN-gamma, IL-18, GM-CSF, IL-5, and MCP-1. Groups of mice were vaccinated with various lipid nanoparticles (LNP)-formulated saRNA replicons expressing GoI+IIPs alone (IRF1, E3L or PR34), GoI+ISPs (i.e. CCL2, CCL3, IL-7 or GM-CSF) alone, or combinations of GoI+ISPs (i.e. CCL2, CCL3, IL-7 or GM-CSF)+IIPs (IRF1, E3L or PR34) and the murine sera harvested 24 hours post injection.

As can be seen, the expressed antigen (COVID spike protein used as the model antigen) and/or the COVID+ISP surprisingly enhanced the circulating expression levels of various cytokines/chemokines.

### Example 6—In Vivo Enhancement of GoI Specific Antibody Immune Responses

Mice were vaccinated with various LNP formulated saRNA replicons expressing GoI+IIPs alone, GoI+ISPs alone or combinations of GoI+ISPs+IIPs and the murine sera was tested for antigen-specific antibodies at day 42 post immunisation. These animals received a prime vaccination at day 0 and a boost vaccination at day 28 with 1 μg of LNP formulated saRNA replicon.

Referring to FIG. 9, there is shown in vivo enhancement of GoI specific antibody immune responses. Groups of mice were vaccinated with various LNP-formulated saRNA replicons expressing GoI+IIPs alone, GoI+ISPs alone or combinations of GoI+ISPs+IIPs and the murine sera was tested for antigen-specific antibodies at day 42 post immunisation. These animals received a prime vaccination at day 0 and a boost vaccination at day 28 with 1 μg of LNP formulated saRNA replicon. The saRNA replicons containing ISPs+IIPs were compared to the immune responses in those animals that had received only the ISP clearly demonstrating the enhancement to antibody immunity provided by the activity of the IIPs on the expression of the both the GoI and/or the functionality of the ISPs. Graphs are presented as fold-change of antibody levels when compared to the corresponding ISP alone.

### CONCLUSIONS

The inventors believe that the constructs described herein display many advantages over those described in the prior art, including:
i) the RNA constructs of the invention, as shown in FIGS. 1 and 2, combine an saRNA or mRNA, the GOI (e.g. an antigen) and the ISP, which enhances or modifies the immune response of the GOI in the subject. The ISP may be a cytokine.
ii) insertion of nucleotide sequences encoding any of the innate modulatory proteins directly into the RNA construct, such as mRNA or saRNA, enabling simultaneous protein expression of the ISP, the IMP/IIP protein and the biotherapeutic molecule encoded by the gene of interest;
iii) as opposed to delivering two different and separate strands of RNA, one encoding the gene of interest (GOI), i.e. the therapeutic biomolecule, and one encoding the ISP, a single strand is delivered, thus ensuring colocalization of the RNA, the ISP and the IMP/IIP;
iv) the IMP/IIP inhibits innate sensing of RNA, thus enabling higher protein expression;
v) when the RNA construct is a saRNA, the IMP/IIP expression itself is self-amplified by virtue of being co-expressed on the sub-genome strand with the GOI;
vi) an increase in both the magnitude and duration of protein expression compared to conventional VEEV RNA replicon constructs;
vii) there is an enhancement of the GOI in the presence of the ISP alone, and there is a marked enhancement by the ISP and the IIP in combination;
(viii) The expressed GOI+ISP enhances the circulating expression levels of various cytokines/chemokines;
(ix) saRNA replicons containing ISPs+IIPs were compared to the immune responses in animals that had received only the ISP clearly demonstrating the enhancement to antibody immunity provided by the activity of the IIPs on the expression of the both the GoI and/or the functionality of the ISPs.

1. A recombinant RNA construct encoding:
(a) (i) at least one therapeutic biomolecule;
(ii) at least one immune stimulatory protein (ISP), and
optionally (iii) at least one human innate modulatory protein (IMP) and/or at least one viral immune inhibitor protein (IIP); or
(b) (i) at least one immune stimulatory protein (ISP); and/or
(ii) at least one human innate modulatory protein (IMP) and/or at least one viral immune inhibitor protein (IIP).

2. The RNA construct according to claim 1, wherein the construct comprises mRNA or saRNA, and preferably saRNA.

3. The RNA construct according to claim 1, wherein the RNA construct comprises or is derived from a positive stranded RNA virus selected from the group of genus consisting of: alphavirus; picornavirus; flavivirus; rubivirus; pestivirus; hepacivirus; calicivirus and coronavirus, preferably an alphavirus, optionally VEEV.

4. The RNA construct according to claim 1, wherein the immune stimulatory protein (ISP) is a mammalian ISP, preferably a human ISP.

5. The RNA construct according claim 1, wherein the ISP is a cytokine or a chemokine.

6. The RNA construct according to claim 1, wherein the ISP is an alpha chemokine selected from: CXCL 1; CXCL 8; CXCL 11; and CXCL12, or an orthologue thereof.

7. The RNA construct according to claim 1, wherein the ISP is a beta chemokine selected from: CCL1; CCL2; CCL3; CCL20; and CCL21, or an orthologue thereof.

8. The RNA construct according to claim 1, wherein the ISP is a delta chemokine, optionally CX3CL1, or an orthologue thereof.

9. The RNA construct according to claim 1, wherein the ISP is a gamma chemokine, optionally XCL1 or XCL2, or an orthologue thereof.

10. The RNA construct according to claim 1, wherein the ISP is an interleukin, optionally wherein the interleukin is IL-7 or IL1-38, or an orthologue thereof.

11. The RNA construct according to claim 10, where in the interleukin is selected from: IL-1b; IL-2; IL-18(37-93); IL-19; IL-20; IL-21; IL-22; IL-33 and IL-36 alpha, or an orthologue thereof.

12. The RNA construct according to claim 1, wherein the ISP is selected from: granulocyte-macrophage colony stimulating factor (GM-CSF); Tumour Necrosis Factor (TNF) alpha; Tumour Necrosis Factor, Membrane Form; Tumour Necrosis Factor, Soluble Form; TNF beta; BAFF; CD30 ligand; CD40 ligand; CD27 ligand; and TNFFS10, or an orthologue thereof.

13. The RNA construct according to claim 1, wherein the ISP is selected from: Transforming Growth Factor α (TGFα); Transforming Growth Factor β (TGFβ); colony-stimulating factor (CSF) 1; CSF2; and CSF3, or an orthologue thereof.

14. The RNA construct according to claim 1, wherein the ISP is selected from: Prothymosin alpha; Thymosin alpha1; Transmembrane Flt3L; and Soluble sFlt3L.

15. The RNA construct according to claim 1, wherein the saRNA comprises a nucleotide sequence encoding an ISP, and a nucleotide sequence encoding an IIP.

16. The RNA construct according to claim 15, wherein the ISP is selected from a group consisting of: CCL2, CCL3, IL-7 and GM-CSF.

17. The RNA construct according to claim 15 or 16, wherein the IIP is selected from a group consisting of: IRF-1, E3L and PR34.

18. The RNA construct according to any one of claims 15-17, wherein the nucleotide sequence encoding the ISP is disposed 5′ of the nucleotide sequence encoding the IIP.

19. The RNA construct according to claim 1, wherein the therapeutic biomolecule comprises a therapeutic protein, preferably the protein or peptide is an antigen, and more preferably a viral antigen.

20. A nucleic acid sequence encoding the RNA construct according to claim 1.

21-29. (canceled)
